(19) United States
(12) Patent Application Publication
(10) Pub. No.: US 2025/0215104 A1
(43) Pub. Date: Jul. 3, 2025

(54) NOVEL SOLUBLE UROKINASE PLASMINOGEN ACTIVATOR RECEPTOR (SUPAR) BINDING MOLECULES AND USES THEREOF

(71) Applicant: Walden Biosciences, Inc., Cambridge, MA (US)

(72) Inventors: Jean-Philippe Bürckert, Belmont, CA (US); Kathrin Zuberbühler, Pacifica, CA (US); Alex Duncan, Boston, MA (US)

(73) Assignee: Walden Biosciences, Inc., Cambridge, MA (US)

(21) Appl. No.: 18/843,017

(22) PCT Filed: Mar. 2, 2023
(86) PCT No.: PCT/US2023/063619

§ 371 Date: Aug. 30, 2024

Related U.S. Application Data
(60) Provisional application No. 63/315,693, filed on Mar. 2, 2022.

(51) Int. Cl. C07K 16/28; A61K 39/00; A61P 13/12

(52) U.S. Cl. CPC C07K 16/2896; A61P 13/12; A61K 2039/505; C07K 2317/24; C07K 2317/92

(57) ABSTRACT

Disclosed are novel urokinase plasminogen activator receptor (uPAR) binding molecules and soluble urokinase plasminogen activator receptor (suPAR) binding molecules and methods of their use.

### I. CROSS-REFERENCE TO RELATED APPLICATIONS

This application claims the benefit of U.S. Provisional Application No. 63/315,693, filed on Mar. 2, 2022, which is incorporated herein by reference in its entirety.

### II. BACKGROUND

In mouse experiments it was shown that mice challenged with LPS had an increase in suPAR and developed proteinuria. Additionally, transgenic expression of murine suPAR 1 or 2 lead to elevated ACR with significant changes in serum markers of kidney function. Blood soluble urokinase plasminogen activator receptor (suPAR) levels are strongly predictive of incident kidney disease in different patient populations. Moreover, proteinuria severity seems to depend on the suPAR isoform, duration of exposure and the presence of additional risk factors. The higher the suPAR level, the more severe the disease. What are needed are suPAR antagonists that can decrease blood suPAR levels.

### III. SUMMARY

Disclosed are urokinase plasminogen activator receptor (uPAR) binding molecules and methods of their use.

In one aspect, disclosed herein are urokinase plasminogen activator receptor (uPAR) binding molecules; including, but not limited to soluble urokinase plasminogen activator receptor (suPAR) binding molecules (such as, for example a chimeric antigen receptor (CAR) T cell, CAR NK cell, CAR Macrophage (CARMA), immunotoxin, bispecific antibody, diabody, triabody, Bispecific T cell engager (BiTE), antibody, or antibody fragment) comprising a light chain variable domain, wherein the light chain variable domain comprises 3 complementarity determining regions (CDRs), CDR1, CDR2, and CDR3 as set forth in SEQ ID NOs 71-75 and 1901-2500; SEQ ID NOs: 76, 77, and 2501-3100; and SEQ ID NO: 78 and 3100-3700, respectively. In one aspect, the light chin can comprise a CDR1, CDR2, and CD3, as set forth in SEQ ID Nos 71, 76, and 78, respectively; SEQ ID Nos 72, 76, and 78, respectively; SEQ ID Nos 72, 77, and 78, respectively; SEQ ID Nos 73, 76, and 78, respectively; SEQ ID Nos 74, 76, and 78, respectively; or SEQ ID Nos 75, 76, and 78, respectively. For example, the urokinase plasminogen activator receptor (uPAR) binding molecule can comprise a light chain variable domain (V_{L}) comprising the amino acid sequence as set forth in SEQ ID NOs: 2, 25-44, 4300-4900, and 4904.

Also disclosed herein are urokinase plasminogen activator receptor (uPAR) binding molecules of any preceding aspect (including, but not limited to a soluble urokinase plasminogen activator receptor (suPAR) binding molecule), wherein the uPAR binding molecule further comprises a heavy chain variable domain; wherein the heavy chain variable domain comprises 3 complementarity determining regions (CDRs), CDR1, CDR2, and CDR3 as set forth in SEQ ID NOs 45-57 and 101-700; SEQ ID NOs: 58-68 and 701-1300; and SEQ ID NOs: 69, 70, and 1301-1900, respectively. In one aspect, the heavy chain can comprise a CDR1, CDR2, and CD3, as set forth in SEQ ID Nos: 45, 58, and 69, respectively; SEQ ID Nos: 45, 59, and 69, respectively; SEQ ID Nos: 46, 60, and 69, respectively; SEQ ID Nos: 46, 61, and 69, respectively; SEQ ID Nos: 47, 62, and 69, respectively; SEQ ID Nos: 48, 62, and 70, respectively; SEQ ID Nos: 49, 62, and 69, respectively; SEQ ID Nos: 50, 62, and 69, respectively, SEQ ID Nos. 51, 62, and 69, respectively; SEQ ID Nos: 52, 62, and 69, respectively; SEQ ID Nos: 53, 63, and 69, respectively; SEQ ID Nos: 53, 64, and 69, respectively; SEQ ID Nos: 54, 65, and 69, respectively; SEQ ID Nos: 54, 66, and 69, respectively; SEQ ID Nos: 55, 62, and 69, respectively; SEQ ID Nos: 53, 62, and 69, respectively; SEQ ID Nos: 56, 67, and 69, respectively; SEQ ID Nos: 57, 68, and 69, respectively; or SEQ ID Nos: 53, 64, and 69, respectively. For example, the urokinase plasminogen activator receptor (uPAR) binding molecule can comprise a heavy chain variable domain (V_{H}) comprising the amino acid sequence as set forth in SEQ ID NOs: 1, 5-24, 3701- and 4903. Thus, in one aspect, the urokinase plasminogen activator receptor (uPAR) binding molecule can comprise a heavy chain CDR1, CDR2 and CDR 3 as set forth in SEQ ID NOs: 48, 62, and 70, respectively; and a light chain CDR1, CDR2, and CDR3 as set forth in SEQ ID Nos 72, 76, and 78, respectively; a heavy chain CDR1, CDR2 and CDR 3 as set forth in SEQ ID NOs: 53, 64, and 69, respectively; and a light chain CDR1, CDR2, and CDR3 as set forth in SEQ ID Nos 71, 76, and 78, respectively. For example, the urokinase plasminogen activator receptor (uPAR) binding molecule can comprise a heavy chain as set forth in SEQ ID NO: 4903 and a light chain as set forth in SEQ ID NO: 4904; a heavy chain as set forth in SEQ ID NO: 1 and a light chain as set forth in SEQ ID NO: 2; a heavy chain as set forth in SEQ ID NO: 10 and a light chain as set forth in SEQ ID NO: 30; a heavy chain as set forth in SEQ ID NO. 12 and a light chain as set forth in SEQ ID NO: 32; a heavy chain as set forth in SEQ ID NO: 24 and a light chain as set forth in SEQ ID NO: 44; a heavy chain as set forth in SEQ ID NO: 5 and a light chain as set forth in SEQ ID NO: 25; a heavy chain as set forth in SEQ ID NO: and a light chain as set forth in SEQ ID NO: 26; a heavy chain as set forth in SEQ ID NO: 7 and a light chain as set forth in SEQ ID NO: 27; a heavy chain as set forth in SEQ ID NO: 8 and a light chain as set forth in SEQ ID NO: 28; a heavy chain as set forth in SEQ ID NO: 9 and a light chain as set forth in SEQ ID NO: 29; a heavy chain as set forth in SEQ ID NO: 11 and a light chain as set forth in SEQ ID NO. 31; a heavy chain as set forth in SEQ ID NO: 13 and a light chain as set forth in SEQ ID NO: 33; a heavy chain as set forth in SEQ ID NO: 14 and a light chain as set forth in SEQ ID NO: 34; a heavy chain as set forth in SEQ ID NO: 15 and a light chain as set forth in SEQ ID NO: 35; a heavy chain as set forth in SEQ ID NO: 16 and a light chain as set forth in SEQ ID NO: 36; a heavy chain as set forth in SEQ ID NO: 17 and a light chain as set forth in SEQ ID NO: 37; a heavy chain as set forth in SEQ ID NO: 18 and a light chain as set forth in SEQ ID NO: 38; a heavy chain as set forth in SEQ ID NO: 19 and a light chain as set forth in SEQ ID NO: 39; a heavy chain as set forth in SEQ ID NO: 20 and a light chain as set forth in SEQ ID NO: 40; a heavy chain as set forth in SEQ ID NO: 21 and a light chain as set forth in SEQ ID NO. 41; a heavy chain as set forth in SEQ ID NO: 22 and a light chain as set forth in SEQ ID NO: 42; a heavy chain as set forth in SEQ ID NO: 23 and a light chain as set forth in SEQ ID NO: 43.

In one aspect, disclosed herein are urokinase plasminogen activator receptor (uPAR) binding molecules including, but not limited to soluble urokinase plasminogen activator receptor (suPAR) binding molecules (such as, for example a chimeric antigen receptor (CAR) T cell. CAR NK cell, CAR Macrophage (CARMA), immunotoxin, bispecific antibody, diabody, triabody, Bispecific T cell engager (BiTE), antibody, or antibody fragment) comprising a heavy chain variable domain; wherein the heavy chain variable domain comprises 3 complementarity determining regions (CDRs), CDR1, CDR2, and CDR3 as set forth in SEQ ID NOs 45-57 and 101-700; SEQ ID NOs: 58-68 and 701-1300; and SEQ ID NOs: 69, 70, and 1301-1900, respectively, respectively. In one aspect, the heavy chain variable domain can comprise a CDR1, CDR2, and CD3, as set forth in SEQ ID Nos: 45, 58, and 69, respectively; SEQ ID Nos: 45, 59, and 69, respectively; SEQ ID Nos: 46, 60, and 69, respectively; SEQ ID Nos: 46, 61, and 69, respectively; SEQ ID Nos: 47, 62, and 69, respectively; SEQ ID Nos: 48, 62, and 70, respectively; SEQ ID Nos: 49, 62, and 69, respectively; SEQ ID Nos: 50, 62, and 69, respectively; SEQ ID Nos: 51, 62, and 69, respectively; SEQ ID Nos: 52, 62, and 69, respectively; SEQ ID Nos: 53, 63, and 69, respectively; SEQ ID Nos: 53, 64, and 69, respectively, SEQ ID Nos. 54, 65, and 69, respectively; SEQ ID Nos: 54, 66, and 69, respectively; SEQ ID Nos: 55, 62, and 69, respectively; SEQ ID Nos: 53, 62, and 69, respectively; SEQ ID Nos: 56, 67, and 69, respectively; SEQ ID Nos: 57, 68, and 69, respectively; or SEQ ID Nos: 53, 64, and 69, respectively. For example, the urokinase plasminogen activator receptor (uPAR) binding molecule (including, but not limited to a soluble urokinase plasminogen activator receptor (suPAR) binding molecule) can comprise a heavy chain variable domain (VA) comprising the amino acid sequence as set forth in SEQ ID NOs: 1, 5-24, 3701-4300 and 4903.

Also disclosed herein are urokinase plasminogen activator receptor (uPAR) binding molecules of any preceding aspect (including, but not limited to a soluble urokinase plasminogen activator receptor (suPAR) binding molecule), wherein the binding molecule further comprises a light chain constant domain as set forth in SEQ ID NO: 4 or SEQ ID NO: 4906.

Also disclosed herein are urokinase plasminogen activator receptor (uPAR) binding molecules of any preceding aspect (including, but not limited to a soluble urokinase plasminogen activator receptor (suPAR) binding molecule), wherein the binding molecule further comprises a heavy chain constant domain as set forth in SEQ ID NO: 3 or SEQ ID NO: 4905.

In one aspect, disclosed herein are methods of treating, decreasing, inhibiting, reducing, ameliorating, and/or preventing an inflammatory kidney disease or condition in a subject (such as, for example, proteinuric kidney disease; Focal segmental glomerulosclerosis (FSGS); IgA nephropathy; membranous nephropathy; lupus nephritis; diabetic nephropathy; Autosomal dominant polycystic kidney disease (ADPKD); Alport syndrome, acute kidney injury (AKI) (including, but not limited to COVID-19 AKI), glomerulonephritis, preeclampsia; systemic lupus erythematosus; multiple myeloma; or kidney injury as the result of trauma, contrast agents, infection, surgery, ischemia/reperfusion injury, transplant, or medication) or the symptoms thereof comprising administering to the subject the urokinase plasminogen activator receptor (uPAR) binding molecule of any preceding aspect (such as, for example, any soluble urokinase plasminogen activator receptor (suPAR) binding molecule of any preceding aspect).

Also disclosed herein are methods of treating, decreasing, inhibiting, reducing, ameliorating and/or preventing an inflammatory kidney disease or condition in a subject or the symptoms thereof any preceding aspect, wherein the soluble urokinase plasminogen activator receptor (suPAR) binding molecule is administered when suPAR levels are elevated relative to a normal control. In some aspects, the soluble urokinase plasminogen activator receptor (suPAR) binding molecule is administered prior to the onset of symptoms.

In one aspect, disclosed herein are methods of treating, decreasing, inhibiting, reducing, ameliorating and/or preventing an inflammatory kidney disease or condition in a subject or the symptoms thereof any preceding aspect, further comprising obtaining a biological sample (such as, for example, whole blood, plasma, serum, or urine) from the subject and measuring suPAR levels in the sample; wherein 1 ng/ml of suPAR indicates a healthy subject; 2-ng/ml of suPAR indicates an acute kidney disease or acute inflammation; 4 ng/ml of suPAR indicates the subject likely has or will develop chronic kidney disease; and 5 ng/ml or greater of suPAR indicates that the subject has chronic kidney disease.

### IV. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1 shows that an anti-suPAR antibody is efficacious in nephrotoxic model of kidney injury.

FIG. 2 shows normalized ACR and body weight overtime between nonspecific antibody and anti-suPAR antibody WAb008.

FIG. 3 shows the mean normalized ACR between nonspecific antibody and anti-suPAR antibody WAb008.

FIG. 4 shows WAb0014 binds to hsuPAR Isoform 1-D2D3 Fragment with Nanomolar Affinity. Concentration response binding curves for hsuPAR isoform 1-D2D3 fragment (2.5, 7.41, 22.2, 67 nM; colored traces, bottom to top) binding to immobilized WAb0014.

FIG. 5 shows WAb0014 binds to hsuPAR Isoform 3 with Nanomolar Affinity. Concentration response binding curves for hsuPAR isoform 3 (2.5, 7.41, 22.2, 66.7, 200 nM; colored traces, bottom to top) binding to immobilized WAb0014.

FIG. 6 shows WAb0014 Binds to Cynomolgus suPAR with Nanomolar Affinity. Concentration response binding curves for cynomolgus suPAR (2.5, 7.4, 22.2, 67, 200 nM; colored traces, bottom to top) binding to immobilized WAb0014.

FIG. 7 shows WAb0014 Binds Picomolar Binding Affinity at Acidic pH to FcRn Receptors. Concentration response binding curves for WAb0014 (24.7, 74.1, 222.2, 666.7, nM; colored traces, bottom to top) binding to immobilized FcRn receptors at pH 6.

FIG. 8 shows WAb0014 Exhibits a Comparatively Weaker, High Nanomolar Binding Affinity at Physiological pH to FcRn Receptors. Concentration response binding curves for WAb0014 (24.7, 74.1, 222.2, 666.7, 2000 nM; colored traces, bottom to top) binding to immobilized FcRn receptors at pH 7.2.

FIG. 9 shows WAb0014 Exhibits Avidity Driven Picomolar Binding Affinity to hsuPAR. Concentration response binding curves for WAb0014 (0.3, 1, 3.3, 10 μg/mL; colored traces, bottom to top) binding to immobilized hsuPAR.

FIG. 10 shows WAb0014 Exhibits Low Nanomolar Binding Affinity to hsuPAR. Concentration response binding curves for hsuPAR (1.23, 3.7, 11.1, 33.3, 100 nM; colored traces, bottom to top) binding to immobilized WAb0014.

FIG. 11 shows WAb0014 Binds to Cell Surface huPAR with Nanomolar Affinity in a Flow Cytometry Assay.

FIG. 12 shows uPAR Gene Expression.

FIG. 13 shows Mean Fluorescence Intensity.

FIG. 14 shows Hybridoma Approach to Generating Anti-human (anti-hsuPAR) Antibodies.

FIG. 15 shows an absence of an Effect of WAb0014 Treatment on HK2 and MDA-MB231 Cell Migration/Motility.

FIG. 16 shows an absence of an Effect of WAb0014 Treatment on HK2 Cell Proliferation/Viability.

FIG. 17 shows an absence of an Effect of WAb0014 Treatment on MDA-MB231 Cell Proliferation/Viability.

FIG. 18 shows the effect of Anti-suPAR Antibody, PP13, Treatment on PMA-induced Increase in uPAR on U-937 Cell Surface.

FIG. 19 shows uPA Strongly Binds to hsuPAR with Nanomolar Binding Affinity Concentration response binding curves for uPA (11.1, 33.3, 100, 300 nM; colored traces, bottom to top) binding to immobilized hsuPAR. Inset-full, unprocessed, raw binding traces.

FIG. 20 shows uPA Binds with Nanomolar Affinity to hsuPAR in Presence of WAb0014. Concentration response binding curves for uPA (11.1, 33, 100, 300 nM; colored traces, bottom to top) binding to immobilized hsuPAR in presence of WAb0014. Inset-full, unprocessed, raw binding traces.

FIG. 21 shows Vitronectin Strongly Binds to hsuPAR with Nanomolar Binding Affinity. Concentration response binding curves for vitronectin (2.1, 6.4, 19, 57 nM; colored traces, bottom to top) binding to immobilized hsuPAR. Inset-full, unprocessed, raw binding traces.

FIG. 22 shows Vitronectin Binds with Nanomolar Affinity to hsuPAR in Presence of WAb0014. Concentration response binding curves for Vitronectin (10.5, 105, 316 nM, colored traces, bottom to top) binding to immobilized hsuPAR in presence of WAb0014. Inset-full, unprocessed, raw binding traces.

FIG. 23 shows rescue of hsuPAR Induced Src Kinase Phosphorylation by WAb0014 Treatment.

FIG. 24 shows rescue of hsuPAR Induced NOX2 Protein Expression by WAb0006 Treatment.

FIG. 25 shows WAL0921-ΔK Exhibits Weak Binding to Fc-gammaR1 Receptor. Concentration response binding curves for WAL0921-ΔK (39.5, 118.5, 355.6, 1067, nM; colored traces, bottom to top) binding to immobilized Fc-gammaR1 receptors.

FIG. 26 shows WAL0921-ΔK Does Not Bind High Affinity Fc-gammaR2a Receptor. Concentration response binding curves for WAL0921-ΔK (39.5, 118.5, 355.6, 1067, nM; colored traces, bottom to top) binding to immobilized high affinity Fc-gammaR2a receptors.

FIG. 27 shows WAL0921-ΔK Does Not Bind High Affinity Fc-gammaR3a Receptor. Concentration response binding curves for WAL0921-ΔK (39.5, 118.5, 355.6, 1067, nM; colored traces, bottom to top) binding to immobilized high affinity Fc-gammaR3a receptors.

FIG. 28 shows the anti-suPAR Antibody Lineage.

FIGS. 29A and 29B show WAb0014 Treatment Induces Endocytosis of Cell Surface uPAR.

FIGS. 30A and 30B show WAb0014 Exhibits Target Mediated Endocytosis in Undifferentiated Human Podocytes.

FIG. 31 shows the effects of WAb008 on ACR.

FIG. 32 shows the effects of WAb008 on ACR in Plaur−/− Mice.

FIG. 33 shows the effects of WAb008 on ACR in hsuPAR Tg Mice.

FIG. 34 shows the effects of WAL0921mu on ACR in hsuPAR Tg Mice.

FIG. 35 shows the effects of WAL0921mu on ACR hsuPAR Tg Mice (1 copy Matched Pairs).

FIG. 36 shows antibody exposure graphs.

FIG. 37 shows suPAR binding data to WAL0921 in a bilayer interference assay on an Octet platform comparing WAL0921 antibody manufactured at WuXi Biologics and Evitria SA. In both experiments, we loaded 3 μg/ml of antibody on AHC sensors and titrated suPAR across a range of concentrations (100, 33.3, 11.1, 3.7 and 1.24 nM; blue, red, turquoise, green and orange traces respectively).

### V. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder Such amelioration only requires a reduction or alteration, not necessarily elimination.

The term “treatment,” “treat,” and “treating” refer to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. Treatments can include prophylactic treatments.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily, weekly, monthly, semi-annually, annually, or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g., a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Compositions

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular uPAR binding molecule or suPAR binding molecule is disclosed and discussed and a number of modifications that can be made to a number of molecules including the uPAR binding molecule or suPAR binding molecule are discussed, specifically contemplated is each and every combination and permutation of uPAR binding molecule or suPAR binding molecule and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

uPAR refers to urokinase plasminogen activator receptor, all fragments thereof, and all post-translational glycosylation, genetic mutation, and different isoforms derived from alternative splicing. uPAR, also known as CD87, is encoded by the PLAUR gene and belongs to the lymphocyte antigen-6 superfamily. The protein moiety consists of three Ly6/uPAR/alpha-neurotoxin-like (LU) homologous domains denoted DI (residues 1-92), DII (residues 93-191) and DIII (residues 192-283), as numbered from the N-terminus. It is expressed and either tethered to a cell membrane as a glycosylphosphatidylinositol (GPI)-anchored membrane bound protein or cleaved at the GPI anchor by phospholipases to generate the soluble form of uPAR (suPAR). uPAR involved in many physiological and pathological events. It acts as a receptor for urokinase-type plasminogen activator (uPA), facilitating the generation of activated plasmin, thus playing a role in the directional invasion of migrating cells. uPAR is expressed on a variety of cells, including monocyte, lymphocyte and endothelial cells but baseline expression of uPAR is low in most tissues except bone-marrow cells. uPAR is upregulated in tissues undergoing active remodeling or in cancer cells (as measured by the number of cells expressing cell surface uPAR).

suPAR refers to soluble urokinase plasminogen receptor, all fragments thereof, and all post-translational glycosylation, genetic mutation, and different isoforms derived from alternative splicing. suPAR is derived from the cell membrane tethered receptor uPAR post enzymatic cleavage and initially comprises the identical three ectodomains of uPAR. suPAR is the soluble form of urokinase plasminogen activator receptor. It has been documented that cleavage of the GPI anchor releases full-length suPAR from membrane-bound uPAR. Numerous studies have indicated that full-length suPAR is functional. It retains uPAR's ability to bind to uPA, and suPAR binds vitronectin and integrins as well. As suPAR and uPAR can be cleaved at the linker region between DI and DII by a variety of enzymes, they may generate a DI fragment and a DIIDIII fragment. Both fragments have been detected in body fluids. It can be measured by ELISA or similar tests in the plasma or urine. In healthy individuals, plasma suPAR levels are reported to be <3 ng/ml. suPAR containing DI, DII, and DIII domains can compete with cell surface uPAR receptor for uPA binding and may modulate uPAR's promigratory signaling cascade. suPAR can be found in various other body fluids including urine, saliva, and cerebrospinal fluid (CSF) in different concentrations. Elevated levels of plasma suPAR are closely linked to inflammation, organ damage, and immune activation in a variety of different disease states. Circulating suPAR may, in turn, undergo proteolytic cleavage of the linker between DI and DII domains, thus generating free DI and DIIDIII domains with different biologic properties. In addition to different suPAR fragments, there are other modifications that could impact circulating suPAR composition and function as well, including post-translational glycosylation, genetic mutation, and different isoforms derived from alternative splicing. For example, Wei summarizes four human uPAR isoforms: human isoform 1 (huPAR1) has three intact Ly6/uPAR domains and a GPI anchor; human isoform 2 (huPAR2) has a deletion of exon and lacks a GPI anchor sequence; human isoform 3 (huPAR3) has a deletion of exon 5 and hence lacks the three C-terminal β-strands in DII; human isoform 4 (huPAR4) has an in-frame deletion of exon 6, which contributes the N-terminal sheet assembly to DIII, but retains the 3 C-terminal strands of DIII and the GPI anchor.

Blood suPAR levels are strongly predictive of incident kidney disease in different patient populations. Moreover, proteinuria severity seems to depend on the suPAR isoform, duration of exposure and the presence of additional risk factors. The higher the suPAR level, the more severe the disease. By reducing blood suPAR levels, normal function is restored. Thus, we sought to generate potent ‘antagonist’ anti-human suPAR monoclonal antibody for the treatment of patients with kidney diseases including proteinuric kidney disease; Focal segmental glomerulosclerosis (FSGS); IgA nephropathy; membranous nephropathy; lupus nephritis; diabetic nephropathy; Autosomal dominant polycystic kidney disease (ADPKD); Alport syndrome, acute kidney injury (AKI) (including, but not limited to COVID-19 AKI); glomerulonephritis, preeclampsia; systemic lupus erythematosus; multiple myeloma; or kidney injury as the result of trauma, contrast agents, infection, surgery, ischemia/reperfusion injury, transplant, or medication. The drug compound will bind uPAR or suPAR and thereby removing suPAR and as a result treating, inhibiting, reducing, decreasing, ameliorating, and/or preventing kidney disease.

To create a human anti-uPAR binding molecule (including, but not limited to a suPAR binding molecule), a mouse binding molecule was generated by immunizing mice with human uPAR (such as, for example, suPAR). Hybridomas were generated and clones isolated. Clones with the best functional characteristics were utilized to generate full length chimeric mouse-human IgG antibodies. We then humanized an antibody by grafting the mouse complementarity determining regions (CDRs) on 16 different framework scaffolds and checked for binding to human and cynomolgus suPAR. One of these 16 was selected for suPAR affinity optimization. A single chain Fv library of approximately 0.5×10∧10 amino acid variants was constructed in a phage display library and selected over several rounds. A screening ELISA was performed to identify positive clones and 1200 clones were sequenced. The screening performed resulted in the identification of approximately 700 unique clones (see Table 3) which bound to suPAR; these were ranked by using their dissociation rates as shown in Table 6. More specifically, the screening was performed by comparing kdis (also called k_off). 66 single chain Fv's were reformatted as full length human IgG antibodies.

TABLE 3
Clones with CDRs and full variable heavy chain and variable light chain sequences

| Clone | | | | | | | | |
|---|---|---|---|---|---|---|---|---|
| ID | | | | | | | | |
| DB_ID | CDR-L1 | CDR-H2 | CDR-H3 | CDR-L1 | CDR-L2 | CDR-L3 | fullVH | fullVL |
| suPAR_ | FNIKD | GWIDH | CARG | SARS | RTSNL | CQQYH | EVQLEESGAELVRPGALVKLSCKASGFNIKDYYIH | QIVLTQSPAIMSASPGEKVTIYCSARSSVSYMYWY |
| DB01_ | YYIH | ENGNTI | YDV | SVSY | AS | SYPPTF | WVKQRPEQGLEWIGWIDHENGNTIYDPKFQGKAS | QQKPGSSPKPWIYRTSNLASGVPARFSGSGSGTSYS |
| A02 | | YD | DWF | MY | | | ITLDTSSNTAYLQLNSLTSEDTAVYYCARGYDVD | LTISSMEAEDAATYYCQQYHSYPPTFGGGTKLEIK |
| | | | VYW | | | | WFVYWGQGTLVTVSS | |
| suPAR_ | YTFTS | GWIDH | CARG | SARS | RTSNL | CQQYH | QVQLVQSGAEVKKPGASVKVSCKASGYTFTSNYI | DIQMTQSPSSLSASVGDRVTITCSARSSVSYMYWY |
| DB01_ | NYIH | ENGSTS | YDV | SVSY | AS | SYPPTF | HWVRQAPGQGLEWMGWIDHENGSTSYDQKFQG | QQKPGKAPKLLIYRTSNLASGVPSRFSGSGSGTDFT |
| A03 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDH | CARG | SASQ | RTSNL | CQQYH | QVQLVQSGAEVKKPGASVKVSCKASGYTFTGYY | DIQMTQSPSSLSASVGDRVTITCSASQSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| A04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | RARQ | RTSNL | CQQYH | QVQLVQSGAEVKKPGASVKVSCKASGYTFTGYY | DIQMTQSPSSLSASVGDRVTITCRARQSVSYMYWY |
| DB01_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| A05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RARQ | RTSNL | CQQYH | QVQLVQSGAEVKKPGASVKVSCKASGYTFTSYY | DIQMTQSPSSLSASVGDRVTITCRARQSVSYMYWY |
| DB01_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| A06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTS | GWIDH | CARG | SARS | RTSNL | CQQYH | QVQLVQSGAEVKKPGASVKVSCKASGGTFTSYY | DIQMTQSPSSLSASVGDRVTITCSARSSVSYMYWY |
| DB01_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| A07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFSS | GWIDH | CARG | SARS | RTSNL | CQQYH | QVQLVQSGAEVKKPGASVKVSCKASGDTFSSYY | DIQMTQSPSSLSASVGDRVTITCSARSSVSYMYWY |
| DB01_ | YYMH | SNGNT | YDV | SVSY | AS | SYPPTF | MHWVRQAPGQGLEWMGWIDHSNGNTSYDQKFQ | QQKPGKAPKLLIYRTSNLASGVPSRFSGSGSGTDFT |
| A08 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTA | GWIDH | CARG | SARS | RTSNL | CQQYH | QVQLVQSGAEVKKPGASVKVSCKASGYTFTAYY | DIQMTQSPSSLSASVGDRVTITCSARSSVSYMYWY |
| DB01_ | YYMH | EGGNTI | YDV | SVSY | AS | SYPPTF | MHWVRQAPGQGLEWMGWIDHEGGNTIYAQKFQ | QQKPGKAPKLLIYRTSNLASGVPSRFSGSGSGTDFT |
| A09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSN | GWIDH | CARG | SARS | RTSNL | CQQYH | QVQLVQSGAEVKKPGASVKVSCKASGYTFSNYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| A10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SASQ | RTSNL | CQQYH | QVQLVQSGAEVKKPGASVKVSCKASGYTFTNYY | DIQMTQSPSSLSASVGDRVTITCSASQSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| A11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB01_ | YYMH | EGGNTI | YDV | SVSY | AS | SYPPTF | MHWVRQAPGQGLEWMGWIDPEGGNTIYAQKFQ | QQKPGKAPKLLIYRTSNLASGVPSRFSGSGSGTDFT |
| A12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SASQ | RTSNL | CQQYH | QVQLVQSGAEVKKPGASVKVSCKASGYTFTDYY | DIQMTQSPSSLSASVGDRVTITCSASQSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| B01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| B02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | RASS | RTSNL | CQQYH | QVQLVQSGAEVKKPGASVKVSCKASGFNIKDYYI | DIQMTQSPSSLSASVGDRVTITCRASSSVSYMYWY |
| DB01_ | YYIH | ENGNTI | YDV | SVSY | AS | SYPPTF | HWVRQAPGQGLEWMGWIDPENGNTIYAQKFQG | QQKPGKAPKLLIYRTSNLASGVPSRFSGSGSGTDFT |
| B03 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDH | CARG | SASS | RTSNL | CQQYH | QVQLVQSGAEVKKPGASVKVSCKASGYTFTSYY | DIQMTQSPSSLSASVGDRVTITCSASSSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| B04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | RASS | RTSNL | CQQYH | QVQLVQSGAEVKKPGASVKVSCKASGYTFTNYY | DIQMTQSPSSLSASVGDRVTITCRASSSVSYMYWY |
| DB01_ | YYMH | ENQNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| B05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | RARS | RTSNL | CQQYH | QVQLVQSGAEVKKPGASVKVSCKASGYTFTDYY | DIQMTQSPSSLSASVGDRVTITCRARSSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| B06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSNYI | DIQMTQSPSSLSASVGDRVTITCSARSSVSYMYWY |
| DB01_ | NYIH | EGGSTI | YDV | SVSY | AS | SYPPTF | HWVRQAPGQGLEWMGWIDHEGGSTIYAQKFQG | QQKPGKAPKLLIYRTSNLASGVPSRFSGSGSGTDFT |
| B07 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB01_ | YYMH | ENQSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| B08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SARS | RTSNL | CQQYH | QVQLVQSGAEVKKPGASVKVSCKASGYTFTDYY | DIQMTQSPSSLSASVGDRVTITCSARSSVSYMYWY |
| DB01_ | YYMH | EGGNT | YDV | SVSY | AS | SYPPTF | MHWVRQAPGQGLEWMGWIDHEGGNTSYDQKFQ | QQKPGKAPKLLIYRTSNLASGVPSRFSGSGSGTDFT |
| B09 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSS | GWIDP | CARG | SARQ | RTSNL | CQQYH | QVQLVQSGAEVKKPGASVKVSCKASGYTFSSYY | DIQMTQSPSSLSASVGDRVTITCSARQSVSSMYWY |
| DB01_ | YYMH | ENQSTI | YDV | SVSS | AS | SYPPTF | MHWVRQAPGQGLEWMGWIDPENGSTIYDQKFQ | QQKPGKAPKLLIYRTSNLASGVPSRFSGSGSGTDFT |
| B10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SARS | RTSNL | CQQYH | QVQLVQSGAEVKKPGASVKVSCKASGYTFTDYY | DIQMTQSPSSLSASVGDRVTITCSARSSVSYMYWY |
| DB01_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| B11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| B12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNI | CQQYH | QVQLVQSGAEVKKPGASVKVSCKASGYTFTGYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| C01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFNT | GWIDP | CARG | SASS | RTSSLQ | CQQYH | QVQLVQSGAEVKKPGASVKVSCKASGYTFNTYY | DIQMTQSPSSLSASVGDRVTITCSASSSVSYMYWY |
| DB01_ | YYMH | ENGNTI | YDV | SVSY | S | SYPPTF | MHWVRQAPGQGLEWMGWIDPENGNTIYDQKFQ | QQKPGKAPKLLIYRTSSLQSGVPSRFSGSGSGTDFT |
| C02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB01_ | YYMH | SNGNTI | YDV | SVSY | AS | SYPPTF | MHWVRQAPGQGLEWMGWIDHSNGNTIYDQKFQ | QQKPGKAPKLLIYRTSNLASGVPSRFSGSGSGTDFT |
| C03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB01_ | YYMH | EGGSTI | YDV | SVSY | AS | SYPPTF | MHWVRQAPGQGLEWMGWIDPEGGSTIYDQKFQ | QQKPGKAPKLLIYRTSNLASGVPSRFSGSGSGTDFT |
| C04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB01_ | YYMH | ENGSTI | YDV | SVSY | QS | SYPPTF | MHWVRQAPGQGLEWMGWIDHENGSTIYDQKFQ | QQKPGKAPKLLIYRTSNLQSGVPSRFSGSGSGTDFT |
| C05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDH | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB01_ | YYIH | ENQNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| C06 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDH | CARG | RARQ | RTSNL | CQQYH | QVQLVQSGAEVKKPGASVKVSCKASGYTFTSYY | DIQMTQSPSSLSASVGDRVTITCRARQSVSYMYWY |
| DB01_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| C07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDHENGNTIYAQKFQ | QQKPGKAPKLLIYRTSNLASGVPSRFSGSGSGTDFT |
| C08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB01_ | YYIH | ENQNTI | YDV | SVSY | AS | SYPPTF | HWVRQAPGQGLEWMGWIDPENGNTIYAQKFQG | QQKPGKAPKLLIYRTSNLASGVPSRFSGSGSGTDFT |
| C09 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDP | CARG | RARS | RTSNL | CQQYH | QVQLVQSGAEVKKPGASVKVSCKASGYTFTNYY | DIQMTQSPSSLSASVGDRVTITCRARSSISYMYWYQ |
| DB01_ | YYMH | ENGNTI | YDV | SISY | AS | SYPPTF | MHWVRQAPGQGLEWMGWIDPENGNTIYAQKFQ | QKPGKAPKLLIYRTSNLASGVPSRFSGSGSGTDFTL |
| C10 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | GTFTS | GWIDP | CARG | SARS | RTSNL | CQQYH | QVQLVQSGAEVKKPGASVKVSCKASGGTFTSYY | DIQMTQSPSSLSASVGDRVTITCSARSSVSYMYWY |
| DB01_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| C11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTS | GWIDH | CARG | SARS | RTSNL | CQQYH | QVQLVQSGAEVKKPGASVKVSCKASGDTFTSYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| C12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB01_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| D01 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | RARS | RTSNL | CQQYH | QVQLVQSGAEVKKPGASVKVSCKASGYTFTSYY | DIQMTQSPSSLSASVGDRVTITCRARSSVSYMYWY |
| DB01_ | YYMH | NTGDT | YDV | SVSY | AS | SYPPTF | MHWVRQAPGQGLEWMGWIDPNTGDTNYAQKFQ | QQKPGKAPKLLIYRTSNLASGVPSRFSGSGSGTDFT |
| D02 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTN | GWIDH | CARG | SARS | RTSNL | CQQYH | QVQLVQSGAEVKKPGASVKVSCKASGGTFTNYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB01_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| D04 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARQ | RTSNL | CQQYH | QVQLVQSGAEVKKPGASVKVSCKASGYTFTGSY | DIQMTQSPSSLSASVGDRVTITCSARQSVSYMYWY |
| DB01_ | SYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| D05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB01_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| D06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RARS | RTSNL | CQQYH | QVQLVQSGAEVKKPGASVKVSCKASGYTFTSYY | DIQMTQSPSSLSASVGDRVTITCRARSSISYMYWY |
| DB01_ | YYMH | ENGSTI | YDV | SISY | AS | SYPPTF | MHWVRQAPGQGLEWMGWIDHENGSTIYAQKFQ | QQKPGKAPKLLIYRTSNLASGVPSRFSGSGSGTDFTL |
| D07 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RASS | RTSNL | CQQYH | QVQLVQSGAEVKKPGASVKVSCKASGYTFTSYY | DIQMTQSPSSLSASVGDRVTITCRASSSVSYMYWY |
| DB01_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| D08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB01_ | YYMH | SNGNTI | YDV | SVSY | AS | SYPPTF | MHWVRQAPGQGLEWMGWIDHSNGNTIYAQKFQ | QQKPGKAPKLLIYRTSNLASGVPSRFSGSGSGTDFT |
| D09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARQ | RTSNL | CQQYH | QVQLVQSGAEVKKPGASVKVSCKASGYTFTGYY | DIQMTQSPSSLSASVGDRVTITCSARQSVSYMYWY |
| DB01_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| D10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTNY | GWIDY | CARG | SARQ | RTSNL | CQQYH | QVQLVQSGAEVKKPGASVKVSCKASGYTNYYFH | DIQMTQSPSSLSASVGDRVTITCSARQSISYMYWYQ |
| DB01_ | YFH | NSGGT | YDV | SISY | AS | SYPPTF | WVRQAPGQGLEWMGWIDPNSGGTNYAQKFQGR | QKPGKAPKLLIYRTSNLASGVPSRFSGSGSGTDFTL |
| D11 | | NYA | DWF | MY | | | VIMTRDTSTSTVYMELSSLRSEDTAVYYCARGYD | TISSLQPEDFATYYCQQYHSYPPTFGGGTKVEIKR |
| | | | VYW | | | | VDWFVYWGQGTLVTVSS | |
| suPAR_ | YTFTS | GWIDP | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB01_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| D12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTS | GWIQP | CARG | SARS | RTSNL | CQQYH | QVQLVQSGAEVKKPGASVKVSCKASGYSFTSYY | DIQMTQSPSSLSASVGDRVTITCSARSSVSYMYWY |
| DB01_ | YYMH | EGGSTI | YDV | SVSY | AS | SYPPTF | MHWVRQAPGQGLEWMGWIDPEGGSTIYDQKFQ | QQKPGKAPKLLIYRTSNLASGVPSRFSGSGSGTDFT |
| E01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB01_ | YYMH | EGGSTI | YDV | SVSY | AS | SYPPTF | MHWVRQAPGQGLEWMGWIDPEGGSTIYDQKFQ | QQKPGKAPKLLIYRTSNLASGVPSRFSGSGSGTDFT |
| E02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SARS | RTSNL | CQQYH | QVQLVQSGAEVKKPGASVKVSCKASGYTFTDYY | DIQMTQSPSSLSASVGDRVTITCSARSSVSYMYWY |
| DB01_ | YYMH | ENGNT | YDV | SVSY | AS | SYPPTF | MHWVRQAPGQGLEWMGWIDHENGNTSYAQKFQ | QQKPGKAPKLLIYRTSNLASGVPSRFSGSGSGTDFT |
| E03 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARQ | RTSNL | CQQYH | QVQLVQSGAEVKKPGASVKVSCKASGYTFTGYY | DIQMTQSPSSLSASVGDRVTITCSARQSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | STENS | GWIDH | CARG | SARS | RTSNL | CQQYH | QVQLVQSGAEVKKPGASVKVSCKASGSTENSYY | DIQMTQSPSSLSASVGDRVTITCSARSSVSYMYWY |
| DB01_ | YYMH | ENGNTI | NDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | NDVDWFVYWGQGTLVTVSS | |
| suPAR_ | YTFIN | GWIDP | CARG | SARS | RTSNL | CQQYH | QVQLVQSGAEVKKPGASVKVSCKASGYTFINYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| E06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RASS | RTSNL | CQQYH | QVQLVQSGAEVKKPGASVKVSCKASGYTFTSYY | DIQMTQSPSSLSASVGDRVTITCRASSSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYYI | DIQMTQSPSSLSASVGDRVTITCSARSSVSYMYWY |
| DB01_ | YYIH | ENGSTI | YDV | SVSY | AS | SYPPTF | HWVRQAPGQGLEWMGWIDHENGSTIYDQKFQG | QQKPGKAPKLLIYRTSNLASGVPSRFSGSGSGTDFT |
| E08 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSISYMYWYQ |
| DB01_ | YYMH | ENGNTI | YDV | SISY | QS | SYPPTF | MHWVRQAPGQGLEWMGWIDHENGNTIYDQKFQ | QKPGKAPKLLIYRTSNLQSGVPSRFSGSGSGTDFTL |
| E09 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | RARQ | RTSNL | CQQYH | QVQLVQSGAEVKKPGASVKVSCKASGYTFTGYY | DIQMTQSPSSLSASVGDRVTITCRARQSVSYMYWY |
| DB01_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| E10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB01_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| E11 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFSS | GVINPG | CARG | QASQ | DASNL | CQQYH | QVQLVQSGAEVKKPGASVKVSCKASGGTFSSYAI | DIQMTQSPSSLSASVGDRVTITCQASQDISNYINWY |
| DB01_ | YAIS | GGGTN | YDV | DISN | AS | SYPPTF | SWVRQAPGQGLEWLGVINPGGGGTNYAQKFQGR | QQKPGKAPKLLIYDASNLASGVPSRFSGSGSGTDFT |
| E12 | | YA | DWF | YIN | | | VTMTRDTSTSTVYMELSSLRSEDTAVYYCARGYD | LTISSLQPEDFATYYCQQYHSYPPTFGGGTKVEIKR |
| | | | VTW | | | | VDWFVTWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB01_ | YYVH | ENGSTI | YDV | SVSY | AS | SYPPTF | VHWVRQAPGQGLEWMGWIDHENGSTIYAQKFQ | QQKPGKAPKLLIYRTSNLASGVPSRFSGSGSGTDFT |
| F01 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB01_ | YYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| F02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARQ | RTSNL | CQQYH | QVQLVQSGAEVKKPGASVKVSCKASGYTFTSYY | DIQMTQSPSSLSASVGDRVTITCSARQSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| F03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SARS | RTSNL | CQQYH | QVQLVQSGAEVKKPGASVKVSCKASGYTFTDYY | DIQMTQSPSSLSASVGDRVTITCSARSSVSYMYWY |
| DB01_ | YYMH | SNQNTI | YDV | SVSY | AS | SYPPTF | MHWVRQAPGQGLEWMGWIDHSNGNTIYAQKFQ | QQKPGKAPKLLIYRTSNLASGVPSRFSGSGSGTDFT |
| F04 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSN | GWIDH | CARG | SARS | RTSNL | CQQYH | QVQLVQSGAEVKKPGASVKVSCKASGYTFSNYY | DIQMTQSPSSLSASVGDRVTITCSARSSVSYMYWY |
| DB01_ | YYMH | EGGSTI | YDV | SVSY | AS | SYPPTF | MHWVRQAPGQGLEWMGWIDHEGGSTIYDQKFQ | QQKPGKAPKLLIYRTSNLASGVPSRFSGSGSGTDFT |
| F05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARQ | RTSNL | CQQYH | QVQLVQSGAEVKKPGASVKVSCKASGYTFTSYY | DIQMTQSPSSLSASVGDRVTITCSARQSISYMYWYQ |
| DB01_ | YYMH | ENGNTI | YDV | SISY | QS | SYPPTF | MHWVRQAPGQGLEWMGWIDHENGNTIYAQKFQ | QKPGKAPKLLIYRTSNLQSGVPSRFSGSGSGTDFTL |
| F06 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB01_ | YYVH | ENGSTS | YDV | SVSY | AS | SYPPTF | VHWVRQAPGQGLEWMGWIDHENGSTSYDQKFQ | QQKPGKAPKLLIYRTSNLASGVPSRFSGSGSGTDFT |
| F07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | RARQ | RTSNL | CQQYH | QVQLVQSGAEVKKPGASVKVSCKASGYTFTGYY | DIQMTQSPSSLSASVGDRVTITCRARQSVSYMYWY |
| DB01_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| F08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKS | GWIDP | CARG | SARS | RTSNL | CQQYH | QVQLVQSGAEVKKPGASVKVSCKASGENFKSYYI | DIQMTQSPSSLSASVGDRVTITCSARSSVSYMYWY |
| DB01_ | YYIH | ENGSTI | YDV | SVSY | AS | SYPPTF | HWVRQAPGQGLEWMGWIDPENGSTIYDQKFQGR | QQKPGKAPKLLIYRTSNLASGVPSRFSGSGSGTDFT |
| F09 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB01_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| F10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RASS | RTSNL | CQQYH | QVQLVQSGAEVKKPGASVKVSCKASGYTFTSYY | DIQMTQSPSSLSASVGDRVTITCRASSSVSYMYWY |
| DB01_ | YYMH | EGGNT | YDV | SVSY | AS | SYPPTF | MHWVRQAPGQGLEWMGWIDHEGGNTSYAQKFQ | QQKPGKAPKLLIYRTSNLASGVPSRFSGSGSGTDFT |
| F11 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | RARQ | RTSNL | CQQYH | QVQLVQSGAEVKKPGASVKVSCKASGFNIKDYYI | DIQMTQSPSSLSASVGDRVTITCRARQSVSYMYWY |
| DB01_ | YYIH | ENQSTI | YDV | SVSY | AS | SYPPTF | HWVRQAPGQGLEWMGWIDPENGSTIYDQKFQGR | QQKPGKAPKLLIYRTSNLASGVPSRFSGSGSGTDFT |
| F12 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | DTFSS | GWIDH | CARG | SARS | RTSNL | CQQYH | QVQLVQSGAEVKKPGASVKVSCKASGDTFSSYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTD | GWIDH | CARG | SARS | RTSNL | CQQYH | QVQLVQSGAEVKKPGASVKVSCKASGYSFTDYY | DIQMTQSPSSLSASVGDRVTITCSARSSVSYMYWY |
| DB01_ | YYMH | ENQNTI | YDV | SVSY | AS | SYPPTF | MHWVRQAPGQGLEWMGWIDHENGNTIYAQKFQ | QQKPGKAPKLLIYRTSNLASGVPSRFSGSGSGTDFT |
| G02 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | STFNS | GWIDH | CARG | RARQ | RTSNL | CQQYH | QVQLVQSGAEVKKPGASVKVSCKASGSTENSYY | DIQMTQSPSSLSASVGDRVTITCRARQSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTLSG | GWIDP | CARG | RARQ | RTSNL | CQQYH | QVQLVQSGAEVKKPGASVKVSCKASGYTLSGYY | DIQMTQSPSSLSASVGDRVTITCRARQSVSYMYWY |
| DB01_ | YYMH | NSGGT | YDV | SVSY | AS | SYPPTF | MHWVRQAPGQGLEWMGWIDPNSGGTKYAQKFQ | QQKPGKAPKLLIYRTSNLASGVPSRFSGSGSGTDFT |
| G04 | | KYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTLTN | GWIDH | CARG | SARQ | RTSNL | CQQYH | QVQLVQSGAEVKKPGASVKVSCKASGDTLINYY | DIQMTQSPSSLSASVGDRVTITCSARQSVSYMYWY |
| DB01_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKD | GWIDP | CARG | SARS | RTSNL | CQQYH | QVQLVQSGAEVKKPGASVKVSCKASGENFKDYYI | DIQMTQSPSSLSASVGDRVTITCSARSSVSYMYWY |
| DB01_ | YYIH | EGGNTI | YDV | SVSY | AS | SYPPTF | HWVRQAPGQGLEWMGWIDPEGGNTIYAQKFQG | QQKPGKAPKLLIYRTSNLASGVPSRFSGSGSGTDFT |
| G06 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | NTFTS | GWIDP | CARG | SARS | RTSNL | CQQYH | QVQLVQSGAEVKKPGASVKVSCKASGNTFTSYY | DIQMTQSPSSLSASVGDRVTITCSARSSVSYMYWY |
| DB01_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| G07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTN | GWIDH | CARG | SARQ | RTSSLA | CQQYH | QVQLVQSGAEVKKPGASVKVSCKASGYSFTNYY | DIQMTQSPSSLSASVGDRVTITCSARQSVSYMYWY |
| DB01_ | YYMH | ENGNT | YDV | SVSY | S | SYPPTF | MHWVRQAPGQGLEWMGWIDHENGNTSYAQKFQ | QQKPGKAPKLLIYRTSSLASGVPSRFSGSGSGTDFT |
| G08 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTLNS | GWIDP | CARG | SARQ | RTSNL | CQQYH | QVQLVQSGAEVKKPGASVKVSCKASGHTLNSYY | DIQMTQSPSSLSASVGDRVTITCSARQSVSYMYWY |
| DB01_ | YYMH | ENQNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| G09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB01_ | YYMH | EGGNT | YDV | SVSY | AS | SYPPTF | MHWVRQAPGQGLEWMGWIDHEGGNTSYAQKFQ | QQKPGKAPKLLIYRTSNLASGVPSRFSGSGSGTDFT |
| G10 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DSFNN | GWIDH | CARG | SARS | RTSNL | CQQYH | QVQLVQSGAEVKKPGASVKVSCKASGDSENNYY | DIQMTQSPSSLSASVGDRVTITCSARSSVSYMYWY |
| DB01_ | YYFH | EGGSTI | YDV | SVSY | AS | SYPPTF | FHWVRQAPGQGLEWMGWIDHEGGSTIYAQKFQG | QQKPGKAPKLLIYRTSNLASGVPSRFSGSGSGTDFT |
| G11 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | DTFSN | GWIDH | CARG | SARS | RTSNL | CQQYH | QVQLVQSGAEVKKPGASVKVSCKASGDTFSNYY | DIQMTQSPSSLSASVGDRVTITCSARSSISYMYWYQ |
| DB01_ | YYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| G12 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | DTFTS | GWIDP | CARG | SARS | RTSNL | CQQYH | QVQLVQSGAEVKKPGASVKVSCKASGDTFTSYY | DIQMTQSPSSLSASVGDRVTITCSARSSVSYMYWY |
| DB01_ | YYMH | ENGNTI | YDV | SVSY | AS | SYPPTE | MHWVRQAPGQGLEWMGWIDPENGNTIYDQKFQ | QQKPGKAPKLLIYRTSNLASGVPSRFSGSGSGTDFT |
| H01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFTD | GWIDP | CARG | SARS | RTSNL | CQQYH | QVQLVQSGAEVKKPGASVKVSCKASGENFTDYYI | DIQMTQSPSSLSASVGDRVTITCSARSSVSYMYWY |
| DB01_ | YYIH | ENGNTI | YDV | SVSY | AS | SYPPTF | HWVRQAPGQGLEWMGWIDPENGNTIYAQKFQG | QQKPGKAPKLLIYRTSNLASGVPSRFSGSGSGTDFT |
| H02 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | DTITN | GWIDT | CARG | SARS | RTSNL | CQQYH | QVQLVQSGAEVKKPGASVKVSCKASGDTITNYY | DIQMTQSPSSLSASVGDRVTITCSARSSVSYMYWY |
| DB01_ | YYMH | NSGGT | YDV | SVSY | AS | SYPPTF | MHWVRQAPGQGLEWMGWIDTNSGGTQYAQKFQ | QQKPGKAPKLLIYRTSNLASGVPSRFSGSGSGTDFT |
| H03 | | QYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB01_ | YYMH | NSGGT | YDV | SVSY | AS | SYPPTF | MHWVRQAPGQGLEWMGWIDPNSGGTNYAQKFQ | QQKPGKAPKLLIYRTSNLASGVPSRFSGSGSGTDFT |
| H04 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGNY | DIQMTQSPSSLSASVGDRVTITCSARSSVSYMYWY |
| DB01_ | NYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| H05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | SARQ | RTSNL | CQQYH | QVQLVQSGAEVKKPGASVKVSCKASGFNIKDYY | DIQMTQSPSSLSASVGDRVTITCSARQSISYMYWYQ |
| DB01_ | YYMH | ENGNTI | YDV | SISY | AS | SYPPTF | MHWVRQAPGQGLEWMGWIDPENGNTIYDQKFQ | QKPGKAPKLLIYRTSNLASGVPSRFSGSGSGTDFTL |
| H06 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | DTFSN | GWIDH | CARG | SARS | RTSNL | CQQYH | QVQLVQSGAEVKKPGASVKVSCKASGDTFSNYY | DIQMTQSPSSLSASVGDRVTITCSARSSVSYMYWY |
| DB01_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| H07 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWEN | CARG | RASS | RTSNL | CQQYH | QVQLVQSGAEVKKPGASVKVSCKASGFNIKDYYI | DIQMTQSPSSLSASVGDRVTITCRASSSVSYMYWY |
| DB01_ | YYIH | NGGGT | YDV | SVSY | QS | SYPPTF | HWVRQAPGQGLEWMGWIDPNGGGTQYAQKFQG | QQKPGKAPKLLIYRTSNLQSGVPSRFSGSGSGTDFT |
| H08 | | QYA | DWF | MY | | | RVTMTRDTSTSTVYMELSSLRSEDTAVYYCARGY | LTISSLQPEDFATYYCQQYHSYPPTFGGGTKVEIKR. |
| | | | VYW | | | | DVDWFVYWGQGTLVTVSS | |
| suPAR_ | GTFST | GWIDH | CARG | SASQ | RTSNL | CQQYH | QVQLVQSGAEVKKPGASVKVSCKASGGTESTYY | DIQMTQSPSSLSASVGDRVTITCSASQSVSYMYWY |
| DB01_ | YYMH | EGGNT | YDV | SVSY | AS | SYPPTF | MHWVRQAPGQGLEWMGWIDHEGGNTSYAQKFQ | QQKPGKAPKLLIYRTSNLASGVPSRFSGSGSGTDFT |
| H09 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARQ | RTSNL | CQQYH | QVQLVQSGAEVKKPGASVKVSCKASGYTFTNYY | DIQMTQSPSSLSASVGDRVTITCSARQSVSYMYWY |
| DB01_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| H10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDP | CARG | SARS | RTSNL | CQQYH | QVQLVQSGAEVKKPGASVKVSCKASGYTFTDYYI | DIQMTQSPSSLSASVGDRVTITCSARSSVSYMYWY |
| DB02_ | YYIH | ENGNTI | YDV | SVSY | AS | SYPPTF | HWVRQAPGQGLEWMGWIDPENGNTIYAQKFQG | QQKPGKAPKLLIYRTSNLASGVPSRFSGSGSGTDFT |
| A03 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDH | CARG | RASS | RTSNL | CQQYH | QVQLVQSGAEVKKPGASVKVSCKASGYTFTGYY | DIQMTQSPSSLSASVGDRVTITCRASSSVSYMYWY |
| DB02_ | YYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| A04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTS | GWIDP | CARG | SARQ | RTSNL | CQQYH | QVQLVQSGAEVKKPGASVKVSCKASGDTFTSYY | DIQMTQSPSSLSASVGDRVTITCSARQSVSYMYWY |
| DB02_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| A05 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTG | GWIDP | CARG | RARS | RTSNL | CQQYH | QVQLVQSGAEVKKPGASVKVSCKASGGTFTGYY | DIQMTQSPSSLSASVGDRVTITCRARSSVSYMYWY |
| DB02_ | YYMH | NSGVT | YDV | SVSY | AS | SYPPTF | MHWVRQAPGQGLEWMGWIDPNSGVTNYAQKFQ | QQKPGKAPKLLIYRTSNLASGVPSRFSGSGSGTDFT |
| A06 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNIKD | GWISP | CARG | SARQ | RTSNL | CQQYH | QVQLVQSGAEVKKPGASVKVSCKASGYNIKDYYI | DIQMTQSPSSLSASVGDRVTITCSARQSVSYMYWY |
| DB02_ | YYIH | DSGVT | YDV | SVSY | AS | SYPPTF | HWVRQAPGQGLEWMGWISPDSGVTNYAQKFQG | QQKPGKAPKLLIYRTSNLASGVPSRFSGSGSGTDFT |
| A07 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTT | GWID | CARG | SARS | RTSNL | CQQYH | QVQLVQSGAEVKKPGASVKVSCKASGYTFTTYY | DIQMTQSPSSLSASVGDRVTITCSARSSVSYMYWY |
| DB02_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| A08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | SARQ | RTSNL | CQQYH | QVQLVQSGAEVKKPGASVKVSCKASGYTFTNYY | DIQMTQSPSSLSASVGDRVTITCSARQSISYMYWYQ |
| DB02_ | YYMH | EGGSTI | YDV | SISY | AS | SYPPTF | MHWVRQAPGQGLEWMGWIDPEGGSTIYDQKFQ | QKPGKAPKLLIYRTSNLASGVPSRFSGSGSGTDFTL |
| A09 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGNY | DIQMTQSPSSLSASVGDRVTITCSARSSVSYMYWY |
| DB02_ | NYMH | ENGNTI | SDVD | SVSY | AS | SYPPTF | MHWVRQAPGQGLEWMGWIDHENGNTIYDQKFQ | QQKPGKAPKLLIYRTSNLASGVPSRFSGSGSGTDFT |
| A10 | | YD | WFV | MY | | | GRVTMTRDTSTSTVYMELSSLRSEDTAVYYCARG | LTISSLQPEDFATYYCQQYHSYPPTFGGGTKVEIKR |
| | | | YW | | | | SDVDWFVYWGQGTLVTVSS | |
| suPAR_ | GTFSS | GWIDP | CARG | SARQ | RTSNL | CQQYH | QVQLVQSGAEVKKPGASVKVSCKASGGTFSSYY | DIQMTQSPSSLSASVGDRVTITCSARQSVSYMYWY |
| DB02_ | YYMH | SNGNTI | YDV | SVSY | AS | SYPPTF | MHWVRQAPGQGLEWMGWIDPSNGNTIYDQKFQ | QQKPGKAPKLLIYRTSNLASGVPSRFSGSGSGTDFT |
| A11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB02_ | YYMH | ENQSTI | YDM | SVSY | AS | SYPPTF | MHWVRQAPGQGLEWMGWIDHENGSTIYAQKFQ | QQKPGKAPKLLIYRTSNLASGVPSRFSGSGSGTDFT |
| A12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDMDWFVYWGQGTLVTVSS | |
| suPAR_ | YTFTG | GWIDH | CARG | SARQ | RTSNL | CQQYH | QVQLVQSGAEVKKPGASVKVSCKASGYTFTGHY | DIQMTQSPSSLSASVGDRVTITCSARQSVSYMYWY |
| DB02_ | HYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| B01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB02_ | YYIH | ENQSTI | YDID | SVSY | AS | SYPPTF | HWVRQAPGQGLEWMGWIDPENGSTIYDQKFQGR | QQKPGKAPKLLIYRTSNLASGVPSRFSGSGSGTDFT |
| B02 | | YD | WFV | MY | | | VTMTRDTSTSTVYMELSSLRSEDTAVYYCARGYD | LTISSLQPEDFATYYCQQYHSYPPTFGGGTKVEIKR |
| | | | YW | | | | DDWFVYWGQGTLVTVSS | |
| suPAR_ | YTFIG | GWIDH | CARG | SARS | RTSNL | CQQYH | QVQLVQSGAEVKKPGASVKVSCKASGYTFIGNYI | DIQMTQSPSSLSASVGDRVTITCSARSSVSYMYWY |
| DB02_ | NYIH | ENGSTI | SDVD | SVSY | AS | SYPPTF | HWVRQAPGQGLEWMGWIDHENGSTIYDQKFQG | QQKPGKAPKLLIYRTSNLASGVPSRFSGSGSGTDFT |
| B03 | | YD | WFV | MY | | | RVTMTRDTSTSTVYMELSSLRSEDTAVYYCARGS | LTISSLQPEDFATYYCQQYHSYPPTFGGGTKVEIKR |
| | | | YW | | | | DVDWFVYWGQGTLVTVSS | |
| suPAR_ | YTFTG | GWIDH | CARG | SARS | RTSNL | CQQYH | QVQLVQSGAEVKKPGASVKVSCKASGYTFTGHY | DIQMTQSPSSLSASVGDRVTITCSARSSVSYMYWY |
| DB02_ | HYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| B04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RARQ | RTSNL | CQQYH | QVQLVQSGAEVKKPGASVKVSCKASGYTFTSYY | DIQMTQSPSSLSASVGDRVTITCRARQSVSYMYWY |
| DB02_ | YYMH | ENGNT | YDV | SVSY | AS | SYPPTF | MHWVRQAPGQGLEWMGWIDHENGNTSYAQKFQ | QQKPGKAPKLLIYRTSNLASGVPSRFSGSGSGTDFT |
| B05 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTS | GWIDH | CARG | SARS | RTSNL | CQQYH | QVQLVQSGAEVKKPGASVKVSCKASGDTFTSYY | DIQMTQSPSSLSASVGDRVTITCSARSSVSYMYWY |
| DB02_ | YYMH | EGGSTI | YDV | SVSY | AS | SYPPTF | MHWVRQAPGQGLEWMGWIDHEGGSTIYAQQFQ | QQKPGKAPKLLIYRTSNLASGVPSRFSGSGSGTDFT |
| B06 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTFSS | GWIDP | CARG | SARQ | RTSNL | CQQYH | QVQLVQSGAEVKKPGASVKVSCKASGHTFSSYY | DIQMTQSPSSLSASVGDRVTITCSARQSISYMYWYQ |
| DB02_ | YYMH | ENGNTI | YDV | SISY | AS | SYPPTF | MHWVRQAPGQGLEWMGWIDPENGNTIYAQKFQ | QKPGKAPKLLIYRTSNLASGVPSRFSGSGSGTDFTL |
| B07 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YSFTN | GWIDH | CARG | SARS | RTSNL | CQQYH | QVQLVQSGAEVKKPGASVKVSCKASGYSFTNYY | DIQMTQSPSSLSASVGDRVTITCSARSSVSYMYWY |
| DB02_ | | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| B08 | YYMH | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CASG | SARS | RTSNL | CQQYH | QVQLVQSGAEVKKPGASVKVSCKASGYTFTGYY | DIQMTQSPSSLSASVGDRVTITCSARSSVSYMYWY |
| DB02_ | YYVH | SNGNTI | YDV | SVSY | AS | SYPPTF | VHWVRQAPGQGLEWMGWIDHSNGNTIYDQKFQ | QQKPGKAPKLLIYRTSNLASGVPSRFSGSGSGTDFT |
| B09 | | YD | DWF | MY | | | GRVTMTRDTSTSTVYMELSSLRSEDTAVYYCASG | LTISSLQPEDFATYYCQQYHSYPPTFGGGTKVEIKR |
| | | | VYW | | | | YDVDWFVYWGQGTLVTVSS | |
| suPAR_ | YTFTN | GWID | CARG | RARQ | RTSNL | CQQYH | QVQLVQSGAEVKKPGASVKVSCKASGYTFTNYY | DIQMTQSPSSLSASVGDRVTITCRARQSVSYMYWY |
| DB02_ | YYMH | ENGNT | YDV | SVSY | AS | SYPPTF | MHWVRQAPGQGLEWMGWIDPENGNTSYDQKFQ | QQKPGKAPKLLIYRTSNLASGVPSRFSGSGSGTDFT |
| B10 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSS | GWIDH | CARG | SARQ | RTSNL | CQQYH | QVQLVQSGAEVKKPGASVKVSCKASGYTFSSYY | DIQMTQSPSSLSASVGDRVTITCSARQSISYMYWYC |
| DB02_ | YYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| B11 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | FNEKS | GWIDE | CARG | SARS | RTSNL | CQQYH | QVQLVQSGAEVKKPGASVKVSCKASGENFKSYYI | DIQMTQSPSSLSASVGDRVTITCSARSSVSYMYWY |
| DB02_ | YYIH | NSGGT | YDV | SVSY | AS | SYPPTF | HWVRQAPGQGLEWMGWIDPNSGGTNYAQKFQG | QQKPGKAPKLLIYRTSNLASGVPSRFSGSGSGTDFT |
| B12 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDP | CARG | RARQ | RTSNL | CQQYH | QVQLVQSGAEVKKPGASVKVSCKASGYTFTGYY | DIQMTQSPSSLSASVGDRVTITCRARQSVSYMYWY |
| DB02_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| C01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFKS | GWIDH | CASG | SARS | RTSNL | CQQYH | QVQLVQSGAEVKKPGASVKVSCKASGYTFKSYY | DIQMTQSPSSLSASVGDRVTITCSARSSVSYMYWY |
| DB02_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| C02 | | YD | DWF | MY | | | GRVTMTRDTSTSTVYMELSSLRSEDTAVYYCASG | LTISSLQPEDFATYYCQQYHSYPPTFGGGTKVEIKR |
| | | | VYW | | | | YDVDWFVYWGQGTLVTVSS | |
| suPAR_ | FNIKD | GWIDY | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB02_ | YYIH | ENGSTI | YDV | SVSY | AS | SYPPTF | HWVRQAPGQGLEWMGWIDPENGSTIYDQKFQGR | QQKPGKAPKLLIYRTSNLASGVPSRFSGSGSGTDFT |
| C03 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | FNIKD | GWIDH | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB02_ | YYIH | EGGSTS | YDV | SVSY | AS | SYPPTF | HWVRQAPGQGLEWMGWIDHEGGSTSYDQKFQG | QQKPGKAPKLLIYRTSNLASGVPSRFSGSGSGTDFT |
| C04 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | GTFSS | GWIDH | CARG | SARS | RTSNL | CQQYH | QVQLVQSGAEVKKPGASVKVSCKASGGTFSSYY | DIQMTQSPSSLSASVGDRVTITCSARSSVSYMYWY |
| DB02_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| C05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNITD | GWIDH | CARG | SASQ | RTSNL | CQQYH | QVQLVQSGAEVKKPGASVKVSCKASGFNITDYY | DIQMTQSPSSLSASVGDRVTITCSASQSVSYMYWY |
| DB02_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| C06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDA | CARG | SARS | RTSNL | CQQYH | QVQLVQSGAEVKKPGASVKVSCKASGYTFTDSYI | DIQMTQSPSSLSASVGDRVTITCSARSSVSYMYWY |
| DB02_ | SYIH | NNGDT | YDV | SVSY | AS | SYPPTF | HWVRQAPGQGLEWMGWIDANNGDTNYAERFQG | QQKPGKAPKLLIYRTSNLASGVPSRFSGSGSGTDFT |
| C07 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFSN | GWIDH | CARG | SARS | RTSNL | CQQYH | QVQLVQSGAEVKKPGASVKVSCKASGYTFSNYYI | DIQMTQSPSSLSASVGDRVTITCSARSSVSYMYWY |
| DB02_ | YYIH | SNGSTI | YDV | SVSY | AS | SYPPTF | HWVRQAPGQGLEWMGWIDHSNGSTIYDQKFQGR | QQKPGKAPKLLIYRTSNLASGVPSRFSGSGSGTDFT |
| C08 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFTG | GLINPS | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB02_ | YYMH | DGYTT | YDV | SVSY | AS | SYPPTF | MHWVRQAPGQGLEWVGLINPSDGYTTYAQKFQG | QQKPGKAPKLLIYRTSNLASGVPSRFSGSGSGTDFT |
| C09 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTD | GWIDH | CARG | SARS | RTSNL | CQQYH | QVQLVQSGAEVKKPGASVKVSCKASGYTFTDYY | DIQMTQSPSSLSASVGDRVTITCSARSSVSYMYWY |
| DB02_ | YYVH | EGGSTI | YDV | SVSY | AS | SYPPTF | VHWVRQAPGQGLEWMGWIDHEGGSTIYDQKFQ | QQKPGKAPKLLIYRTSNLASGVPSRFSGSGSGTDFT |
| C10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNFK | GWIDH | CARG | SARQ | RTSNL | CQQYH | QVQLVQSGAEVKKPGASVKVSCKASGYNFKDYY | DIQMTQSPSSLSASVGDRVTITCSARQSVSYMYWY |
| DB02_ | DYYIH | ENGSTI | YDV | SVSY | AS | SYPPTF | IHWVRQAPGQGLEWMGWIDHENGSTIYAQKFQG | QQKPGKAPKLLIYRTSNLASGVPSRFSGSGSGTDFT |
| C11 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTLTD | GWIDP | CARG | SARS | RTSNL | CQQYH | QVQLVQSGAEVKKPGASVKVSCKASGYTLTDYY | DIQMTQSPSSLSASVGDRVTITCSARSSVSYMYWY |
| DB02_ | YYMH | EGGNT | YDV | SVSY | AS | SYPPTF | MHWVRQAPGQGLEWMGWIDPEGGNTSYDQKFQ | QQKPGKAPKLLIYRTSNLASGVPSRFSGSGSGTDFT |
| C12 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SARS | RTSNL | CQQYH | QVQLVQSGAEVKKPGASVKVSCKASGYTFTDYY | DIQMTQSPSSLSASVGDRVTITCSARSSVSYMYWY |
| DB02_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTS | GWIDH | CARG | SARS | RTSNL | CQQYH | QVQLVQSGAEVKKPGASVKVSCKASGDTFTSYY | DIQMTQSPSSLSASVGDRVTITCSARSSVSYMYWY |
| DB02_ | YYMH | EGGNT | YDV | SVSY | AS | SYPPTF | MHWVRQAPGQGLEWMGWIDHEGGNTSYAQKFQ | QQKPGKAPKLLIYRTSNLASGVPSRFSGSGSGTDFT |
| D02 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSHY | DIQMTQSPSSLSASVGDRVTITCSARSSVSYMYWY |
| DB02_ | HYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FTIKD | GWIDP | CARG | RARQ | RTSNL | CQQYH | QVQLVQSGAEVKKPGASVKVSCKASGFTIKDYY | DIQMTQSPSSLSASVGDRVTITCRARQSVSYMYWY |
| DB02_ | YYMH | DEGGT | YDV | SVSY | AS | SYPPTF | MHWVRQAPGQGLEWMGWIDPDSGGTNYAEKFQ | QQKPGKAPKLLIYRTSNLASGVPSRFSGSGSGTDFT |
| D04 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB02_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFAN | GWIIPN | CARG | SARS | RTSSLQ | CQQYH | QVQLVQSGAEVKKPGASVKVSCKASGDTFANYY | DIQMTQSPSSLSASVGDRVTITCSARSSVSYMYWY |
| DB02_ | YYMH | SGDTN | YDV | SVSY | S | SYPPTF | MHWVRQAPGQGLEWMGWIIPNSGDTNYAQKFQ | QQKPGKAPKLLIYRTSSLQSGVPSRFSGSGSGTDFT |
| D06 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKD | GWIDH | CARG | SASS | RTSNL | CQQYH | QVQLVQSGAEVKKPGASVKVSCKASGENFKDYY | DIQMTQSPSSLSASVGDRVTITCSASSSVSYMYWY |
| DB02_ | YYMH | SGGSTI | YDV | SVSY | AS | SYPPTF | MHWVRQAPGQGLEWMGWIDHSGGSTIYDQKFQ | QQKPGKAPKLLIYRTSNLASGVPSRFSGSGSGTDFT |
| D07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | RARQ | RTSNL | CQQYH | QVQLVQSGAEVKKPGASVKVSCKASGYTFTNYY | DIQMTQSPSSLSASVGDRVTITCRARQSVSYMYWY |
| DB02_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFINNYI | DIQMTQSPSSLSASVGDRVTITCSARSSVSYMYWY |
| DB02_ | NYIH | ENGNTI | FDVD | SVSY | AS | SYPPTF | HWVRQAPGQGLEWMGWIDHENGNTIYDQKFQG | QQKPGKAPKLLIYRTSNLASGVPSRFSGSGSGTDFT |
| D09 | | YD | WFV | MY | | | RVTMTRDTSTSTVYMELSSLRSEDTAVYYCARGF | LTISSLQPEDFATYYCQQYHSYPPTFGGGTKVEIKR |
| | | | YW | | | | DVDWFVYWGQGTLVTVSS | |
| suPAR_ | YTFTG | GWIDY | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB02_ | YYMH | EGGNTI | YDV | SVSY | AS | SYPPTF | MHWVRQAPGQGLEWMGWIDPEGGNTIYDQKFQ | QQKPGKAPKLLIYRTSNLASGVPSRFSGSGSGTDFT |
| D10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB02_ | YYIH | ENGNTI | YDV | SVSY | AS | SYPPTF | HWVRQAPGQGLEWMGWIDPENGNTIYDQKFQG | QQKPGKAPKLLIYRTSNLASGVPSRFSGSGSGTDFT |
| D11 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFSN | GWIDH | CARG | SARS | RTSNL | CQQSH | QVQLVQSGAEVKKPGASVKVSCKASGYTFSNYY | DIQMTQSPSSLSASVGDRVTITCSARSSVSYMYWY |
| DB02_ | YYMH | EGGSTI | YDV | SVSY | AS | SYPPTF | MHWVRQAPGQGLEWMGWIDHEGGSTIYAQKFQ | QQKPGKAPKLLIYRTSNLASGVPSRFSGSGSGTDFT |
| D12 | | YA | DWF | MY | | | GRVTMTRDTSTSTVYMELSSLRSEDTAVYYCARG | LTISSLQPEDFATYYCQQSHSYPPTFGGGTKVEIKR |
| | | | VYW | | | | YDVDWFVYWGQGTLVTVSS | |
| suPAR_ | FNIKD | GWIDH | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB02_ | YYIH | SNGNTI | YDV | SVSY | AS | SYPPTF | HWVRQAPGQGLEWMGWIDHSNGNTIYAQKFQG | QQKPGKAPKLLIYRTSNLASGVPSRFSGSGSGTDFT |
| E03 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTD | GWIDH | CARG | SARS | RTSSLQ | CQQYH | QVQLVQSGAEVKKPGASVKVSCKASGYTFTDYY | DIQMTQSPSSLSASVGDRVTITCSARSSVSYMYWY |
| DB02_ | YYMH | ENGNTI | YDV | SVSY | S | SYPPTF | MHWVRQAPGQGLEWMGWIDHENGNTIYDQKFQ | QQKPGKAPKLLIYRTSSLQSGVPSRFSGSGSGTDFT |
| E02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNEKD | GWISP | CARG | SARS | RTSNL | | QVQLVQSGAEVKKPGASVKVSCKASGENFKDYYI | |
| DB02_ | YYIH | NZGAT | YDV | SVSY | AS | | HWVRQAPGQGLEWMGWISPNZGATNYAQRFQG | |
| E03 | | NYA | DWF | MY | | | RVTMTRDTSTSTVYMELSSLRSEDTAVYYCARGY | |
| | | | VYW | | | | DVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYYI | DIQMTQSPSSLSASVGDRVTITCSARSSVSYMYWY |
| DB02_ | YYIQ | ENGNTI | YDV | SVSY | AS | SYPPTF | QWVRQAPGQGLEWMGWIDHENGNTIYDQKFQG | QQKPGKAPKLLIYRTSNLASGVPSRFSGSGSGTDFT |
| E04 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | FNIKD | GWVDP | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB02_ | YYIH | SSGAT | YDV | SVSY | AS | SYPPTF | HWVRQAPGQGLEWMGWVDPSSGATDYAQKFQG | QQKPGKAPKLLIYRTSNLASGVPSRFSGSGSGTDFT |
| E05 | | DYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWID | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB02_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| E06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNFN | GWIDY | CARG | SARS | RTSNL | CQQYH | QVQLVQSGAEVKKPGASVKVSCKASGYNFNAYY | DIQMTQSPSSLSASVGDRVTITCSARSSVSYMYWY |
| DB02_ | AYYM | HSGGT | YDV | SVSY | AS | SYPPTF | MHWVRQAPGQGLEWMGWIDPHSGGTNYAQKFQ | QQKPGKAPKLLIYRTSNLASGVPSRFSGSGSGTDFT |
| E07 | H | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNIKS | GWIDH | CAA | SARS | RTSNL | CQQYH | QVQLVQSGAEVKKPGASVKVSCKASGYNIKSYYI | DIQMTQSPSSLSASVGDRVTITCSARSSVSYMYWY |
| DB02_ | YYIH | ENQNTI | GYD | SVSY | AS | SYPPTF | HWVRQAPGQGLEWMGWIDHENGNTIYDQKFQG | QQKPGKAPKLLIYRTSNLASGVPSRFSGSGSGTDFT |
| E08 | | YD | VDW | MY | | | RVTMTRDTSTSTVYMELSSLRSEDTAVYYCAAGY | LTISSLQPEDFATYYCQQYHSYPPTFGGGTKVEIKR |
| | | | FVY | | | | DVDWFVYWGQGTLVTVSS | |
| | | | W | | | | | |
| suPAR_ | FNIKD | GWIDP | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB02_ | YYIH | NSGGT | YDV | SVSY | AS | SYPPTF | HWVRQAPGQGLEWMGWIDPNSGGTNYAQKFQG | QQKPGKAPKLLIYRTSNLASGVPSRFSGSGSGTDFT |
| E09 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYF | DIQMTQSPSSLSASVGDRVTITCSARSSVSYMYWY |
| DB02_ | YFMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFSN | GWIDH | CARG | RARQ | RTSSLA | CQQYH | QVQLVQSGAEVKKPGASVKVSCKASGGTFSNYY | DIQMTQSPSSLSASVGDRVTITCRARQSVSYMYWY |
| DB02_ | YYMH | ENGNTI | YDV | SVSY | S | SYPPTF | MHWVRQAPGQGLEWMGWIDHENGNTIYDQKFQ | QQKPGKAPKLLIYRTSSLASGVPSRFSGSGSGTDFT |
| E11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SASQ | RTSNL | CQQYH | QVQLVQSGAEVKKPGASVKVSCKASGYTFTSNY | DIQMTQSPSSLSASVGDRVTITCSASQSVSYMYWY |
| DB02_ | NYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNITD | GWID | CARG | SARS | RTSNL | CQQYH | QVQLVQSGAEVKKPGASVKVSCKASGFNITDYYI | DIQMTQSPSSLSASVGDRVTITCSARSSVSYMYWY |
| DB02_ | YYIH | SNGNTI | YDV | SVSY | AS | SYPPTF | HWVRQAPGQGLEWMGWIDPSNGNTIYDQKFQGR | QQKPGKAPKLLIYRTSNLASGVPSRFSGSGSGTDFT |
| F01 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFTD | GWIDP | CARG | SARS | RTSNL | CQQYH | QVQLVQSGAEVKKPGASVKVSCKASGYTFTDYY | DIQMTQSPSSLSASVGDRVTITCSARSSVSYMYWY |
| DB02_ | YYMH | ENGNTI | YDV | SVSY | QS | SYPPTF | MHWVRQAPGQGLEWMGWIDPENGNTIYDQKFQ | QQKPGKAPKLLIYRTSNLQSGVPSRFSGSGSGTDFT |
| F02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SARS | RTSSLQ | CQQYH | QVQLVQSGAEVKKPGASVKVSCKASGYTFTSYY | DIQMTQSPSSLSASVGDRVTITCSARSSVSYMYWY |
| DB02_ | YYVH | EGGSTI | YDV | SVSY | S | SYPPTF | VHWVRQAPGQGLEWMGWIDPEGGSTIYDQKFQG | QQKPGKAPKLLIYRTSSLQSGVPSRFSGSGSGTDFT |
| F03 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTLTS | GWIDP | CARG | SARS | RTSNL | CQQYH | QVQLVQSGAEVKKPGASVKVSCKASGYTLTSYY | DIQMTQSPSSLSASVGDRVTITCSARSSVSYMYWY |
| DB02_ | YYVH | KSGDT | YDV | SVSY | AS | SYPPTF | VHWVRQAPGQGLEWMGWIDPKSGDTKYAHKFQ | QQKPGKAPKLLIYRTSNLASGVPSRFSGSGSGTDFT |
| F04 | | KYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGNY | DIQMTQSPSSLSASVGDRVTITCSARSSVSYMYWY |
| DB02_ | NYMQ | ENQNTI | YDV | SVSY | AS | SYPPTF | MQWVRQAPGQGLEWMGWIDHENGNTIYDQKFQ | QQKPGKAPKLLIYRTSNLASGVPSRFSGSGSGTDFT |
| F05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFTD | GWIDP | CARG | SARS | RTSNL | CQQYH | QVQLVQSGAEVKKPGASVKVSCKASGFNFTDYYI | DIQMTQSPSSLSASVGDRVTITCSARSSVSYMYWY |
| DB02_ | YYIH | NSGST | YMV | SVSY | AS | SYPPTF | HWVRQAPGQGLEWMGWIDPNSGSTNYAQNFQG | QQKPGKAPKLLIYRTSNLASGVPSRFSGSGSGTDFT |
| F06 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | MVDWFVYWGQGTLVTVSS | |
| suPAR_ | YTFTG | GWIDP | CARG | RARQ | RTSNL | CQQYH | QVQLVQSGAEVKKPGASVKVSCKASGYTFTGYY | DIQMTQSPSSLSASVGDRVTITCRARQSVSYMYWY |
| DB02_ | YYVH | EGGNTI | YDV | SVSY | QS | SYPPTF | VHWVRQAPGQGLEWMGWIDPEGGNTIYDQKFQ | QQKPGKAPKLLIYRTSNLQSGVPSRFSGSGSGTDFT |
| F07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| SUPAR | YTFTG | GWIDP | CARG | SASS | RTSNL | CQQYH | QVQLVQSGAEVKKPGASVKVSCKASGYTFTGYYI | DIQMTQSPSSLSASVGDRVTITCSASSSISYMYWYQ |
| DB02_ | YYIH | ENGNTI | YDV | SISY | AS | SYPPTF | HWVRQAPGQGLEWMGWIDPENGNTIYAQKFQG | QKPGKAPKLLIYRTSNLASGVPSRFSGSGSGTDFT |
| F08 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDP | CAG | SARS | RTSNL | CQQYH | QVQLVQSGAEVKKPGASVKVSCKASGYTFTGYY | DIQMTQSPSSLSASVGDRVTITCSARSSVSYMYWY |
| DB02_ | YYMH | EGGSTI | GYD | SVSY | AS | SYPPTF | MHWVRQAPGQGLEWMGWIDPEGGSTIYDQKFQ | QQKPGKAPKLLIYRTSNLASGVPSRFSGSGSGTDFT |
| F09 | | YD | VDW | MY | | | GRVTMTRDTSTSTVYMELSSLRSEDTAVYYCAGG | LTISSLQPEDFATYYCQQYHSYPPTFGGGTKVEIKR |
| | | | FVY | | | | YDVDWFVYWGQGTLVTVSS | |
| | | | W | | | | | |
| suPAR_ | YTFTT | GWIDP | CARG | SARS | RTSNL | CQQYH | QVQLVQSGAEVKKPGASVKVSCKASGYTFTTYY | DIQMTQSPSSLSASVGDRVTITCSARSSVSYMYWY |
| DB02_ | YYMH | EGGNT | YDV | SVSY | AS | SYPPTF | MHWVRQAPGQGLEWMGWIDPEGGNTSYDQKFQ | QQKPGKAPKLLIYRTSNLASGVPSRFSGSGSGTDFT |
| F10 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RARS | RTSNL | CQQYH | QVQLVQSGAEVKKPGASVKVSCKASGYTFTSSY | DIQMTQSPSSLSASVGDRVTITCRARSSISYMYWYQ |
| DB02_ | SYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| F11 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | DMFT | GWIDH | CARG | SARS | RTSSLA | CQQYH | QVQLVQSGAEVKKPGASVKVSCKASGDMFTGYY | DIQMTQSPSSLSASVGDRVTITCSARSSVSYMYWY |
| DB02_ | GYYL | ENGNTI | YDV | SVSY | S | SYPPTF | LHWVRQAPGQGLEWMGWIDHENGNTIYDQKFQ | QQKPGKAPKLLIYRTSSLASGVPSRFSGSGSGTDFT |
| F12 | H | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTFTD | GWISP | CARG | SARS | RTSNL | CQQYH | QVQLVQSGAEVKKPGASVKVSCKASGHTFTDYY | DIQMTQSPSSLSASVGDRVTITCSARSSVSYMYWY |
| DB02_ | YYMH | NSGGT | YDV | SVSY | AS | SYPPTF | MHWVRQAPGQGLEWMGWISPNSGGTNYAQKFQ | QQKPGKAPKLLIYRTSNLASGVPSRFSGSGSGTDFT |
| G01 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTR | GWIDH | CARG | SARS | RTSSLQ | CQQYH | QVQLVQSGAEVKKPGASVKVSCKASGYTFTRYY | DIQMTQSPSSLSASVGDRVTITCSARSSVSYMYWY |
| DB02_ | YYMH | ENQSTI | YDV | SVSY | S | SYPPTF | MHWVRQAPGQGLEWMGWIDHENGSTIYAQKFQ | QQKPGKAPKLLIYRTSSLQSGVPSRFSGSGSGTDFT |
| G02 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SASQ | RTSNL | CQQYH | QVQLVQSGAEVKKPGASVKVSCKASGYTFTSYY | DIQMTQSPSSLSASVGDRVTITCSASQSVSYMYWY |
| DB02_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| G03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTFTG | GWIDH | CARG | SARS | RTSNL | CQQYH | QVQLVQSGAEVKKPGASVKVSCKASGHTFTGYYI | DIQMTQSPSSLSASVGDRVTITCSARSSVSYMYWY |
| DB02_ | YYIH | ENQNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| G04 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDP | CARG | SASS | RTSNL | CQQYH | QVQLVQSGAEVKKPGASVKVSCKASGYTFTGHY | DIQMTQSPSSLSASVGDRVTITCSASSSVSYMYWY |
| DB02_ | HYMH | EGGNTI | YDV | SVSY | AS | SYPPTF | MHWVRQAPGQGLEWMGWIDPEGGNTIYAQKFQ | QQKPGKAPKLLIYRTSNLASGVPSRFSGSGSGTDFT. |
| G05 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSISYMYWYQ |
| DB02_ | YYMH | EGGSTI | YDV | SISY | AS | SYPPTF | MHWVRQAPGQGLEWMGWIDHEGGSTIYAQKFQ | QKPGKAPKLLIYRTSNLASGVPSRFSGSGSGTDFTL |
| G06 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SASQ | RTSNL | CQQYH | QVQLVQSGAEVKKPGASVKVSCKASGYTFTSYY | DIQMTQSPSSLSASVGDRVTITCSASQSVSYMYWY |
| DB02_ | YYMH | ENQSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| G07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTS | GWIDH | CARG | SARS | RTSNL | CQQYH | QVQLVQSGAEVKKPGASVKVSCKASGDTFTSYY | DIQMTQSPSSLSASVGDRVTITCSARSSVSYMYWY |
| DB02_ | YYMH | EGGNT | YDV | SVSY | AS | SYPPTF | MHWVRQAPGQGLEWMGWIDHEGGNTSYEQKFQ | QQKPGKAPKLLIYRTSNLASGVPSRFSGSGSGTDFT |
| G08 | | SYE | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FTIKD | GWIDP | CARG | RASS | RTSNL | CQQYH | QVQLVQSGAEVKKPGASVKVSCKASGFTIKDYY | DIQMTQSPSSLSASVGDRVTITCRASSSVSYMYWY |
| DB02_ | YYMH | SNGAT | YDV | SVSY | AS | SYPPTF | MHWVRQAPGQGLEWMGWIDPSNGATNSAQNFQ | QQKPGKAPKLLIYRTSNLASGVPSRFSGSGSGTDFT |
| G09 | | NSA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWID | CARG | RARQ | RTSNLE | CQQYH | QVQLVQSGAEVKKPGASVKVSCKASGYTFTNYY | DIQMTQSPSSLSASVGDRVTITCRARQSVSYMYWY |
| DB02_ | YYMH | ENGSTI | YDV | SVSY | T | SYPPTF | MHWVRQAPGQGLEWMGWIDPENGSTIYDQKFQ | QQKPGKAPKLLIYRTSNLETGVPSRFSGSGSGTDFT |
| G10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTG | GWIDH | CARG | SARS | RTSNL | CQQYH | QVQLVQSGAEVKKPGASVKVSCKASGDTFTGYY | DIQMTQSPSSLSASVGDRVTITCSARSSVSYMYWY |
| DB02_ | YYMH | EGGNTI | YDV | SVSY | AS | SYPPTF | MHWVRQAPGQGLEWMGWIDHEGGNTIYAQKFQ | QQKPGKAPKLLIYRTSNLASGVPSRFSGSGSGTDFT |
| G11 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDY | CARG | SASS | RTSSLQ | CQQYH | QVQLVQSGAEVKKPGASVKVSCKASGFNIKDYYI | DIQMTQSPSSLSASVGDRVTITCSASSSVSYMYWY |
| DB02_ | YYIH | ENQSTI | YDV | SVSY | S | SYPPTF | HWVRQAPGQGLEWMGWIDPENGSTIYDQKFQGR | QQKPGKAPKLLIYRTSSLQSGVPSRFSGSGSGTDFT |
| G12 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFTG | GWIDH | CAG | SARS | RTSSLA | CQQYH | QVQLVQSGAEVKKPGASVKVSCKASGYTFTGQYI | DIQMTQSPSSLSASVGDRVTITCSARSSVSYMYWY |
| DB02_ | QYIH | SGGSTI | GYD | SVSY | S | SYPPTF | HWVRQAPGQGLEWMGWIDHSGGSTIYDQKFQGR | QQKPGKAPKLLIYRTSSLASGVPSRFSGSGSGTDFT |
| H01 | | YD | VDW | MY | | | VTMTRDTSTSTVYMELSSLRSEDTAVYYCAGGYD | LTISSLQPEDFATYYCQQYHSYPPTFGGGTKVEIKR |
| | | | FVY | | | | VDWFVYWGQGTLVTVSS | |
| | | | W | | | | | |
| suPAR_ | YTFTG | GWIDP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB02_ | YYLH | ENGSTI | YDV | SVSY | AS | SYPPTF | LHWVRQAPGQGLEWMGWIDPENGSTIYDQKFQG | QQKPGKAPKLLIYRTSNLASGVPSRFSGSGSGTDFT |
| H02 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTT | GWIDP | CARG | SARS | RTSNL | CQQYH | QVQLVQSGAEVKKPGASVKVSCKASGYTFTTYY | DIQMTQSPSSLSASVGDRVTITCSARSSVSYMYWY |
| DB02_ | YYMH | EGGSTI | YDV | SVSY | AS | SYPPTF | MHWVRQAPGQGLEWMGWIDPEGGSTIYDQKFQ | QQKPGKAPKLLIYRTSNLASGVPSRFSGSGSGTDFT |
| H03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKS | GWIDP | CARG | SARS | RTSNL | CQQYH | QVQLVQSGAEVKKPGASVKVSCKASGENFKSYYI | DIQMTQSPSSLSASVGDRVTITCSARSSVSYMYWY |
| DB02_ | YYIH | SSGVM | YDV | SVSY | AS | SYPPTF | HWVRQAPGQGLEWMGWIDPSSGVMKNAQKFQG | QQKPGKAPKLLIYRTSNLASGVPSRFSGSGSGTDFT |
| H04 | | KNA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YFFTA | GWIDP | CARG | SARS | RTSNL | CQQYH | QVQLVQSGAEVKKPGASVKVSCKASGYFFTAYY | DIQMTQSPSSLSASVGDRVTITCSARSSVSYMYWY |
| DB02_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| H05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | RARS | RTSNL | CQQYH | QVQLVQSGAEVKKPGASVKVSCKASGFNIKDYYI | DIQMTQSPSSLSASVGDRVTITCRARSSVSYMYWY |
| DB02_ | YYIH | EGGNTI | YDV | SVSY | AS | SYPPTF | HWVRQAPGQGLEWMGWIDPEGGNTIYAQKFQG | QQKPGKAPKLLIYRTSNLASGVPSRFSGSGSGTDFT |
| H06 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWVDP | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB02_ | YYVH | SSGAT | YDV | SVSY | AS | SYPPTF | VHWVRQAPGQGLEWMGWVDPSSGATDYAQKFQ | QQKPGKAPKLLIYRTSNLASGVPSRFSGSGSGTDFT |
| H07 | | DYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDP | CARG | SARS | RTSNL | CQQYH | QVQLVQSGAEVKKPGASVKVSCKASGYTFTGYYI | DIQMTQSPSSLSASVGDRVTITCSARSSVSYMYWY |
| DB02_ | YYIH | ENGSTI | YDV | SVSY | AS | SYPPTF | HWVRQAPGQGLEWMGWIDPENGSTIYDQKFQGR | QQKPGKAPKLLIYRTSNLASGVPSRFSGSGSGTDFT |
| H08 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFTS | GWIDP | CARG | SARS | ATSNL | CQQYH | QVQLVQSGAEVKKPGASVKVSCKASGYTFTSYY | DIQMTQSPSSLSASVGDRVTITCSARSSVSYMYWY |
| DB02_ | YYMH | ENGNTI | YDV | SVSY | QS | SYPPTF | MHWVRQAPGQGLEWMGWIDPENGNTIYAQKFQ | QQKPGKAPKLLIYATSNLQSGVPSRFSGSGSGTDFT |
| H09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VFW | | | | YDVDWFVFWGQGTLVTVSS | |
| suPAR_ | YTFTS | GWIDH | CARG | SARQ | RTSNL | CQQYH | QVQLVQSGAEVKKPGASVKVSCKASGYTFTSYY | DIQMTQSPSSLSASVGDRVTITCSARQSVSYMYWY |
| DB02_ | YYMH | SNQNTI | YDV | SVSY | AS | SYPPTF | MHWVRQAPGQGLEWMGWIDHSNGNTIYDQKFQ | QQKPGKAPKLLIYRTSNLASGVPSRFSGSGSGTDFT |
| H10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CAV | SARS | RTSNL | CQQYH | QVQLVQSGAEVKKPGASVKVSCKASGYTFTGYY | DIQMTQSPSSLSASVGDRVTITCSARSSVSYMYWY |
| DB03_ | YYMH | ENQSTI | GYD | SVSY | AS | SYPPTF | MHWVRQAPGQGLEWMGWIDHENGSTIYDQKFQ | QQKPGKAPKLLIYRTSNLASGVPSRFSGSGSGTDFT |
| A03 | | YD | VDW | MY | | | GRVTMTRDTSTSTVYMELSSLRSEDTAVYYCAVG | LTISSLQPEDFATYYCQQYHSYPPTFGGGTKVEIKR |
| | | | FVY | | | | YDVDWFVYWGQGTLVTVSS | |
| | | | W | | | | | |
| suPAR_ | DTFSG | GWIDP | CARG | SARS | RTSNL | CQQYH | QVQLVQSGAEVKKPGASVKVSCKASGDTESGYY | DIQMTQSPSSLSASVGDRVTITCSARSSVSYMYWY |
| DB03_ | YYMH | EGGNT | YDV | SVSY | AS | SYPPTF | MHWVRQAPGQGLEWMGWIDPEGGNTSYDQKFQ | QQKPGKAPKLLIYRTSNLASGVPSRFSGSGSGTDFT |
| A04 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSS | GWIDH | CARG | SARS | RTSSLA | CQQYH | QVQLVQSGAEVKKPGASVKVSCKASGYTFSSYY | DIQMTQSPSSLSASVGDRVTITCSARSSVSYMYWY |
| DB03_ | YYMH | ENGNTI | YDV | SVSY | S | SYPPTF | MHWVRQAPGQGLEWMGWIDHENGNTIYAQKFQ | QQKPGKAPKLLIYRTSSLASGVPSRFSGSGSGTDFT |
| A05 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNITD | GWID | CARG | SARQ | RTSNL | CQQYH | QVQLVQSGAEVKKPGASVKVSCKASGFNITDYYI | DIQMTQSPSSLSASVGDRVTITCSARQSISYMYWYQ |
| DB03_ | YYIH | ENGSTI | YDV | SISY | AS | SYPPTF | HWVRQAPGQGLEWMGWIDPENGSTIYAQKFQGR | QKPGKAPKLLIYRTSNLASGVPSRFSGSGSGTDFTL |
| A06 | | YA | DWF | MY | | | VTMTRDTSTSTVYMELSSLRSEDTAVYYCARGYD | TISSLQPEDFATYYCQQYHSYPPTFGGGTKVEIKR |
| | | | VYW | | | | VDWFVYWGQGTLVTVSS | |
| suPAR_ | YTFTD | GWIDH | CARG | SARQ | RTSNL | CQQYH | QVQLVQSGAEVKKPGASVKVSCKASGYTFTDYY | DIQMTQSPSSLSASVGDRVTITCSARQSISYMYWYC |
| DB03_ | YYMH | ENGNTI | YDV | SISY | AS | SYPPTF | MHWVRQAPGQGLEWMGWIDHENGNTIYAQKFQ | QKPGKAPKLLIYRTSNLASGVPSRFSGSGSGTDFTL |
| A07 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SASS | RTSNL | CQQYH | QVQLVQSGAEVKKPGASVKVSCKASGYTFTGYY | DIQMTQSPSSLSASVGDRVTITCSASSSVSYMYWY |
| DB03_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| A08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARQ | RTSNL | CQQYH | QVQLVQSGAEVKKPGASVKVSCKASGYTFTSHY | DIQMTQSPSSLSASVGDRVTITCSARQSISYMYWYQ |
| DB03_ | HYVH | SNGSTI | YDV | SISY | AS | SYPPTF | VHWVRQAPGQGLEWMGWIDHSNGSTIYDQKFQ | QKPGKAPKLLIYRTSNLASGVPSRFSGSGSGTDFTL |
| A09 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFSN | GWIDH | CARG | SARS | RTSNL | CQQYH | QVQLVQSGAEVKKPGASVKVSCKASGYTFSNYY | DIQMTQSPSSLSASVGDRVTITCSARSSVSYMYWY |
| DB03_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| A10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWMD | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB03_ | YYMH | PNSGG | YDV | SVSY | AS | SYPPTF | MHWVRQAPGQGLEWVGWMDPNSGGTSYAQKF | QQKPGKAPKLLIYRTSNLASGVPSRFSGSGSGTDFT |
| A11 | | TSYA | DWF | MY | | | QGRVTMTRDTSTSTVYMELSSLRSEDTAVYYCAR | LTISSLQPEDFATYYCQQYHSYPPTFGGGTKVEIKR |
| | | | VYW | | | | GYDVDWFVYWGQGTLVTVSS | |
| suPAR_ | YTFSR | GWMN | CARG | RASQ | DASSL | CQQSY | QVQLVQSGAEVKKPGASVKVSCKASGYTFSRYY | DIQMTQSPSSLSASVGDRVTITCRASQGISGWLAW |
| DB03_ | YYMH | PNTGD | YDV | GISG | QS | SDPLTF | MHWVRQAPGQGLEWIGWMNPNTGDTGYAQKFQ | YQQKPGKAPKLLIYDASSLQSGVPSRFSGSGSGTDF |
| A12 | | TGYA | DFFV | WLA | | | GRVTMTRDTSTSTVYMELSSLRSEDTAVYYCARG | TLTISSLQPEDFATYYCQQSYSDPLTFGGGTKVEIK |
| | | | YW | | | | YDVDFFVYWGQGTLVTVSS | R |
| suPAR_ | NTFTG | GWIDH | CARG | SARS | RTSNL | CQQYH | QVQLVQSGAEVKKPGASVKVSCKASGNTFTGHY | DIQMTQSPSSLSASVGDRVTITCSARSSVSYMYWY |
| DB03_ | HYMQ | ENGNT | YDV | SVSY | AS | SYPPTF | MQWVRQAPGQGLEWMGWIDHENGNTSYAQKFQ | QQKPGKAPKLLIYRTSNLASGVPSRFSGSGSGTDFT |
| B01 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| SUPAR | DTFTS | GWIDH | CARG | SARS | RTSNL | CQQYH | QVQLVQSGAEVKKPGASVKVSCKASGDTFTSYY | DIQMTQSPSSLSASVGDRVTITCSARSSVSYMYWY |
| DB03_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| B02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIQP | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB03_ | YYMH | ENQSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| B03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWITP | CARG | SASS | RTSNL | CQQYH | QVQLVQSGAEVKKPGASVKVSCKASGYTFTGYY | DIQMTQSPSSLSASVGDRVTITCSASSSISYMYWYQ |
| DB03_ | YYMH | DSGGT | YDV | SISY | AS | SYPPTF | MHWVRQAPGQGLEWIGWITPDSGGTVYAQKFQG | QKPGKAPKLLIYRTSNLASGVPSRFSGSGSGTDFTL |
| B04 | | VYA | DWF | MY | | | RVTMTRDTSTSTVYMELSSLRSEDTAVYYCARGY | TISSLQPEDFATYYCQQYHSYPPTFGGGTKVEIKR |
| | | | VYW | | | | DVDWFVYWGQGTLVTVSS | |
| suPAR_ | YTFTS | GWIDP | CARG | SARQ | RTSNL | CQQYH | QVQLVQSGAEVKKPGASVKVSCKASGYTFTSYY | DIQMTQSPSSLSASVGDRVTITCSARQSVSYMYWY |
| DB03_ | YYMH | ENQNTI | YDID | SVSY | AS | SYPPTF | MHWVRQAPGQGLEWMGWIDPENGNTIYDQKFQ | QQKPGKAPKLLIYRTSNLASGVPSRFSGSGSGTDFT |
| B05 | | YD | WFV | MY | | | GRVTMTRDTSTSTVYMELSSLRSEDTAVYYCARG | LTISSLQPEDFATYYCQQYHSYPPTFGGGTKVEIKR |
| | | | YW | | | | YDIDWFVYWGQGTLVTVSS | |
| suPAR_ | YNITD | GWIDP | CARG | SARS | RTSNL | CQQYH | QVQLVQSGAEVKKPGASVKVSCKASGYNITDYYI | DIQMTQSPSSLSASVGDRVTITCSARSSVSYMYWY |
| DB03_ | YYIH | ENGSTI | YDV | SVSY | AS | SYPPTF | HWVRQAPGQGLEWMGWIDPENGSTIYDQKFQGR | QQKPGKAPKLLIYRTSNLASGVPSRFSGSGSGTDFT |
| B06 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | FNIKD | GWMD | CARQ | SARS | RTSNL | CQQYH | QVQLVQSGAEVKKPGASVKVSCKASGFNIKDYYI | DIQMTQSPSSLSASVGDRVTITCSARSSVSYMYWY |
| DB03_ | YYIH | PSSGNT | YDV | SVSY | AS | SYPPTF | HWVRQAPGQGLEWMGWMDPSSGNTAYAQKFQ | QQKPGKAPKLLIYRTSNLASGVPSRFSGSGSGTDFT |
| B07 | | AYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YAFTG | GWIDH | CARG | RARQ | RTSNL | CQQYH | QVQLVQSGAEVKKPGASVKVSCKASGYAFTGYY | DIQMTQSPSSLSASVGDRVTITCRARQSVSYMYWY |
| DB03_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| B08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNIKS | GWIDP | CARG | SARS | RTSNL | CQQYH | QVQLVQSGAEVKKPGASVKVSCKASGYNIKSYYI | DIQMTQSPSSLSASVGDRVTITCSARSSVSYMYWY |
| DB03_ | YYIH | EGGSTI | YDV | SVSY | AS | SYPPTF | HWVRQAPGQGLEWMGWIDPEGGSTIYDQKFQGR | QQKPGKAPKLLIYRTSNLASGVPSRFSGSGSGTDFT |
| B09 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFTG | GWIDH | CARG | SASS | RTSNL | CQQYH | QVQLVQSGAEVKKPGASVKVSCKASGYTFTGYY | DIQMTQSPSSLSASVGDRVTITCSASSSISYMYWYQ |
| DB03_ | YYMH | ENQNTI | TDVD | SISY | AS | SYPPTF | MHWVRQAPGQGLEWMGWIDHENGNTIYDQKFQ | QKPGKAPKLLIYRTSNLASGVPSRFSGSGSGTDFTL |
| B10 | | YD | WFV | MY | | | GRVTMTRDTSTSTVYMELSSLRSEDTAVYYCARG | TISSLQPEDFATYYCQQYHSYPPTFGGGTKVEIKR |
| | | | YW | | | | TDVDWFVYWGQGTLVTVSS | |
| suPAR_ | YTENN | GWIDH | CARG | RARS | RTSNL | CQQYH | QVQLVQSGAEVKKPGASVKVSCKASGYTENNYY | DIQMTQSPSSLSASVGDRVTITCRARSSVSYMYWY |
| DB03_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| B11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFIN | GWIDS | CARG | SARS | RTSNL | CQQYH | QVQLVQSGAEVKKPGASVKVSCKASGDTFINYY | DIQMTQSPSSLSASVGDRVTITCSARSSVSYMYWY |
| DB03_ | YYMH | HSGVT | YDV | SVSY | AS | SYPPTF | MHWVRQAPGQGLEWMGWIDSHSGVINYGQEFQ | QQKPGKAPKLLIYRTSNLASGVPSRFSGSGSGTDFT |
| B12 | | NYG | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | STSTD | GWIDH | CARG | SARS | RTSNL | CQQYH | QVQLVQSGAEVKKPGASVKVSCKASGSTSTDHY | DIQMTQSPSSLSASVGDRVTITCSARSSVSYMYWY |
| DB03_ | HYVH | EGGSTI | YDV | SVSY | AS | SYPPTF | VHWVRQAPGQGLEWMGWIDHEGGSTIYAQKFQ | QQKPGKAPKLLIYRTSNLASGVPSRFSGSGSGTDFT |
| C01 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTN | GWIDH | CARG | SARS | RTSNL | CQQYH | QVQLVQSGAEVKKPGASVKVSCKASGYSFTNYYI | DIQMTQSPSSLSASVGDRVTITCSARSSVSYMYWY |
| DB03_ | YYIH | ENGNTI | NDV | SVSY | AS | SYPPTF | HWVRQAPGQGLEWMGWIDHENGNTIYDQKFQG | QQKPGKAPKLLIYRTSNLASGVPSRFSGSGSGTDFT |
| C02 | | YD | DWF | MY | | | RVTMTRDTSTSTVYMELSSLRSEDTAVYYCARGN | LTISSLQPEDFATYYCQQYHSYPPTFGGGTKVEIKR |
| | | | VYW | | | | DVDWFVYWGQGTLVTVSS | |
| suPAR_ | YTFTG | GWIDH | CASG | SARS | GASNL | CQQYH | QVQLVQSGAEVKKPGASVKVSCKASGYTFTGYY | DIQMTQSPSSLSASVGDRVTITCSARSSVSYMYWY |
| DB03_ | YYMH | ENGSTS | YDV | SVSY | QS | SYPPTF | MHWVRQAPGQGLEWMGWIDHENGSTSYDQKFQ | QQKPGKAPKLLIYGASNLQSGVPSRFSGSGSGTDFT |
| C03 | | YD | DWF | MY | | | GRVTMTRDTSTSTVYMELSSLRSEDTAVYYCASG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | SASS | RTSSLA | CQQYH | QVQLVQSGAEVKKPGASVKVSCKASGFNIKDYYI | DIQMTQSPSSLSASVGDRVTITCSASSSISYMYWYQ |
| DB03_ | YYIH | ENGSTI | YDV | SISY | S | SYPPTF | HWVRQAPGQGLEWMGWIDPENGSTIYDQKFQGR | QKPGKAPKLLIYRTSSLASGVPSRFSGSGSGTDFT |
| C04 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YAFTT | GWIDP | CARG | SASS | RTSNL | CQQYH | QVQLVQSGAEVKKPGASVKVSCKASGYAFTTYY | DIQMTQSPSSLSASVGDRVTITCSASSSVSYMYWY |
| DB03_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFI |
| C05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SASS | RTSNL | CQQYH | QVQLVQSGAEVKKPGASVKVSCKASGYTFTDYY | DIQMTQSPSSLSASVGDRVTITCSASSSVSYMYWY |
| DB03_ | YYVH | EGGSTS | YDV | SVSY | AS | SYPPTF | VHWVRQAPGQGLEWMGWIDHEGGSTSYDQKFQ | QQKPGKAPKLLIYRTSNLASGVPSRFSGSGSGTDFT |
| C06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB03_ | YYVH | ENGSTI | YDV | SVSY | AS | SYPPTF | VHWVRQAPGQGLEWMGWIDHENGSTIYDQKFQ | QQKPGKAPKLLIYRTSNLASGVPSRFSGSGSGTDFT |
| C07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTN | GWIDH | CARG | SARS | RTSSLA | CQQYH | QVQLVQSGAEVKKPGASVKVSCKASGGTFTNYY | DIQMTQSPSSLSASVGDRVTITCSARSSVSYMYWY |
| DB03_ | YYMH | ENQSTI | YDV | SVSY | S | SYPPTF | MHWVRQAPGQGLEWMGWIDHENGSTIYAQKFQ | QQKPGKAPKLLIYRTSSLASGVPSRFSGSGSGTDFT |
| C08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARQ | RTSNL | CQQYH | QVQLVQSGAEVKKPGASVKVSCKASGYTFTSYY | DIQMTQSPSSLSASVGDRVTITCSARQSVSYMYWY |
| DB03_ | YYMH | ENGSTI | YDV | SVSY | AS | SYPPTF | MHWVRQAPGQGLEWMGWIDHENGSTIYDQKFQ | QQKPGKAPKLLIYRTSNLASGVPSRFSGSGSGTDFT |
| C09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CASG | SARS | RTSNL | CQQYH | QVQLVQSGAEVKKPGASVKVSCKASGYTFTSYY | DIQMTQSPSSLSASVGDRVTITCSARSSVSYMYWY |
| DB03_ | YYMH | EGGSTI | YDV | SVSY | AS | SYPPTF | MHWVRQAPGQGLEWMGWIDHEGGSTIYAQKFQ | QQKPGKAPKLLIYRTSNLASGVPSRFSGSGSGTDFT |
| C10 | | YA | DWF | MY | | | GRVTMTRDTSTSTVYMELSSLRSEDTAVYYCASG | LTISSLQPEDFATYYCQQYHSYPPTFGGGTKVEIKR |
| | | | VYW | | | | YDVDWFVYWGQGTLVTVSS | |
| suPAR_ | DTFTR | GWIDH | CARG | SARS | RTSSLA | CQQYH | QVQLVQSGAEVKKPGASVKVSCKASGDTFTRHY | DIQMTQSPSSLSASVGDRVTITCSARSSVSYMYWY |
| DB03_ | HYVH | ENGNTI | YDV | SVSY | S | SYPPTF | VHWVRQAPGQGLEWMGWIDHENGNTIYDQKFQ | QQKPGKAPKLLIYRTSSLASGVPSRFSGSGSGTDFT |
| C11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | NTFTN | GWIDH | CARG | SARS | RTSNL | CQQYH | QVQLVQSGAEVKKPGASVKVSCKASGNTFTNYY | DIQMTQSPSSLSASVGDRVTITCSARSSVSYMYWY |
| DB03_ | YYMH | ENGNT | YDV | SVSY | AS | SYPPTF | MHWVRQAPGQGLEWMGWIDHENGNTSYDQKFQ | QQKPGKAPKLLIYRTSNLASGVPSRFSGSGSGTDFT |
| C12 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWMD | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB03_ | YYIH | PNSGG | YDV | SVSY | AS | SYPPTF | HWVRQAPGQGLEWMGWMDPNSGGTNYAQKFQ | QQKPGKAPKLLIYRTSNLASGVPSRFSGSGSGTDFT |
| D01 | | TNYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTR | GWIDY | CARG | SARS | RTSNL | CQQYH | QVQLVQSGAEVKKPGASVKVSCKASGYTFTRYY | DIQMTQSPSSLSASVGDRVTITCSARSSVSYMYWY |
| DB03_ | YYLH | ENGNTI | YDV | SVSY | AS | SYPPTF | LHWVRQAPGQGLEWMGWIDPENGNTIYAQKFQG | QQKPGKAPKLLIYRTSNLASGVPSRFSGSGSGTDFT |
| D02 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDP | CARG | RARQ | RTSNL | CQQYH | QVQLVQSGAEVKKPGASVKVSCKASGYTFTGYYI | DIQMTQSPSSLSASVGDRVTITCRARQSVSYMYWY |
| DB03_ | YYIH | ENGNTI | YDV | SVSY | AS | SYPPTF | HWVRQAPGQGLEWMGWIDPENGNTIYAQKFQG | QQKPGKAPKLLIYRTSNLASGVPSRFSGSGSGTDFT |
| D03 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDP | CARG | SARS | RTSNL | CQQSH | QVQLVQSGAEVKKPGASVKVSCKASGYTFTNYY | DIQMTQSPSSLSASVGDRVTITCSARSSVSYMYWY |
| DB03_ | YYMH | ENQSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| D04 | | YD | DWF | MY | | | GRVTMTRDTSTSTVYMELSSLRSEDTAVYYCARG | LTISSLQPEDFATYYCQQSHSYPPTFGGGTKVEIKR |
| | | | VYW | | | | YDVDWFVYWGQGTLVTVSS | |
| suPAR_ | DNFTT | GWIDP | CARG | SARS | RTSNL | CQQYH | QVQLVQSGAEVKKPGASVKVSCKASGDNFTTYY | DIQMTQSPSSLSASVGDRVTITCSARSSVSYMYWY |
| DB03_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| D05 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTN | GWIDH | CARG | SARS | RTSNL | CQQYH | QVQLVQSGAEVKKPGASVKVSCKASGYSFTNYY | DIQMTQSPSSLSASVGDRVTITCSARSSVSYMYWY |
| DB03_ | YYMH | ENGNT | YDV | SVSY | AS | SYPPTF | MHWVRQAPGQGLEWMGWIDHENGNTSYAQKFQ | QQKPGKAPKLLIYRTSNLASGVPSRFSGSGSGTDFT |
| D06 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | NTFTN | GWID | CARG | SARS | RTSNL | CQQYH | QVQLVQSGAEVKKPGASVKVSCKASGNTFTNYY | DIQMTQSPSSLSASVGDRVTITCSARSSVSYMYWY |
| DB03_ | YYVH | SNGNTI | YDV | SVSY | AS | SYPPTF | VHWVRQAPGQGLEWMGWIDPSNGNTIYDQKFQ | QQKPGKAPKLLIYRTSNLASGVPSRFSGSGSGTDFT |
| D07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDP | CARG | SARQ | RTSNL | CQQYH | QVQLVQSGAEVKKPGASVKVSCKASGYTFTGYY | DIQMTQSPSSLSASVGDRVTITCSARQSVSYMYWY |
| DB03_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| D08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB03_ | YYMH | ENGSTI | YDV | SVSY | QS | SYPPTF | MHWVRQAPGQGLEWMGWIDHENGSTIYAQKFQ | QQKPGKAPKLLIYRTSNLQSGVPSRFSGSGSGTDFT |
| D09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB03_ | YYMH | ENGNTI | YDV | SVSY | QS | SYPPTF | MHWVRQAPGQGLEWMGWIDPENGNTIYAQKFQ | QQKPGKAPKLLIYRTSNLQSGVPSRFSGSGSGTDFT |
| D10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YVESG | GWIDP | CARG | SASS | RTSSLQ | CQQYH | QVQLVQSGAEVKKPGASVKVSCKASGYVESGYYI | DIQMTQSPSSLSASVGDRVTITCSASSSVSYMYWY |
| DB03_ | YYIH | EGGSTI | YDV | SVSY | S | SYPPTF | HWVRQAPGQGLEWMGWIDPEGGSTIYDQKFQGR | QQKPGKAPKLLIYRTSSLQSGVPSRESGSGSGTDFT |
| D11 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFSN | GWIDP | CARG | RARS | RTSNL | CQQYH | QVQLVQSGAEVKKPGASVKVSCKASGYTFSNYY | DIQMTQSPSSLSASVGDRVTITCRARSSVSYMYWY |
| DB03_ | YYMH | ENGNTI | YDV | SVSY | QS | SYPPTF | MHWVRQAPGQGLEWMGWIDPENGNTIYAQKFQ | QQKPGKAPKLLIYRTSNLQSGVPSRFSGSGSGTDFT |
| D12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARQ | RTSNL | CQQYH | QVQLVQSGAEVKKPGASVKVSCKASGYTFTNYY | DIQMTQSPSSLSASVGDRVTITCSARQSISYMYWYC |
| DB03_ | YYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| E01 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YNETG | GWIDH | CARG | SARS | RTSNL | CQQYH | QVQLVQSGAEVKKPGASVKVSCKASGYNFTGYYI | DIQMTQSPSSLSASVGDRVTITCSARSSVSYMYWY |
| DB03_ | YYIH | ENGNTI | YDV | SVSY | AS | SYPPTF | HWVRQAPGQGLEWMGWIDHENGNTIYAQKFQG | QQKPGKAPKLLIYRTSNLASGVPSRFSGSGSGTDFT |
| E02 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTVTN | GWIDH | CARG | RARS | RTSNL | CQQYH | QVQLVQSGAEVKKPGASVKVSCKASGYTVTNYY | DIQMTQSPSSLSASVGDRVTITCRARSSVSYMYWY |
| DB03_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDP | CARG | SARS | RTSNL | CQQYH | QVQLVQSGAEVKKPGASVKVSCKASGYTFTGHY | DIQMTQSPSSLSASVGDRVTITCSARSSVSYMYWY |
| DB03_ | HYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| E04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSD | GWIDH | CARG | SARS | RTSNL | CQQYH | QVQLVQSGAEVKKPGASVKVSCKASGYTESDYY | DIQMTQSPSSLSASVGDRVTITCSARSSVSYMYWY |
| DB03_ | YYMH | SNQNTI | YDV | SVSY | AS | SYPPTF | MHWVRQAPGQGLEWMGWIDHSNGNTIYDQKFQ | QQKPGKAPKLLIYRTSNLASGVPSRFSGSGSGTDFT |
| E05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGQY | DIQMTQSPSSLSASVGDRVTITCSARSSVSYMYWY |
| DB03_ | QYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| E06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | NTFTS | GWIDH | CARG | SARQ | RTSNL | CQQYH | QVQLVQSGAEVKKPGASVKVSCKASGNTFTSYY | DIQMTQSPSSLSASVGDRVTITCSARQSISYMYWYQ |
| DB03_ | YYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| E07 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTH | GWIDP | CARG | RASS | RTSNL | CQQYH | QVQLVQSGAEVKKPGASVKVSCKASGYTFTHYY | DIQMTQSPSSLSASVGDRVTITCRASSSVSYMYWY |
| DB03_ | YYMH | EGGSTI | YDV | SVSY | AS | SYPPTF | MHWVRQAPGQGLEWMGWIDPEGGSTIYDQKFQ | QQKPGKAPKLLIYRTSNLASGVPSRFSGSGSGTDFT |
| E08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWID | CARG | RARQ | RTSNL | CQQYH | QVQLVQSGAEVKKPGASVKVSCKASGFNIKDYYI | DIQMTQSPSSLSASVGDRVTITCRARQSVSYMYWY |
| DB03_ | YYIH | NSGGT | YDV | SVSY | AS | SYPPTF | HWVRQAPGQGLEWMGWIDPNSGGTNYAQKFQG | QQKPGKAPKLLIYRTSNLASGVPSRFSGSGSGTDFT |
| E09 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | FNFKS | GWIDH | CARG | SARS | RTSNL | CQQYH | QVQLVQSGAEVKKPGASVKVSCKASGFNFKSYYI | DIQMTQSPSSLSASVGDRVTITCSARSSVSYMYWY |
| DB03_ | YYIH | ENGSTS | YDV | SVSY | AS | SYPPTF | HWVRQAPGQGLEWMGWIDHENGSTSYDQKFQG | QQKPGKAPKLLIYRTSNLASGVPSRFSGSGSGTDFT? |
| E10 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDH | CARG | SASQ | RTSNL | CQQYH | QVQLVQSGAEVKKPGASVKVSCKASGYTFTSYY | DIQMTQSPSSLSASVGDRVTITCSASQSVSYMYWY |
| DB03_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARQ | RTSNL | CQQYH | QVQLVQSGAEVKKPGASVKVSCKASGYTFTGYY | DIQMTQSPSSLSASVGDRVTITCSARQSVSYMYWY |
| DB03_ | YYMH | ENGNTI | NDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | NDVDWFVYWGQGTLVTVSS | |
| suPAR_ | DTFTR | GWIDH | CARG | SARS | RTSNL | CQQYH | QVQLVQSGAEVKKPGASVKVSCKASGDTFTRHY | DIQMTQSPSSLSASVGDRVTITCSARSSVSYMYWY |
| DB03_ | HYVH | ENQNTI | YDV | SVSY | AS | SYPPTF | VHWVRQAPGQGLEWMGWIDHENGNTIYAQKFQ | QQKPGKAPKLLIYRTSNLASGVPSRFSGSGSGTDFT |
| F01 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SASQ | RTSSLA | CQQYH | QVQLVQSGAEVKKPGASVKVSCKASGYTFTNYY | DIQMTQSPSSLSASVGDRVTITCSASQSVSYMYWY |
| DB03_ | YYMH | ENGSTS | YDV | SVSY | S | SYPPTF | MHWVRQAPGQGLEWMGWIDHENGSTSYDQKFQ | QQKPGKAPKLLIYRTSSLASGVPSRFSGSGSGTDFT |
| F02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYYI | DIQMTQSPSSLSASVGDRVTITCSARSSVSYMYWY |
| DB03_ | YYIH | ENGSTS | YDV | SVSY | QS | SYPPTF | HWVRQAPGQGLEWMGWIDHENGSTSYDQKFQG | QQKPGKAPKLLIYRTSNLQSGVPSRFSGSGSGTDFT |
| F03 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTVTS | GWIDP | CARG | SASS | RTSNL | CQQYH | QVQLVQSGAEVKKPGASVKVSCKASGYTVTSYY | DIQMTQSPSSLSASVGDRVTITCSASSSVSYMYWY |
| DB03_ | YYMH | ENQNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| F04 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSG | GWIDH | CARG | SARS | RTSNL | CQQYH | QVQLVQSGAEVKKPGASVKVSCKASGYTFSGYY | DIQMTQSPSSLSASVGDRVTITCSARSSVSYMYWY |
| DB03_ | YYMH | ENGNTI | NDV | SVSY | AS | SYPPTF | MHWVRQAPGQGLEWMGWIDHENGNTIYDQKFQ | QQKPGKAPKLLIYRTSNLASGVPSRFSGSGSGTDFT |
| F05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | NDVDWFVYWGQGTLVTVSS | |
| suPAR_ | FNIKD | GWIDP | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB03_ | YYIH | ENGSTI | YDV | SVSY | AS | SYPPTF | HWVRQAPGQGLEWMGWIDPENGSTIYAQKFQGR | QQKPGKAPKLLIYRTSNLASGVPSRFSGSGSGTDFT |
| F06 | | YA | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | FNITD | GWIDH | CARG | SARQ | RTSNL | CQQYH | QVQLVQSGAEVKKPGASVKVSCKASGFNITDYY | DIQMTQSPSSLSASVGDRVTITCSARQSVSYMYWY |
| DB03_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| F07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTEND | GWIDH | CARG | RASS | RTSNL | CQQYH | QVQLVQSGAEVKKPGASVKVSCKASGYTENDYYI | DIQMTQSPSSLSASVGDRVTITCRASSSVSYMYWY |
| DB03_ | YYIH | EGGNT | TDVD | SVSY | AS | SYPPTF | HWVRQAPGQGLEWMGWIDHEGGNTSYAQKFQG | QQKPGKAPKLLIYRTSNLASGVPSRFSGSGSGTDFT |
| F08 | | SYA | WFV | MY | | | RVTMTRDTSTSTVYMELSSLRSEDTAVYYCARGT | LTISSLQPEDFATYYCQQYHSYPPTFGGGTKVEIKR |
| | | | YW | | | | DVDWFVYWGQGTLVTVSS | |
| suPAR_ | YTFTS | GWIDH | CARG | SARQ | RTSNL | CQQYH | QVQLVQSGAEVKKPGASVKVSCKASGYTFTSYY | DIQMTQSPSSLSASVGDRVTITCSARQSISYMYWYC |
| DB03_ | YYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| F09 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFSN | GWIDH | CARG | SARS | RTSNL | CQQYH | QVQLVQSGAEVKKPGASVKVSCKASGYTFSNYY | DIQMTQSPSSLSASVGDRVTITCSARSSVSYMYWY |
| DB03_ | YYMH | ENGNT | YDV | SVSY | AS | SYPPTF | MHWVRQAPGQGLEWMGWIDHENGNTSYDQKFQ | QQKPGKAPKLLIYRTSNLASGVPSRFSGSGSGTDFT |
| F10 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNFK | GWIDP | CARG | SARS | RTSNL | CQQYH | QVQLVQSGAEVKKPGASVKVSCKASGYNFKDYY | DIQMTQSPSSLSASVGDRVTITCSARSSVSYMYWY |
| DB03_ | DYYIH | ENGSTI | YDV | SVSY | QS | SYPPTF | IHWVRQAPGQGLEWMGWIDPENGSTIYDQKFQG | QQKPGKAPKLLIYRTSNLQSGVPSRFSGSGSGTDFT |
| F11 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDH | CARG | RARS | RTSNL | CQQYH | QVQLVQSGAEVKKPGASVKVSCKASGYTFTGYY | DIQMTQSPSSLSASVGDRVTITCRARSSVSYMYWY |
| DB03_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| F12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | RARQ | RTSNL | CQQYH | QVQLVQSGAEVKKPGASVKVSCKASGYTFTGYY | DIQMTQSPSSLSASVGDRVTITCRARQSVSYMYWY |
| DB03_ | YYMH | ENGNTI | DDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | DDVDWFVYWGQGTLVTVSS | |
| suPAR_ | YTFTN | GWIDP | CARG | RARS | RTSNL | CQQYH | QVQLVQSGAEVKKPGASVKVSCKASGYTFTNYY | DIQMTQSPSSLSASVGDRVTITCRARSSISYMYWYQ |
| DB03_ | YYMH | YNGDT | YDV | SISY | AS | SYPPTF | MHWVRQAPGQGLEWMGWIDPYNGDTNYAQKF | QKPGKAPKLLIYRTSNLASGVPSRFSGSGSGTDFTL |
| G02 | | NYA | DWF | MY | | | QGRVTMTRDTSTSTVYMELSSLRSEDTAVYYCAR | TISSLQPEDFATYYCQQYHSYPPTFGGGTKVEIKR |
| | | | VYW | | | | GYDVDWFVYWGQGTLVTVSS | |
| suPAR_ | FNIKD | GWIDP | CARG | RASS | RTSSLQ | CQQYH | QVQLVQSGAEVKKPGASVKVSCKASGFNIKDYYI | DIQMTQSPSSLSASVGDRVTITCRASSSVSYMYWY |
| DB03_ | YYIH | ENGNTI | YDV | SVSY | S | SYPPTF | HWVRQAPGQGLEWMGWIDPENGNTIYAQKFQG | QQKPGKAPKLLIYRTSSLQSGVPSRFSGSGSGTDFT |
| G03 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTD | GWIDH | CARG | SARS | RTSNL | CQQYH | QVQLVQSGAEVKKPGASVKVSCKASGYTFTDYY | DIQMTQSPSSLSASVGDRVTITCSARSSVSYMYWY |
| DB03_ | YYMH | ENGSTI | YDV | SVSY | QS | SYPPTF | MHWVRQAPGQGLEWMGWIDHENGSTIYDQKFQ | QQKPGKAPKLLIYRTSNLQSGVPSRFSGSGSGTDFT |
| G04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTN | GWIDH | CARG | RASS | RTSNL | CQQYH | QVQLVQSGAEVKKPGASVKVSCKASGYSFTNYY | DIQMTQSPSSLSASVGDRVTITCRASSSVSYMYWY |
| DB03_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SASQ | RTSNL | CQQYH | QVQLVQSGAEVKKPGASVKVSCKASGYTFTGYY | DIQMTQSPSSLSASVGDRVTITCSASQSVSYMYWY |
| DB03_ | YYMH | EGGNTI | YDV | SVSY | AS | SYPPTF | MHWVRQAPGQGLEWMGWIDHEGGNTIYAQKFQ | QQKPGKAPKLLIYRTSNLASGVPSRFSGSGSGTDFT |
| G06 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARQ | RTSNL | CQQYH | QVQLVQSGAEVKKPGASVKVSCKASGYTFTSYY | DIQMTQSPSSLSASVGDRVTITCSARQSVSYMYWY |
| DB03_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| G07 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FTFTG | GWIDH | CARG | SARS | RTSNL | CQQYH | QVQLVQSGAEVKKPGASVKVSCKASGFTFTGHY | DIQMTQSPSSLSASVGDRVTITCSARSSVSYMYWY |
| DB03_ | HYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| G08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | RASS | RTSNL | CQQYH | QVQLVQSGAEVKKPGASVKVSCKASGYTFTDYYI | DIQMTQSPSSLSASVGDRVTITCRASSSVSYMYWY |
| DB03_ | YYIH | ENQNT | YDV | SVSY | AS | SYPPTF | HWVRQAPGQGLEWMGWIDHENGNTSYAQKFQG | QQKPGKAPKLLIYRTSNLASGVPSRFSGSGSGTDFT |
| G09 | | SYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | STENS | GWIDH | CARG | RASS | RTSNL | CQQYH | QVQLVQSGAEVKKPGASVKVSCKASGSTENSYY | DIQMTQSPSSLSASVGDRVTITCRASSSVSYMYWY |
| DB03_ | YYMH | EGGSTI | YDV | SVSY | AS | SYPPTF | MHWVRQAPGQGLEWMGWIDHEGGSTIYAQKFQ | QQKPGKAPKLLIYRTSNLASGVPSRFSGSGSGTDFT |
| G10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SASQ | RTSNL | CQQYH | QVQLVQSGAEVKKPGASVKVSCKASGYTFTSYY | DIQMTQSPSSLSASVGDRVTITCSASQSVSYMYWY |
| DB03_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| G11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTT | GWIDP | CARG | RARQ | RTSNL | CQQYH | QVQLVQSGAEVKKPGASVKVSCKASGDTFTTYY | DIQMTQSPSSLSASVGDRVTITCRARQSVSYMYWY |
| DB03_ | YYMH | ENGSTI | YDV | SVSY | AS | SYPPTF | MHWVRQAPGQGLEWMGWIDPENGSTIYAQKFQ | QQKPGKAPKLLIYRTSNLASGVPSRFSGSGSGTDFT |
| G12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SASS | RTSNL | CQQYH | QVQLVQSGAEVKKPGASVKVSCKASGYTFTNYY | DIQMTQSPSSLSASVGDRVTITCSASSSVSYMYWY |
| DB03_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| H01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGHY | DIQMTQSPSSLSASVGDRVTITCSARSSVSYMYWY |
| DB03_ | HYVH | EGGSTI | YDV | SVSY | AS | SYPPTF | VHWVRQAPGQGLEWMGWIDHEGGSTIYAQKFQ | QQKPGKAPKLLIYRTSNLASGVPSRFSGSGSGTDFT |
| H02 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SARQ | RTSNL | CQQYH | QVQLVQSGAEVKKPGASVKVSCKASGYTFTDYY | DIQMTQSPSSLSASVGDRVTITCSARQSVSYMYWY |
| DB03_ | YYMH | ENGNTI | YDV | SVSY | QS | SYPPTF | MHWVRQAPGQGLEWMGWIDHENGNTIYDQKFQ | QQKPGKAPKLLIYRTSNLQSGVPSRFSGSGSGTDFT |
| H03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDP | CARG | SARQ | RTSNL | CQQYH | QVQLVQSGAEVKKPGASVKVSCKASGYTFTGYY | DIQMTQSPSSLSASVGDRVTITCSARQSVSYMYWY |
| DB03_ | YYMH | EGGSTI | YDV | SVSY | AS | SYPPTF | MIIWVRQAPGQGLEWMGWIDPEGGSTIYDQKFQ | QQKPGKAPKLLIYRTSNLASGVPSRFSGSGSGTDFT |
| H04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDP | CARG | RARS | RTSNL | CQQYH | QVQLVQSGAEVKKPGASVKVSCKASGYTFTDYYI | DIQMTQSPSSLSASVGDRVTITCRARSSISYMYWYCH |
| DB03_ | YYIH | ENGSTI | YDV | SISY | QS | SYPPTF | HWVRQAPGQGLEWMGWIDPENGSTIYDQKFQGR | QKPGKAPKLLIYRTSNLQSGVPSRFSGSGSGTDFTL |
| H05 | | YD | DWF | MY | | | VTMTRDTSTSTVYMELSSLRSEDTAVYYCARGYD | TISSLQPEDFATYYCQQYHSYPPTFGGGTKVEIKR |
| | | | VYW | | | | VDWFVYWGQGTLVTVSS | |
| suPAR_ | YTFTS | GWMD | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB03_ | | PDSGA | YDV | SVSY | | | MHWVRQAPGQGLEWMGWMDPDSGASNYARKF | QQKPGKAPKLLIYRTSNLASGVPSRFSGSGSGTDFT |
| H06 | YYMH | SNYA | DWF | MY | AS | SYPPTF | QGRVTMTRDTSTSTVYMELSSLRSEDTAVYYCAR | LTISSLQPEDFATYYCQQYHSYPPTFGGGTKVEIKR |
| | | | VYW | | | | GYDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB03_ | YYMH | ENQSTI | YDV | SVSY | AS | SYPPTF | MHWVRQAPGQGLEWMGWIDHENGSTIYDQKFQ | QQKPGKAPKLLIYRTSNLASGVPSRFSGSGSGTDFT |
| H07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB03_ | YYVH | NTGDT | YDV | SVSY | QS | SYPPTF | VHWVRQAPGQGLEWMGWIDPNTGDTNYAQKFQ | QQKPGKAPKLLIYRTSNLQSGVPSRFSGSGSGTDFT |
| H08 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTN | GWIDH | CARG | RARQ | RTSNL | CQQSH | QVQLVQSGAEVKKPGASVKVSCKASGYSFTNYY | DIQMTQSPSSLSASVGDRVTITCRARQSVSYMYWY |
| DB03_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| H09 | | YA | DWF | MY | | | GRVTMTRDTSTSTVYMELSSLRSEDTAVYYCARG | LTISSLQPEDFATYYCQQSHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB03_ | YYMH | SNGNTI | YDV | SVSY | AS | SYPPTF | MHWVRQAPGQGLEWMGWIDHSNGNTIYAQKFQ | QQKPGKAPKLLIYRTSNLASGVPSRFSGSGSGTDFT |
| H10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSD | GWIDP | CARG | SARS | RTSNL | CQQYH | QVQLVQSGAEVKKPGASVKVSCKASGYTFSDYY | DIQMTQSPSSLSASVGDRVTITCSARSSVSYMYWY |
| DB04_ | YYMH | NTGDTI | YDV | SVSY | AS | SYPPTF | MHWVRQAPGQGLEWMGWIDPNTGDTIYAQKFQ | QQKPGKAPKLLIYRTSNLASGVPSRFSGSGSGTDFT |
| A03 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTN | GWIDH | CARG | SARS | RTSNL | CQQYH | QVQLVQSGAEVKKPGASVKVSCKASGGTFTNYY | DIQMTQSPSSLSASVGDRVTITCSARSSVSYMYWY |
| DB04_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| A04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFKD | GWIDH | CARG | SARS | RTSNL | CQQYH | QVQLVQSGAEVKKPGASVKVSCKASGYTFKDYYI | DIQMTQSPSSLSASVGDRVTITCSARSSVSYMYWY |
| DB04_ | YYIH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| A05 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDH | CARG | SARQ | RTSNL | CQQYH | QVQLVQSGAEVKKPGASVKVSCKASGYTFTSYY | DIQMTQSPSSLSASVGDRVTITCSARQSVSYMYWY |
| DB04_ | YYMH | ENGNT | YDV | SVSY | AS | SYPPTF | MHWVRQAPGQGLEWMGWIDHENGNTSYDQKFQ | QQKPGKAPKLLIYRTSNLASGVPSRFSGSGSGTDFT |
| A06 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSSLQ | CQQYH | QVQLVQSGAEVKKPGASVKVSCKASGYTFTGYY | DIQMTQSPSSLSASVGDRVTITCSARSSVSYMYWY |
| DB04_ | YYMH | ENGNTI | YDV | SVSY | S | SYPPTF | MHWVRQAPGQGLEWMGWIDHENGNTIYDQKFQ | QQKPGKAPKLLIYRTSSLQSGVPSRFSGSGSGTDFT |
| A07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTG | GWIDP | CARG | SARS | RTSNL | CQQYH | QVQLVQSGAEVKKPGASVKVSCKASGDTFTGYY | DIQMTQSPSSLSASVGDRVTITCSARSSVSYMYWY |
| DB04_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| A08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKS | GWIYP | CARG | RTSQ | KASSLE | CQQSFS | QVQLVQSGAEVKKPGASVKVSCKASGFNIKSYYI | DIQMTQSPSSLSASVGDRVTITCRTSQRIGSYLNWY |
| DB04_ | YYIH | NTGGT | YDW | RIGS | S | TPLTF | HWVRQAPGQGLEWMGWIYPNTGGTTYAQKFQG | QQKPGKAPKLLIYKASSLESGVPSRFSGSGSGTDFT |
| A09 | | TYA | DWF | YLN | | | RVTMTRDTSTSTVYMELSSLRSEDTAVYYCARGY | LTISSLQPEDFATYYCQQSFSTPLTFGGGTKVEIKR |
| | | | VYW | | | | DWDWFVYWGQGTLVTVSS | |
| suPAR_ | YTFTG | GWTQP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB04_ | YYMH | NEGAT | YDV | SVSY | AS | SYPPTF | MHWVRQAPGQGLEWMGWTDPNSGATNYAQKF | QQKPGKAPKLLIYRTSNLASGVPSRFSGSGSGTDFT |
| A10 | | NYA | DWF | MY | | | QGRVTMTRDTSTSTVYMELSSLRSEDTAVYYCAR | LTISSLQPEDFATYYCQQYHSYPPTFGGGTKVEIKR |
| | | | VYW | | | | GYDVDWFVYWGQGTLVTVSS | |
| suPAR_ | GTFTG | GWIDH | CAD | SARS | RTSNL | CQQYH | QVQLVQSGAEVKKPGASVKVSCKASGGTFTGYY | DIQMTQSPSSLSASVGDRVTITCSARSSVSYMYWY |
| DB04_ | YYMH | ENGNTI | GYD | SVSY | AS | SYPPTF | MHWVRQAPGQGLEWMGWIDHENGNTIYDQKFQ | QQKPGKAPKLLIYRTSNLASGVPSRFSGSGSGTDFT |
| A11 | | YD | VDW | MY | | | GRVTMTRDTSTSTVYMELSSLRSEDTAVYYCADG | LTISSLQPEDFATYYCQQYHSYPPTFGGGTKVEIKR |
| | | | FVY | | | | YDVDWFVYWGQGTLVTVSS | |
| | | | W | | | | | |
| suPAR_ | YTFTD | GWIDH | CARG | RASS | RTSNL | CQQYH | QVQLVQSGAEVKKPGASVKVSCKASGYTFTDYY | DIQMTQSPSSLSASVGDRVTITCRASSSVSYMYWY |
| DB04_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| A12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSR | GVINPT | CARG | RASQ | DASNL | CQQSY | QVQLVQSGAEVKKPGASVKVSCKASGYTFSRYY | DIQMTQSPSSLSASVGDRVTITCRASQGISDYLAWY |
| DB04_ | YYMH | GGSTT | YDV | GISD | ET | SDPPTF | MHWVRQAPGQGLEWMGVINPTGGSTTYAQKFQ | QQKPGKAPKLLIYDASNLETGVPSRFSGSGSGTDFT |
| B01 | | YA | DWF | YLA | | | GRVTMTRDTSTSTVYMELSSLRSEDTAVYYCARG | LTISSLQPEDFATYYCQQSYSDPPTFGGGTKVEIKR |
| | | | VYW | | | | YDVDWFVYWGQGTLVTVSS | |
| suPAR_ | YTFTN | GWIDP | CARG | SARS | RTSSLQ | CQQYH | QVQLVQSGAEVKKPGASVKVSCKASGYTFTNYY | DIQMTQSPSSLSASVGDRVTITCSARSSVSYMYWY |
| DB04_ | YYMH | ENQSTI | YDV | SVSY | S | SYPPTF | MHWVRQAPGQGLEWMGWIDPENGSTIYDQKFQ | QQKPGKAPKLLIYRTSSLQSGVPSRFSGSGSGTDFT |
| B02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTS | GWIDH | CARG | SASQ | RTSNL | CQQYH | QVQLVQSGAEVKKPGASVKVSCKASGYSFTSYY | DIQMTQSPSSLSASVGDRVTITCSASQSVSYMYWY |
| DB04_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| B03 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | STFTG | GWIDH | CARG | RARQ | RTSNL | CQQYH | QVQLVQSGAEVKKPGASVKVSCKASGSTFTGYY | DIQMTQSPSSLSASVGDRVTITCRARQSVSYMYWY |
| DB04_ | YYMH | ENGNT | YDV | SVSY | AS | SYPPTF | MHWVRQAPGQGLEWMGWIDHENGNTSYDQKFQ | QQKPGKAPKLLIYRTSNLASGVPSRFSGSGSGTDFT |
| B04 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTENS | GWID | CARG | SARS | RTSNL | CQQYH | QVQLVQSGAEVKKPGASVKVSCKASGYTENSYY | DIQMTQSPSSLSASVGDRVTITCSARSSVSYMYWY |
| DB04_ | YYLH | NEGGT | YDV | SVSY | AS | SYPPTF | LHWVRQAPGQGLEWMGWIDPNSGGTNYAQKFQ | QQKPGKAPKLLIYRTSNLASGVPSRFSGSGSGTDFT |
| B05 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB04_ | YYMH | ENGNT | NDV | SVSY | AS | SYPPTF | MHWVRQAPGQGLEWMGWIDHENGNTSYDQKFQ | QQKPGKAPKLLIYRTSNLASGVPSRFSGSGSGTDFT |
| B06 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | NDVDWFVYWGQGTLVTVSS | |
| suPAR_ | YTFTN | GWIDY | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB04_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| B07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RASS | RTSNL | CQQYH | QVQLVQSGAEVKKPGASVKVSCKASGYTFTSYY | DIQMTQSPSSLSASVGDRVTITCRASSSVSYMYWY |
| DB04_ | YYMH | ENGSTS | TDVD | SVSY | AS | SYPPTF | MHWVRQAPGQGLEWMGWIDHENGSTSYDQKFQ | QQKPGKAPKLLIYRTSNLASGVPSRFSGSGSGTDFT |
| B08 | | YD | WFV | MY | | | GRVTMTRDTSTSTVYMELSSLRSEDTAVYYCARG | LTISSLQPEDFATYYCQQYHSYPPTFGGGTKVEIKR |
| | | | YW | | | | TDVDWFVYWGQGTLVTVSS | |
| suPAR_ | YMFT | GWIDY | CARG | RARS | RTSSLQ | CQQYH | QVQLVQSGAEVKKPGASVKVSCKASGYMFTGYY | DIQMTQSPSSLSASVGDRVTITCRARSSVSYMYWY |
| DB04_ | GYYIH | ENGSTI | YDV | SVSY | S | SYPPTF | IHWVRQAPGQGLEWMGWIDPENGSTIYDQKFQG | QQKPGKAPKLLIYRTSSLQSGVPSRFSGSGSGTDFT |
| B09 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDP | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB04_ | YYMQ | NSGSTS | YDV | SVSY | AS | SYPPTF | MQWVRQAPGQGLEWMGWIDPNSGSTSYAQKFQ | QQKPGKAPKLLIYRTSNLASGVPSRFSGSGSGTDFT |
| B10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTR | GVINPS | CARG | RASQ | DASNL | CQQSY | QVQLVQSGAEVKKPGASVKVSCKASGYTFTRYYI | DIQMTQSPSSLSASVGDRVTITCRASQGISNYLAWY |
| DB04_ | YYIH | GGSTS | YDV | GISN | ET | GDPLTF | HWVRQAPGQGLEWMGVINPSGGSTSYAQKFQGR | QQKPGKAPKLLIYDASNLETGVPSRFSGSGSGTDFT |
| B11 | | YA | DWF | YLA | | | VTMTRDTSTSTVYMELSSLRSEDTAVYYCARGYD | LTISSLQPEDFATYYCQQSYGDPLTFGGGTKVEIKR |
| | | | VYW | | | | VDWFVYWGQGTLVTVSS | |
| suPAR_ | DTFTS | GWIDH | CARG | RARS | RTSNL | CQQYH | QVQLVQSGAEVKKPGASVKVSCKASGDTFTSYY | DIQMTQSPSSLSASVGDRVTITCRARSSISYMYWYQ |
| DB04_ | YYVH | EGGSTI | YDV | SISY | AS | SYPPTF | VHWVRQAPGQGLEWMGWIDHEGGSTIYAQKFQ | QKPGKAPKLLIYRTSNLASGVPSRFSGSGSGTDFTL |
| B12 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB04_ | YYMH | EGGSTI | YDV | SVSY | AS | SYPPTF | MHWVRQAPGQGLEWMGWIDHEGGSTIYAQKFQ | QQKPGKAPKLLIYRTSNLASGVPSRFSGSGSGTDFT |
| C01 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | NTFIG | GWIDH | CARG | SARS | RTSNL | CQQYH | QVQLVQSGAEVKKPGASVKVSCKASGNTFIGYYV | DIQMTQSPSSLSASVGDRVTITCSARSSVSYMYWY |
| DB04_ | YYVH | ENGNT | NDV | SVSY | AS | SYPPTF | HWVRQAPGQGLEWMGWIDHENGNTSYDQKFQG | QQKPGKAPKLLIYRTSNLASGVPSRFSGSGSGTDFT |
| C02 | | SYD | DWF | MY | | | RVTMTRDTSTSTVYMELSSLRSEDTAVYYCARGN | LTISSLQPEDFATYYCQQYHSYPPTFGGGTKVEIKR |
| | | | VYW | | | | DVDWFVYWGQGTLVTVSS | |
| suPAR_ | YTFTS | GWIDP | CASG | SARS | RTSNL | CQQYH | QVQLVQSGAEVKKPGASVKVSCKASGYTFTSYY | DIQMTQSPSSLSASVGDRVTITCSARSSVSYMYWY |
| DB04_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| C03 | | YD | DWF | MY | | | GRVTMTRDTSTSTVYMELSSLRSEDTAVYYCASG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SASQ | RTSNL | CQQYH | QVQLVQSGAEVKKPGASVKVSCKASGYTFTGYY | DIQMTQSPSSLSASVGDRVTITCSASQSVSYMYWY |
| DB04_ | YYMH | ENQNT | YDV | SVSY | AS | SYPPTF | MHWVRQAPGQGLEWMGWIDHENGNTSYDQKFQ | QQKPGKAPKLLIYRTSNLASGVPSRFSGSGSGTDFT |
| C04 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTLNS | GWIDH | CARG | SARS | RTSNL | CQQYH | QVQLVQSGAEVKKPGASVKVSCKASGHTLNSYY | DIQMTQSPSSLSASVGDRVTITCSARSSVSYMYWY |
| DB04_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| C05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSA | GWIDH | CARG | SARQ | RTSNL | CQQYH | QVQLVQSGAEVKKPGASVKVSCKASGYTFSAYYI | DIQMTQSPSSLSASVGDRVTITCSARQSVSYMYWY |
| DB04_ | YYIH | ENQNTI | NDV | SVSY | QS | SYPPTF | HWVRQAPGQGLEWMGWIDHENGNTIYDQKFQG | QQKPGKAPKLLIYRTSNLQSGVPSRFSGSGSGTDFT |
| C06 | | YD | DWF | MY | | | RVTMTRDTSTSTVYMELSSLRSEDTAVYYCARGN | LTISSLQPEDFATYYCQQYHSYPPTFGGGTKVEIKR |
| | | | VYW | | | | DVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB04_ | YYMH | SNGNTI | DDV | SVSY | AS | SYPPTF | MHWVRQAPGQGLEWMGWIDHSNGNTIYDQKFQ | QQKPGKAPKLLIYRTSNLASGVPSRFSGSGSGTDFT |
| C07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | DDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB04_ | YYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| C08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFST | GWIDP | CARG | SARS | RASNL | CQQYH | QVQLVQSGAEVKKPGASVKVSCKASGYTFSTYY | DIQMTQSPSSLSASVGDRVTITCSARSSVSYMYWY |
| DB04_ | YYMQ | ENQSTI | YDV | SVSY | AS | SYPPTF | MQWVRQAPGQGLEWMGWIDPENGSTIYDQKFQ | QQKPGKAPKLLIYRASNLASGVPSRFSGSGSGTDFT |
| C09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKD | GWIDP | CARG | SARS | RTSNL | CQQYH | QVQLVQSGAEVKKPGASVKVSCKASGENEKDYY | DIQMTQSPSSLSASVGDRVTITCSARSSVSYMYWY |
| DB04_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| C10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTD | GWIDP | CARG | SARS | RTSNL | CQQYH | QVQLVQSGAEVKKPGASVKVSCKASGYSFTDYY | DIQMTQSPSSLSASVGDRVTITCSARSSVSYMYWY |
| DB04_ | YYMH | ENQSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| C11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTITS | GWIDH | CARG | RASS | RTSNL | CQQYH | QVQLVQSGAEVKKPGASVKVSCKASGHTITSYY | DIQMTQSPSSLSASVGDRVTITCRASSSVSYMYWY |
| DB04_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| C12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTS | GWIDH | CARG | RASQ | RTSNL | CGQHN | QVQLVQSGAEVKKPGASVKVSCKASGYSFTSYY | DIQMTQSPSSLSASVGDRVTITCRASQDISSYLNWY |
| DB04_ | YYMH | SNQNTI | YDV | DISS | AS | TFPPTF | MHWVRQAPGQGLEWMGWIDHSNGNTIYDQKFQ | QQKPGKAPKLLIYRTSNLASGVPSRFSGSGSGTDFT |
| D01 | | YD | DWF | YLN | | | GRVTMTRDTSTSTVYMELSSLRSEDTAVYYCARG | LTISSLQPEDFATYYCGQHNTFPPTFGGGTKVEIKR |
| | | | VYW | | | | YDVDWFVYWGQGTLVTVSS | |
| suPAR_ | GTFSS | GIINPS | CARG | RASQ | DATNL | CQQYY | QVQLVQSGAEVKKPGASVKVSCKASGGTFSSYAI | DIQMTQSPSSLSASVGDRVTITCRASQSISSELNWY |
| DB04_ | YAIS | GGSTS | YDV | SISSF | AT | STPPTF | SWVRQAPGQGLEWMGIINPSGGSTSYAQKFQGRV | QQKPGKAPKLLIYDATNLATGVPSRFSGSGSGTDFT |
| D02 | | YA | DWF | LN | | | TMTRDTSTSTVYMELSSLRSEDTAVYYCARGYDV | LTISSLQPEDFATYYCQQYYSTPPTFGGGTKVEIKR |
| | | | VTW | | | | DWFVTWGQGTLVTVSS | |
| suPAR_ | YTFTS | GWIDP | CARG | RARQ | RTSNL | CQQYH | QVQLVQSGAEVKKPGASVKVSCKASGYTFTSYY | DIQMTQSPSSLSASVGDRVTITCRARQSVSYMYWY |
| DB04_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| D03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTIKD | GWIDH | CARG | SARS | RTSNL | CQQYH | QVQLVQSGAEVKKPGASVKVSCKASGYTIKDYY | DIQMTQSPSSLSASVGDRVTITCSARSSVSYMYWY |
| DB04_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| D04 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTT | GWIDP | CARG | SARS | RTSNL | CQQYH | QVQLVQSGAEVKKPGASVKVSCKASGYTFTTYY | DIQMTQSPSSLSASVGDRVTITCSARSSVSYMYWY |
| DB04_ | YYMH | HSGGT | YDV | SVSY | AS | SYPPTF | MHWVRQAPGQGLEWVGWIDPHSGGTNYAQKFQ | QQKPGKAPKLLIYRTSNLASGVPSRFSGSGSGTDFT |
| D05 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNETS | GWIDY | CARG | SARS | RTSNL | CQQYH | QVQLVQSGAEVKKPGASVKVSCKASGENITSYYI | DIQMTQSPSSLSASVGDRVTITCSARSSVSYMYWY |
| DB04_ | YYIH | ENGNTI | YDV | SVSY | AS | SYPPTF | HWVRQAPGQGLEWMGWIDPENGNTIYAQKFQG | QQKPGKAPKLLIYRTSNLASGVPSRFSGSGSGTDFT |
| D06 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFGS | GWIDH | CARG | RARQ | RTSNL | CQQYH | QVQLVQSGAEVKKPGASVKVSCKASGYTFGSYY | DIQMTQSPSSLSASVGDRVTITCRARQSVSYMYWY |
| DB04_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTLSG | GWIDP | CARG | SARS | RTSNL | CQQYH | QVQLVQSGAEVKKPGASVKVSCKASGYTLSGYY | DIQMTQSPSSLSASVGDRVTITCSARSSVSYMYWY |
| DB04_ | YYMH | NEGAT | YDV | SVSY | AS | SYPPTF | MHWVRQAPGQGLEWMGWIDPNSGATNDAPKFQ | QQKPGKAPKLLIYRTSNLASGVPSRFSGSGSGTDFT |
| D08 | | NDA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTA | GWIDH | CARG | SARS | RTSNL | CQQYH | QVQLVQSGAEVKKPGASVKVSCKASGYTFTAYY | DIQMTQSPSSLSASVGDRVTITCSARSSVSYMYWY |
| DB04_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CATG | SARS | RTSNL | CQQYH | QVQLVQSGAEVKKPGASVKVSCKASGYTFTSYY | DIQMTQSPSSLSASVGDRVTITCSARSSVSYMYWY |
| DB04_ | YYMH | EGGNT | YDV | SVSY | AS | SYPPTF | MHWVRQAPGQGLEWMGWIDPEGGNTSYDQKFQ | QQKPGKAPKLLIYRTSNLASGVPSRFSGSGSGTDFT |
| D10 | | SYD | DWF | MY | | | GRVTMTRDTSTSTVYMELSSLRSEDTAVYYCATG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SASS | RTSNL | CQQYH | QVQLVQSGAEVKKPGASVKVSCKASGYTFTDYYI | DIQMTQSPSSLSASVGDRVTITCSASSSVSYMYWY |
| DB04_ | YYIH | ENGSTS | YDV | SVSY | AS | SYPPTF | HWVRQAPGQGLEWMGWIDHENGSTSYDQKFQG | QQKPGKAPKLLIYRTSNLASGVPSRFSGSGSGTDFT |
| D11 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | FNITD | GWIDP | CARG | SARS | RTSNL | CQQYH | QVQLVQSGAEVKKPGASVKVSCKASGENITDYY | DIQMTQSPSSLSASVGDRVTITCSARSSVSYMYWY |
| DB04_ | YYMH | NGGGT | YDV | SVSY | AS | SYPPTF | MHWVRQAPGQGLEWMGWIDPNGGGTQYAQKF | QQKPGKAPKLLIYRTSNLASGVPSRFSGSGSGTDFT |
| D12 | | QYA | DWF | MY | | | QGRVTMTRDTSTSTVYMELSSLRSEDTAVYYCAR | LTISSLQPEDFATYYCQQYHSYPPTFGGGTKVEIKR |
| | | | VYW | | | | GYDVDWFVYWGQGTLVTVSS | |
| suPAR_ | GTFTT | GWIQP | CARG | SARS | RTSNL | CQQYH | QVQLVQSGAEVKKPGASVKVSCKASGGTFTTYY | DIQMTQSPSSLSASVGDRVTITCSARSSVSYMYWY |
| DB04_ | YYMH | SSGDTS | YDV | SVSY | AS | SYPPTF | MHWVRQAPGQGLEWMGWIDPSSGDTSYAQKFQ | QQKPGKAPKLLIYRTSNLASGVPSRFSGSGSGTDFT |
| E01 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWEN | CARG | RARS | ATSNL | CQQYH | QVQLVQSGAEVKKPGASVKVSCKASGYTFTSYY | DIQMTQSPSSLSASVGDRVTITCRARSSVSYMYWY |
| DB04_ | YYMH | ENGSTI | YDV | SVSY | AS | SYPPTF | MHWVRQAPGQGLEWMGWIDPENGSTIYDQKFQ | QQKPGKAPKLLIYATSNLASGVPSRFSGSGSGTDFT |
| E02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB04_ | YYMH | EGGSTI | YDV | SVSY | AS | SYPPTF | MHWVRQAPGQGLEWMGWIDHEGGSTIYAQKFQ | QQKPGKAPKLLIYRTSNLASGVPSRFSGSGSGTDFT |
| E03 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RARQ | RTSNL | CQQYH | QVQLVQSGAEVKKPGASVKVSCKASGYTFTSYY | DIQMTQSPSSLSASVGDRVTITCRARQSVSYMYWY |
| DB04_ | YYVH | ENQNTI | TDVD | SVSY | AS | SYPPTF | VHWVRQAPGQGLEWMGWIDHENGNTIYDQKFQ | QQKPGKAPKLLIYRTSNLASGVPSRFSGSGSGTDFT |
| E04 | | YD | WFV | MY | | | GRVTMTRDTSTSTVYMELSSLRSEDTAVYYCARG | LTISSLQPEDFATYYCQQYHSYPPTFGGGTKVEIKR |
| | | | YW | | | | TDVDWFVYWGQGTLVTVSS | |
| suPAR_ | FNIKD | GWIDP | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB04_ | YYIH | HSGGT | YDV | SVSY | AS | SYPPTF | HWVRQAPGQGLEWVGWIDPHSGGTNYAQKFQG | QQKPGKAPKLLIYRTSNLASGVPSRFSGSGSGTDFT |
| E05 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YSFTS | GWIDH | CARG | RARS | RTSNL | CQQYH | QVQLVQSGAEVKKPGASVKVSCKASGYSFTSHY | DIQMTQSPSSLSASVGDRVTITCRARSSVSYMYWY |
| DB04_ | HYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTS | GWIDH | CARG | SARS | RTSNL | CQQYH | QVQLVQSGAEVKKPGASVKVSCKASGDTFTSYYI | DIQMTQSPSSLSASVGDRVTITCSARSSVSYMYWY |
| DB04_ | YYIH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| E07 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB04_ | YYMH | ENGNT | YDV | SVSY | AS | SYPPTF | MHWVRQAPGQGLEWMGWIDHENGNTSYAQKFQ | QQKPGKAPKLLIYRTSNLASGVPSRFSGSGSGTDFT |
| E08 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTD | GWIDH | CARG | RARQ | RTSNL | CQQYH | QVQLVQSGAEVKKPGASVKVSCKASGDTFTDYYI | DIQMTQSPSSLSASVGDRVTITCRARQSVSYMYWY |
| DB04_ | YYIH | ENGSTS | YDV | SVSY | AS | SYPPTF | HWVRQAPGQGLEWMGWIDHENGSTSYDQKFQG | QQKPGKAPKLLIYRTSNLASGVPSRFSGSGSGTDFT |
| E09 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| SUPAR | YTFSS | GWIDP | CARG | SARS | RTSNL | CQQYH | QVQLVQSGAEVKKPGASVKVSCKASGYTFSSYYF | DIQMTQSPSSLSASVGDRVTITCSARSSVSYMYWY |
| DB04_ | YYFH | NTGDTI | YDV | SVSY | AS | SYPPTF | HWVRQAPGQGLEWMGWIDPNTGDTIYAQKFQG | QQKPGKAPKLLIYRTSNLASGVPSRFSGSGSGTDFT |
| E10 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDH | CARG | SARQ | RTSNL | CQQYH | QVQLVQSGAEVKKPGASVKVSCKASGYTFTNYY | DIQMTQSPSSLSASVGDRVTITCSARQSVSYMYWY |
| DB04_ | YYVH | ENQNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| E11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTT | GWID | CARG | SASQ | RTSNL | CQQYH | QVQLVQSGAEVKKPGASVKVSCKASGYTFTTYY | DIQMTQSPSSLSASVGDRVTITCSASQSVSYMYWY |
| DB04_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| E12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SASQ | RTSNL | CQQYH | QVQLVQSGAEVKKPGASVKVSCKASGYTFTSYY | DIQMTQSPSSLSASVGDRVTITCSASQSVSYMYWY |
| DB04_ | YYMH | EGGNTI | DDV | SVSY | AS | SYPPTF | MHWVRQAPGQGLEWMGWIDHEGGNTIYAQKFQ | QQKPGKAPKLLIYRTSNLASGVPSRFSGSGSGTDFT |
| F01 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | DDVDWFVYWGQGTLVTVSS | |
| suPAR_ | NTFTD | GWIDH | CARG | SARQ | RTSNL | CQQYH | QVQLVQSGAEVKKPGASVKVSCKASGNTFTDYY | DIQMTQSPSSLSASVGDRVTITCSARQSVSYMYWY |
| DB04_ | YYMH | ENQNTI | YDV | SVSY | AS | SYPPTF | MHWVRQAPGQGLEWMGWIDHENGNTIYAQKFQ | QQKPGKAPKLLIYRTSNLASGVPSRFSGSGSGTDFT |
| F02 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTIKD | GWIDY | CARG | SARS | RTSNL | CQQYH | QVQLVQSGAEVKKPGASVKVSCKASGYTIKDYYI | DIQMTQSPSSLSASVGDRVTITCSARSSVSYMYWY |
| DB04_ | YYIH | RSGDT | YDV | SVSY | AS | SYPPTF | HWVRQAPGQGLEWMGWIDPRSGDTNYGKNFQG | QQKPGKAPKLLIYRTSNLASGVPSRFSGSGSGTDFT |
| F03 | | NYG | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB04_ | YYMH | ENGNT | YDV | SVSY | AS | SYPPTF | MHWVRQAPGQGLEWMGWIDHENGNTSYDQKFQ | QQKPGKAPKLLIYRTSNLASGVPSRFSGSGSGTDFT |
| F04 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSD | GWIDH | CARG | SARQ | RTSNL | CQQYH | QVQLVQSGAEVKKPGASVKVSCKASGYTFSDYY | DIQMTQSPSSLSASVGDRVTITCSARQSISYMYWYQ |
| DB04_ | YYVH | EGGSTS | YDV | SISY | AS | SYPPTF | VHWVRQAPGQGLEWMGWIDHEGGSTSYAQKFQ | QKPGKAPKLLIYRTSNLASGVPSRFSGSGSGTDFTL |
| F05 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARQ | RTSNL | CQQYH | QVQLVQSGAEVKKPGASVKVSCKASGYTFTGYY | DIQMTQSPSSLSASVGDRVTITCSARQSISYMYWYQ |
| DB04_ | YYMH | ENGNT | YDV | SISY | AS | SYPPTF | MHWVRQAPGQGLEWMGWIDHENGNTSYAQKFQ | QKPGKAPKLLIYRTSNLASGVPSRFSGSGSGTDFTL |
| F06 | | SYA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB04_ | YYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| F07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTS | GWIDH | CARG | SARS | RTSNL | CQQYH | QVQLVQSGAEVKKPGASVKVSCKASGGTFTSYY | DIQMTQSPSSLSASVGDRVTITCSARSSVSYMYWY |
| DB04_ | YYMH | SNGSTS | YDV | SVSY | AS | SYPPTF | MHWVRQAPGQGLEWMGWIDHSNGSTSYAQEFQ | QQKPGKAPKLLIYRTSNLASGVPSRFSGSGSGTDFT |
| F08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB04_ | YYFH | ENGNT | YDV | SVSY | AS | SYPPTF | FHWVRQAPGQGLEWMGWIDHENGNTSYDQKFQ | QQKPGKAPKLLIYRTSNLASGVPSRFSGSGSGTDFT |
| F09 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWIDH | CARG | SARS | RTSNL | CQQYH | QVQLVQSGAEVKKPGASVKVSCKASGYTFTDYY | DIQMTQSPSSLSASVGDRVTITCSARSSVSYMYWY |
| DB04_ | YYMH | SGGNTI | YDV | SVSY | AS | SYPPTF | MHWVRQAPGQGLEWMGWIDHSGGNTIYAQKFQ | QQKPGKAPKLLIYRTSNLASGVPSRFSGSGSGTDFT |
| F10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSHY | DIQMTQSPSSLSASVGDRVTITCSARSSVSYMYWY |
| DB04_ | HYMH | EGGNT | YDV | SVSY | AS | SYPPTF | MHWVRQAPGQGLEWMGWIDHEGGNTSYAQKFQ | QQKPGKAPKLLIYRTSNLASGVPSRFSGSGSGTDFT |
| F11 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DSENT | GWIDH | CARG | SARS | RTSNL | CQQYH | QVQLVQSGAEVKKPGASVKVSCKASGDSENTYY | DIQMTQSPSSLSASVGDRVTITCSARSSVSYMYWY |
| DB04_ | YYFH | ENQNTI | YDV | SVSY | AS | SYPPTF | FHWVRQAPGQGLEWMGWIDHENGNTIYAQKFQ | QQKPGKAPKLLIYRTSNLASGVPSRFSGSGSGTDFT |
| F12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSD | GWIDH | CARG | SARS | RTSNL | CQQYH | QVQLVQSGAEVKKPGASVKVSCKASGYTFSDYY | DIQMTQSPSSLSASVGDRVTITCSARSSVSYMYWY |
| DB04_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSSLA | CQQYH | QVQLVQSGAEVKKPGASVKVSCKASGYTFTNYY | DIQMTQSPSSLSASVGDRVTITCSARSSVSYMYWY |
| DB04_ | YYMH | ENQNTI | YDV | SVSY | S | SYPPTF | MHWVRQAPGQGLEWMGWIDHENGNTIYDQKFQ | QQKPGKAPKLLIYRTSSLASGVPSRFSGSGSGTDFT |
| G02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| SUPAR | YTFTG | GWIDH | CARG | SASS | RTSNL | CQQYH | QVQLVQSGAEVKKPGASVKVSCKASGYTFTGYY | DIQMTQSPSSLSASVGDRVTITCSASSSVSYMYWY |
| DB04_ | YYMH | ENGNTI | YDV | SVSY | QS | SYPPTF | MHWVRQAPGQGLEWMGWIDHENGNTIYDQKFQ | QQKPGKAPKLLIYRTSNLQSGVPSRFSGSGSGTDFT |
| G03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTS | GWIDH | CARG | SASQ | RTSNL | CQQYH | QVQLVQSGAEVKKPGASVKVSCKASGYSFTSYYI | DIQMTQSPSSLSASVGDRVTITCSASQSVSYMYWY |
| DB04_ | YYIH | ENGSTI | YDV | SVSY | AS | SYPPTF | HWVRQAPGQGLEWMGWIDHENGSTIYAQKFQG | QQKPGKAPKLLIYRTSNLASGVPSRFSGSGSGTDFT |
| G04 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YNITD | GWID | CARG | SARS | RTSNL | CQQYH | QVQLVQSGAEVKKPGASVKVSCKASGYNITDYYI | DIQMTQSPSSLSASVGDRVTITCSARSSVSYMYWY |
| DB04_ | YYIH | ENGSTI | YDV | SVSY | QS | SYPPTF | HWVRQAPGQGLEWMGWIDPENGSTIYDQKFQGR | QQKPGKAPKLLIYRTSNLQSGVPSRFSGSGSGTDFT |
| G05 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | DTFTS | GWIDH | CARG | SASQ | RTSNL | CQQYH | QVQLVQSGAEVKKPGASVKVSCKASGDTFTSYY | DIQMTQSPSSLSASVGDRVTITCSASQSVSYMYWY |
| DB04_ | YYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| G06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FSFTT | GWIDP | CARG | SARS | RTSNL | CQQYH | QVQLVQSGAEVKKPGASVKVSCKASGFSFTTYY | DIQMTQSPSSLSASVGDRVTITCSARSSVSYMYWY |
| DB04_ | YYMH | ENQNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| G07 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTLTG | GWIDH | CARG | SARS | RTSNL | CQQYH | QVQLVQSGAEVKKPGASVKVSCKASGHTLTGYY | DIQMTQSPSSLSASVGDRVTITCSARSSVSYMYWY |
| DB04_ | YYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| G08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTSTT | GWIDH | CARG | SAKS | RTSNL | CQQYH | QVQLVQSGAEVKKPGASVKVSCKASGDTSTTYY | DIQMTQSPSSLSASVGDRVTITCSAKSSVSYMYWY |
| DB04_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | RASS | RTSNL | CQQYH | QVQLVQSGAEVKKPGASVKVSCKASGYTFTGYY | DIQMTQSPSSLSASVGDRVTITCRASSSVSYMYWY |
| DB04_ | YYMH | ENGNTI | NDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | NDVDWFVYWGQGTLVTVSS | |
| suPAR_ | FTFKD | GWIDP | CARG | SARQ | RTSNL | CQQYH | QVQLVQSGAEVKKPGASVKVSCKASGFTFKDYYI | DIQMTQSPSSLSASVGDRVTITCSARQSVSYMYWY |
| DB04_ | YYIH | DSGGT | YDV | SVSY | AS | SYPPTF | HWVRQAPGQGLEWMGWIDPDSGGTNYAQKFQG | QQKPGKAPKLLIYRTSNLASGVPSRFSGSGSGTDFT |
| G11 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFSD | GWIDH | CARG | SARS | RTSNL | CQQYH | QVQLVQSGAEVKKPGASVKVSCKASGYTFSDYY | DIQMTQSPSSLSASVGDRVTITCSARSSVSYMYWY |
| DB04_ | YYMH | ENQNT | YDV | SVSY | AS | SYPPTF | MHWVRQAPGQGLEWMGWIDHENGNTSYAQKFQ | QQKPGKAPKLLIYRTSNLASGVPSRFSGSGSGTDFT |
| G12 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFIG | GWIDH | CARG | SARS | RTSSLA | CQQYH | QVQLVQSGAEVKKPGASVKVSCKASGYTFIGSYV | DIQMTQSPSSLSASVGDRVTITCSARSSVSYMYWY |
| DB04_ | SYVH | ENGNTI | YDV | SVSY | S | SYPPTF | HWVRQAPGQGLEWMGWIDHENGNTIYDQKFQG | QQKPGKAPKLLIYRTSSLASGVPSRFSGSGSGTDFT |
| H01 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | DTFSN | GWIDP | CARG | SARS | RTSSLQ | CQQYH | QVQLVQSGAEVKKPGASVKVSCKASGDTFSNYY | DIQMTQSPSSLSASVGDRVTITCSARSSVSYMYWY |
| DB04_ | YYMH | ENQNTI | YDV | SVSY | S | SYPPTF | MHWVRQAPGQGLEWMGWIDPENGNTIYAQKFQ | QQKPGKAPKLLIYRTSSLQSGVPSRFSGSGSGTDFT |
| H02 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SASS | RTSNL | CQQYH | QVQLVQSGAEVKKPGASVKVSCKASGYTFTGNY | DIQMTQSPSSLSASVGDRVTITCSASSSVSYMYWY |
| DB04_ | NYLH | SNGSTI | SDVD | SVSY | AS | SYPPTF | LHWVRQAPGQGLEWMGWIDHSNGSTIYDQKFQG | QQKPGKAPKLLIYRTSNLASGVPSRFSGSGSGTDFT |
| H03 | | YD | WFV | MY | | | RVTMTRDTSTSTVYMELSSLRSEDTAVYYCARGS | LTISSLQPEDFATYYCQQYHSYPPTFGGGTKVEIKR |
| | | | YW | | | | DVDWFVYWGQGTLVTVSS | |
| suPAR_ | YTFNG | GWIDE | CARG | SARS | RTSNL | CQQYH | QVQLVQSGAEVKKPGASVKVSCKASGYTENGYYI | DIQMTQSPSSLSASVGDRVTITCSARSSVSYMYWY |
| DB04_ | YYIQ | ENGNTI | YDV | SVSY | AS | SYPPTF | QWVRQAPGQGLEWMGWIDPENGNTIYDQKFQG | QQKPGKAPKLLIYRTSNLASGVPSRFSGSGSGTDFT |
| H04 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | HTLNS | GWIDY | CARG | SARS | RTSNL | CQQYH | QVQLVQSGAEVKKPGASVKVSCKASGHTLNSYY | DIQMTQSPSSLSASVGDRVTITCSARSSVSYMYWY |
| DB04_ | YYMH | EGGNTI | YDV | SVSY | AS | SYPPTF | MHWVRQAPGQGLEWMGWIDPEGGNTIYAQKFQ | QQKPGKAPKLLIYRTSNLASGVPSRFSGSGSGTDFT |
| H05 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB04_ | YYVH | ENGNT | YDV | SVSY | QS | SYPPTF | VHWVRQAPGQGLEWMGWIDHENGNTSYAQKFQ | QQKPGKAPKLLIYRTSNLQSGVPSRFSGSGSGTDFT |
| H06 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RARS | RTSNL | CQQYH | QVQLVQSGAEVKKPGASVKVSCKASGYTFTSYY | DIQMTQSPSSLSASVGDRVTITCRARSSVSYMYWY |
| DB04_ | YYMH | ENQNT | YDV | SVSY | AS | SYPPTF | MHWVRQAPGQGLEWMGWIDHENGNTSYDQKFQ | QQKPGKAPKLLIYRTSNLASGVPSRFSGSGSGTDFT |
| H07 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSD | GWISP | CARG | SASQ | RTSNL | CQQYH | QVQLVQSGAEVKKPGASVKVSCKASGYTFSDYYI | DIQMTQSPSSLSASVGDRVTITCSASQSVSYMYWY |
| DB04_ | YYIH | KSGVT | YDV | SVSY | AS | SYPPTF | HWVRQAPGQGLEWMGWISPKSGVTNYAQKFQG | QQKPGKAPKLLIYRTSNLASGVPSRFSGSGSGTDFT |
| H08 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | GTFSG | GWITP | CARG | SARQ | RTSNL | CQQYH | QVQLVQSGAEVKKPGASVKVSCKASGGTFSGYY | DIQMTQSPSSLSASVGDRVTITCSARQSVSYMYWY |
| DB04_ | YYMH | DSGAT | YDV | SVSY | AS | SYPPTF | MHWVRQAPGQGLEWVGWITPDSGATAYAQKFQ | QQKPGKAPKLLIYRTSNLASGVPSRFSGSGSGTDFT |
| H09 | | AYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWISP | CARG | RARS | RTSNL | CQQYH | QVQLVQSGAEVKKPGASVKVSCKASGYTFTSYY | DIQMTQSPSSLSASVGDRVTITCRARSSVSYMYWY |
| DB04_ | YYMH | DSGGT | YDV | SVSY | AS | SYPPTF | MHWVRQAPGQGLEWMGWISPDSGGTNYAQKFQ | QQKPGKAPKLLIYRTSNLASGVPSRFSGSGSGTDFT |
| H10 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFSN | GWIDH | CARG | SARS | RTSNL | CQQYH | QVQLVQSGAEVKKPGASVKVSCKASGGTFSNYIH | DIQMTQSPSSLSASVGDRVTITCSARSSVSYMYWY |
| DB05_ | YIH | ENGSTI | YDV | SVSY | AS | SYPPTF | WVRQAPGQGLEWMGWIDHENGSTIYAQKFQGR | QQKPGKAPKLLIYRTSNLASGVPSRFSGSGSGTDFT |
| A03 | | YA | DWF | MY | | | VTMTRDTSTSTVYMELSSLRSEDTAVYYCARGYD | LTISSLQPEDFATYYCQQYHSYPPTFGGGTKVEIKR |
| | | | VLW | | | | VDWFVLWGQGTLVTVSS | |
| suPAR_ | YTFTS | GWIDP | CARG | SARQ | RTSNL | CQQYH | QVQLVQSGAEVKKPGASVKVSCKASGYTFTSYY | DIQMTQSPSSLSASVGDRVTITCSARQSISYMYWYC |
| DB05_ | YYMH | ENGNTI | YDV | SISY | AS | SYPPTF | MHWVRQAPGQGLEWMGWIDPENGNTIYAQKFQ | QKPGKAPKLLIYRTSNLASGVPSRFSGSGSGTDFTL |
| A04 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | RARQ | RTSNL | CQQYH | QVQLVQSGAEVKKPGASVKVSCKASGYTFTNYY | DIQMTQSPSSLSASVGDRVTITCRARQSVSYMYWY |
| DB05_ | YYMH | SGGSTS | YDV | SVSY | AS | SYPPTF | MHWVRQAPGQGLEWMGWIDPSGGSTSYAQKFQ | QQKPGKAPKLLIYRTSNLASGVPSRFSGSGSGTDFT |
| A05 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSHY | DIQMTQSPSSLSASVGDRVTITCSARSSVSYMYWY |
| DB05_ | HYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| A06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARQ | RTSNL | CQQYH | QVQLVQSGAEVKKPGASVKVSCKASGYTFTNYY | DIQMTQSPSSLSASVGDRVTITCSARQSVSYMYWY |
| DB05_ | YYMQ | ENGNTI | YDV | SVSY | AS | SYPPTF | MQWVRQAPGQGLEWMGWIDHENGNTIYAQKFQ | QQKPGKAPKLLIYRTSNLASGVPSRFSGSGSGTDFT |
| A07 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDS | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB05_ | YYMH | NSGGT | YDV | SVSY | AS | SYPPTF | MHWVRQAPGQGLEWMGWIDSNSGGTNYAQKFQ | QQKPGKAPKLLIYRTSNLASGVPSRFSGSGSGTDFT |
| A08 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNFN | GWIDH | CARG | SARS | RTSNL | CQQYH | QVQLVQSGAEVKKPGASVKVSCKASGYNENGYY | DIQMTQSPSSLSASVGDRVTITCSARSSVSYMYWY |
| DB05_ | GYYM | EGGNTI | YDV | SVSY | AS | SYPPTF | MHWVRQAPGQGLEWMGWIDHEGGNTIYAQKFQ | QQKPGKAPKLLIYRTSNLASGVPSRFSGSGSGTDFT |
| A09 | H | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | SARS | RTSNL | CQQYH | QVQLVQSGAEVKKPGASVKVSCKASGYTFTNYYI | DIQMTQSPSSLSASVGDRVTITCSARSSVSYMYWY |
| DB05_ | YYIH | SNGNTI | YDV | SVSY | AS | SYPPTF | HWVRQAPGQGLEWMGWIDPSNGNTIYDQKFQGR | QQKPGKAPKLLIYRTSNLASGVPSRFSGSGSGTDFT |
| A10 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFTG | GWIDP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB05_ | YYMH | NSGGT | YDV | SVSY | AS | SYPPTF | MHWVRQAPGQGLEWMGWIDPNSGGTNYAQKFQ | QQKPGKAPKLLIYRTSNLASGVPSRFSGSGSGTDFT |
| A11 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | SARS | RTSNL | CQQYH | QVQLVQSGAEVKKPGASVKVSCKASGENIKDYYI | DIQMTQSPSSLSASVGDRVTITCSARSSVSYMYWY |
| DB05_ | YYIH | NSGGTI | YDV | SVSY | AS | SYPPTF | HWVRQAPGQGLEWMGWIDPNSGGTIYAQNFQGR | QQKPGKAPKLLIYRTSNLASGVPSRFSGSGSGTDFT |
| A12 | | YA | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YPFTS | GWIDH | CARG | SASS | RTSNL | CQQYH | QVQLVQSGAEVKKPGASVKVSCKASGYPFTSYY | DIQMTQSPSSLSASVGDRVTITCSASSSVSYMYWY |
| DB05_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| B01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTIKD | GWIDP | CARG | SARS | RTSNL | CQQYH | QVQLVQSGAEVKKPGASVKVSCKASGYTIKDYYI | DIQMTQSPSSLSASVGDRVTITCSARSSVSYMYWY |
| DB05_ | YYIH | ENGNTI | YDV | SVSY | AS | SYPPTF | HWVRQAPGQGLEWMGWIDPENGNTIYAQKFQG | QQKPGKAPKLLIYRTSNLASGVPSRFSGSGSGTDFT |
| B02 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWMD | CARG | SARS | RTSNL | CQQYH | QVQLVQSGAEVKKPGASVKVSCKASGYTFTSYYI | DIQMTQSPSSLSASVGDRVTITCSARSSVSYMYWY |
| DB05_ | YYIH | PNSGS | YDV | SVSY | AS | SYPPTF | HWVRQAPGQGLEWIGWMDPNSGSAAYAQKFQG | QQKPGKAPKLLIYRTSNLASGVPSRFSGSGSGTDFT |
| B03 | | AAYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTT | GWIDP | CARG | SARS | RTSNL | CQQYH | QVQLVQSGAEVKKPGASVKVSCKASGYTFTTYY | DIQMTQSPSSLSASVGDRVTITCSARSSVSYMYWY |
| DB05_ | YYMH | SNGNTI | YDV | SVSY | AS | SYPPTF | MHWVRQAPGQGLEWMGWIDPSNGNTIYDQKFQ | QQKPGKAPKLLIYRTSNLASGVPSRFSGSGSGTDFT |
| B04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | NTFSG | GWIDP | CARG | SARS | RTSNL | CQQYH | QVQLVQSGAEVKKPGASVKVSCKASGNTFSGYY | DIQMTQSPSSLSASVGDRVTITCSARSSVSYMYWY |
| DB05_ | YYVH | SNGNTI | YDV | SVSY | AS | SYPPTF | VHWVRQAPGQGLEWMGWIDPSNGNTIYDQKFQ | QQKPGKAPKLLIYRTSNLASGVPSRFSGSGSGTDFT |
| B05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YPFTN | GWIDH | CARG | SASQ | RTSNL | CQQYH | QVQLVQSGAEVKKPGASVKVSCKASGYPFTNYY | DIQMTQSPSSLSASVGDRVTITCSASQSVSYMYWY |
| DB05_ | YYMH | EGGSTI | YDV | SVSY | AS | SYPPTF | MHWVRQAPGQGLEWMGWIDHEGGSTIYDQKFQ | QQKPGKAPKLLIYRTSNLASGVPSRFSGSGSGTDFT |
| B06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNITD | GWIDH | CARG | SARS | RTSNL | CQQYH | QVQLVQSGAEVKKPGASVKVSCKASGENITDYY | DIQMTQSPSSLSASVGDRVTITCSARSSVSYMYWY |
| DB05_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| B07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | NAFSG | GWIDH | CARG | SARQ | RTSNL | CQQYH | QVQLVQSGAEVKKPGASVKVSCKASGNAFSGHY | DIQMTQSPSSLSASVGDRVTITCSARQSVSYMYWY |
| DB05_ | HYVH | ENGNTI | YDV | SVSY | AS | SYPPTF | VHWVRQAPGQGLEWMGWIDHENGNTIYAQKFQ | QQKPGKAPKLLIYRTSNLASGVPSRFSGSGSGTDFT |
| B08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB05_ | YYMH | SGGNTI | YDV | SVSY | AS | SYPPTF | MHWVRQAPGQGLEWMGWIDPSGGNTIYDQKFQ | QQKPGKAPKLLIYRTSNLASGVPSRFSGSGSGTDFT |
| B09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFSN | GWIDE | CARG | SARS | RTSNL | CQQYH | QVQLVQSGAEVKKPGASVKVSCKASGDTFSNYY | DIQMTQSPSSLSASVGDRVTITCSARSSVSYMYWY |
| DB05_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| B10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSA | GWIDH | CARG | SARS | RTSNL | CQQYH | QVQLVQSGAEVKKPGASVKVSCKASGYTFSAYY | DIQMTQSPSSLSASVGDRVTITCSARSSVSYMYWY |
| DB05_ | YYMH | ENGNTI | YDV | SVSY | AS | SYPPTF | MHWVRQAPGQGLEWMGWIDHENGNTIYAQKFQ | QQKPGKAPKLLIYRTSNLASGVPSRFSGSGSGTDFT |
| B11 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDE | CARG | SARQ | RTSNL | CQQYH | QVQLVQSGAEVKKPGASVKVSCKASGYTFTNYY | DIQMTQSPSSLSASVGDRVTITCSARQSVSYMYWY |
| DB05_ | YYMH | ENQNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| B12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKS | GWIDP | CARG | SARS | RTSNL | CQQYH | QVQLVQSGAEVKKPGASVKVSCKASGENFKSYY | DIQMTQSPSSLSASVGDRVTITCSARSSVSYMYWY |
| DB05_ | YYMH | DSGVT | YDV | SVSY | AS | SYPPTF | MHWVRQAPGQGLEWMGWIDPDSGVTNYAQKFQ | QQKPGKAPKLLIYRTSNLASGVPSRFSGSGSGTDFT |
| C01 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTS | GWIDP | CARG | SARS | RTSNL | CQQYH | QVQLVQSGAEVKKPGASVKVSCKASGDTFTSYY | DIQMTQSPSSLSASVGDRVTITCSARSSVSYMYWY |
| DB05_ | YYMH | SNGNTI | YDV | SVSY | AS | SYPPTF | MHWVRQAPGQGLEWMGWIDPSNGNTIYDQKFQ | QQKPGKAPKLLIYRTSNLASGVPSRFSGSGSGTDFT |
| C02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | RARS | RTSNL | CQQYH | QVQLVQSGAEVKKPGASVKVSCKASGYTFTNYY | DIQMTQSPSSLSASVGDRVTITCRARSSVSYMYWY |
| DB05_ | YYMH | EGGNT | YDV | SVSY | AS | SYPPTF | MHWVRQAPGQGLEWMGWIDHEGGNTSYAQKFQ | QQKPGKAPKLLIYRTSNLASGVPSRFSGSGSGTDFT |
| C03 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | SARQ | RTSNL | CQQYH | QVQLVQSGAEVKKPGASVKVSCKASGFNIKDYYI | DIQMTQSPSSLSASVGDRVTITCSARQSVSYMYWY |
| DB05_ | YYIH | ENGNTI | YDV | SVSY | QS | SYPPTF | HWVRQAPGQGLEWMGWIDPENGNTIYDQKFQG | QQKPGKAPKLLIYRTSNLQSGVPSRFSGSGSGTDFT |
| C04 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YNIKS | GWIDY | CARG | SARS | RTSNL | CQQYH | QVQLVQSGAEVKKPGASVKVSCKASGYNIKSYYI | DIQMTQSPSSLSASVGDRVTITCSARSSVSYMYWY |
| DB05_ | YYIH | NSGGT | YDV | SVSY | AS | SYPPTF | HWVRQAPGQGLEWMGWIDPNSGGTNYAQNFQG | QQKPGKAPKLLIYRTSNLASGVPSRFSGSGSGTDFT |
| C05 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | FNIKS | GWIDP | CARG | SARS | RTSNL | CQQYH | QVQLVQSGAEVKKPGASVKVSCKASGFNIKSYYI | DIQMTQSPSSLSASVGDRVTITCSARSSVSYMYWY |
| DB05_ | YYIH | NSGGT | YDV | SVSY | AS | SYPPTF | HWVRQAPGQGLEWMGWIDPNSGGTNYAQKFQG | QQKPGKAPKLLIYRTSNLASGVPSRFSGSGSGTDFT |
| C06 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDH | CARG | RARQ | RTSNL | CQQYH | QVQLVQSGAEVKKPGASVKVSCKASGYTFTSHY | DIQMTQSPSSLSASVGDRVTITCRARQSVSYMYWY |
| DB05_ | HYVH | EGGSTS | YDV | SVSY | AS | SYPPTF | VHWVRQAPGQGLEWMGWIDHEGGSTSYDQKFQ | QQKPGKAPKLLIYRTSNLASGVPSRFSGSGSGTDFT |
| C07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | RARQ | RTSNL | CQQYH | QVQLVQSGAEVKKPGASVKVSCKASGYTFTSYY | DIQMTQSPSSLSASVGDRVTITCRARQSVSYMYWY |
| DB05_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| C08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWISP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB05_ | YYMH | DSGAT | YDV | SVSY | AS | SYPPTF | MHWVRQAPGQGLEWMGWISPDSGATKVAQKFQ | QQKPGKAPKLLIYRTSNLASGVPSRFSGSGSGTDFT |
| C09 | | KVA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSS | GWIDP | CARG | RARS | RTSNL | CQQYH | QVQLVQSGAEVKKPGASVKVSCKASGYTFSSYY | DIQMTQSPSSLSASVGDRVTITCRARSSISYMYWYC |
| DB05_ | YYMH | YSGAT | YDV | SISY | AS | SYPPTF | MHWVRQAPGQGLEWMGWIDPYSGATNYAQKFQ | QKPGKAPKLLIYRTSNLASGVPSRFSGSGSGTDFTL |
| C10 | | NYA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YIFTS | GWIDP | CARG | RARS | RTSNL | CQQYH | QVQLVQSGAEVKKPGASVKVSCKASGYIFTSYYM | DIQMTQSPSSLSASVGDRVTITCRARSSISYMYWYQ |
| DB05_ | YYMH | YSGAT | YDV | SISY | AS | SYPPTF | HWVRQAPGQGLEWMGWIDPYSGATNYAQKFQG | QKPGKAPKLLIYRTSNLASGVPSRFSGSGSGTDFTL |
| C11 | | NYA | DWF | | | | RVTMTRDTSTSTVYMELSSLRSEDTAVYYCARGY | TISSLQPEDFATYYCQQYHSYPPTFGGGTKVEIKR |
| | | | VYW | | | | DVDWFVYWGQGTLVTVSS | |
| suPAR_ | GTFSS | GVINPG | CARG | QTSQ | DATNL | CQQYH | QVQLVQSGAEVKKPGASVKVSCKASGGTESSYAI | DIQMTQSPSSLSASVGDRVTITCQTSQDISNHLNWY |
| DB05_ | YAIS | GGSTT | YDV | DISN | AT | SYPPTF | SWVRQAPGQGLEWMGVINPGGGSTTYAQKFQGR | QQKPGKAPKLLIYDATNLATGVPSRFSGSGSGTDF |
| C12 | | YA | DWF | HLN | | | VTMTRDTSTSTVYMELSSLRSEDTAVYYCARGYD | LTISSLQPEDFATYYCQQYHSYPPTFGGGTKVEIKR |
| | | | VNW | | | | VDWFVNWGQGTLVTVSS | |
| suPAR_ | HTSTN | GWIDH | CARG | SARS | RTSNL | CQQYH | QVQLVQSGAEVKKPGASVKVSCKASGHTSTNYY | DIQMTQSPSSLSASVGDRVTITCSARSSVSYMYWY |
| DB05_ | YYMH | EGGNT | YDV | SVSY | AS | SYPPTF | MHWVRQAPGQGLEWMGWIDHEGGNTSYDQKFQ | QQKPGKAPKLLIYRTSNLASGVPSRFSGSGSGTDFT |
| D01 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTITG | GWIDH | CARG | SARQ | RTSNL | CQQYH | QVQLVQSGAEVKKPGASVKVSCKASGYTITGNY | DIQMTQSPSSLSASVGDRVTITCSARQSISYMYWYQ |
| DB05_ | NYMH | ENGSTI | YDV | SISY | AS | SYPPTF | MHWVRQAPGQGLEWMGWIDHENGSTIYDQKFQ | QKPGKAPKLLIYRTSNLASGVPSRFSGSGSGTDFTL |
| D02 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFGN | GWIDH | CARG | SARS | RTSNL | CQQYH | QVQLVQSGAEVKKPGASVKVSCKASGYTFGNYY | DIQMTQSPSSLSASVGDRVTITCSARSSVSYMYWY |
| DB05_ | YYMH | ENQNTI | NDV | SVSY | AS | SYPPTF | MHWVRQAPGQGLEWMGWIDHENGNTIYAQKFQ | QQKPGKAPKLLIYRTSNLASGVPSRFSGSGSGTDFT |
| D03 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | NDVDWFVYWGQGTLVTVSS | |
| suPAR_ | FNIKD | GWTYP | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB05_ | YYIH | NSGGT | YDV | SVSY | AS | SYPPTF | HWVRQAPGQGLEWMGWIYPNSGGTNYAQKFQG | QQKPGKAPKLLIYRTSNLASGVPSRFSGSGSGTDFT |
| D04 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDH | CARG | RARQ | RTSNL | CQQYH | QVQLVQSGAEVKKPGASVKVSCKASGYTFTNYY | DIQMTQSPSSLSASVGDRVTITCRARQSVSYMYWY |
| DB05_ | YYMH | SNQSTI | YDV | SVSY | AS | SYPPTF | MHWVRQAPGQGLEWMGWIDHSNGSTIYDQKFQ | QQKPGKAPKLLIYRTSNLASGVPSRFSGSGSGTDFT |
| D05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSS | GWIDH | CARG | SARS | RTSNL | CQQYH | QVQLVQSGAEVKKPGASVKVSCKASGYTFSSYY | DIQMTQSPSSLSASVGDRVTITCSARSSVSYMYWY |
| DB05_ | YYMH | ENGNTI | DDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | DDVDWFVYWGQGTLVTVSS | |
| suPAR_ | FNFTD | GWIDP | CARG | SARS | RTSNL | CQQYH | QVQLVQSGAEVKKPGASVKVSCKASGENFTDYYI | DIQMTQSPSSLSASVGDRVTITCSARSSVSYMYWY |
| DB05_ | YYIH | NNGGT | YDV | SVSY | AS | SYPPTF | HWVRQAPGQGLEWMGWIDPNNGGTKSAQKFQG | QQKPGKAPKLLIYRTSNLASGVPSRFSGSGSGTDFT |
| D07 | | KSA | DWF | MY | | | RVTMTRDTSTSTVYMELSSLRSEDTAVYYCARGY | LTISSLQPEDFATYYCQQYHSYPPTFGGGTKGGDQ |
| | | | VYW | | | | DVDWFVYWGQGTLVTVSS | T |
| suPAR_ | YTFTR | GWIQP | CARG | SARQ | RTSNL | CQQYH | QVQLVQSGAEVKKPGASVKVSCKASGYTFTRYY | DIQMTQSPSSLSASVGDRVTITCSARQSVSYMYWY |
| DB05_ | YYMH | HSGGT | YDID | SVSY | AS | SYPPTF | MHWVRQAPGQGLEWVGWIDPHSGGTNYAQKFQ | QQKPGKAPKLLIYRTSNLASGVPSRFSGSGSGTDFT |
| D08 | | NYA | WFV | MY | | | GRVTMTRDTSTSTVYMELSSLRSEDTAVYYCARG | LTISSLQPEDFATYYCQQYHSYPPTFGGGTKVEIKR |
| | | | YW | | | | YDIDWFVYWGQGTLVTVSS | |
| suPAR_ | YTFTS | GWIDP | CARG | SARS | RTSSLA | CQQYH | QVQLVQSGAEVKKPGASVKVSCKASGYTFTSYY | DIQMTQSPSSLSASVGDRVTITCSARSSVSYMYWY |
| DB05_ | YYMH | ENGNTI | YDV | SVSY | S | SYPPTF | MHWVRQAPGQGLEWMGWIDPENGNTIYAQKFQ | QQKPGKAPKLLIYRTSSLASGVPSRFSGSGSGTDFT |
| D09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSN | GWIDH | CARG | RARQ | RTSNL | CQQYH | QVQLVQSGAEVKKPGASVKVSCKASGYTFSNYYI | DIQMTQSPSSLSASVGDRVTITCRARQSVSYMYWY |
| DB05_ | YYIH | ENGNTI | TDVD | SVSY | AS | SYPPTF | HWVRQAPGQGLEWMGWIDHENGNTIYDQKFQG | QQKPGKAPKLLIYRTSNLASGVPSRFSGSGSGTDFT |
| D10 | | YD | WFV | MY | | | RVTMTRDTSTSTVYMELSSLRSEDTAVYYCARGT | LTISSLQPEDFATYYCQQYHSYPPTFGGGTKVEIKR |
| | | | YW | | | | DVDWFVYWGQGTLVTVSS | |
| suPAR_ | YTLTG | GWIDP | CARG | SASS | RTSNL | CQQYH | QVQLVQSGAEVKKPGASVKVSCKASGYTLTGYY | DIQMTQSPSSLSASVGDRVTITCSASSSVSYMYWY |
| DB05_ | YYMQ | ENGSTI | YDV | SVSY | AS | SYPPTF | MQWVRQAPGQGLEWMGWIDPENGSTIYDQKFQ | QQKPGKAPKLLIYRTSNLASGVPSRFSGSGSGTDFT |
| D11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FTFTN | GWIDP | CARG | SARS | RTSNL | CQQYH | QVQLVQSGAEVKKPGASVKVSCKASGFTFTNYYF | DIQMTQSPSSLSASVGDRVTITCSARSSVSYMYWY |
| DB05_ | YYFH | ENGNTI | YDV | SVSY | AS | SYPPTF | HWVRQAPGQGLEWMGWIDPENGNTIYDQKFQG | QQKPGKAPKLLIYRTSNLASGVPSRFSGSGSGTDFT |
| D12 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDH | CARG | RARS | RTSNL | CQQYH | QVQLVQSGAEVKKPGASVKVSCKASGYTFTNYY | DIQMTQSPSSLSASVGDRVTITCRARSSVSYMYWY |
| DB05_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDL | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB05_ | YYMH | NSGGA | YDV | SVSY | AS | SYPPTF | MHWVRQAPGQGLEWMGWIDLNSGGAKYAQRFQ | QQKPGKAPKLLIYRTSNLASGVPSRFSGSGSGTDFT |
| E02 | | KYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTLNS | GWIDP | CARG | SARS | RTSNL | CQQYH | QVQLVQSGAEVKKPGASVKVSCKASGHTLNSYYI | DIQMTQSPSSLSASVGDRVTITCSARSSVSYMYWY |
| DB05_ | YYIH | SGGNTI | YDV | SVSY | AS | SYPPTF | HWVRQAPGQGLEWMGWIDPSGGNTIYDQKFQGR | QQKPGKAPKLLIYRTSNLASGVPSRFSGSGSGTDFT |
| E03 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YSFTS | GWIDH | CARG | SARS | RTSNL | CQQYH | QVQLVQSGAEVKKPGASVKVSCKASGYSFTSNY | DIQMTQSPSSLSASVGDRVTITCSARSSVSYMYWY |
| DB05_ | NYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNFTN | GWIDH | CARG | SARS | RTSNL | CQQYH | QVQLVQSGAEVKKPGASVKVSCKASGYNFTNYY | DIQMTQSPSSLSASVGDRVTITCSARSSVSYMYWY |
| DB05_ | YYMH | SNGSTI | YDV | SVSY | AS | SYPPTF | MHWVRQAPGQGLEWMGWIDHSNGSTIYDQKFQ | QQKPGKAPKLLIYRTSNLASGVPSRFSGSGSGTDFT |
| E05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFST | GWIDH | CARG | SARS | RTSNL | CQQYH | QVQLVQSGAEVKKPGASVKVSCKASGGTFSTYYI | DIQMTQSPSSLSASVGDRVTITCSARSSVSYMYWY |
| DB05_ | YYIH | SNGNTI | YDV | SVSY | AS | SYPPTF | HWVRQAPGQGLEWMGWIDHSNGNTIYAQKFQG | QQKPGKAPKLLIYRTSNLASGVPSRFSGSGSGTDFT |
| E06 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | GTFTS | GWIDY | CARG | SARS | RTSNL | CQQYH | QVQLVQSGAEVKKPGASVKVSCKASGGTFTSYY | DIQMTQSPSSLSASVGDRVTITCSARSSVSYMYWY |
| DB05_ | YYMH | EGGNTI | YDV | SVSY | AS | SYPPTF | MHWVRQAPGQGLEWMGWIDPEGGNTIYAQKFQ | QQKPGKAPKLLIYRTSNLASGVPSRFSGSGSGTDFT |
| E07 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CAM | SARS | RTSNL | CQQYH | QVQLVQSGAEVKKPGASVKVSCKASGYTFTGYY | DIQMTQSPSSLSASVGDRVTITCSARSSVSYMYWY |
| DB05_ | YYMH | ENQNTI | GYD | SVSY | AS | SYPPTF | MHWVRQAPGQGLEWMGWIDHENGNTIYDQKFQ | QQKPGKAPKLLIYRTSNLASGVPSRFSGSGSGTDFT |
| E08 | | YD | VDW | MY | | | GRVTMTRDTSTSTVYMELSSLRSEDTAVYYCAM | LTISSLQPEDFATYYCQQYHSYPPTFGGGTKVEIKR |
| | | | FVY | | | | GYDVDWFVYWGQGTLVTVSS | |
| | | | W | | | | | |
| suPAR_ | FNIKS | GWIDP | CARG | SARQ | RTSNL | CQQYH | QVQLVQSGAEVKKPGASVKVSCKASGFNIKSYYI | DIQMTQSPSSLSASVGDRVTITCSARQSVSYMYWY |
| DB05_ | YYIH | ENGSTI | YDV | SVSY | AS | SYPPTF | HWVRQAPGQGLEWMGWIDPENGSTIYDQKFQGR | QQKPGKAPKLLIYRTSNLASGVPSRFSGSGSGTDFT |
| E09 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | DTFTN | GWIDP | CARG | SARS | RTSNL | CQQYH | QVQLVQSGAEVKKPGASVKVSCKASGDTFTNYY | DIQMTQSPSSLSASVGDRVTITCSARSSVSYMYWY |
| DB05_ | YYMH | ENQSTI | YDV | SVSY | QS | SYPPTF | MHWVRQAPGQGLEWMGWIDPENGSTIYDQKFQ | QQKPGKAPKLLIYRTSNLQSGVPSRFSGSGSGTDFT |
| E10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSHY | DIQMTQSPSSLSASVGDRVTITCSARSSVSYMYWY |
| DB05_ | HYVH | ENGSTI | YDV | SVSY | AS | SYPPTF | VHWVRQAPGQGLEWMGWIDHENGSTIYAQKFQ | QQKPGKAPKLLIYRTSNLASGVPSRFSGSGSGTDFT |
| E11 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTR | GWIDH | CARG | SARS | RTSNL | CQQYH | QVQLVQSGAEVKKPGASVKVSCKASGDTFTRHY | DIQMTQSPSSLSASVGDRVTITCSARSSVSYMYWY |
| DB05_ | HYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| E12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTS | GWIDP | CARG | SARS | RTSNL | CQQYH | QVQLVQSGAEVKKPGASVKVSCKASGDTFTSYY | DIQMTQSPSSLSASVGDRVTITCSARSSVSYMYWY |
| DB05_ | YYMH | NEGGT | YDV | SVSY | AS | SYPPTF | MHWVRQAPGQGLEWMGWIDPNSGGTNYAQKFQ | QQKPGKAPKLLIYRTSNLASGVPSRFSGSGSGTDFT |
| F01 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTR | GWIDP | CARG | RARQ | RTSNL | CQQYH | QVQLVQSGAEVKKPGASVKVSCKASGYTFTRYY | DIQMTQSPSSLSASVGDRVTITCRARQSVSYMYWY |
| DB05_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| F02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSA | GWIDH | CARG | RARQ | RTSNL | CQQYH | QVQLVQSGAEVKKPGASVKVSCKASGYTFSAYY | DIQMTQSPSSLSASVGDRVTITCRARQSVSYMYWY |
| DB05_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| F03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSG | GWIDH | CARG | SARS | RTSNL | CQQYH | QVQLVQSGAEVKKPGASVKVSCKASGYTFSGYY | DIQMTQSPSSLSASVGDRVTITCSARSSVSYMYWY |
| DB05_ | YYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| F04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | NINSY | GWIQP | CARG | SARQ | RTSNL | CQQYH | QVQLVQSGAEVKKPGASVKVSCKASGNINSYYM | DIQMTQSPSSLSASVGDRVTITCSARQSVSYMYWY |
| DB05_ | YMH | ENQNT | YDV | SVSY | AS | SYPPTF | HWVRQAPGQGLEWMGWIDPENGNTSYDQKFQG | QQKPGKAPKLLIYRTSNLASGVPSRFSGSGSGTDFT |
| F05 | | SYD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | FNITS | GWIDP | CARG | SARQ | RTSNL | CQQYH | QVQLVQSGAEVKKPGASVKVSCKASGFNITSYYI | DIQMTQSPSSLSASVGDRVTITCSARQSISYMYWYC |
| DB05_ | YYIH | SNGNTI | YDV | SISY | AS | SYPPTF | HWVRQAPGQGLEWMGWIDPSNGNTIYDQKFQGR | QKPGKAPKLLIYRTSNLASGVPSRFSGSGSGTDFTL |
| F06 | | YD | DWF | MY | | | VTMTRDTSTSTVYMELSSLRSEDTAVYYCARGYD | TISSLQPEDFATYYCQQYHSYPPTFGGGTKVEIKR |
| | | | VYW | | | | VDWFVYWGQGTLVTVSS | |
| suPAR_ | YTFSN | GWIDH | CARG | SARS | RTSNL | CQQYH | QVQLVQSGAEVKKPGASVKVSCKASGYTFSNYY | DIQMTQSPSSLSASVGDRVTITCSARSSVSYMYWY |
| DB05_ | YYMH | ENQNT | NDV | SVSY | AS | SYPPTF | MHWVRQAPGQGLEWMGWIDHENGNTSYAQKFQ | QQKPGKAPKLLIYRTSNLASGVPSRFSGSGSGTDFT |
| F07 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | NDVDWFVYWGQGTLVTVSS | |
| suPAR_ | FNFKS | GWIDP | CARG | SARS | RTSNL | CQQYH | QVQLVQSGAEVKKPGASVKVSCKASGENFKSYYI | DIQMTQSPSSLSASVGDRVTITCSARSSVSYMYWY |
| DB05_ | YYIH | NSGGT | YDV | SVSY | AS | SYPPTF | HWVRQAPGQGLEWMGWIDPNSGGTMFAQKFQG | QQKPGKAPKLLIYRTSNLASGVPSRFSGSGSGTDFT |
| F08 | | MFA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | FNIKD | GWIDP | CARG | RARQ | RTSNL | CQQYH | QVQLVQSGAEVKKPGASVKVSCKASGFNIKDYYI | DIQMTQSPSSLSASVGDRVTITCRARQSVSYMYWY |
| DB05_ | YYIH | NSGGT | YDV | SVSY | AS | SYPPTF | HWVRQAPGQGLEWMGWIDPNSGGTMFAQKFQG | QQKPGKAPKLLIYRTSNLASGVPSRFSGSGSGTDFT |
| F09 | | MFA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDH | CARG | SARQ | RTSNL | CQQYH | QVQLVQSGAEVKKPGASVKVSCKASGYTFTGYY | DIQMTQSPSSLSASVGDRVTITCSARQSISYMYWYQ |
| DB05_ | YYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| F10 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | GTFTS | GWIDH | CARG | SARS | RTSNL | CQQYH | QVQLVQSGAEVKKPGASVKVSCKASGGTFTSYYI | DIQMTQSPSSLSASVGDRVTITCSARSSVSYMYWY |
| DB05_ | YYIH | ENGSTS | YDV | SVSY | AS | SYPPTF | HWVRQAPGQGLEWMGWIDHENGSTSYDQKFQG | QQKPGKAPKLLIYRTSNLASGVPSRFSGSGSGTDFT |
| F11 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | NSFTS | GWIDR | CARG | SARS | RTSNL | CQQYH | QVQLVQSGAEVKKPGASVKVSCKASGNSFTSYY | DIQMTQSPSSLSASVGDRVTITCSARSSVSYMYWY |
| DB05_ | YYVH | ENQNTI | YDV | SVSY | AS | SYPPTF | VHWVRQAPGQGLEWMGWIDPENGNTIYAQKFQ | QQKPGKAPKLLIYRTSNLASGVPSRFSGSGSGTDFT |
| F12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| SUPAR | YTFTT | GWIDP | CARG | SARS | RTSNL | CQQYH | QVQLVQSGAEVKKPGASVKVSCKASGYTFTTYY | DIQMTQSPSSLSASVGDRVTITCSARSSVSYMYWY |
| DB05_ | YYMH | NSGGT | YDV | SVSY | AS | SYPPTF | MHWVRQAPGQGLEWMGWIDPNSGGTNYAQKFQ | QQKPGKAPKLLIYRTSNLASGVPSRFSGSGSGTDFT |
| G01 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | NTFTS | GWIDP | CARG | SARS | RTSNL | CQQYH | QVQLVQSGAEVKKPGASVKVSCKASGNTFTSYYL | DIQMTQSPSSLSASVGDRVTITCSARSSVSYMYWY |
| DB05_ | YYLH | SGGSTS | YDV | SVSY | AS | SYPPTF | HWVRQAPGQGLEWMGWIDPSGGSTSYAQKFQG | QQKPGKAPKLLIYRTSNLASGVPSRFSGSGSGTDFT |
| G02 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDH | CARG | SASS | RTSNL | CQQYH | QVQLVQSGAEVKKPGASVKVSCKASGYTFTNYY | DIQMTQSPSSLSASVGDRVTITCSASSSISYMYWYQ- |
| DB05_ | YYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| G03 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTG | GWIDH | CATG | SARS | RTSNL | CQQYH | QVQLVQSGAEVKKPGASVKVSCKASGYTFTGHY | DIQMTQSPSSLSASVGDRVTITCSARSSVSYMYWY |
| DB05_ | HYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| G04 | | YA | DWF | MY | | | GRVTMTRDTSTSTVYMELSSLRSEDTAVYYCATG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSHYI | DIQMTQSPSSLSASVGDRVTITCSARSSVSYMYWY |
| DB05_ | HYIH | ENQNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| G05 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWISP | CARG | SARS | RTSNL | CQQYH | QVQLVQSGAEVKKPGASVKVSCKASGYTFTNYYI | DIQMTQSPSSLSASVGDRVTITCSARSSVSYMYWY |
| DB05_ | YYIH | DSGGT | YDV | SVSY | AS | SYPPTF | HWVRQAPGQGLEWMGWISPDSGGTKYAQKFQG | QQKPGKAPKLLIYRTSNLASGVPSRFSGSGSGTDFT |
| G06 | | KYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTD | GWIDH | CARG | RARQ | RTSNL | CQQYH | QVQLVQSGAEVKKPGASVKVSCKASGYTFTDYY | DIQMTQSPSSLSASVGDRVTITCRARQSVSYMYWY |
| DB05_ | YYMH | ENQNTI | YDV | SVSY | AS | SYPPTF | MHWVRQAPGQGLEWMGWIDHENGNTIYAQKFQ | QQKPGKAPKLLIYRTSNLASGVPSRFSGSGSGTDFT |
| G07 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGHY | DIQMTQSPSSLSASVGDRVTITCSARSSVSYMYWY |
| DB05_ | HYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| G08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTR | GWIDP | CARG | SARS | RTSSLQ | CQQYH | QVQLVQSGAEVKKPGASVKVSCKASGDTFTRYY | DIQMTQSPSSLSASVGDRVTITCSARSSVSYMYWY |
| DB05_ | YYMH | NSGAT | YDV | SVSY | S | SYPPTF | MHWVRQAPGQGLEWMGWIDPNSGATNYAQKFQ | QQKPGKAPKLLIYRTSSLQSGVPSRFSGSGSGTDFT |
| G09 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNIKD | GWIDP | CARG | SARQ | RTSNL | CQQYH | QVQLVQSGAEVKKPGASVKVSCKASGYNIKDYYI | DIQMTQSPSSLSASVGDRVTITCSARQSVSYMYWY |
| DB05_ | YYIH | SGGNTI | YDV | SVSY | AS | SYPPTF | HWVRQAPGQGLEWMGWIDPSGGNTIYDQKFQGR | QQKPGKAPKLLIYRTSNLASGVPSRFSGSGSGTDFT |
| G10 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YKFTS | GWIDH | CARG | RARQ | RTSNL | CQQYH | QVQLVQSGAEVKKPGASVKVSCKASGYKFTSYY | DIQMTQSPSSLSASVGDRVTITCRARQSVSYMYWY |
| DB05_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFSN | GWIDP | CARG | SARS | RTSNL | CQQYH | QVQLVQSGAEVKKPGASVKVSCKASGGTFSNYY | DIQMTQSPSSLSASVGDRVTITCSARSSVSYMYWY |
| DB05_ | YYMH | EGGNTI | YDV | SVSY | AS | SYPPTF | MHWVRQAPGQGLEWMGWIDPEGGNTIYAQKFQ | QQKPGKAPKLLIYRTSNLASGVPSRFSGSGSGTDFT |
| G12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YAFTS | GWIDP | CARG | RARS | RTSNL | CQQYH | QVQLVQSGAEVKKPGASVKVSCKASGYAFTSYY | DIQMTQSPSSLSASVGDRVTITCRARSSVSYMYWY |
| DB05_ | YYMH | NSGGT | YDV | SVSY | AS | SYPPTF | MHWVRQAPGQGLEWMGWIDPNSGGTNYAQKFQ | QQKPGKAPKLLIYRTSNLASGVPSRFSGSGSGTDFT |
| H01 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTA | GWIDP | CARG | SARS | RTSNL | CQQYH | QVQLVQSGAEVKKPGASVKVSCKASGYTFTAYYI | DIQMTQSPSSLSASVGDRVTITCSARSSVSYMYWY |
| DB05_ | YYIH | HSGGT | YDV | SVSY | AS | SYPPTF | HWVRQAPGQGLEWVGWIDPHSGGTNYAQKFQG | QQKPGKAPKLLIYRTSNLASGVPSRFSGSGSGTDFT |
| H02 | | NYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDH | CARG | SASS | RTSNL | CQQYH | QVQLVQSGAEVKKPGASVKVSCKASGYTFTSYY | DIQMTQSPSSLSASVGDRVTITCSASSSVSYMYWY |
| DB05_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| H03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKD | GWIDY | CARG | SARS | RTSNL | CQQYH | QVQLVQSGAEVKKPGASVKVSCKASGFNFKDYY | DIQMTQSPSSLSASVGDRVTITCSARSSVSYMYWY |
| DB05_ | YYMH | EGGNTI | YDV | SVSY | AS | SYPPTF | MHWVRQAPGQGLEWMGWIDPEGGNTIYAQKFQ | QQKPGKAPKLLIYRTSNLASGVPSRFSGSGSGTDFT |
| H04 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFIG | GWIDH | CARG | SARS | RTSNL | CQQYH | QVQLVQSGAEVKKPGASVKVSCKASGYTFIGHYV | DIQMTQSPSSLSASVGDRVTITCSARSSVSYMYWY |
| DB05_ | HYVH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| H05 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDY | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB05_ | YYMH | HSGSTS | YDV | SVSY | AS | SYPPTF | MHWVRQAPGQGLEWVGWIDPHSGSTSYAQKFQ | QQKPGKAPKLLIYRTSNLASGVPSRFSGSGSGTDFT |
| H06 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTFTS | GWIDH | CARG | SARS | RTSNL | CQQYH | QVQLVQSGAEVKKPGASVKVSCKASGHTFTSYYT | DIQMTQSPSSLSASVGDRVTITCSARSSVSYMYWY |
| DB05_ | YYTH | SGGNTI | YDV | SVSY | AS | SYPPTF | HWVRQAPGQGLEWMGWIDHSGGNTIYDQKFQG | QQKPGKAPKLLIYRTSNLASGVPSRFSGSGSGTDFT |
| H07 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB05_ | YYMH | SNGNTI | YDV | SVSY | AS | SYPPTF | MHWVRQAPGQGLEWMGWIDPSNGNTIYDQKFQ | QQKPGKAPKLLIYRTSNLASGVPSRFSGSGSGTDFT |
| H08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTLTS | GWIDP | CARG | SARS | RTSNL | CQQYH | QVQLVQSGAEVKKPGASVKVSCKASGYTLTSYY | DIQMTQSPSSLSASVGDRVTITCSARSSVSYMYWY |
| DB05_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| H09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | RARQ | RTSNL | CQQYH | QVQLVQSGAEVKKPGASVKVSCKASGYTFTNYY | DIQMTQSPSSLSASVGDRVTITCRARQSVSYMYWY |
| DB05_ | YYVH | NSGGT | YDV | SVSY | AS | SYPPTF | VHWVRQAPGQGLEWMGWIDPNSGGTKYAQKFQ | QQKPGKAPKLLIYRTSNLASGVPSRFSGSGSGTDFT |
| H10 | | KYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFIN | GWIDH | CARG | SARS | RTSNL | CQQYH | QVQLVQSGAEVKKPGASVKVSCKASGDTFINYY | DIQMTQSPSSLSASVGDRVTITCSARSSVSYMYWY |
| DB06_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| A03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SASQ | RTSNL | CQQYH | QVQLVQSGAEVKKPGASVKVSCKASGYTFTNYY | DIQMTQSPSSLSASVGDRVTITCSASQSVSYMYWY |
| DB06_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| A04 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB06_ | YYVH | NSGGT | YDV | SVSY | AS | SYPPTF | VHWVRQAPGQGLEWVGWIDPNSGGTNYAQKFQ | QQKPGKAPKLLIYRTSNLASGVPSRFSGSGSGTDFT |
| A05 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDP | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB06_ | YYVH | ENGSTI | YDV | SVSY | AS | SYPPTF | VHWVRQAPGQGLEWMGWIDPENGSTIYDQKFQG | QQKPGKAPKLLIYRTSNLASGVPSRFSGSGSGTDFT |
| A06 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIDY | CARG | SARS | RTSNL | CQQYH | QVQLVQSGAEVKKPGASVKVSCKASGYTFTSYYI | DIQMTQSPSSLSASVGDRVTITCSARSSVSYMYWY |
| DB06_ | YYIH | ENQSTI | YDV | SVSY | AS | SYPPTF | HWVRQAPGQGLEWMGWIDPENGSTIYDQKFQGR | QQKPGKAPKLLIYRTSNLASGVPSRFSGSGSGTDFT |
| A07 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFTG | GWIDS | CARG | SASS | RTSNL | CQQYH | QVQLVQSGAEVKKPGASVKVSCKASGYTFTGYYI | DIQMTQSPSSLSASVGDRVTITCSASSSVSYMYWY |
| DB06_ | YYIH | HSGGT | YDV | SVSY | AS | SYPPTF | HWVRQAPGQGLEWMGWIDSHSGGTKSAQKFQG | QQKPGKAPKLLIYRTSNLASGVPSRFSGSGSGTDFT |
| A08 | | KSA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | DTFNN | GWIDP | CARG | SARQ | RTSNL | CQQYH | QVQLVQSGAEVKKPGASVKVSCKASGDTENNYY | DIQMTQSPSSLSASVGDRVTITCSARQSVSYMYWY |
| DB06_ | YYVH | EGGNT | YDID | SVSY | AS | SYPPTF | VHWVRQAPGQGLEWMGWIDPEGGNTSYDQKFQ | QQKPGKAPKLLIYRTSNLASGVPSRFSGSGSGTDFT |
| A09 | | SYD | WFV | MY | | | GRVTMTRDTSTSTVYMELSSLRSEDTAVYYCARG | LTISSLQPEDFATYYCQQYHSYPPTFGGGTKVEIKR |
| | | | YW | | | | YDIDWFVYWGQGTLVTVSS | |
| suPAR_ | DTFTS | GWID | CARG | SARS | RTSNL | CQQYH | QVQLVQSGAEVKKPGASVKVSCKASGDTFTSYY | DIQMTQSPSSLSASVGDRVTITCSARSSVSYMYWY |
| DB06_ | YYMH | SSGAT | YDV | SVSY | AS | SYPPTF | MHWVRQAPGQGLEWMGWIDPSSGATNYAQKLQ | QQKPGKAPKLLIYRTSNLASGVPSRFSGSGSGTDFT |
| A10 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DAFTS | GWIDH | CARG | SARS | RTSNL | CQQYH | QVQLVQSGAEVKKPGASVKVSCKASGDAFTSYY | DIQMTQSPSSLSASVGDRVTITCSARSSVSYMYWY |
| DB06_ | YYYH | ENGNTI | YDV | SVSY | AS | SYPPTF | YHWVRQAPGQGLEWMGWIDHENGNTIYDQKFQ | QQKPGKAPKLLIYRTSNLASGVPSRFSGSGSGTDFT |
| A11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTD | GWVSP | CASG | SASS | RTSNL | CQQYH | QVQLVQSGAEVKKPGASVKVSCKASGYTFTDYY | DIQMTQSPSSLSASVGDRVTITCSASSSVSYMYWY |
| DB06_ | YYMH | SSGNT | YDV | SVSY | AS | SYPPTF | MHWVRQAPGQGLEWMGWVSPSSGNTAYAQKFQ | QQKPGKAPKLLIYRTSNLASGVPSRFSGSGSGTDFT |
| A12 | | AYA | DWF | MY | | | GRVTMTRDTSTSTVYMELSSLRSEDTAVYYCASG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSHY | DIQMTQSPSSLSASVGDRVTITCSARSSVSYMYWY |
| DB06_ | HYVH | ENGSTI | YDV | SVSY | AS | SYPPTF | VHWVRQAPGQGLEWMGWIDHENGSTIYDQKFQ | QQKPGKAPKLLIYRTSNLASGVPSRFSGSGSGTDFT |
| B01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | RARQ | RTSNL | CQQYH | QVQLVQSGAEVKKPGASVKVSCKASGYTFTNYY | DIQMTQSPSSLSASVGDRVTITCRARQSVSYMYWY |
| DB06_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| B02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | DB06 | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB06_ | YYMH | HSGGT | YDV | SVSY | AS | SYPPTF | MHWVRQAPGQGLEWMGWIDPHSGGTNYAQKFQ | QQKPGKAPKLLIYRTSNLASGVPSRFSGSGSGTDFT |
| B03 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDY | CARG | SASS | RTSNL | CQQYH | QVQLVQSGAEVKKPGASVKVSCKASGYTFTGYY | DIQMTQSPSSLSASVGDRVTITCSASSSVSYMYWY |
| DB06_ | YYMH | ENGSTI | YDV | SVSY | AS | SYPPTF | MHWVRQAPGQGLEWMGWIDPENGSTIYDQKFQ | QQKPGKAPKLLIYRTSNLASGVPSRFSGSGSGTDFT |
| B04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNETD | GWIDP | CARG | SARS | RTSNL | CQQYH | QVQLVQSGAEVKKPGASVKVSCKASGENITDYYE | DIQMTQSPSSLSASVGDRVTITCSARSSVSYMYWY |
| DB06_ | YYIH | ENQSTI | YDV | SVSY | QS | SYPPTF | HWVRQAPGQGLEWMGWIDPENGSTIYDQKFQGR | QQKPGKAPKLLIYRTSNLQSGVPSRFSGSGSGTDFT |
| B05 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | FTFTS | GWIDH | CARG | RARQ | RTSNL | CQQYH | QVQLVQSGAEVKKPGASVKVSCKASGFTFTSYYV | DIQMTQSPSSLSASVGDRVTITCRARQSVSYMYWYQ |
| DB06_ | YYVH | ENGNTI | TDVD | SVSY | AS | SYPPTF | HWVRQAPGQGLEWMGWIDHENGNTIYDQKFQG | QKPGKAPKLLIYRTSNLASGVPSRFSGSGSGTDFT |
| B06 | | YD | WFV | MY | | | RVTMTRDTSTSTVYMELSSLRSEDTAVYYCARGT | LTISSLQPEDFATYYCQQYHSYPPTFGGGTKVEIKR |
| | | | YW | | | | DVDWFVYWGQGTLVTVSS | |
| suPAR_ | YTFTS | GWIDH | CARG | SARS | RTSNL | CQQYH | QVQLVQSGAEVKKPGASVKVSCKASGYTFTSNY | DIQMTQSPSSLSASVGDRVTITCSARSSVSYMYWY |
| DB06_ | NYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| B07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTA | GWIDY | CARG | SARQ | RTSNL | CQQYH | QVQLVQSGAEVKKPGASVKVSCKASGYTFTAYY | DIQMTQSPSSLSASVGDRVTITCSARQSISYMYWYQ |
| DB06_ | YYMH | ENGNTI | YDV | SISY | AS | SYPPTF | MHWVRQAPGQGLEWMGWIDPENGNTIYAQKFQ | QKPGKAPKLLIYRTSNLASGVPSRFSGSGSGTDFTL |
| B08 | | YA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTS | GWIDH | CASG | SARS | RTSNL | CQQYH | QVQLVQSGAEVKKPGASVKVSCKASGYTFTSYY | DIQMTQSPSSLSASVGDRVTITCSARSSISYMYWYQ |
| DB06_ | YYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| B09 | | YD | DWF | MY | | | GRVTMTRDTSTSTVYMELSSLRSEDTAVYYCASG | TISSLQPEDFATYYCQQYHSYPPTFGGGTKVEIKR |
| | | | VYW | | | | YDVDWFVYWGQGTLVTVSS | |
| suPAR_ | DTLTN | GWIDH | CARG | SARS | RTSNL | CQQYH | QVQLVQSGAEVKKPGASVKVSCKASGDTLTNYY | DIQMTQSPSSLSASVGDRVTITCSARSSVSYMYWY |
| DB06_ | YYMH | ENQSTI | YDV | SVSY | QT | SYPPTF | MHWVRQAPGQGLEWMGWIDHENGSTIYAQKFQ | QQKPGKAPKLLIYRTSNLQTGVPSRFSGSGSGTDFT |
| B10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CAEG | RARQ | RTSNL | CQQYH | QVQLVQSGAEVKKPGASVKVSCKASGYTFTSNYI | DIQMTQSPSSLSASVGDRVTITCRARQSVSYMYWY |
| DB06_ | NYIH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| B11 | | YD | DWF | MY | | | RVTMTRDTSTSTVYMELSSLRSEDTAVYYCAEGY | LTISSLQPEDFATYYCQQYHSYPPTFGGGTKVEIKR |
| | | | VYW | | | | DVDWFVYWGQGTLVTVSS | |
| suPAR_ | YTFTN | GWIDE | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB06_ | YYMH | EGGNTI | YDV | SVSY | AS | SYPPTF | MHWVRQAPGQGLEWMGWIDPEGGNTIYDQKFQ | QQKPGKAPKLLIYRTSNLASGVPSRFSGSGSGTDFT |
| B12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDQW | CARG | SARQ | RTSNL | CQQYH | QVQLVQSGAEVKKPGASVKVSCKASGYTFTGYY | DIQMTQSPSSLSASVGDRVTITCSARQSISYMYWYQ |
| DB06_ | YYMH | SNGNTI | YDV | SISY | AS | SYPPTF | MHWVRQAPGQGLEWMGWIDPSNGNTIYDQKFQ | QKPGKAPKLLIYRTSNLASGVPSRFSGSGSGTDFTL |
| C01 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | SARS | RTSNL | CQQYH | QVQLVQSGAEVKKPGASVKVSCKASGYTFTNYYI | DIQMTQSPSSLSASVGDRVTITCSARSSVSYMYWY |
| DB06_ | YYIH | EGGNTI | YDV | SVSY | AS | SYPPTF | HWVRQAPGQGLEWMGWIDPEGGNTIYAQKFQG | QQKPGKAPKLLIYRTSNLASGVPSRFSGSGSGTDFT |
| C02 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGILVTVSS | |
| suPAR_ | YTFTS | GWIDP | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB06_ | YYMH | NSGGT | YDV | SVSY | AS | SYPPTF | MHWVRQAPGQGLEWMGWIDPNSGGTNYAQKFQ | QQKPGKAPKLLIYRTSNLASGVPSRFSGSGSGTDFT |
| C03 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKS | GWIDY | CARG | SASS | RTSNL | CQQYH | QVQLVQSGAEVKKPGASVKVSCKASGFNFKSYYI | DIQMTQSPSSLSASVGDRVTITCSASSSVSYMYWY |
| DB06_ | YYIH | EGGSTI | YDV | SVSY | AS | SYPPTF | HWVRQAPGQGLEWMGWIDPEGGSTIYDQKFQGR | QQKPGKAPKLLIYRTSNLASGVPSRFSGSGSGTDFT |
| C04 | | YD | DWF | MY | | | VIMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | GTFSS | GVIDPS | CARG | RTSQ | DVSHL | CQQYH | QVQLVQSGAEVKKPGASVKVSCKASGGTFSSYAI | DIQMTQSPSSLSASVGDRVTITCRTSQSISSFLNWY |
| DB06_ | YAIS | GGTTD | YDV | SISSF | ES | SYPPTF | SWVRQAPGQGLEWMGVIDPSGGTTDYAQKFQGR | QQKPGKAPKLLIYDVSHLESGVPSRFSGSGSGTDFTL |
| C05 | | YA | DWF | LN | | | VTMTRDTSTSTVYMELSSLRSEDTAVYYCARGYD | TISSLQPEDFATYYCQQYHSYPPTFGGGTKVEIKR |
| | | | VYW | | | | VDWFVYWGQGTLVTVSS | |
| suPAR_ | YTFSN | GWIDH | CARG | SARS | RTSNL | CQQYH | QVQLVQSGAEVKKPGASVKVSCKASGYTFSNYY | DIQMTQSPSSLSASVGDRVTITCSARSSVSYMYWY |
| DB06_ | YYMH | EGGNT | YDV | SVSY | AS | SYPPTF | MHWVRQAPGQGLEWMGWIDHEGGNTSYAQKFQ | QQKPGKAPKLLIYRTSNLASGVPSRFSGSGSGTDFT |
| C06 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB06_ | YYMH | NGGGT | YDV | SVSY | AS | SYPPTF | MHWVRQAPGQGLEWMGWIDPNGGGTQYAQKF | QQKPGKAPKLLIYRTSNLASGVPSRFSGSGSGTDFT |
| C07 | | QYA | DWF | MY | | | QGRVTMTRDTSTSTVYMELSSLRSEDTAVYYCAR | LTISSLQPEDFATYYCQQYHSYPPTFGGGTKVEIKR |
| | | | VYW | | | | GYDVDWFVYWGQGTLVTVSS | |
| suPAR_ | YTFTG | GWIDH | CARG | RARS | RTSNL | CQQYH | QVQLVQSGAEVKKPGASVKVSCKASGYTFTGNY | DIQMTQSPSSLSASVGDRVTITCRARSSVSYMYWY |
| DB06_ | NYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| C08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWID | CARG | RARS | RTSNL | CQQYH | QVQLVQSGAEVKKPGASVKVSCKASGYTFTSYY | DIQMTQSPSSLSASVGDRVTITCRARSSVSYMYWY |
| DB06_ | YYMH | ENGNTI | YDV | SVSY | AS | SYPPTF | MHWVRQAPGQGLEWMGWIDPENGNTIYDQKFQ | QQKPGKAPKLLIYRTSNLASGVPSRFSGSGSGTDFT |
| C09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSA | GWIDP | CARG | SARS | RTSNL | CQQYH | QVQLVQSGAEVKKPGASVKVSCKASGYTFSAYY | DIQMTQSPSSLSASVGDRVTITCSARSSVSYMYWY |
| DB06_ | YYMH | SNQNTI | YDV | SVSY | AS | SYPPTF | MHWVRQAPGQGLEWMGWIDPSNGNTIYDQKFQ | QQKPGKAPKLLIYRTSNLASGVPSRFSGSGSGTDFT |
| C10 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARQ | RTSNL | CQQYH | QVQLVQSGAEVKKPGASVKVSCKASGYTFTNYY | DIQMTQSPSSLSASVGDRVTITCSARQSVSYMYWY |
| DB06_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| C11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTA | GWIDH | CAN | SARS | RTSNL | CQQYH | QVQLVQSGAEVKKPGASVKVSCKASGYSFTANYI | DIQMTQSPSSLSASVGDRVTITCSARSSVSYMYWY |
| DB06_ | NYIH | ENGNTI | GYD | SVSY | AS | SYPPTF | HWVRQAPGQGLEWMGWIDHENGNTIYDQKFQG | QQKPGKAPKLLIYRTSNLASGVPSRFSGSGSGTDFT |
| C12 | | YD | VDW | MY | | | RVTMTRDTSTSTVYMELSSLRSEDTAVYYCANGY | LTISSLQPEDFATYYCQQYHSYPPTFGGGTKVEIKR |
| | | | FVY | | | | DVDWFVYWGQGTLVTVSS | |
| | | | W | | | | | |
| suPAR_ | FNFKD | GWIDY | CARG | SARS | RTSNL | CQQYH | QVQLVQSGAEVKKPGASVKVSCKASGENFKDYYI | DIQMTQSPSSLSASVGDRVTITCSARSSVSYMYWY |
| DB06_ | YYIH | ENGNTI | YDV | SVSY | AS | SYPPTF | HWVRQAPGQGLEWMGWIDPENGNTIYAQKFQG | QQKPGKAPKLLIYRTSNLASGVPSRFSGSGSGTDFT |
| D01 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| SUPAR | YTFTS | GWIDH | CARG | SASS | RTSNL | CQQYH | QVQLVQSGAEVKKPGASVKVSCKASGYTFTSNY | DIQMTQSPSSLSASVGDRVTITCSASSSVSYMYWY |
| DB06_ | NYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFTD | GWISP | CARG | SARS | RTSNL | CQQYH | QVQLVQSGAEVKKPGASVKVSCKASGENFTDYYI | DIQMTQSPSSLSASVGDRVTITCSARSSVSYMYWY |
| DB06_ | YYIH | DSGAT | YDV | SVSY | AS | SYPPTF | HWVRQAPGQGLEWMGWISPDSGATKVAQKFQG | QQKPGKAPKLLIYRTSNLASGVPSRFSGSGSGTDFT |
| D03 | | KVA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFSS | GWIDH | CARG | SARS | RTSNL | CQQYH | QVQLVQSGAEVKKPGASVKVSCKASGYTESSYY | DIQMTQSPSSLSASVGDRVTITCSARSSVSYMYWY |
| DB06_ | YYMH | ENGNTI | YDV | SVSY | AS | SYPPTF | MHWVRQAPGQGLEWMGWIDHENGNTIYAQKFQ | QQKPGKAPKLLIYRTSNLASGVPSRFSGSGSGTDFT |
| D04 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTG | GWIDP | CARG | SARQ | RTSNL | CQQYH | QVQLVQSGAEVKKPGASVKVSCKASGGTFTGYY | DIQMTQSPSSLSASVGDRVTITCSARQSVSYMYWY |
| DB06_ | YYMH | SGGNTI | YDV | SVSY | AS | SYPPTF | MHWVRQAPGQGLEWMGWIDPSGGNTIYDQKFQ | QQKPGKAPKLLIYRTSNLASGVPSRFSGSGSGTDFT |
| D05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB06_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| D06 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB06_ | YYMH | EGGNTI | YDV | SVSY | AS | SYPPTF | MHWVRQAPGQGLEWMGWIDHEGGNTIYAQKFQ | QQKPGKAPKLLIYRTSNLASGVPSRFSGSGSGTDFT |
| D07 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | DB06 | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB06_ | YYMH | EGGNT | YDV | SVSY | AS | SYPPTF | MHWVRQAPGQGLEWMGWIDPEGGNTSYDQKFQ | QQKPGKAPKLLIYRTSNLASGVPSRFSGSGSGTDFT |
| D08 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTS | GWIDH | CARG | SASS | RTSNL | CQQYH | QVQLVQSGAEVKKPGASVKVSCKASGGTFTSYY | DIQMTQSPSSLSASVGDRVTITCSASSSVSYMYWY |
| DB06_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| D09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB06_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| D10 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFAN | GMIDE | CARG | RARQ | RTSNL | CQQYH | QVQLVQSGAEVKKPGASVKVSCKASGYTFANYY | DIQMTQSPSSLSASVGDRVTITCRARQSVSYMYWY |
| DB06_ | YYVH | GGGTT | YDV | SVSY | AS | SYPPTF | VHWVRQAPGQGLEWVGMIDPGGGTTNYAQKFQ | QQKPGKAPKLLIYRTSNLASGVPSRFSGSGSGTDFT |
| D11 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFSS | GWIDP | CARG | SARQ | RTSSLA | CQQYH | QVQLVQSGAEVKKPGASVKVSCKASGGTFSSYY | DIQMTQSPSSLSASVGDRVTITCSARQSVSYMYWY |
| DB06_ | YYMH | ENQNTI | YDV | SVSY | S | SYPPTF | MHWVRQAPGQGLEWMGWIDPENGNTIYAQKFQ | QQKPGKAPKLLIYRTSSLASGVPSRFSGSGSGTDFT |
| D12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKD | GWMD | CARG | SARQ | RTSNL | CQQYH | QVQLVQSGAEVKKPGASVKVSCKASGFNFKDYYI | DIQMTQSPSSLSASVGDRVTITCSARQSISYMYWYQ |
| DB06_ | YYIH | PSSGNT | YDV | SISY | AS | SYPPTF | HWVRQAPGQGLEWMGWMDPSSGNTAYAQKFQ | QKPGKAPKLLIYRTSNLASGVPSRFSGSGSGTDFTL |
| E01 | | AYA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YSFTN | GWIDH | CARG | SARS | RTSNL | CQQYH | QVQLVQSGAEVKKPGASVKVSCKASGYSFTNYY | DIQMTQSPSSLSASVGDRVTITCSARSSVSYMYWY |
| DB06_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| E02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB06_ | YYIH | ENQNT | YDV | SVSY | AS | SYPPTF | HWVRQAPGQGLEWMGWIDPENGNTSYDQKFQG | QQKPGKAPKLLIYRTSNLASGVPSRFSGSGSGTDFT |
| E03 | | SYD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | DTFSS | GWIDH | CARG | RARS | RTSNL | CQQYH | QVQLVQSGAEVKKPGASVKVSCKASGDTFSSYY | DIQMTQSPSSLSASVGDRVTITCRARSSVSYMYWY |
| DB06_ | YYMH | ENGNTI | YDM | SVSY | QS | SYPPTF | MHWVRQAPGQGLEWMGWIDHENGNTIYDQKFQ | QQKPGKAPKLLIYRTSNLQSGVPSRFSGSGSGTDFT |
| E04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDMDWFVYWGQGTLVTVSS | |
| suPAR_ | YTFTS | GWIDH | CARG | SARS | RTSNL | CQQYH | QVQLVQSGAEVKKPGASVKVSCKASGYTFTSHYI | DIQMTQSPSSLSASVGDRVTITCSARSSVSYMYWY |
| DB06_ | HYIQ | ENGNTI | TDVD | SVSY | AS | SYPPTF | QWVRQAPGQGLEWMGWIDHENGNTIYDQKFQG | QQKPGKAPKLLIYRTSNLASGVPSRFSGSGSGTDFT |
| E05 | | YD | WFV | MY | | | RVTMTRDTSTSTVYMELSSLRSEDTAVYYCARGT | LTISSLQPEDFATYYCQQYHSYPPTFGGGTKVEIKR |
| | | | YW | | | | DVDWFVYWGQGTLVTVSS | |
| suPAR_ | DTSTS | GWIDH | CARG | SARS | RTSNL | CQQYH | QVQLVQSGAEVKKPGASVKVSCKASGDTSTSYY | DIQMTQSPSSLSASVGDRVTITCSARSSVSYMYWY |
| DB06_ | YYMH | ENGSTI | YDV | SVSY | AS | SYPPTF | MHWVRQAPGQGLEWMGWIDHENGSTIYDQKFQ | QQKPGKAPKLLIYRTSNLASGVPSRFSGSGSGTDFT |
| E06 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CASG | SARS | RTSNL | CQQYH | QVQLVQSGAEVKKPGASVKVSCKASGYTFTNYY | DIQMTQSPSSLSASVGDRVTITCSARSSVSYMYWY |
| DB06_ | YYLH | HSGGT | YDV | SVSY | AS | SYPPTF | LHWVRQAPGQGLEWMGWIDPHSGGTNYAQKFQ | QQKPGKAPKLLIYRTSNLASGVPSRFSGSGSGTDFT |
| E07 | | NYA | DWF | MY | | | GRVTMTRDTSTSTVYMELSSLRSEDTAVYYCASG | LTISSLQPEDFATYYCQQYHSYPPTFGGGTKVEIKR |
| | | | VYW | | | | YDVDWFVYWGQGTLVTVSS | |
| suPAR_ | YGFTD | GWIDH | CARG | SARQ | RTSNL | CQQYH | QVQLVQSGAEVKKPGASVKVSCKASGYGFTDYY | DIQMTQSPSSLSASVGDRVTITCSARQSVSYMYWY |
| DB06_ | YYMH | SGGNTI | YDV | SVSY | AS | SYPPTF | MHWVRQAPGQGLEWMGWIDHSGGNTIYAQKFQ | QQKPGKAPKLLIYRTSNLASGVPSRFSGSGSGTDFT |
| E08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTFTN | GWIDH | CARG | SARS | RTSNL | CQQYH | QVQLVQSGAEVKKPGASVKVSCKASGHTFTNYY | DIQMTQSPSSLSASVGDRVTITCSARSSVSYMYWY |
| DB06_ | YYVH | ENGSTS | YDV | SVSY | AS | SYPPTF | VHWVRQAPGQGLEWMGWIDHENGSTSYDQKFQ | QQKPGKAPKLLIYRTSNLASGVPSRFSGSGSGTDFT |
| E09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFIS | GWIDP | CARG | SARS | RTSNL | CQQYH | QVQLVQSGAEVKKPGASVKVSCKASGDTFISYYM | DIQMTQSPSSLSASVGDRVTITCSARSSVSYMYWY |
| DB06_ | YYMH | ENQNTI | YDV | SVSY | AS | SYPPTF | HWVRQAPGQGLEWMGWIDPENGNTIYDQKFQG | QQKPGKAPKLLIYRTSNLASGVPSRFSGSGSGTDFT |
| E10 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDH | CARG | SASS | RTSNL | CQQYH | QVQLVQSGAEVKKPGASVKVSCKASGYTFTGYY | DIQMTQSPSSLSASVGDRVTITCSASSSVSYMYWY |
| DB06_ | YYMH | ENGNTI | YDV | SVSY | AS | SYPPTF | MHWVRQAPGQGLEWMGWIDHENGNTIYAQKFQ | QQKPGKAPKLLIYRTSNLASGVPSRFSGSGSGTDFT |
| E11 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RARS | RTSNL | CQQYH | QVQLVQSGAEVKKPGASVKVSCKASGYTFTSYY | DIQMTQSPSSLSASVGDRVTITCRARSSVSYMYWY |
| DB06_ | YYMH | ENGNT | YDV | SVSY | AS | SYPPTF | MHWVRQAPGQGLEWMGWIDHENGNTSYAQKFQ | QQKPGKAPKLLIYRTSNLASGVPSRFSGSGSGTDFT |
| E12 | | SYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTS | GWIDP | CARG | SARS | RTSNL | CQQYH | QVQLVQSGAEVKKPGASVKVSCKASGGTFTSYY | DIQMTQSPSSLSASVGDRVTITCSARSSVSYMYWYQQ |
| DB06_ | YYVH | EGGNT | YDV | SVSY | AS | SYPPTF | VHWVRQAPGQGLEWMGWIDPEGGNTSYDQKFQ | KPGKAPKLLIYRTSNLASGVPSRFSGSGSGTDFT |
| F01 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | RARQ | RTSNL | CQQYH | QVQLVQSGAEVKKPGASVKVSCKASGYTFTNYY | DIQMTQSPSSLSASVGDRVTITCRARQSVSYMYWY |
| DB06_ | YYMH | ENGSTS | YDV | SVSY | AS | SYPPTF | MHWVRQAPGQGLEWMGWIDHENGSTSYDQKFQ | QQKPGKAPKLLIYRTSNLASGVPSRFSGSGSGTDFT |
| F02 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTN | GWIDH | CARG | SASQ | RTSNL | CQQYH | QVQLVQSGAEVKKPGASVKVSCKASGYSFTNYY | DIQMTQSPSSLSASVGDRVTITCSASQSVSYMYWY |
| DB06_ | YYMH | ENGNTI | NDV | SVSY | AS | SYPPTF | MHWVRQAPGQGLEWMGWIDHENGNTIYDQKFQ | QQKPGKAPKLLIYRTSNLASGVPSRFSGSGSGTDFT |
| F03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | NDVDWFVYWGQGTLVTVSS | |
| suPAR_ | YRFTS | GWIDP | CARG | SARQ | RTSNL | CQQYH | QVQLVQSGAEVKKPGASVKVSCKASGYRFTSYY | DIQMTQSPSSLSASVGDRVTITCSARQSVSYMYWY |
| DB06_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| F04 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DSFTG | GWIDH | CARG | SARS | RTSNL | CQQYH | QVQLVQSGAEVKKPGASVKVSCKASGDSFTGYYI | DIQMTQSPSSLSASVGDRVTITCSARSSVSYMYWY |
| DB06_ | YYIH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| F05 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | GTFIG | GWIDH | CARG | SARS | RTSNL | CQQYH | QVQLVQSGAEVKKPGASVKVSCKASGGTFIGHYV | DIQMTQSPSSLSASVGDRVTITCSARSSVSYMYWY |
| DB06_ | HYVH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| F06 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB06_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| F07 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGNY | DIQMTQSPSSLSASVGDRVTITCSARSSVSYMYWY |
| DB06_ | NYVH | SNGNTI | YDV | SVSY | AS | SYPPTF | VHWVRQAPGQGLEWMGWIDHSNGNTIYDQKFQ | QQKPGKAPKLLIYRTSNLASGVPSRFSGSGSGTDFT |
| F08 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTT | GWIDY | CARG | SASQ | RTSNL | CQQYH | QVQLVQSGAEVKKPGASVKVSCKASGYTFTTYY | DIQMTQSPSSLSASVGDRVTITCSASQSVSYMYWY |
| DB06_ | YYMH | EGGNTI | YDV | SVSY | AS | SYPPTF | MHWVRQAPGQGLEWMGWIDPEGGNTIYDQKFQ | QQKPGKAPKLLIYRTSNLASGVPSRFSGSGSGTDFT |
| F09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSN | GWIDH | CARG | SARQ | RTSNL | CQQYH | QVQLVQSGAEVKKPGASVKVSCKASGYTFSNYY | DIQMTQSPSSLSASVGDRVTITCSARQSISYMYWYC |
| DB06_ | YYMH | ENGNTI | YDV | SISY | AS | SYPPTF | MHWVRQAPGQGLEWMGWIDHENGNTIYDQKFQ | QKPGKAPKLLIYRTSNLASGVPSRFSGSGSGTDFTL |
| F10 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB06_ | YYMH | ENGSTI | YDV | SVSY | QS | SYPPTF | MHWVRQAPGQGLEWMGWIDHENGSTIYAQKFQ | QQKPGKAPKLLIYRTSNLQSGVPSRFSGSGSGTDFT |
| F11 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSS | GWIDP | CARG | RARQ | RTSNL | CQQYH | QVQLVQSGAEVKKPGASVKVSCKASGYTFSSYY | DIQMTQSPSSLSASVGDRVTITCRARQSVSYMYWY |
| DB06_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| F12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTG | GWIDH | CARG | SARS | RTSNL | CQQYH | QVQLVQSGAEVKKPGASVKVSCKASGDTFTGYY | DIQMTQSPSSLSASVGDRVTITCSARSSVSYMYWY |
| DB06_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| G01 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YNITD | GWIDP | CARG | RARS | RTSNL | CQQYH | QVQLVQSGAEVKKPGASVKVSCKASGYNITDYYI | DIQMTQSPSSLSASVGDRVTITCRARSSVSYMYWY |
| DB06_ | YYIH | ENGSTI | YDV | SVSY | AS | SYPPTF | HWVRQAPGQGLEWMGWIDPENGSTIYDQKFQGR | QQKPGKAPKLLIYRTSNLASGVPSRFSGSGSGTDFT |
| G02 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFSS | GWIDY | CARG | SARS | RTSNL | CQQYH | QVQLVQSGAEVKKPGASVKVSCKASGYTFSSYY | DIQMTQSPSSLSASVGDRVTITCSARSSVSYMYWY |
| DB06_ | YYMH | EGGNTI | YDV | SVSY | AS | SYPPTF | MHWVRQAPGQGLEWMGWIDPEGGNTIYAQKFQ | QQKPGKAPKLLIYRTSNLASGVPSRFSGSGSGTDFT |
| G03 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWINP | CARG | SARY | RTSNL | CQQYH | QVQLVQSGAEVKKPGASVKVSCKASGYTFTGYY | DIQMTQSPSSLSASVGDRVTITCSARYSVSYMYWY |
| DB06_ | YYMH | EGGST | YDV | SVSY | AS | SYPPTF | MHWVRQAPGQGLEWMGWINPEGGSTNYEQKFQ | QQKPGKAPKLLIYRTSNLASGVPSRFSGSGSGTDFT |
| G04 | | NYE | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFIN | GWIDH | CARG | SARQ | RTSNL | CQQYH | QVQLVQSGAEVKKPGASVKVSCKASGYTFTNYY | DIQMTQSPSSLSASVGDRVTITCSARQSISYMYWYQ |
| DB06_ | YYMH | ENGNT | YDV | SISY | AS | SYPPTF | MHWVRQAPGQGLEWMGWIDHENGNTSYAQKFQ | QKPGKAPKLLIYRTSNLASGVPSRFSGSGSGTDFTL |
| G05 | | SYA | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | FTFTA | GWEN | CARG | SASQ | RTSNL | CQQYH | QVQLVQSGAEVKKPGASVKVSCKASGFTFTAYY | DIQMTQSPSSLSASVGDRVTITCSASQSVSYMYWY |
| DB06_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| G06 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB06_ | YYMH | ENGNTI | YDV | SVSY | AS | SYPPTF | MHWVRQAPGQGLEWMGWIDHENGNTIYAQKFQ | QQKPGKAPKLLIYRTSNLASGVPSRFSGSGSGTDFT |
| G07 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDP | CARG | SARS | RTSNL | CQQYH | QVQLVQSGAEVKKPGASVKVSCKASGYTFTGYYI | DIQMTQSPSSLSASVGDRVTITCSARSSVSYMYWY |
| DB06_ | YYIH | EGGNTI | YDV | SVSY | AS | SYPPTF | HWVRQAPGQGLEWMGWIDPEGGNTIYDQKFQG | QQKPGKAPKLLIYRTSNLASGVPSRFSGSGSGTDFT |
| G08 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB06_ | YYMH | SNGNTI | DDV | SVSY | AS | SYPPTF | MHWVRQAPGQGLEWMGWIDHSNGNTIYAQKFQ | QQKPGKAPKLLIYRTSNLASGVPSRFSGSGSGTDFT |
| G09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | DDVDWFVYWGQGTLVTVSS | |
| suPAR_ | YTFTS | GWIDH | CARG | SARQ | RTSNL | CQQYH | QVQLVQSGAEVKKPGASVKVSCKASGYTFTSHYI | DIQMTQSPSSLSASVGDRVTITCSARQSVSYMYWY |
| DB06_ | HYIH | ENGNT | YDV | SVSY | AS | SYPPTF | HWVRQAPGQGLEWMGWIDHENGNTSYDQKFQG | QQKPGKAPKLLIYRTSNLASGVPSRFSGSGSGTDFT |
| G10 | | SYD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFNN | GWIDH | CARG | SARS | RTSNL | CQQYH | QVQLVQSGAEVKKPGASVKVSCKASGYTENNYY | DIQMTQSPSSLSASVGDRVTITCSARSSVSYMYWY |
| DB06_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| G11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YAFTA | GWIDH | CARG | SARS | RTSNL | CQQYH | QVQLVQSGAEVKKPGASVKVSCKASGYAFTANYI | DIQMTQSPSSLSASVGDRVTITCSARSSVSYMYWY |
| DB06_ | NYIH | ENGSTI | SDVD | SVSY | AS | SYPPTF | HWVRQAPGQGLEWMGWIDHENGSTIYAQKFQG | QQKPGKAPKLLIYRTSNLASGVPSRFSGSGSGTDFT |
| G12 | | YA | WFV | MY | | | RVTMTRDTSTSTVYMELSSLRSEDTAVYYCARGS | LTISSLQPEDFATYYCQQYHSYPPTFGGGTKVEIKR |
| | | | YW | | | | DVDWFVYWGQGTLVTVSS | |
| suPAR_ | HTFTS | GWIDH | CARG | SARS | RTSNL | CQQYH | QVQLVQSGAEVKKPGASVKVSCKASGHTFTSHYI | DIQMTQSPSSLSASVGDRVTITCSARSSVSYMYWY |
| DB06_ | HYIH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| H01 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTS | GWIST | CARG | SARS | RTSNL | CQQYH | QVQLVQSGAEVKKPGASVKVSCKASGYTFTSYY | DIQMTQSPSSLSASVGDRVTITCSARSSVSYMYWY |
| DB06_ | YYMH | DNGNT | YDV | SVSY | AS | SYPPTF | MHWVRQAPGQGLEWMGWISTDNGNTNYAQKFQ | QQKPGKAPKLLIYRTSNLASGVPSRFSGSGSGTDFT |
| H02 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GWIDP | CARG | SASS | RTSNL | CQQYH | QVQLVQSGAEVKKPGASVKVSCKASGFNIKDYY | DIQMTQSPSSLSASVGDRVTITCSASSSVSYMYWY |
| DB06_ | YYMH | ENGSTI | YDV | SVSY | QS | SYPPTF | MHWVRQAPGQGLEWMGWIDPENGSTIYDQKFQ | QQKPGKAPKLLIYRTSNLQSGVPSRFSGSGSGTDFT |
| H03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFIN | GWIDH | CARG | SARS | RTSSLA | CQQYH | QVQLVQSGAEVKKPGASVKVSCKASGDTFINYY | DIQMTQSPSSLSASVGDRVTITCSARSSVSYMYWY |
| DB06_ | YYMH | ENGNTI | YDV | SVSY | S | SYPPTF | MHWVRQAPGQGLEWMGWIDHENGNTIYDQKFQ | QQKPGKAPKLLIYRTSSLASGVPSRFSGSGSGTDFT |
| H04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSN | GWIDH | CARG | RARQ | RTSNL | CQQYH | QVQLVQSGAEVKKPGASVKVSCKASGYTFSNYY | DIQMTQSPSSLSASVGDRVTITCRARQSVSYMYWY |
| DB06_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| H05 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSG | GWIDH | CARG | SARS | RTSSLA | CQQYH | QVQLVQSGAEVKKPGASVKVSCKASGYTFSGHYI | DIQMTQSPSSLSASVGDRVTITCSARSSVSYMYWY |
| DB06_ | HYIH | ENQNTI | YDV | SVSY | S | SYPPTF | HWVRQAPGQGLEWMGWIDHENGNTIYDQKFQG | QQKPGKAPKLLIYRTSSLASGVPSRFSGSGSGTDFT |
| H06 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YSFTN | GWISP | CARG | SASS | RTSNL | CQQYH | QVQLVQSGAEVKKPGASVKVSCKASGYSFTNYY | DIQMTQSPSSLSASVGDRVTITCSASSSVSYMYWY |
| DB06_ | YYMH | DNGNT | YDV | SVSY | AS | SYPPTF | MHWVRQAPGQGLEWMGWISPDNGNTQYAQKFQ | QQKPGKAPKLLIYRTSNLASGVPSRFSGSGSGTDFT |
| H07 | | QYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTD | GWIDP | CARG | SARS | RTSNL | CQQYH | QVQLVQSGAEVKKPGASVKVSCKASGYSFTDYYI | DIQMTQSPSSLSASVGDRVTITCSARSSVSYMYWY |
| DB06_ | YYIH | ENQSTI | YDV | SVSY | AS | SYPPTF | HWVRQAPGQGLEWMGWIDPENGSTIYDQKFQGR | QQKPGKAPKLLIYRTSNLASGVPSRFSGSGSGTDFT |
| H08 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YTFTG | GWIDH | CAM | SARS | RTSNL | CQQYH | QVQLVQSGAEVKKPGASVKVSCKASGYTFTGYY | DIQMTQSPSSLSASVGDRVTITCSARSSVSYMYWY |
| DB06_ | YYVH | ENGNTI | GYD | SVSY | AS | SYPPTF | VHWVRQAPGQGLEWMGWIDHENGNTIYDQKFQ | QQKPGKAPKLLIYRTSNLASGVPSRFSGSGSGTDFT |
| H09 | | YD | VDW | MY | | | GRVTMTRDTSTSTVYMELSSLRSEDTAVYYCAM | LTISSLQPEDFATYYCQQYHSYPPTFGGGTKVEIKR |
| | | | FVY | | | | GYDVDWFVYWGQGTLVTVSS | |
| | | | W | | | | | |
| suPAR_ | NTFTS | GWIDH | CARG | SARQ | RTSNL | CQQYH | QVQLVQSGAEVKKPGASVKVSCKASGNTFTSYYT | DIQMTQSPSSLSASVGDRVTITCSARQSVSYMYWY |
| DB06_ | YYTH | ENQSTI | YDV | SVSY | AS | SYPPTF | HWVRQAPGQGLEWMGWIDHENGSTIYAQKFQG | QQKPGKAPKLLIYRTSNLASGVPSRFSGSGSGTDFT |
| H10 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | DTFTG | GWIDH | CARG | SARQ | RTSNL | CQQYH | QVQLVQSGAEVKKPGASVKVSCKASGDTFTGYY | DIQMTQSPSSLSASVGDRVTITCSARQSVSYMYWY |
| DB07_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| A03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTA | GWIDH | CARG | SARS | RTSNL | CQQYH | QVQLVQSGAEVKKPGASVKVSCKASGDTFTAYY | DIQMTQSPSSLSASVGDRVTITCSARSSVSYMYWY |
| DB07_ | YYFH | ENGNTI | YDV | SVSY | AS | SYPPTF | FHWVRQAPGQGLEWMGWIDHENGNTIYDQKFQ | QQKPGKAPKLLIYRTSNLASGVPSRFSGSGSGTDFT |
| A04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTG | GWIDH | CARG | SARS | RTSNL | CQQYH | QVQLVQSGAEVKKPGASVKVSCKASGGTFTGYY | DIQMTQSPSSLSASVGDRVTITCSARSSVSYMYWY |
| DB07_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| A05 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CAEG | SARS | RTSNL | CQQYH | QVQLVQSGAEVKKPGASVKVSCKASGYTFTGYY | DIQMTQSPSSLSASVGDRVTITCSARSSVSYMYWY |
| DB07_ | YYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| A06 | | YD | DWF | MY | | | GRVTMTRDTSTSTVYMELSSLRSEDTAVYYCAEG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SARS | RTSNL | CQQYH | QVQLVQSGAEVKKPGASVKVSCKASGYTFTSYYL | DIQMTQSPSSLSASVGDRVTITCSARSSVSYMYWY |
| DB07_ | YYLH | SSGAT | YDV | SVSY | AS | SYPPTF | HWVRQAPGQGLEWMGWIDPSSGATDYAQKFQG | QQKPGKAPKLLIYRTSNLASGVPSRFSGSGSGTDFT |
| A07 | | DYA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTN | GWIDH | CARG | SARS | RTSSLQ | CQQYH | QVQLVQSGAEVKKPGASVKVSCKASGYTFTNYY | DIQMTQSPSSLSASVGDRVTITCSARSSVSYMYWY |
| DB07_ | YYMH | ENGSTI | YDV | SVSY | S | SYPPTF | MHWVRQAPGQGLEWMGWIDHENGSTIYAQKFQ | QQKPGKAPKLLIYRTSSLQSGVPSRFSGSGSGTDFT |
| A08 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTT | GWIDP | CARG | SASS | RTSNL | CQQYH | QVQLVQSGAEVKKPGASVKVSCKASGYTFTTYY | DIQMTQSPSSLSASVGDRVTITCSASSSVSYMYWY |
| DB07_ | YYMH | ENQNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| A09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDH | CARG | SARS | RTSNL | CQQYH | QVQLVQSGAEVKKPGASVKVSCKASGYTFTGYY | DIQMTQSPSSLSASVGDRVTITCSARSSVSYMYWY |
| DB07_ | YYVH | ENGNTI | SDVD | SVSY | AS | SYPPTF | VHWVRQAPGQGLEWMGWIDHENGNTIYDQKFQ | QQKPGKAPKLLIYRTSNLASGVPSRFSGSGSGTDFT |
| A10 | | YD | WFV | MY | | | GRVTMTRDTSTSTVYMELSSLRSEDTAVYYCARG | LTISSLQPEDFATYYCQQYHSYPPTFGGGTKVEIKR |
| | | | YW | | | | SDVDWFVYWGQGTLVTVSS | |
| suPAR_ | YTFSN | GWIDH | CARG | RARQ | RTSNL | CQQYH | QVQLVQSGAEVKKPGASVKVSCKASGYTFSNYY | DIQMTQSPSSLSASVGDRVTITCRARQSVSYMYWY |
| DB07_ | YYVH | ENGNTI | TDVD | SVSY | AS | SYPPTF | VHWVRQAPGQGLEWMGWIDHENGNTIYDQKFQ | QQKPGKAPKLLIYRTSNLASGVPSRFSGSGSGTDFT |
| A11 | | YD | WFV | MY | | | GRVTMTRDTSTSTVYMELSSLRSEDTAVYYCARG | LTISSLQPEDFATYYCQQYHSYPPTFGGGTKVEIKR |
| | | | YW | | | | TDVDWFVYWGQGTLVTVSS | |
| suPAR_ | GIFTS | GWIDH | CARG | SARS | RTSNL | CQQYH | QVQLVQSGAEVKKPGASVKVSCKASGGTFTSYY | DIQMTQSPSSLSASVGDRVTITCSARSSVSYMYWY |
| DB07_ | YYMH | SNGNTI | YDV | SVSY | AS | SYPPTF | MHWVRQAPGQGLEWMGWIDHSNGNTIYDQKFQ | QQKPGKAPKLLIYRTSNLASGVPSRFSGSGSGTDFT |
| A12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDP | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB07_ | YYLH | ENGSTI | YDV | SVSY | AS | SYPPTF | LHWVRQAPGQGLEWMGWIDPENGSTIYAQKFQG | QQKPGKAPKLLIYRTSNLASGVPSRFSGSGSGTDFT |
| B01 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFTG | GWIDH | CARG | SASQ | RTSNL | CQQYH | QVQLVQSGAEVKKPGASVKVSCKASGYTFTGYY | DIQMTQSPSSLSASVGDRVTITCSASQSVSYMYWY |
| DB07_ | YYMH | ENGSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| B02 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSN | GWIDH | CARG | SARS | RTSNL | CQQYH | QVQLVQSGAEVKKPGASVKVSCKASGYTFSNYY | DIQMTQSPSSLSASVGDRVTITCSARSSVSYMYWY |
| DB07_ | YYMH | ENGSTS | YDV | SVSY | AS | SYPPTF | MHWVRQAPGQGLEWMGWIDHENGSTSYAQKFQ | QQKPGKAPKLLIYRTSNLASGVPSRFSGSGSGTDFT |
| B03 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FTGHY | GWIDH | CARG | SARS | RTSNL | CQQYH | QVQLVQSGAEVKKPGASVKVSCKASGFTGHYIH | DIQMTQSPSSLSASVGDRVTITCSARSSVSYMYWY |
| DB07_ | IH | ENGNTI | YDV | SVSY | AS | SYPPTF | WVRQAPGQGLEWMGWIDHENGNTIYDQKFQGR | QQKPGKAPKLLIYRTSNLASGVPSRFSGSGSGTDFT |
| B04 | | YD | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | YSFTG | GWIDH | CARG | SARS | RTSNL | CQQYH | QVQLVQSGAEVKKPGASVKVSCKASGYSFTGYY | DIQMTQSPSSLSASVGDRVTITCSARSSVSYMYWY |
| DB07_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| B05 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFAN | GWIDH | CARG | SARS | RTSNL | CQQYH | QVQLVQSGAEVKKPGASVKVSCKASGYSFANNYI | DIQMTQSPSSLSASVGDRVTITCSARSSVSYMYWY |
| DB07_ | NYIH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| B06 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | YTFAS | GWIDH | CARG | SARS | RTSNL | CQQYH | QVQLVQSGAEVKKPGASVKVSCKASGYTFASYYI | DIQMTQSPSSLSASVGDRVTITCSARSSVSYMYWY |
| DB07_ | YYIH | ENQSTI | YDV | SVSY | AS | SYPPTF | HWVRQAPGQGLEWMGWIDHENGSTIYAQKFQG | QQKPGKAPKLLIYRTSNLASGVPSRFSGSGSGTDFT |
| B07 | | YA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | FNIKD | GWID | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB07_ | YYIH | SNGNTI | NDV | SVSY | AS | SYPPTF | HWVRQAPGQGLEWMGWIDPSNGNTIYDQKFQGR | QQKPGKAPKLLIYRTSNLASGVPSRFSGSGSGTDFT |
| B08 | | YD | DWF | MY | | | VTMTRDTSTSTVYMELSSLRSEDTAVYYCARGND | LTISSLQPEDFATYYCQQYHSYPPTFGGGTKVEIKR |
| | | | VYW | | | | VDWFVYWGQGTLVTVSS | |
| suPAR_ | YTLTN | GWIDY | CARG | SASS | RTSNL | CQQYH | QVQLVQSGAEVKKPGASVKVSCKASGYTLTNYY | DIQMTQSPSSLSASVGDRVTITCSASSSVSYMYWY |
| DB07_ | YYMH | ENQNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| B09 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| SUPAR | FNIKD | GWMD | CARG | SARQ | RTSNL | CQQYH | QVQLVQSGAEVKKPGASVKVSCKASGFNIKDYYI | DIQMTQSPSSLSASVGDRVTITCSARQSVSYMYWY |
| DB07_ | YYIH | PSSGNT | YDV | SVSY | AS | SYPPTF | HWVRQAPGQGLEWMGWMDPSSGNTAYAQKFQ | QQKPGKAPKLLIYRTSNLASGVPSRFSGSGSGTDFT |
| B10 | | AYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFRN | GWIDH | CARG | SARS | RTSNL | CQQYH | QVQLVQSGAEVKKPGASVKVSCKASGDTFRNYY | DIQMTQSPSSLSASVGDRVTITCSARSSVSYMYWY |
| DB07_ | YYMH | ENGSTI | YDVE | SVSY | AS | SYPPTF | MHWVRQAPGQGLEWMGWIDHENGSTIYAQKFQ | QQKPGKAPKLLIYRTSNLASGVPSRFSGSGSGTDFT |
| B11 | | YA | WFV | MY | | | GRVTMTRDTSTSTVYMELSSLRSEDTAVYYCARG | LTISSLQPEDFATYYCQQYHSYPPTFGGGTKVEIKR |
| | | | YW | | | | YDVEWFVYWGQGTLVTVSS | |
| suPAR_ | YTFTG | GWIDH | CARG | RASS | RTSNL | CQQYH | QVQLVQSGAEVKKPGASVKVSCKASGYTFTGHY | DIQMTQSPSSLSASVGDRVTITCRASSSVSYMYWY |
| DB07_ | HYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| B12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTS | GWIDP | CARG | SARS | RTSNL | CQQYH | QVQLVQSGAEVKKPGASVKVSCKASGGTFTSYY | DIQMTQSPSSLSASVGDRVTITCSARSSVSYMYWY |
| DB07_ | YYMH | SNQNTI | YDV | SVSY | AS | SYPPTF | MHWVRQAPGQGLEWMGWIDPSNGNTTYDQKFQ | QQKPGKAPKLLIYRTSNLASGVPSRFSGSGSGTDFT |
| C01 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKS | GWIDH | CARG | SARS | RTSNL | CQQYH | QVQLVQSGAEVKKPGASVKVSCKASGFNIKSYY | DIQMTQSPSSLSASVGDRVTITCSARSSVSYMYWY |
| DB07_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| C02 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDH | CARG | RARQ | RTSNL | CQQYH | QVQLVQSGAEVKKPGASVKVSCKASGYTFTNYY | DIQMTQSPSSLSASVGDRVTITCRARQSVSYMYWY |
| DB07_ | YYVH | ENGNTI | YDV | SVSY | AS | SYPPTF | VHWVRQAPGQGLEWMGWIDHENGNTIYDQKFQ | QQKPGKAPKLLIYRTSNLASGVPSRFSGSGSGTDFT |
| C03 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTS | GWIDH | CARG | SARS | RTSNL | CQQYH | QVQLVQSGAEVKKPGASVKVSCKASGGTFTSYY | DIQMTQSPSSLSASVGDRVTITCSARSSVSYMYWY |
| DB07_ | YYMH | ENQSTI | YDV | SVSY | AS | SYPPTF | MHWVRQAPGQGLEWMGWIDHENGSTIYAQKFQ | QQKPGKAPKLLIYRTSNLASGVPSRFSGSGSGTDFT |
| C04 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTN | GWIDE | CARG | SARS | RTSNL | CQQYH | QVQLVQSGAEVKKPGASVKVSCKASGYTFTNYY | DIQMTQSPSSLSASVGDRVTITCSARSSVSYMYWY |
| DB07_ | YYMH | YSGAT | YDV | SVSY | AS | SYPPTF | MHWVRQAPGQGLEWMGWIDPYSGATNYAQKLQ | QQKPGKAPKLLIYRTSNLASGVPSRFSGSGSGTDFT |
| C05 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTS | GWIDY | CARG | RARQ | RTSNL | CQQYH | QVQLVQSGAEVKKPGASVKVSCKASGGTFTSYY | DIQMTQSPSSLSASVGDRVTITCRARQSVSYMYWY |
| DB07_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| C06 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | HTFNT | GWIDP | CARG | SARS | RTSNL | CQQYH | QVQLVQSGAEVKKPGASVKVSCKASGHTENTYY | DIQMTQSPSSLSASVGDRVTITCSARSSVSYMYWY |
| DB07_ | YYVH | ENQNTI | YDV | SVSY | AS | SYPPTF | VHWVRQAPGQGLEWMGWIDPENGNTIYAQKFQ | QQKPGKAPKLLIYRTSNLASGVPSRFSGSGSGTDFT |
| C07 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNIKD | GLIDPR | CARG | SARS | RTSNL | CQQYH | QVQLVQSGAEVKKPGASVKVSCKASGFNIKDYYI | DIQMTQSPSSLSASVGDRVTITCSARSSVSYMYWY |
| DB07_ | YYIH | NDYTS | YDV | SVSY | AS | SYPPTF | HWVRQAPGQGLEWMGLIDPRNDYTSYAPQFQGR | QQKPGKAPKLLIYRTSNLASGVPSRFSGSGSGTDFT |
| C08 | | YA | DWF | MY | | | VTMTRDTSTSTVYMELSSLRSEDTAVYYCARGYD | LTISSLQPEDFATYYCQQYHSYPPTFGGGTKVEIKR |
| | | | VYW | | | | VDWFVYWGQGTLVTVSS | |
| suPAR_ | FTFTN | GWIDH | CARG | SARS | RTSNL | CQQYH | QVQLVQSGAEVKKPGASVKVSCKASGFTFTNYY | DIQMTQSPSSLSASVGDRVTITCSARSSVSYMYWY |
| DB07_ | YYVH | ENGSTS | YDV | SVSY | AS | SYPPTF | VHWVRQAPGQGLEWMGWIDHENGSTSYDQKFQ | QQKPGKAPKLLIYRTSNLASGVPSRFSGSGSGTDFT |
| C09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDP | CARG | SARQ | RTSNL | CQQYH | QVQLVQSGAEVKKPGASVKVSCKASGYTFTSHW | DIQMTQSPSSLSASVGDRVTITCSARQSISYMYWYQ |
| DB07_ | HWMH | ENGSTI | YDV | SISY | AS | SYPPTF | MHWVRQAPGQGLEWMGWIDPENGSTIYDQKFQ | QKPGKAPKLLIYRTSNLASGVPSRFSGSGSGTDFTL |
| C10 | | YD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSNYI | DIQMTQSPSSLSASVGDRVTITCSARSSVSYMYWY |
| DB07_ | NYIH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| C11 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | GTFTG | GWIDH | CARG | SARS | RTSNL | CQQYH | QVQLVQSGAEVKKPGASVKVSCKASGGTFTGYY | DIQMTQSPSSLSASVGDRVTITCSARSSVSYMYWY |
| DB07_ | YYMH | ENQNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| C12 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | SARS | RTSNL | CQQYH | QVQLVQSGAEVKKPGASVKVSCKASGYTFTSHY | DIQMTQSPSSLSASVGDRVTITCSARSSVSYMYWY |
| DB07_ | HYMH | ENGNTI | YDV | SVSY | AS | SYPPTF | MHWVRQAPGQGLEWMGWIDHENGNTIYAQKFQ | QQKPGKAPKLLIYRTSNLASGVPSRFSGSGSGTDFT |
| D01 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTG | GWIDE | CARG | SASQ | RTSNL | CQQYH | QVQLVQSGAEVKKPGASVKVSCKASGYTFTGYY | DIQMTQSPSSLSASVGDRVTITCSASQSVSYMYWY |
| DB07_ | YYMH | ENGNTI | YDV | SVSY | AS | SYPPTF | MHWVRQAPGQGLEWMGWIDPENGNTIYAQKFQ | QQKPGKAPKLLIYRTSNLASGVPSRFSGSGSGTDFT |
| D02 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFSN | GWIDH | CARG | SARQ | RTSNL | CQQYH | QVQLVQSGAEVKKPGASVKVSCKASGYTFSNYY | DIQMTQSPSSLSASVGDRVTITCSARQSVSYMYWY |
| DB07_ | YYMH | ENGNTI | YDV | SVSY | AS | SYPPTF | MHWVRQAPGQGLEWIGWIDHENGNTIYDQKFQG | QQKPGKAPKLLIYRTSNLASGVPSRFSGSGSGTDFT |
| D03 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | FAFTT | GWIDP | CARG | RARS | RTSNL | CQQYH | QVQLVQSGAEVKKPGASVKVSCKASGFAFTTYY | DIQMTQSPSSLSASVGDRVTITCRARSSVSYMYWY |
| DB07_ | YYMH | SNGNTI | YDV | SVSY | AS | SYPPTF | MHWVRQAPGQGLEWMGWIDPSNGNTIYDQKFQ | QQKPGKAPKLLIYRTSNLASGVPSRFSGSGSGTDFT |
| D04 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | DTFTS | GWIDE | CARG | SARQ | RTSNL | CQQYH | QVQLVQSGAEVKKPGASVKVSCKASGDTFTSYY | DIQMTQSPSSLSASVGDRVTITCSARQSISYMYWYC |
| DB07_ | YYMH | ENQNT | YDV | SISY | QS | SYPPTF | MHWVRQAPGQGLEWMGWIDPENGNTSYDQKFQ | QKPGKAPKLLIYRTSNLQSGVPSRFSGSGSGTDFTL |
| D05 | | SYD | DWF | MY | | | GRVTMTRDTSTSTVYMELSSLRSEDTAVYYCARG | TISSLQPEDFATYYCQQYHSYPPTFGGGTKVEIKR |
| | | | VYW | | | | YDVDWFVYWGQGTLVTVSS | |
| suPAR_ | DTFTS | GWIYP | CARG | SARS | RTSSLA | CQQYH | QVQLVQSGAEVKKPGASVKVSCKASGDTFTSYY | DIQMTQSPSSLSASVGDRVTITCSARSSVSYMYWY |
| DB07_ | YYMH | NEGDT | YDV | SVSY | S | SYPPTF | MHWVRQAPGQGLEWMGWIYPNSGDTNYAQKFQ | QQKPGKAPKLLIYRTSSLASGVPSRFSGSGSGTDFT |
| D06 | | NYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YTFTS | GWIDH | CARG | RARS | RTSNL | CQQYH | QVQLVQSGAEVKKPGASVKVSCKASGYTFTSNYI | DIQMTQSPSSLSASVGDRVTITCRARSSVSYMYWY |
| DB07_ | NYIH | ENGNTI | YDV | SVSY | AS | SYPPTF | HWVRQAPGQGLEWMGWIDHENGNTIYDQKFQG | QQKPGKAPKLLIYRTSNLASGVPSRFSGSGSGTDFT |
| D07 | | YD | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | STFTT | GWIDP | CARG | SASQ | RTSNL | CQQYH | QVQLVQSGAEVKKPGASVKVSCKASGSTFTTYY | DIQMTQSPSSLSASVGDRVTITCSASQSVSYMYWY |
| DB07_ | YYMH | ENQNT | YDV | SVSY | AS | SYPPTF | MHWVRQAPGQGLEWMGWIDPENGNTSYDQKFQ | QQKPGKAPKLLIYRTSNLASGVPSRFSGSGSGTDFT |
| D08 | | SYD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | GTFTN | GWIDH | CARG | SARS | RTSNL | CQQYH | QVQLVQSGAEVKKPGASVKVSCKASGGTFTNYY | DIQMTQSPSSLSASVGDRVTITCSARSSVSYMYWY |
| DB07_ | YYMH | EGGNTI | YDV | SVSY | AS | SYPPTF | MHWVRQAPGQGLEWMGWIDHEGGNTIYDQKFQ | QQKPGKAPKLLIYRTSNLASGVPSRFSGSGSGTDFT |
| D09 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | FNFKD | GWIDP | CARG | SARS | RTSNL | CQQYH | QVQLVQSGAEVKKPGASVKVSCKASGENFKDYYI | DIQMTQSPSSLSASVGDRVTITCSARSSVSYMYWY |
| DB07_ | YYIH | NEGGT | YDV | SVSY | AS | SYPPTF | HWVRQAPGQGLEWMGWIDPNSGGTKFAQKFQG | QQKPGKAPKLLIYRTSNLASGVPSRFSGSGSGTDFT |
| D10 | | KFA | DWF | MY | | | RVTMTRDTSTSTVYMELSSLRSEDTAVYYCARGY | LTISSLQPEDFATYYCQQYHSYPPTFGGGTKVEIKR |
| | | | VYW | | | | DVDWFVYWGQGTLVTVSS | |
| suPAR_ | GTFTS | GWIDH | CARG | SARQ | RTSNL | CQQYH | QVQLVQSGAEVKKPGASVKVSCKASGGTFTSYY | DIQMTQSPSSLSASVGDRVTITCSARQSVSYMYWY |
| DB07_ | YYMH | ENGNTI | YDV | SVSY | AS | SYPPTF | MHWVRQAPGQGLEWMGWIDHENGNTIYDQKFQ | QQKPGKAPKLLIYRTSNLASGVPSRFSGSGSGTDFT |
| D11 | | YD | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YAFTS | GWIDH | CARG | RARQ | RTSNL | CQQYH | QVQLVQSGAEVKKPGASVKVSCKASGYAFTSYY | DIQMTQSPSSLSASVGDRVTITCRARQSVSYMYWY |
| DB07_ | YYMH | ENGNTI | YDV | SVSY | QS | SYPPTF | MHWVRQAPGQGLEWMGWIDHENGNTIYAQKFQ | QQKPGKAPKLLIYRTSNLQSGVPSRFSGSGSGTDFT |
| D12 | | YA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |
| suPAR_ | YSFTD | GWISP | CARG | SARS | RTSNL | CQQYH | QVQLVQSGAEVKKPGASVKVSCKASGYSFTDYY | DIQMTQSPSSLSASVGDRVTITCSARSSVSYMYWY |
| DB07_ | YYMH | DSGGT | YDV | SVSY | AS | SYPPTF | MHWVRQAPGQGLEWMGWISPDSGGTKYAQKFQ | QQKPGKAPKLLIYRTSNLASGVPSRFSGSGSGTDFT |
| E01 | | KYA | DWF | MY | | | GRVTMTRDTSTSTVYMELSSLRSEDTAVYYCARG | LTISSLQPEDFATYYCQQYHSYPPTFGGGTKVEIKR |
| | | | VYW | | | | YDVDWFVYWGQGTLVTVSS | |

TABLE 6
Binding dissociation for antibodies in Table 3

| | cynomologus | human | SEQ ID NOs | | | | | | | |
|---|---|---|---|---|---|---|---|---|---|---|
| DB_ID | kdis (1/s) | kdis (1/s) | H1 | H2 | H3 | L1 | L1 | L1 | HC | LC |
| suPAR_DB01_A02 | NA | 3.54E−07 | 101 | 701 | 1301 | 1901 | 2501 | 3101 | 3701 | 4301 |
| suPAR_DB01_A03 | 2.31E−04 | 2.56E−03 | 102 | 702 | 1302 | 1902 | 2502 | 3102 | 3702 | 4302 |
| suPAR_DB01_A04 | 2.10E−04 | 9.11E−04 | 103 | 703 | 1303 | 1903 | 2503 | 3103 | 3703 | 4303 |
| suPAR_DB01_A05 | 3.26E−04 | 1.74E−03 | 104 | 704 | 1304 | 1904 | 2504 | 3104 | 3704 | 4304 |
| suPAR_DB01_A06 | 4.27E−07 | 2.53E−03 | 105 | 705 | 1305 | 1905 | 2505 | 3105 | 3705 | 4305 |
| suPAR_DB01_A07 | 1.56E−03 | 5.71E−03 | 106 | 706 | 1306 | 1906 | 2506 | 3106 | 3706 | 4306 |
| suPAR_DB01_A08 | 5.93E−07 | 2.68E−03 | 107 | 707 | 1307 | 1907 | 2507 | 3107 | 3707 | 4307 |
| suPAR_DB01_A09 | 3.60E−07 | 1.70E−03 | 108 | 708 | 1308 | 1908 | 2508 | 3108 | 3708 | 4308 |
| suPAR_DB01_A10 | 2.99E−04 | 1.94E−03 | 109 | 709 | 1309 | 1909 | 2509 | 3109 | 3709 | 4309 |
| suPAR_DB01_A11 | 4.79E−04 | 1.50E−03 | 110 | 710 | 1310 | 1910 | 2510 | 3110 | 3710 | 4310 |
| suPAR_DB01_A12 | 1.42E−04 | 1.28E−03 | 111 | 711 | 1311 | 1911 | 2511 | 3111 | 3711 | 4311 |
| suPAR_DB01_B01 | 4.88E−07 | 2.80E−03 | 112 | 712 | 1312 | 1912 | 2512 | 3112 | 3712 | 4312 |
| suPAR_DB01_B02 | 1.77E−04 | 1.42E−03 | 113 | 713 | 1313 | 1913 | 2513 | 3113 | 3713 | 4313 |
| suPAR_DB01_B03 | 3.50E−07 | 3.29E−03 | 114 | 714 | 1314 | 1914 | 2514 | 3114 | 3714 | 4314 |
| suPAR_DB01_B04 | 8.48E−05 | 2.06E−03 | 115 | 715 | 1315 | 1915 | 2515 | 3115 | 3715 | 4315 |
| suPAR_DB01_B05 | 1.81E−04 | 4.07E−03 | 116 | 716 | 1316 | 1916 | 2516 | 3116 | 3716 | 4316 |
| suPAR_DB01_B06 | 5.62E−07 | 3.48E−03 | 117 | 717 | 1317 | 1917 | 2517 | 3117 | 3717 | 4317 |
| suPAR_DB01_B07 | 6.97E−07 | 2.28E−03 | 118 | 718 | 1318 | 1918 | 2518 | 3118 | 3718 | 4318 |
| suPAR_DB01_B08 | 2.11E−04 | 3.71E−03 | 119 | 719 | 1319 | 1919 | 2519 | 3119 | 3719 | 4319 |
| suPAR_DB01_B09 | 4.00E−07 | 3.29E−03 | 120 | 720 | 1320 | 1920 | 2520 | 3120 | 3720 | 4320 |
| suPAR_DB01_B10 | 1.08E−03 | 1.05E−03 | 121 | 721 | 1321 | 1921 | 2521 | 3121 | 3721 | 4321 |
| suPAR_DB01_B11 | 8.79E−05 | 2.73E−03 | 122 | 722 | 1322 | 1922 | 2522 | 3122 | 3722 | 4322 |
| suPAR_DB01_B12 | 2.38E−04 | 2.98E−03 | 123 | 723 | 1323 | 1923 | 2523 | 3123 | 3723 | 4323 |
| suPAR_DB01_C01 | 2.66E−04 | 1.22E−03 | 124 | 724 | 1324 | 1924 | 2524 | 3124 | 3724 | 4324 |
| suPAR_DB01_C02 | 2.34E−04 | 3.53E−03 | 125 | 725 | 1325 | 1925 | 2525 | 3125 | 3725 | 4325 |
| suPAR_DB01_C03 | 6.41E−07 | 6.63E−04 | 126 | 726 | 1326 | 1926 | 2526 | 3126 | 3726 | 4326 |
| suPAR_DB01_C04 | 3.03E−04 | 4.03E−03 | 127 | 727 | 1327 | 1927 | 2527 | 3127 | 3727 | 4327 |
| suPAR_DB01_C05 | 5.31E−07 | 7.59E−05 | 128 | 728 | 1328 | 1928 | 2528 | 3128 | 3728 | 4328 |
| suPAR_DB01_C06 | 5.09E−07 | 8.96E−04 | 129 | 729 | 1329 | 1929 | 2529 | 3129 | 3729 | 4329 |
| suPAR_DB01_C07 | 2.66E−05 | 1.64E−03 | 130 | 730 | 1330 | 1930 | 2530 | 3130 | 3730 | 4330 |
| suPAR_DB01_C08 | 4.20E−04 | 1.30E−03 | 131 | 731 | 1331 | 1931 | 2531 | 3131 | 3731 | 4331 |
| suPAR_DB01_C09 | 2.03E−04 | 4.76E−03 | 132 | 732 | 1332 | 1932 | 2532 | 3132 | 3732 | 4332 |
| suPAR_DB01_C10 | 2.66E−04 | 3.63E−03 | 133 | 733 | 1333 | 1933 | 2533 | 3133 | 3733 | 4333 |
| suPAR_DB01_C11 | 8.96E−05 | 2.26E−03 | 134 | 734 | 1334 | 1934 | 2534 | 3134 | 3734 | 4334 |
| suPAR_DB01_C12 | 1.05E−04 | 4.93E−03 | 135 | 735 | 1335 | 1935 | 2535 | 3135 | 3735 | 4335 |
| suPAR_DB01_D01 | 3.69E−04 | 5.31E−04 | 136 | 736 | 1336 | 1936 | 2536 | 3136 | 3736 | 4336 |
| suPAR_DB01_D02 | 3.43E−04 | 4.25E−03 | 137 | 737 | 1337 | 1937 | 2537 | 3137 | 3737 | 4337 |
| suPAR_DB01_D03 | 2.03E−04 | 3.62E−03 | 138 | 738 | 1338 | 1938 | 2538 | 3138 | 3738 | 4338 |
| suPAR_DB01_D04 | 2.53E−05 | 8.72E−04 | 139 | 739 | 1339 | 1939 | 2539 | 3139 | 3739 | 4339 |
| suPAR_DB01_D05 | 4.19E−04 | 2.24E−03 | 140 | 740 | 1340 | 1940 | 2540 | 3140 | 3740 | 4340 |
| suPAR_DB01_D06 | 5.17E−07 | 1.18E−03 | 141 | 741 | 1341 | 1941 | 2541 | 3141 | 3741 | 4341 |
| suPAR_DB01_D07 | 3.54E−07 | 1.19E−03 | 142 | 742 | 1342 | 1942 | 2542 | 3142 | 3742 | 4342 |
| suPAR_DB01_D08 | 7.01E−07 | 6.66E−04 | 143 | 743 | 1343 | 1943 | 2543 | 3143 | 3743 | 4343 |
| suPAR_DB01_D09 | 5.06E−04 | 1.09E−03 | 144 | 744 | 1344 | 1944 | 2544 | 3144 | 3744 | 4344 |
| suPAR_DB01_D10 | 1.48E−04 | 1.47E−03 | 145 | 745 | 1345 | 1945 | 2545 | 3145 | 3745 | 4345 |
| suPAR_DB01_D11 | 1.60E−04 | 4.66E−03 | 146 | 746 | 1346 | 1946 | 2546 | 3146 | 3746 | 4346 |
| suPAR_DB01_D12 | 2.32E−04 | 3.82E−03 | 147 | 747 | 1347 | 1947 | 2547 | 3147 | 3747 | 4347 |
| suPAR_DB01_E01 | 2.54E−04 | 4.74E−03 | 148 | 748 | 1348 | 1948 | 2548 | 3148 | 3748 | 4348 |
| suPAR_DB01_E02 | 3.47E−04 | 4.69E−03 | 149 | 749 | 1349 | 1949 | 2549 | 3149 | 3749 | 4349 |
| suPAR_DB01_E03 | 5.44E−05 | 4.02E−03 | 150 | 750 | 1350 | 1950 | 2550 | 3150 | 3750 | 4350 |
| suPAR_DB01_E04 | 3.10E−04 | 1.40E−03 | 151 | 751 | 1351 | 1951 | 2551 | 3151 | 3751 | 4351 |
| suPAR_DB01_E05 | 4.06E−04 | 3.08E−03 | 152 | 752 | 1352 | 1952 | 2552 | 3152 | 3752 | 4352 |
| suPAR_DB01_E06 | 2.18E−04 | 2.24E−03 | 153 | 753 | 1353 | 1953 | 2553 | 3153 | 3753 | 4353 |
| suPAR_DB01_E07 | 6.72E−05 | 2.28E−03 | 154 | 754 | 1354 | 1954 | 2554 | 3154 | 3754 | 4354 |
| suPAR_DB01_E08 | 5.85E−07 | 5.64E−04 | 155 | 755 | 1355 | 1955 | 2555 | 3155 | 3755 | 4355 |
| suPAR_DB01_E09 | 2.46E−04 | 4.46E−03 | 156 | 756 | 1356 | 1956 | 2556 | 3156 | 3756 | 4356 |
| suPAR_DB01_E10 | 1.04E−04 | 1.62E−03 | 157 | 757 | 1357 | 1957 | 2557 | 3157 | 3757 | 4357 |
| suPAR_DB01_E11 | 3.69E−04 | 6.38E−04 | 158 | 758 | 1358 | 1958 | 2558 | 3158 | 3758 | 4358 |
| suPAR_DB01_E12 | 2.12E−02 | 1.17E−02 | 159 | 759 | 1359 | 1959 | 2559 | 3159 | 3759 | 4359 |
| suPAR_DB01_F01 | 2.03E−04 | 6.18E−04 | 160 | 760 | 1360 | 1960 | 2560 | 3160 | 3760 | 4360 |
| suPAR_DB01_F02 | 9.84E−05 | 4.00E−03 | 161 | 761 | 1361 | 1961 | 2561 | 3161 | 3761 | 4361 |
| suPAR_DB01_F03 | 6.17E−07 | 2.51E−03 | 162 | 762 | 1362 | 1962 | 2562 | 3162 | 3762 | 4362 |
| suPAR_DB01_F04 | 4.59E−05 | 3.09E−03 | 163 | 763 | 1363 | 1963 | 2563 | 3163 | 3763 | 4363 |
| suPAR_DB01_F05 | 4.95E−07 | 1.75E−03 | 164 | 764 | 1364 | 1964 | 2564 | 3164 | 3764 | 4364 |
| suPAR_DB01_F06 | 4.98E−05 | 2.67E−03 | 165 | 765 | 1365 | 1965 | 2565 | 3165 | 3765 | 4365 |
| suPAR_DB01_F07 | 6.31E−07 | 1.10E−03 | 166 | 766 | 1366 | 1966 | 2566 | 3166 | 3766 | 4366 |
| suPAR_DB01_F08 | 2.07E−04 | 5.05E−04 | 167 | 767 | 1367 | 1967 | 2567 | 3167 | 3767 | 4367 |
| suPAR_DB01_F09 | 1.93E−04 | 2.21E−03 | 168 | 768 | 1368 | 1968 | 2568 | 3168 | 3768 | 4368 |
| suPAR_DB01_F10 | 3.88E−05 | 1.83E−03 | 169 | 769 | 1369 | 1969 | 2569 | 3169 | 3769 | 4369 |
| suPAR_DB01_F11 | 9.02E−05 | 2.93E−03 | 170 | 770 | 1370 | 1970 | 2570 | 3170 | 3770 | 4370 |
| suPAR_DB01_F12 | 1.92E−04 | 2.82E−03 | 171 | 771 | 1371 | 1971 | 2571 | 3171 | 3771 | 4371 |
| suPAR_DB01_G01 | 1.52E−04 | 4.29E−03 | 172 | 772 | 1372 | 1972 | 2572 | 3172 | 3772 | 4372 |
| suPAR_DB01_G02 | 1.49E−04 | 2.45E−03 | 173 | 773 | 1373 | 1973 | 2573 | 3173 | 3773 | 4373 |
| suPAR_DB01_G03 | 2.90E−04 | 2.46E−03 | 174 | 774 | 1374 | 1974 | 2574 | 3174 | 3774 | 4374 |
| suPAR_DB01_G04 | 7.99E−05 | 3.64E−03 | 175 | 775 | 1375 | 1975 | 2575 | 3175 | 3775 | 4375 |
| suPAR_DB01_G05 | 2.53E−04 | 3.45E−03 | 176 | 776 | 1376 | 1976 | 2576 | 3176 | 3776 | 4376 |
| suPAR_DB01_G06 | 2.04E−04 | 3.71E−03 | 177 | 777 | 1377 | 1977 | 2577 | 3177 | 3777 | 4377 |
| suPAR_DB01_G07 | 1.73E−05 | 4.07E−03 | 178 | 778 | 1378 | 1978 | 2578 | 3178 | 3778 | 4378 |
| suPAR_DB01_G08 | 1.04E−04 | 3.09E−03 | 179 | 779 | 1379 | 1979 | 2579 | 3179 | 3779 | 4379 |
| suPAR_DB01_G09 | 3.47E−04 | 4.33E−03 | 180 | 780 | 1380 | 1980 | 2580 | 3180 | 3780 | 4380 |
| suPAR_DB01_G10 | 1.97E−04 | 3.43E−03 | 181 | 781 | 1381 | 1981 | 2581 | 3181 | 3781 | 4381 |
| suPAR_DB01_G11 | 1.62E−04 | 1.90E−03 | 182 | 782 | 1382 | 1982 | 2582 | 3182 | 3782 | 4382 |
| suPAR_DB01_G12 | 2.47E−04 | 4.66E−03 | 183 | 783 | 1383 | 1983 | 2583 | 3183 | 3783 | 4383 |
| suPAR_DB01_H01 | 1.22E−04 | 4.54E−03 | 184 | 784 | 1384 | 1984 | 2584 | 3184 | 3784 | 4384 |
| suPAR_DB01_H02 | 2.14E−04 | 3.47E−03 | 185 | 785 | 1385 | 1985 | 2585 | 3185 | 3785 | 4385 |
| suPAR_DB01_H03 | 6.77E−04 | 1.00E−03 | 186 | 786 | 1386 | 1986 | 2586 | 3186 | 3786 | 4386 |
| suPAR_DB01_H04 | 2.25E−04 | 2.42E−03 | 187 | 787 | 1387 | 1987 | 2587 | 3187 | 3787 | 4387 |
| suPAR_DB01_H05 | 4.32E−04 | 1.52E−03 | 188 | 788 | 1388 | 1988 | 2588 | 3188 | 3788 | 4388 |
| suPAR_DB01_H06 | 1.29E−04 | 5.20E−03 | 189 | 789 | 1389 | 1989 | 2589 | 3189 | 3789 | 4389 |
| suPAR_DB01_H07 | 2.19E−04 | 1.38E−03 | 190 | 790 | 1390 | 1990 | 2590 | 3190 | 3790 | 4390 |
| suPAR_DB01_H08 | 3.80E−04 | 2.81E−03 | 191 | 791 | 1391 | 1991 | 2591 | 3191 | 3791 | 4391 |
| suPAR_DB01_H09 | 2.39E−04 | 3.10E−03 | 192 | 792 | 1392 | 1992 | 2592 | 3192 | 3792 | 4392 |
| suPAR_DB01_H10 | 3.74E−04 | 6.03E−04 | 193 | 793 | 1393 | 1993 | 2593 | 3193 | 3793 | 4393 |
| suPAR_DB02_A03 | 3.41E−04 | 1.98E−03 | 194 | 794 | 1394 | 1994 | 2594 | 3194 | 3794 | 4394 |
| suPAR_DB02_A04 | 5.21E−04 | 2.36E−03 | 195 | 795 | 1395 | 1995 | 2595 | 3195 | 3795 | 4395 |
| suPAR_DB02_A05 | 1.51E−04 | 5.21E−03 | 196 | 796 | 1396 | 1996 | 2596 | 3196 | 3796 | 4396 |
| suPAR_DB02_A06 | 4.39E−07 | 3.23E−03 | 197 | 797 | 1397 | 1997 | 2597 | 3197 | 3797 | 4397 |
| suPAR_DB02_A07 | 3.52E−04 | 1.14E−03 | 198 | 798 | 1398 | 1998 | 2598 | 3198 | 3798 | 4398 |
| suPAR_DB02_A08 | 7.98E−05 | 3.63E−03 | 199 | 799 | 1399 | 1999 | 2599 | 3199 | 3799 | 4399 |
| suPAR_DB02_A09 | 4.84E−04 | 6.13E−03 | 200 | 800 | 1400 | 2000 | 2600 | 3200 | 3800 | 4400 |
| suPAR_DB02_A10 | 2.40E−04 | 3.19E−03 | 201 | 801 | 1401 | 2001 | 2601 | 3201 | 3801 | 4401 |
| suPAR_DB02_A11 | 2.32E−04 | 4.47E−03 | 202 | 802 | 1402 | 2002 | 2602 | 3202 | 3802 | 4402 |
| suPAR_DB02_A12 | 2.26E−05 | 1.70E−03 | 203 | 803 | 1403 | 2003 | 2603 | 3203 | 3803 | 4403 |
| suPAR_DB02_B01 | 3.63E−04 | 2.53E−03 | 204 | 804 | 1404 | 2004 | 2604 | 3204 | 3804 | 4404 |
| suPAR_DB02_B02 | 1.57E−05 | 1.08E−03 | 205 | 805 | 1405 | 2005 | 2605 | 3205 | 3805 | 4405 |
| suPAR_DB02_B03 | 4.24E−04 | 8.80E−04 | 206 | 806 | 1406 | 2006 | 2606 | 3206 | 3806 | 4406 |
| suPAR_DB02_B04 | 1.85E−04 | 4.77E−03 | 207 | 807 | 1407 | 2007 | 2607 | 3207 | 3807 | 4407 |
| suPAR_DB02_B05 | 1.99E−04 | 4.10E−03 | 208 | 808 | 1408 | 2008 | 2608 | 3208 | 3808 | 4408 |
| suPAR_DB02_B06 | 1.27E−04 | 3.53E−03 | 209 | 809 | 1409 | 2009 | 2609 | 3209 | 3809 | 4409 |
| suPAR_DB02_B07 | 8.12E−05 | 4.47E−03 | 210 | 810 | 1410 | 2010 | 2610 | 3210 | 3810 | 4410 |
| suPAR_DB02_B08 | 8.61E−05 | 1.55E−03 | 211 | 811 | 2011 | 2011 | 2611 | 3211 | 3811 | 4411 |
| suPAR_DB02_B09 | 2.08E−04 | 4.22E−03 | 212 | 812 | 1412 | 2012 | 2612 | 3212 | 3812 | 4412 |
| suPAR_DB02_B10 | 4.34E−04 | 1.89E−03 | 213 | 813 | 1413 | 2013 | 2613 | 3213 | 3813 | 4413 |
| suPAR_DB02_B11 | 2.72E−04 | 3.01E−03 | 214 | 814 | 1414 | 2014 | 2614 | 3214 | 3814 | 4414 |
| suPAR_DB02_B12 | 2.30E−04 | 9.58E−03 | 215 | 815 | 1415 | 2015 | 2615 | 3215 | 3815 | 4415 |
| suPAR_DB02_C01 | 2.50E−04 | 4.65E−03 | 216 | 816 | 1416 | 2016 | 2616 | 3216 | 3816 | 4416 |
| suPAR_DB02_C02 | 3.58E−05 | 4.29E−03 | 217 | 817 | 1417 | 2017 | 2617 | 3217 | 3817 | 4417 |
| suPAR_DB02_C03 | 1.78E−04 | 2.86E−03 | 218 | 818 | 1418 | 2018 | 2618 | 3218 | 3818 | 4418 |
| suPAR_DB02_C04 | 1.32E−03 | 1.28E−03 | 219 | 819 | 1419 | 2019 | 2619 | 3219 | 3819 | 4419 |
| suPAR_DB02_C05 | 2.85E−04 | 5.04E−03 | 220 | 820 | 1420 | 2020 | 2620 | 3220 | 3820 | 4420 |
| suPAR_DB02_C06 | 1.41E−04 | 4.75E−03 | 221 | 821 | 1421 | 2021 | 2621 | 3221 | 3821 | 4421 |
| suPAR_DB02_C07 | NA | 2.60E−03 | 222 | 822 | 1422 | 2022 | 2622 | 3222 | 3822 | 4422 |
| suPAR_DB02_C08 | 4.05E−07 | 2.03E−03 | 223 | 823 | 1423 | 2023 | 2623 | 3223 | 3823 | 4423 |
| suPAR_DB02_C09 | 3.35E−04 | 3.87E−03 | 224 | 824 | 1424 | 2024 | 2624 | 3224 | 3824 | 4424 |
| suPAR_DB02_C10 | 1.98E−04 | 2.57E−03 | 225 | 825 | 1425 | 2025 | 2625 | 3225 | 3825 | 4425 |
| suPAR_DB02_C11 | 4.00E−04 | 4.90E−03 | 226 | 826 | 1426 | 2026 | 2626 | 3226 | 3826 | 4426 |
| suPAR_DB02_C12 | 5.82E−04 | 3.19E−03 | 227 | 827 | 1427 | 2027 | 2627 | 3227 | 3827 | 4427 |
| suPAR_DB02_D01 | 4.70E−07 | 3.00E−03 | 228 | 828 | 1428 | 2028 | 2628 | 3228 | 3828 | 4428 |
| suPAR_DB02_D02 | 2.49E−05 | 4.36E−03 | 229 | 829 | 1429 | 2029 | 2629 | 3229 | 3829 | 4429 |
| suPAR_DB02_D03 | 1.88E−04 | 5.50E−03 | 230 | 830 | 1430 | 2030 | 2630 | 3230 | 3830 | 4430 |
| suPAR_DB02_D04 | 2.81E−04 | 4.10E−03 | 231 | 831 | 1431 | 2031 | 2631 | 3231 | 3831 | 4431 |
| suPAR_DB02_D05 | 3.05E−04 | 3.58E−03 | 232 | 832 | 1432 | 2032 | 2632 | 3232 | 3832 | 4432 |
| suPAR_DB02_D06 | 5.10E−04 | 4.16E−03 | 233 | 833 | 1433 | 2033 | 2633 | 3233 | 3833 | 4433 |
| suPAR_DB02_D07 | NA | 2.60E−03 | 234 | 834 | 1434 | 2034 | 2634 | 3234 | 3834 | 4434 |
| suPAR_DB02_D08 | 3.45E−04 | 2.02E−03 | 235 | 835 | 1435 | 2035 | 2635 | 3235 | 3835 | 4435 |
| suPAR_DB02_D09 | 4.98E−04 | 2.32E−03 | 236 | 836 | 1436 | 2036 | 2636 | 3236 | 3836 | 4436 |
| suPAR_DB02_D10 | 3.81E−04 | 1.78E−03 | 237 | 837 | 1437 | 2037 | 2637 | 3237 | 3837 | 4437 |
| suPAR_DB02_D11 | 2.65E−04 | 4.15E−03 | 238 | 838 | 1438 | 2038 | 2638 | 3238 | 3838 | 4438 |
| suPAR_DB02_D12 | 3.80E−03 | 6.11E−03 | 239 | 839 | 1439 | 2039 | 2639 | 3239 | 3839 | 4439 |
| suPAR_DB02_E01 | 3.90E−07 | 2.96E−03 | 240 | 840 | 1440 | 2040 | 2640 | 3240 | 3840 | 4440 |
| suPAR_DB02_E02 | 1.89E−04 | 4.38E−03 | 241 | 841 | 1441 | 2041 | 2641 | 3241 | 3841 | 4441 |
| suPAR_DB02_E03 | 3.08E−04 | 3.66E−03 | 242 | 842 | 1442 | 2042 | 2642 | 3242 | 3842 | 4442 |
| suPAR_DB02_E04 | 2.17E−04 | 3.11E−03 | 243 | 843 | 1443 | 2043 | 2643 | 3243 | 3843 | 4443 |
| suPAR_DB02_E05 | 1.46E−03 | 2.39E−03 | 244 | 844 | 1444 | 2044 | 2644 | 3244 | 3844 | 4444 |
| suPAR_DB02_E06 | 2.95E−04 | 2.39E−03 | 245 | 845 | 1445 | 2045 | 2645 | 3245 | 3845 | 4445 |
| suPAR_DB02_E07 | 1.77E−04 | 3.92E−03 | 246 | 846 | 1446 | 2046 | 2646 | 3246 | 3846 | 4446 |
| suPAR_DB02_E08 | 3.63E−04 | 3.10E−03 | 247 | 847 | 1447 | 2047 | 2647 | 3247 | 3847 | 4447 |
| suPAR_DB02_E09 | 2.10E−04 | 4.73E−03 | 248 | 848 | 1448 | 2048 | 2648 | 3248 | 3848 | 4448 |
| suPAR_DB02_E10 | 1.89E−04 | 5.52E−03 | 249 | 849 | 1449 | 2049 | 2649 | 3249 | 3849 | 4449 |
| suPAR_DB02_E11 | 3.76E−04 | 4.23E−03 | 250 | 850 | 1450 | 2050 | 2650 | 3250 | 3850 | 4450 |
| suPAR_DB02_E12 | 2.15E−04 | 6.75E−03 | 251 | 851 | 1451 | 2051 | 2651 | 3251 | 3851 | 4451 |
| suPAR_DB02_F01 | 4.05E−07 | 2.19E−03 | 252 | 852 | 1452 | 2052 | 2652 | 3252 | 3852 | 4452 |
| suPAR_DB02_F02 | 4.80E−04 | 1.79E−03 | 253 | 853 | 1453 | 2053 | 2653 | 3253 | 3853 | 4453 |
| suPAR_DB02_F03 | 4.92E−04 | 5.04E−03 | 254 | 854 | 1454 | 2054 | 2654 | 3254 | 3854 | 4454 |
| suPAR_DB02_F04 | 4.53E−04 | 3.84E−03 | 255 | 855 | 1455 | 2055 | 2655 | 3255 | 3855 | 4455 |
| suPAR_DB02_F05 | 1.61E−04 | 6.13E−03 | 256 | 856 | 1456 | 2056 | 2656 | 3256 | 3856 | 4456 |
| suPAR_DB02_F06 | 1.74E−04 | 4.95E−03 | 257 | 857 | 1457 | 2057 | 2657 | 3257 | 3857 | 4457 |
| suPAR_DB02_F07 | 3.84E−04 | 2.00E−03 | 258 | 858 | 1458 | 2058 | 2658 | 3258 | 3858 | 4458 |
| suPAR_DB02_F08 | 2.47E−05 | 2.10E−03 | 259 | 859 | 1459 | 2059 | 2659 | 3259 | 3859 | 4459 |
| suPAR_DB02_F09 | 6.24E−04 | 1.66E−03 | 260 | 860 | 1460 | 2060 | 2660 | 3260 | 3860 | 4460 |
| suPAR_DB02_F10 | 3.75E−04 | 1.06E−03 | 261 | 861 | 1461 | 2061 | 2661 | 3261 | 3861 | 4461 |
| suPAR_DB02_F11 | 6.13E−04 | 1.98E−03 | 262 | 862 | 1462 | 2062 | 2662 | 3262 | 3862 | 4462 |
| suPAR_DB02_F12 | 9.82E−05 | 4.94E−03 | 263 | 863 | 1463 | 2063 | 2663 | 3263 | 3863 | 4463 |
| suPAR_DB02_G01 | 5.19E−05 | 4.38E−03 | 264 | 864 | 1464 | 2064 | 2664 | 3264 | 3864 | 4464 |
| suPAR_DB02_G02 | 1.25E−04 | 2.42E−03 | 265 | 865 | 1465 | 2065 | 2665 | 3265 | 3865 | 4465 |
| suPAR_DB02_G03 | 2.50E−04 | 3.46E−03 | 266 | 866 | 1466 | 2066 | 2666 | 3266 | 3866 | 4466 |
| suPAR_DB02_G04 | 9.86E−05 | 4.48E−03 | 267 | 867 | 1467 | 2067 | 2667 | 3267 | 3867 | 4467 |
| suPAR_DB02_G05 | 5.21E−04 | 2.42E−03 | 268 | 868 | 1468 | 2068 | 2668 | 3268 | 3868 | 4468 |
| suPAR_DB02_G06 | 1.32E−04 | 1.37E−03 | 269 | 869 | 1469 | 2069 | 2669 | 3269 | 3869 | 4469 |
| suPAR_DB02_G07 | 2.86E−04 | 3.67E−03 | 270 | 870 | 1470 | 2070 | 2670 | 3270 | 3870 | 4470 |
| suPAR_DB02_G08 | 1.03E−04 | 3.95E−03 | 271 | 871 | 1471 | 2071 | 2671 | 3271 | 3871 | 4471 |
| suPAR_DB02_G09 | 2.50E−04 | 7.33E−03 | 272 | 872 | 1472 | 2072 | 2672 | 3272 | 3872 | 4472 |
| suPAR_DB02_G10 | 4.30E−04 | 5.62E−03 | 273 | 873 | 1473 | 2073 | 2673 | 3273 | 3873 | 4473 |
| suPAR_DB02_G11 | 1.30E−04 | 5.61E−03 | 274 | 874 | 1474 | 2074 | 2674 | 3274 | 3874 | 4474 |
| suPAR_DB02_G12 | 5.84E−04 | 3.71E−03 | 275 | 875 | 1475 | 2075 | 2675 | 3275 | 3875 | 4475 |
| suPAR_DB02_H01 | 2.18E−04 | 3.15E−03 | 276 | 876 | 1476 | 2076 | 2676 | 3276 | 3876 | 4476 |
| suPAR_DB02_H02 | 4.98E−04 | 5.79E−03 | 277 | 877 | 1477 | 2077 | 2677 | 3277 | 3877 | 4477 |
| suPAR_DB02_H03 | 5.24E−04 | 5.94E−03 | 278 | 878 | 1478 | 2078 | 2678 | 3278 | 3878 | 4478 |
| suPAR_DB02_H04 | 6.93E−04 | 8.11E−03 | 279 | 879 | 1479 | 2079 | 2679 | 3279 | 3879 | 4479 |
| suPAR_DB02_H05 | 2.10E−04 | 4.42E−03 | 280 | 880 | 1480 | 2080 | 2680 | 3280 | 3880 | 4480 |
| suPAR_DB02_H06 | 3.93E−04 | 3.05E−03 | 281 | 881 | 1481 | 2081 | 2681 | 3281 | 3881 | 4481 |
| suPAR_DB02_H07 | 1.46E−03 | 3.17E−03 | 282 | 882 | 1482 | 2082 | 2682 | 3282 | 3882 | 4482 |
| suPAR_DB02_H08 | 2.28E−04 | 4.85E−03 | 283 | 883 | 1483 | 2083 | 2683 | 3283 | 3883 | 4483 |
| suPAR_DB02_H09 | 2.18E−03 | 3.38E−02 | 284 | 884 | 1484 | 2084 | 2684 | 3284 | 3884 | 4484 |
| suPAR_DB02_H10 | 1.89E−04 | 3.44E−03 | 285 | 885 | 1485 | 2085 | 2685 | 3285 | 3885 | 4485 |
| suPAR_DB03_A03 | 3.19E−04 | 1.99E−03 | 286 | 886 | 1486 | 2086 | 2686 | 3286 | 3886 | 4486 |
| suPAR_DB03_A04 | 2.24E−04 | 2.87E−03 | 287 | 887 | 1487 | 2087 | 2687 | 3287 | 3887 | 4487 |
| suPAR_DB03_A05 | 2.59E−04 | 2.60E−03 | 288 | 888 | 1488 | 2088 | 2688 | 3288 | 3888 | 4488 |
| suPAR_DB03_A06 | 6.32E−07 | 3.02E−03 | 289 | 889 | 1489 | 2089 | 2689 | 3289 | 3889 | 4489 |
| suPAR_DB03_A07 | 3.64E−07 | 3.26E−03 | 290 | 890 | 1490 | 2090 | 2690 | 3290 | 3890 | 4490 |
| suPAR_DB03_A08 | 2.69E−04 | 3.06E−04 | 291 | 891 | 1491 | 2091 | 2691 | 3291 | 3891 | 4491 |
| suPAR_DB03_A09 | 2.54E−04 | 1.32E−03 | 292 | 892 | 1492 | 2092 | 2692 | 3292 | 3892 | 4492 |
| suPAR_DB03_A10 | 6.85E−04 | 8.38E−04 | 293 | 893 | 1493 | 2093 | 2693 | 3293 | 3893 | 4493 |
| suPAR_DB03_A11 | 7.99E−05 | 1.81E−03 | 294 | 894 | 1494 | 2094 | 2694 | 3294 | 3894 | 4494 |
| suPAR_DB03_A12 | NA | 5.44E−02 | 295 | 895 | 1495 | 2095 | 2695 | 3295 | 3895 | 4495 |
| suPAR_DB03_B01 | 3.43E−04 | 2.97E−03 | 296 | 896 | 1496 | 2096 | 2696 | 3296 | 3896 | 4496 |
| suPAR_DB03_B02 | 1.48E−04 | 2.40E−03 | 297 | 897 | 1497 | 2097 | 2697 | 3297 | 3897 | 4497 |
| suPAR_DB03_B03 | 2.60E−04 | 3.47E−03 | 298 | 898 | 1498 | 2098 | 2698 | 3298 | 3898 | 4498 |
| suPAR_DB03_B04 | 4.52E−07 | 1.97E−03 | 299 | 899 | 1499 | 2099 | 2699 | 3299 | 3899 | 4499 |
| suPAR_DB03_B05 | 1.26E−04 | 5.36E−03 | 300 | 900 | 1500 | 2100 | 2700 | 3300 | 3900 | 4500 |
| suPAR_DB03_B06 | 1.08E−05 | 3.52E−03 | 301 | 901 | 1501 | 2101 | 2701 | 3301 | 3901 | 4501 |
| suPAR_DB03_B07 | 4.00E−07 | 3.06E−03 | 302 | 902 | 1502 | 2102 | 2702 | 3302 | 3902 | 4502 |
| suPAR_DB03_B08 | 2.26E−04 | 1.33E−03 | 303 | 903 | 1503 | 2103 | 2703 | 3303 | 3903 | 4503 |
| suPAR_DB03_B09 | 1.26E−04 | 4.21E−03 | 304 | 904 | 1504 | 2104 | 2704 | 3304 | 3904 | 4504 |
| suPAR_DB03_B10 | 1.38E−04 | 4.00E−03 | 305 | 905 | 1505 | 2105 | 2705 | 3305 | 3905 | 4505 |
| suPAR_DB03_B11 | 3.75E−07 | 2.25E−03 | 306 | 906 | 1506 | 2106 | 2706 | 3306 | 3906 | 4506 |
| suPAR_DB03_B12 | 4.84E−04 | 3.62E−04 | 307 | 907 | 1507 | 2107 | 2707 | 3307 | 3907 | 4507 |
| suPAR_DB03_C01 | 1.65E−04 | 3.66E−03 | 308 | 908 | 1508 | 2108 | 2708 | 3308 | 3908 | 4508 |
| suPAR_DB03_C02 | 3.66E−04 | 5.34E−03 | 309 | 909 | 1509 | 2109 | 2709 | 3309 | 3909 | 4509 |
| suPAR_DB03_C03 | 1.08E−03 | 4.64E−03 | 310 | 910 | 1510 | 2110 | 2710 | 3310 | 3910 | 4510 |
| suPAR_DB03_C04 | 4.37E−07 | 3.01E−03 | 311 | 911 | 1511 | 2111 | 2711 | 3311 | 3911 | 4511 |
| suPAR_DB03_C05 | 1.17E−04 | 3.56E−03 | 312 | 912 | 1512 | 2112 | 2712 | 3312 | 3912 | 4512 |
| suPAR_DB03_C06 | 1.89E−04 | 3.18E−03 | 313 | 913 | 1513 | 2113 | 2713 | 3313 | 3913 | 4513 |
| suPAR_DB03_C07 | 5.78E−05 | 1.66E−03 | 314 | 914 | 1514 | 2114 | 2714 | 3314 | 3914 | 4514 |
| suPAR_DB03_C08 | 9.98E−05 | 1.03E−03 | 315 | 915 | 1515 | 2115 | 2715 | 3315 | 3915 | 4515 |
| suPAR_DB03_C09 | 3.75E−07 | 1.03E−03 | 316 | 916 | 1516 | 2116 | 2716 | 3316 | 3916 | 4516 |
| suPAR_DB03_C10 | 3.01E−04 | 4.52E−03 | 317 | 917 | 1517 | 2117 | 2717 | 3317 | 3917 | 4517 |
| suPAR_DB03_C11 | 6.63E−07 | 2.59E−03 | 318 | 918 | 1518 | 2118 | 2718 | 3318 | 3918 | 4518 |
| suPAR_DB03_C12 | 7.59E−05 | 3.96E−03 | 319 | 919 | 1519 | 2119 | 2719 | 3319 | 3919 | 4519 |
| suPAR_DB03_D01 | 8.49E−05 | 4.16E−03 | 320 | 920 | 1520 | 2120 | 2720 | 3320 | 3920 | 4520 |
| suPAR_DB03_D02 | 2.41E−04 | 7.55E−03 | 321 | 921 | 1521 | 2121 | 2721 | 3321 | 3921 | 4521 |
| suPAR_DB03_D03 | 1.56E−04 | 2.58E−03 | 322 | 922 | 1522 | 2122 | 2722 | 3322 | 3922 | 4522 |
| suPAR_DB03_D04 | 5.90E−03 | 3.25E−03 | 323 | 923 | 1523 | 2123 | 2723 | 3323 | 3923 | 4523 |
| suPAR_DB03_D05 | 3.55E−05 | 4.82E−03 | 324 | 924 | 1524 | 2124 | 2724 | 3324 | 3924 | 4524 |
| suPAR_DB03_D06 | 3.88E−04 | 2.95E−03 | 325 | 925 | 1525 | 2125 | 2725 | 3325 | 3925 | 4525 |
| suPAR_DB03_D07 | 1.63E−04 | 3.08E−03 | 326 | 926 | 1526 | 2126 | 2726 | 3326 | 3926 | 4526 |
| suPAR_DB03_D08 | 4.82E−04 | 4.58E−03 | 327 | 927 | 1527 | 2127 | 2727 | 3327 | 3927 | 4527 |
| suPAR_DB03_D09 | 5.08E−04 | 7.46E−04 | 328 | 928 | 1528 | 2128 | 2728 | 3328 | 3928 | 4528 |
| suPAR_DB03_D10 | 2.86E−04 | 5.27E−03 | 329 | 929 | 1529 | 2129 | 2729 | 3329 | 3929 | 4529 |
| suPAR_DB03_D11 | 5.69E−07 | 3.98E−03 | 330 | 930 | 1530 | 2130 | 2730 | 3330 | 3930 | 4530 |
| suPAR_DB03_D12 | 2.63E−04 | 5.70E−03 | 331 | 931 | 1531 | 2131 | 2731 | 3331 | 3931 | 4531 |
| suPAR_DB03_E01 | 5.59E−04 | 1.75E−03 | 332 | 932 | 1532 | 2132 | 2732 | 3332 | 3932 | 4532 |
| suPAR_DB03_E02 | 2.58E−04 | 3.58E−03 | 333 | 933 | 1533 | 2133 | 2733 | 3333 | 3933 | 4533 |
| suPAR_DB03_E03 | 1.32E−04 | 2.31E−03 | 334 | 934 | 1534 | 2134 | 2734 | 3334 | 3934 | 4534 |
| suPAR_DB03_E04 | 6.69E−04 | 5.56E−03 | 335 | 935 | 1535 | 2135 | 2735 | 3335 | 3935 | 4535 |
| suPAR_DB03_E05 | 6.45E−07 | 3.38E−03 | 336 | 936 | 1536 | 2136 | 2736 | 3336 | 3936 | 4536 |
| suPAR_DB03_E06 | 7.36E−05 | 4.40E−03 | 337 | 937 | 1537 | 2137 | 2737 | 3337 | 3937 | 4537 |
| suPAR_DB03_E07 | 5.45E−07 | 2.89E−03 | 338 | 938 | 1538 | 2138 | 2738 | 3338 | 3938 | 4538 |
| suPAR_DB03_E08 | 2.50E−04 | 5.00E−03 | 339 | 939 | 1539 | 2139 | 2739 | 3339 | 3939 | 4539 |
| suPAR_DB03_E09 | 1.32E−04 | 4.26E−03 | 340 | 940 | 1540 | 2140 | 2740 | 3340 | 3940 | 4540 |
| suPAR_DB03_E10 | 3.47E−04 | 4.81E−03 | 341 | 941 | 1541 | 2141 | 2741 | 3341 | 3941 | 4541 |
| suPAR_DB03_E11 | 5.75E−07 | 2.33E−03 | 342 | 942 | 1542 | 2142 | 2742 | 3342 | 3942 | 4542 |
| suPAR_DB03_E12 | 2.45E−04 | 3.74E−03 | 343 | 943 | 1543 | 2143 | 2743 | 3343 | 3943 | 4543 |
| suPAR_DB03_F01 | 1.51E−04 | 5.25E−03 | 344 | 944 | 1544 | 2144 | 2744 | 3344 | 3944 | 4544 |
| suPAR_DB03_F02 | 2.08E−04 | 9.99E−04 | 345 | 945 | 1545 | 2145 | 2745 | 3345 | 3945 | 4545 |
| suPAR_DB03_F03 | 3.37E−04 | 4.02E−03 | 346 | 946 | 1546 | 2146 | 2746 | 3346 | 3946 | 4546 |
| suPAR_DB03_F04 | 3.89E−07 | 4.43E−03 | 347 | 947 | 1547 | 2147 | 2747 | 3347 | 3947 | 4547 |
| suPAR_DB03_F05 | 2.00E−04 | 4.53E−03 | 348 | 948 | 1548 | 2148 | 2748 | 3348 | 3948 | 4548 |
| suPAR_DB03_F06 | 5.26E−07 | 2.41E−03 | 349 | 949 | 1549 | 2149 | 2749 | 3349 | 3949 | 4549 |
| suPAR_DB03_F07 | 3.43E−04 | 4.22E−03 | 350 | 950 | 1550 | 2150 | 2750 | 3350 | 3950 | 4550 |
| suPAR_DB03_F08 | 6.79E−04 | 1.28E−03 | 351 | 951 | 1551 | 2151 | 2751 | 3351 | 3951 | 4551 |
| suPAR_DB03_F09 | 1.53E−04 | 3.37E−03 | 352 | 952 | 1552 | 2152 | 2752 | 3352 | 3952 | 4552 |
| suPAR_DB03_F10 | 4.75E−04 | 3.24E−03 | 353 | 953 | 1553 | 2153 | 2753 | 3353 | 3953 | 4553 |
| suPAR_DB03_F11 | 1.12E−04 | 3.00E−03 | 354 | 954 | 1554 | 2154 | 2754 | 3354 | 3954 | 4554 |
| suPAR_DB03_F12 | 3.00E−04 | 4.87E−04 | 355 | 955 | 1555 | 2155 | 2755 | 3355 | 3955 | 4555 |
| suPAR_DB03_G01 | 3.30E−04 | 3.06E−03 | 356 | 956 | 1556 | 2156 | 2756 | 3356 | 3956 | 4556 |
| suPAR_DB03_G02 | 6.11E−04 | 5.29E−03 | 357 | 957 | 1557 | 2157 | 2757 | 3357 | 3957 | 4557 |
| suPAR_DB03_G03 | 3.53E−07 | 3.34E−03 | 358 | 958 | 1558 | 2158 | 2758 | 3358 | 3958 | 4558 |
| suPAR_DB03_G04 | 2.96E−04 | 2.10E−03 | 359 | 959 | 1559 | 2159 | 2759 | 3359 | 3959 | 4559 |
| suPAR_DB03_G05 | 4.93E−04 | 1.35E−03 | 360 | 960 | 1560 | 2160 | 2760 | 3360 | 3960 | 4560 |
| suPAR_DB03_G06 | 4.43E−04 | 1.65E−03 | 361 | 961 | 1561 | 2161 | 2761 | 3361 | 3961 | 4561 |
| suPAR_DB03_G07 | 4.23E−07 | 8.11E−04 | 362 | 962 | 1562 | 2162 | 2762 | 3362 | 3962 | 4562 |
| suPAR_DB03_G08 | 3.53E−05 | 4.19E−03 | 363 | 963 | 1563 | 2163 | 2763 | 3363 | 3963 | 4563 |
| suPAR_DB03_G09 | 1.54E−04 | 3.13E−03 | 364 | 964 | 1564 | 2164 | 2764 | 3364 | 3964 | 4564 |
| suPAR_DB03_G10 | 2.96E−04 | 1.17E−03 | 365 | 965 | 1565 | 2165 | 2765 | 3365 | 3965 | 4565 |
| suPAR_DB03_G11 | 4.75E−07 | 1.16E−03 | 366 | 966 | 1566 | 2166 | 2766 | 3366 | 3966 | 4566 |
| suPAR_DB03_G12 | 3.83E−07 | 1.70E−03 | 367 | 967 | 1567 | 2167 | 2767 | 3367 | 3967 | 4567 |
| suPAR_DB03_H01 | 1.80E−04 | 1.00E−03 | 368 | 968 | 1568 | 2168 | 2768 | 3368 | 3968 | 4568 |
| suPAR_DB03_H02 | 1.22E−04 | 1.06E−03 | 369 | 969 | 1569 | 2169 | 2769 | 3369 | 3969 | 4569 |
| suPAR_DB03_H03 | 6.66E−07 | 3.71E−03 | 370 | 970 | 1570 | 2170 | 2770 | 3370 | 3970 | 4570 |
| suPAR_DB03_H04 | 1.81E−04 | 4.43E−03 | 371 | 971 | 1571 | 2171 | 2771 | 3371 | 3971 | 4571 |
| suPAR_DB03_H05 | 4.46E−07 | 4.00E−03 | 372 | 972 | 1572 | 2172 | 2772 | 3372 | 3972 | 4572 |
| suPAR_DB03_H06 | 6.27E−05 | 3.40E−03 | 373 | 973 | 1573 | 2173 | 2773 | 3373 | 3973 | 4573 |
| suPAR_DB03_H07 | 6.80E−07 | 5.49E−04 | 374 | 974 | 1574 | 2174 | 2774 | 3374 | 3974 | 4574 |
| suPAR_DB03_H08 | 3.92E−04 | 4.41E−03 | 375 | 975 | 1575 | 2175 | 2775 | 3375 | 3975 | 4575 |
| suPAR_DB03_H09 | 1.47E−04 | 3.44E−03 | 376 | 976 | 1576 | 2176 | 2776 | 3376 | 3976 | 4576 |
| suPAR_DB03_H10 | 5.05E−04 | 9.44E−04 | 377 | 977 | 1577 | 2177 | 2777 | 3377 | 3977 | 4577 |
| suPAR_DB04_A03 | 7.90E−04 | 4.36E−03 | 378 | 978 | 1578 | 2178 | 2778 | 3378 | 3978 | 4578 |
| suPAR_DB04_A04 | 2.50E−04 | 2.10E−03 | 379 | 979 | 1579 | 2179 | 2779 | 3379 | 3979 | 4579 |
| suPAR_DB04_A05 | 2.02E−04 | 3.55E−03 | 380 | 980 | 1580 | 2180 | 2780 | 3380 | 3980 | 4580 |
| suPAR_DB04_A06 | 1.60E−04 | 3.70E−03 | 381 | 981 | 1581 | 2181 | 2781 | 3381 | 3981 | 4581 |
| suPAR_DB04_A07 | 4.85E−04 | 1.86E−03 | 382 | 982 | 1582 | 2182 | 2782 | 3382 | 3982 | 4582 |
| suPAR_DB04_A08 | 4.01E−05 | 3.43E−03 | 383 | 983 | 1583 | 2183 | 2783 | 3383 | 3983 | 4583 |
| suPAR_DB04_A09 | 3.65E−02 | 1.74E−02 | 384 | 984 | 1584 | 2184 | 2784 | 3384 | 3984 | 4584 |
| suPAR_DB04_A10 | 1.67E−04 | 6.01E−03 | 385 | 985 | 1585 | 2185 | 2785 | 3385 | 3985 | 4585 |
| suPAR_DB04_A11 | 1.85E−04 | 4.14E−03 | 386 | 986 | 1586 | 2186 | 2786 | 3386 | 3986 | 4586 |
| suPAR_DB04_A12 | 4.42E−07 | 2.64E−03 | 387 | 987 | 1587 | 2187 | 2787 | 3387 | 3987 | 4587 |
| suPAR_DB04_B01 | NA | 7.48E−02 | 388 | 988 | 1588 | 2188 | 2788 | 3388 | 3988 | 4588 |
| suPAR_DB04_B02 | 4.88E−07 | 3.65E−03 | 389 | 989 | 1589 | 2189 | 2789 | 3389 | 3989 | 4589 |
| suPAR_DB04_B03 | 2.26E−04 | 8.03E−04 | 390 | 990 | 1590 | 2190 | 2790 | 3390 | 3990 | 4590 |
| suPAR_DB04_B04 | 2.57E−04 | 5.36E−03 | 391 | 991 | 1591 | 2191 | 2791 | 3391 | 3991 | 4591 |
| suPAR_DB04_B05 | 5.56E−07 | 1.68E−03 | 392 | 992 | 1592 | 2192 | 2792 | 3392 | 3992 | 4592 |
| suPAR_DB04_B06 | 9.67E−05 | 4.47E−03 | 393 | 993 | 1593 | 2193 | 2793 | 3393 | 3993 | 4593 |
| suPAR_DB04_B07 | 1.62E−04 | 3.54E−03 | 394 | 994 | 1594 | 2194 | 2794 | 3394 | 3994 | 4594 |
| suPAR_DB04_B08 | 4.25E−05 | 1.63E−03 | 395 | 995 | 1595 | 2195 | 2795 | 3395 | 3995 | 4595 |
| suPAR_DB04_B09 | 5.24E−04 | 5.17E−03 | 396 | 996 | 1596 | 2196 | 2796 | 3396 | 3996 | 4596 |
| suPAR_DB04_B10 | 2.49E−04 | 6.19E−03 | 397 | 997 | 1597 | 2197 | 2797 | 3397 | 3997 | 4597 |
| suPAR_DB04_B11 | NA | 4.27E−02 | 398 | 998 | 1598 | 2198 | 2798 | 3398 | 3998 | 4598 |
| suPAR_DB04_B12 | 1.72E−05 | 2.88E−03 | 399 | 999 | 1599 | 2199 | 2799 | 3399 | 3999 | 4599 |
| suPAR_DB04_C01 | 5.71E−07 | 9.34E−04 | 400 | 1000 | 1600 | 2200 | 2800 | 3400 | 4000 | 4600 |
| suPAR_DB04_C02 | 1.46E−04 | 4.11E−03 | 401 | 1001 | 1601 | 2201 | 2801 | 3401 | 4001 | 4601 |
| suPAR_DB04_C03 | 1.58E−04 | 3.68E−03 | 402 | 1002 | 1602 | 2202 | 2802 | 3402 | 4002 | 4602 |
| suPAR_DB04_C04 | 3.84E−04 | 2.29E−03 | 403 | 1003 | 1603 | 2203 | 2803 | 3403 | 4003 | 4603 |
| suPAR_DB04_C05 | 4.21E−04 | 2.45E−03 | 404 | 1004 | 1604 | 2204 | 2804 | 3404 | 4004 | 4604 |
| suPAR_DB04_C06 | 4.14E−05 | 4.12E−03 | 405 | 1005 | 1605 | 2205 | 2805 | 3405 | 4005 | 4605 |
| suPAR_DB04_C07 | 2.49E−04 | 3.10E−03 | 406 | 1006 | 1606 | 2206 | 2806 | 3406 | 4006 | 4606 |
| suPAR_DB04_C08 | 7.02E−07 | 1.98E−03 | 407 | 1007 | 1607 | 2207 | 2807 | 3407 | 4007 | 4607 |
| suPAR_DB04_C09 | 1.54E−03 | 1.30E−03 | 408 | 1008 | 1608 | 2208 | 2808 | 3408 | 4008 | 4608 |
| suPAR_DB04_C10 | 3.86E−04 | 2.96E−03 | 409 | 1009 | 1609 | 2209 | 2809 | 3409 | 4009 | 4609 |
| suPAR_DB04_C11 | 4.83E−04 | 5.62E−03 | 410 | 1010 | 1610 | 2210 | 2810 | 3410 | 4010 | 4610 |
| suPAR_DB04_C12 | 2.55E−04 | 6.26E−04 | 411 | 1011 | 1611 | 2211 | 2811 | 3411 | 4011 | 4611 |
| suPAR_DB04_D01 | 4.31E−07 | 2.45E−03 | 412 | 1012 | 1612 | 2212 | 2812 | 3412 | 4012 | 4612 |
| suPAR_DB04_D02 | NA | 9.74E−02 | 413 | 1013 | 1613 | 2213 | 2813 | 3413 | 4013 | 4613 |
| suPAR_DB04_D03 | 3.53E−04 | 4.08E−03 | 414 | 1014 | 1614 | 2214 | 2814 | 3414 | 4014 | 4614 |
| suPAR_DB04_D04 | 1.84E−04 | 1.88E−03 | 415 | 1015 | 1615 | 2215 | 2815 | 3415 | 4015 | 4615 |
| suPAR_DB04_D05 | 5.44E−05 | 4.00E−03 | 416 | 1016 | 1616 | 2216 | 2816 | 3416 | 4016 | 4616 |
| suPAR_DB04_D06 | 4.45E−05 | 4.55E−03 | 417 | 1017 | 1617 | 2217 | 2817 | 3417 | 4017 | 4617 |
| suPAR_DB04_D07 | 4.36E−04 | 2.17E−03 | 418 | 1018 | 1618 | 2218 | 2818 | 3418 | 4018 | 4618 |
| suPAR_DB04_D08 | 3.43E−04 | 4.14E−03 | 419 | 1019 | 1619 | 2219 | 2819 | 3419 | 4019 | 4619 |
| suPAR_DB04_D09 | 2.06E−04 | 2.03E−03 | 420 | 1020 | 1620 | 2220 | 2820 | 3420 | 4020 | 4620 |
| suPAR_DB04_D10 | 4.02E−04 | 5.00E−03 | 421 | 1021 | 1621 | 2221 | 2821 | 3421 | 4021 | 4621 |
| suPAR_DB04_D11 | 3.68E−07 | 4.11E−03 | 422 | 1022 | 1622 | 2222 | 2822 | 3422 | 4022 | 4622 |
| suPAR_DB04_D12 | 1.18E−04 | 4.19E−03 | 423 | 1023 | 1623 | 2223 | 2823 | 3423 | 4023 | 4623 |
| suPAR_DB04_E01 | 4.91E−07 | 2.77E−03 | 424 | 1024 | 1624 | 2224 | 2824 | 3424 | 4024 | 4624 |
| suPAR_DB04_E02 | NA | 4.97E−03 | 425 | 1025 | 1625 | 2225 | 2825 | 3425 | 4025 | 4625 |
| suPAR_DB04_E03 | 2.24E−04 | 1.16E−03 | 426 | 1026 | 1626 | 2226 | 2826 | 3426 | 4026 | 4626 |
| suPAR_DB04_E04 | 4.51E−04 | 3.35E−03 | 427 | 1027 | 1627 | 2227 | 2827 | 3427 | 4027 | 4627 |
| suPAR_DB04_E05 | 3.92E−07 | 3.59E−03 | 428 | 1028 | 1628 | 2228 | 2828 | 3428 | 4028 | 4628 |
| suPAR_DB04_E06 | 1.37E−04 | 5.64E−03 | 429 | 1029 | 1629 | 2229 | 2829 | 3429 | 4029 | 4629 |
| suPAR_DB04_E07 | 2.30E−04 | 3.11E−03 | 430 | 1030 | 1630 | 2230 | 2830 | 3430 | 4030 | 4630 |
| suPAR_DB04_E08 | 1.49E−04 | 2.68E−03 | 431 | 1031 | 1631 | 2231 | 2831 | 3431 | 4031 | 4631 |
| suPAR_DB04_E09 | 4.46E−04 | 5.98E−03 | 432 | 1032 | 1632 | 2232 | 2832 | 3432 | 4032 | 4632 |
| suPAR_DB04_E10 | 1.42E−04 | 1.16E−03 | 433 | 1033 | 1633 | 2233 | 2833 | 3433 | 4033 | 4633 |
| suPAR_DB04_E11 | 1.58E−04 | 5.02E−03 | 434 | 1034 | 1634 | 2234 | 2834 | 3434 | 4034 | 4634 |
| suPAR_DB04_E12 | 8.15E−05 | 3.36E−03 | 435 | 1035 | 1635 | 2235 | 2835 | 3435 | 4035 | 4635 |
| suPAR_DB04_F01 | 4.88E−07 | 2.67E−03 | 436 | 1036 | 1636 | 2236 | 2836 | 3436 | 4036 | 4636 |
| suPAR_DB04_F02 | 5.93E−07 | 4.71E−03 | 437 | 1037 | 1637 | 2237 | 2837 | 3437 | 4037 | 4637 |
| suPAR_DB04_F03 | 8.63E−04 | 3.75E−03 | 438 | 1038 | 1638 | 2238 | 2838 | 3438 | 4038 | 4638 |
| suPAR_DB04_F04 | 1.74E−04 | 3.94E−03 | 439 | 1039 | 1639 | 2239 | 2839 | 3439 | 4039 | 4639 |
| suPAR_DB04_F05 | 2.78E−04 | 4.05E−03 | 440 | 1040 | 1640 | 2240 | 2840 | 3440 | 4040 | 4640 |
| suPAR_DB04_F06 | 1.95E−04 | 2.79E−03 | 441 | 1041 | 1641 | 2241 | 2841 | 3441 | 4041 | 4641 |
| suPAR_DB04_F07 | 3.33E−05 | 2.69E−03 | 442 | 1042 | 1642 | 2242 | 2842 | 3442 | 4042 | 4642 |
| suPAR_DB04_F08 | 4.31E−05 | 2.61E−03 | 443 | 1043 | 1643 | 2243 | 2843 | 3443 | 4043 | 4643 |
| suPAR_DB04_F09 | 1.22E−04 | 4.62E−03 | 444 | 1044 | 1644 | 2244 | 2844 | 3444 | 4044 | 4644 |
| suPAR_DB04_F10 | 2.74E−04 | 4.12E−03 | 445 | 1045 | 1645 | 2245 | 2845 | 3445 | 4045 | 4645 |
| suPAR_DB04_F11 | 1.21E−04 | 5.10E−03 | 446 | 1046 | 1646 | 2246 | 2846 | 3446 | 4046 | 4646 |
| suPAR_DB04_F12 | 9.59E−05 | 2.09E−03 | 447 | 1047 | 2247 | 2247 | 2847 | 3447 | 4047 | 4647 |
| suPAR_DB04_G01 | 4.88E−07 | 2.64E−03 | 448 | 1048 | 1648 | 2248 | 2848 | 3448 | 4048 | 4648 |
| suPAR_DB04_G02 | 3.88E−04 | 1.85E−03 | 449 | 1049 | 1649 | 2249 | 2849 | 3449 | 4049 | 4649 |
| suPAR_DB04_G03 | 7.47E−04 | 1.19E−03 | 450 | 1050 | 1650 | 2250 | 2850 | 3450 | 4050 | 4650 |
| suPAR_DB04_G04 | 3.42E−04 | 7.51E−04 | 451 | 1051 | 1651 | 2251 | 2851 | 3451 | 4051 | 4651 |
| suPAR_DB04_G05 | 8.25E−05 | 3.64E−03 | 452 | 1052 | 1652 | 2252 | 2852 | 3452 | 4052 | 4652 |
| suPAR_DB04_G06 | 2.08E−04 | 2.70E−03 | 453 | 1053 | 1653 | 2253 | 2853 | 3453 | 4053 | 4653 |
| suPAR_DB04_G07 | 5.96E−07 | 4.24E−03 | 454 | 1054 | 1654 | 2254 | 2854 | 3454 | 4054 | 4654 |
| suPAR_DB04_G08 | 2.03E−04 | 3.17E−03 | 455 | 1055 | 1655 | 2255 | 2855 | 3455 | 4055 | 4655 |
| suPAR_DB04_G09 | 6.47E−04 | 3.59E−03 | 456 | 1056 | 1656 | 2256 | 2856 | 3456 | 4056 | 4656 |
| suPAR_DB04_G10 | 4.62E−04 | 3.22E−03 | 457 | 1057 | 1657 | 2257 | 2857 | 3457 | 4057 | 4657 |
| suPAR_DB04_G11 | 2.10E−04 | 3.27E−03 | 458 | 1058 | 1658 | 2258 | 2858 | 3458 | 4058 | 4658 |
| suPAR_DB04_G12 | 8.10E−05 | 5.28E−03 | 459 | 1059 | 1659 | 2259 | 2859 | 3459 | 4059 | 4659 |
| suPAR_DB04_H01 | 1.04E−03 | 1.27E−03 | 460 | 1060 | 1660 | 2260 | 2860 | 3460 | 4060 | 4660 |
| suPAR_DB04_H02 | 3.03E−04 | 4.94E−03 | 461 | 1061 | 1661 | 2261 | 2861 | 3461 | 4061 | 4661 |
| suPAR_DB04_H03 | 2.26E−04 | 1.34E−03 | 462 | 1062 | 1662 | 2262 | 2862 | 3462 | 4062 | 4662 |
| suPAR_DB04_H04 | 2.47E−04 | 1.89E−03 | 463 | 1063 | 1663 | 2263 | 2863 | 3463 | 4063 | 4663 |
| suPAR_DB04_H05 | 1.82E−04 | 5.84E−03 | 464 | 1064 | 1664 | 2264 | 2864 | 3464 | 4064 | 4664 |
| suPAR_DB04_H06 | 2.89E−04 | 9.26E−03 | 465 | 1065 | 1665 | 2265 | 2865 | 3465 | 4065 | 4665 |
| suPAR_DB04_H07 | 2.49E−04 | 5.11E−03 | 466 | 1066 | 1666 | 2266 | 2866 | 3466 | 4066 | 4666 |
| suPAR_DB04_H08 | 5.42E−07 | 3.40E−03 | 467 | 1067 | 1667 | 2267 | 2867 | 3467 | 4067 | 4667 |
| suPAR_DB04_H09 | 8.80E−05 | 4.75E−03 | 468 | 1068 | 1668 | 2268 | 2868 | 3468 | 4068 | 4668 |
| suPAR_DB04_H10 | 3.23E−04 | 3.54E−03 | 469 | 1069 | 1669 | 2269 | 2869 | 3469 | 4069 | 4669 |
| suPAR_DB05_A03 | 3.67E−04 | 1.66E−03 | 470 | 1070 | 1670 | 2270 | 2870 | 3470 | 4070 | 4670 |
| suPAR_DB05_A04 | 4.07E−04 | 3.54E−03 | 471 | 1071 | 1671 | 2271 | 2871 | 3471 | 4071 | 4671 |
| suPAR_DB05_A05 | 6.39E−04 | 5.53E−03 | 472 | 1072 | 1672 | 2272 | 2872 | 3472 | 4072 | 4672 |
| suPAR_DB05_A06 | 2.77E−04 | 3.52E−03 | 473 | 1073 | 1673 | 2273 | 2873 | 3473 | 4073 | 4673 |
| suPAR_DB05_A07 | 2.90E−04 | 4.95E−03 | 474 | 1074 | 1674 | 2274 | 2874 | 3474 | 4074 | 4674 |
| suPAR_DB05_A08 | 9.80E−05 | 3.07E−03 | 475 | 1075 | 1675 | 2275 | 2875 | 3475 | 4075 | 4675 |
| suPAR_DB05_A09 | 9.86E−04 | 6.15E−03 | 476 | 1076 | 1676 | 2276 | 2876 | 3476 | 4076 | 4676 |
| suPAR_DB05_A10 | 4.62E−04 | 5.41E−03 | 477 | 1077 | 1677 | 2277 | 2877 | 3477 | 4077 | 4677 |
| suPAR_DB05_A11 | 3.11E−04 | 1.80E−03 | 478 | 1078 | 1678 | 2278 | 2878 | 3478 | 4078 | 4678 |
| suPAR_DB05_A12 | 1.04E−04 | 4.87E−03 | 479 | 1079 | 1679 | 2279 | 2879 | 3479 | 4079 | 4679 |
| suPAR_DB05_B01 | 2.32E−04 | 3.56E−03 | 480 | 1080 | 1680 | 2280 | 2880 | 3480 | 4080 | 4680 |
| suPAR_DB05_B02 | 4.19E−04 | 5.65E−03 | 481 | 1081 | 1681 | 2281 | 2881 | 3481 | 4081 | 4681 |
| suPAR_DB05_B03 | 3.11E−04 | 3.66E−03 | 482 | 1082 | 1682 | 2282 | 2882 | 3482 | 4082 | 4682 |
| suPAR_DB05_B04 | 6.06E−04 | 6.23E−03 | 483 | 1083 | 1683 | 2283 | 2883 | 3483 | 4083 | 4683 |
| suPAR_DB05_B05 | 1.93E−04 | 4.17E−03 | 484 | 1084 | 1684 | 2284 | 2884 | 3484 | 4084 | 4684 |
| suPAR_DB05_B06 | 2.34E−04 | 2.13E−03 | 485 | 1085 | 1685 | 2285 | 2885 | 3485 | 4085 | 4685 |
| suPAR_DB05_B07 | 3.68E−05 | 5.06E−03 | 486 | 1086 | 1686 | 2286 | 2886 | 3486 | 4086 | 4686 |
| suPAR_DB05_B08 | 1.29E−04 | 4.88E−03 | 487 | 1087 | 1687 | 2287 | 2887 | 3487 | 4087 | 4687 |
| suPAR_DB05_B09 | 5.83E−04 | 2.73E−03 | 488 | 1088 | 1688 | 2288 | 2888 | 3488 | 4088 | 4688 |
| suPAR_DB05_B10 | 3.21E−04 | 4.99E−03 | 489 | 1089 | 1689 | 2289 | 2889 | 3489 | 4089 | 4689 |
| suPAR_DB05_B11 | 4.31E−04 | 2.19E−03 | 490 | 1090 | 1690 | 2290 | 2890 | 3490 | 4090 | 4690 |
| suPAR_DB05_B12 | 3.83E−04 | 4.72E−03 | 491 | 1091 | 2291 | 2291 | 2891 | 3491 | 4091 | 4691 |
| suPAR_DB05_C01 | 4.36E−04 | 3.20E−03 | 492 | 1092 | 1692 | 2292 | 2892 | 3492 | 4092 | 4692 |
| suPAR_DB05_C02 | 6.10E−04 | 3.75E−03 | 493 | 1093 | 1693 | 2293 | 2893 | 3493 | 4093 | 4693 |
| suPAR_DB05_C03 | 4.55E−04 | 3.67E−03 | 494 | 1094 | 1694 | 2294 | 2894 | 3494 | 4094 | 4694 |
| suPAR_DB05_C04 | 4.57E−04 | 4.52E−03 | 495 | 1095 | 1695 | 2295 | 2895 | 3495 | 4095 | 4695 |
| suPAR_DB05_C05 | 2.02E−04 | 5.47E−03 | 496 | 1096 | 1696 | 2296 | 2896 | 3496 | 4096 | 4696 |
| suPAR_DB05_C06 | 1.23E−04 | 5.07E−03 | 497 | 1097 | 1697 | 2297 | 2897 | 3497 | 4097 | 4697 |
| suPAR_DB05_C07 | 2.37E−04 | 4.31E−03 | 498 | 1098 | 1698 | 2298 | 2898 | 3498 | 4098 | 4698 |
| suPAR_DB05_C08 | 5.48E−04 | 2.84E−03 | 499 | 1099 | 1699 | 2299 | 2899 | 3499 | 4099 | 4699 |
| suPAR_DB05_C09 | 4.64E−04 | 3.84E−03 | 500 | 1100 | 1700 | 2300 | 2900 | 3500 | 4100 | 4700 |
| suPAR_DB05_C10 | 2.67E−04 | 4.15E−03 | 501 | 1101 | 1701 | 2301 | 2901 | 3501 | 4101 | 4701 |
| suPAR_DB05_C11 | 1.68E−04 | 3.35E−03 | 502 | 1102 | 1702 | 2302 | 2902 | 3502 | 4102 | 4702 |
| suPAR_DB05_C12 | 2.98E−02 | 1.91E−02 | 503 | 1103 | 1703 | 2303 | 2903 | 3503 | 4103 | 4703 |
| suPAR_DB05_D01 | 3.15E−04 | 5.33E−03 | 504 | 1104 | 1704 | 2304 | 2904 | 3504 | 4104 | 4704 |
| suPAR_DB05_D02 | 4.35E−04 | 1.16E−03 | 505 | 1105 | 1705 | 2305 | 2905 | 3505 | 4105 | 4705 |
| suPAR_DB05_D03 | 1.24E−03 | 3.45E−03 | 506 | 1106 | 1706 | 2306 | 2906 | 3506 | 4106 | 4706 |
| suPAR_DB05_D04 | 3.38E−04 | 2.43E−03 | 507 | 1107 | 1707 | 2307 | 2907 | 3507 | 4107 | 4707 |
| suPAR_DB05_D05 | 6.69E−04 | 1.00E−03 | 508 | 1108 | 1708 | 2308 | 2908 | 3508 | 4108 | 4708 |
| suPAR_DB05_D06 | 3.75E−04 | 3.45E−03 | 509 | 1109 | 1709 | 2309 | 2909 | 3509 | 4109 | 4709 |
| suPAR_DB05_D07 | 2.97E−04 | 5.12E−03 | 510 | 1110 | 1710 | 2310 | 2910 | 3510 | 4110 | 4710 |
| suPAR_DB05_D08 | 7.69E−05 | 4.71E−03 | 511 | 1111 | 1711 | 2311 | 2911 | 3511 | 4111 | 4711 |
| suPAR_DB05_D09 | 6.66E−04 | 3.84E−03 | 512 | 1112 | 1712 | 2312 | 2912 | 3512 | 4112 | 4712 |
| suPAR_DB05_D10 | 8.51E−04 | 6.00E−03 | 513 | 1113 | 1713 | 2313 | 2913 | 3513 | 4113 | 4713 |
| suPAR_DB05_D11 | 7.75E−04 | 5.55E−03 | 514 | 1114 | 1714 | 2314 | 2914 | 3514 | 4114 | 4714 |
| suPAR_DB05_D12 | 3.52E−04 | 4.77E−03 | 515 | 1115 | 1715 | 2315 | 2915 | 3515 | 4115 | 4715 |
| suPAR_DB05_E01 | 7.67E−04 | 2.08E−03 | 516 | 1116 | 1716 | 2316 | 2916 | 3516 | 4116 | 4716 |
| suPAR_DB05_E02 | 8.24E−04 | 5.17E−03 | 517 | 1117 | 1717 | 2317 | 2917 | 3517 | 4117 | 4717 |
| suPAR_DB05_E03 | 2.65E−04 | 5.35E−03 | 518 | 1118 | 1718 | 2318 | 2918 | 3518 | 4118 | 4718 |
| suPAR_DB05_E04 | 3.54E−04 | 5.44E−03 | 519 | 1119 | 1719 | 2319 | 2919 | 3519 | 4119 | 4719 |
| suPAR_DB05_E05 | 2.98E−04 | 1.65E−03 | 520 | 1120 | 1720 | 2320 | 2920 | 3520 | 4120 | 4720 |
| suPAR_DB05_E06 | 1.83E−04 | 2.69E−03 | 521 | 1121 | 1721 | 2321 | 2921 | 3521 | 4121 | 4721 |
| suPAR_DB05_E07 | 6.37E−07 | 4.48E−03 | 522 | 1122 | 1722 | 2322 | 2922 | 3522 | 4122 | 4722 |
| suPAR_DB05_E08 | 8.26E−05 | 3.98E−03 | 523 | 1123 | 1723 | 2323 | 2923 | 3523 | 4123 | 4723 |
| suPAR_DB05_E09 | 4.15E−04 | 2.80E−03 | 524 | 1124 | 1724 | 2324 | 2924 | 3524 | 4124 | 4724 |
| suPAR_DB05_E10 | 3.69E−04 | 2.76E−03 | 525 | 1125 | 1725 | 2325 | 2925 | 3525 | 4125 | 4725 |
| suPAR_DB05_E11 | 4.36E−04 | 1.13E−03 | 526 | 1126 | 1726 | 2326 | 2926 | 3526 | 4126 | 4726 |
| suPAR_DB05_E12 | 6.17E−04 | 5.19E−03 | 527 | 1127 | 1727 | 2327 | 2927 | 3527 | 4127 | 4727 |
| suPAR_DB05_F01 | 3.63E−04 | 5.08E−03 | 528 | 1128 | 1728 | 2328 | 2928 | 3528 | 4128 | 4728 |
| suPAR_DB05_F02 | 4.76E−04 | 3.64E−03 | 529 | 1129 | 1729 | 2329 | 2929 | 3529 | 4129 | 4729 |
| suPAR_DB05_F03 | 5.02E−04 | 2.89E−03 | 530 | 1130 | 1730 | 2330 | 2930 | 3530 | 4130 | 4730 |
| suPAR_DB05_F04 | 3.36E−04 | 3.48E−03 | 531 | 1131 | 1731 | 2331 | 2931 | 3531 | 4131 | 4731 |
| suPAR_DB05_F05 | 4.89E−07 | 3.92E−03 | 532 | 1132 | 1732 | 2332 | 2932 | 3532 | 4132 | 4732 |
| suPAR_DB05_F06 | 5.44E−05 | 5.14E−03 | 533 | 1133 | 1733 | 2333 | 2933 | 3533 | 4133 | 4733 |
| suPAR_DB05_F07 | 3.80E−04 | 5.75E−03 | 534 | 1134 | 1734 | 2334 | 2934 | 3534 | 4134 | 4734 |
| suPAR_DB05_F08 | 3.45E−04 | 2.55E−03 | 535 | 1135 | 1735 | 2335 | 2935 | 3535 | 4135 | 4735 |
| suPAR_DB05_F09 | 5.67E−04 | 4.62E−03 | 536 | 1136 | 1736 | 2336 | 2936 | 3536 | 4136 | 4736 |
| suPAR_DB05_F10 | 7.04E−04 | 1.76E−03 | 537 | 1137 | 1737 | 2337 | 2937 | 3537 | 4137 | 4737 |
| suPAR_DB05_F11 | 3.55E−04 | 2.92E−03 | 538 | 1138 | 1738 | 2338 | 2938 | 3538 | 4138 | 4738 |
| suPAR_DB05_F12 | 6.90E−05 | 4.72E−03 | 539 | 1139 | 1739 | 2339 | 2939 | 3539 | 4139 | 4739 |
| suPAR_DB05_G01 | 2.24E−04 | 3.62E−03 | 540 | 1140 | 1740 | 2340 | 2940 | 3540 | 4140 | 4740 |
| suPAR_DB05_G02 | 1.47E−04 | 3.77E−03 | 541 | 1141 | 1741 | 2341 | 2941 | 3541 | 4141 | 4741 |
| suPAR_DB05_G03 | 6.17E−04 | 1.99E−03 | 542 | 1142 | 1742 | 2342 | 2942 | 3542 | 4142 | 4742 |
| suPAR_DB05_G04 | 4.92E−04 | 3.14E−03 | 543 | 1143 | 1743 | 2343 | 2943 | 3543 | 4143 | 4743 |
| suPAR_DB05_G05 | 1.49E−04 | 4.63E−03 | 544 | 1144 | 1744 | 2344 | 2944 | 3544 | 4144 | 4744 |
| suPAR_DB05_G06 | 6.26E−04 | 1.98E−03 | 545 | 1145 | 1745 | 2345 | 2945 | 3545 | 4145 | 4745 |
| suPAR_DB05_G07 | 4.90E−05 | 4.13E−03 | 546 | 1146 | 1746 | 2346 | 2946 | 3546 | 4146 | 4746 |
| suPAR_DB05_G08 | 4.07E−04 | 2.64E−03 | 547 | 1147 | 1747 | 2347 | 2947 | 3547 | 4147 | 4747 |
| suPAR_DB05_G09 | 3.18E−04 | 4.39E−03 | 548 | 1148 | 1748 | 2348 | 2948 | 3548 | 4148 | 4748 |
| suPAR_DB05_G10 | 3.67E−04 | 5.38E−03 | 549 | 1149 | 1749 | 2349 | 2949 | 3549 | 4149 | 4749 |
| suPAR_DB05_G11 | 5.94E−04 | 6.01E−03 | 550 | 1150 | 1750 | 2350 | 2950 | 3550 | 4150 | 4750 |
| suPAR_DB05_G12 | 8.37E−05 | 4.79E−03 | 551 | 1151 | 1751 | 2351 | 2951 | 3551 | 4151 | 4751 |
| suPAR_DB05_H01 | 1.24E−04 | 1.86E−03 | 552 | 1152 | 1752 | 2352 | 2952 | 3552 | 4152 | 4752 |
| suPAR_DB05_H02 | 6.38E−07 | 2.63E−03 | 553 | 1153 | 1753 | 2353 | 2953 | 3553 | 4153 | 4753 |
| suPAR_DB05_H03 | 2.88E−05 | 1.24E−03 | 554 | 1154 | 1754 | 2354 | 2954 | 3554 | 4154 | 4754 |
| suPAR_DB05_H04 | 3.54E−04 | 3.67E−03 | 555 | 1155 | 1755 | 2355 | 2955 | 3555 | 4155 | 4755 |
| suPAR_DB05_H05 | 7.55E−04 | 2.04E−03 | 556 | 1156 | 1756 | 2356 | 2956 | 3556 | 4156 | 4756 |
| suPAR_DB05_H06 | 6.98E−05 | 3.81E−03 | 557 | 1157 | 1757 | 2357 | 2957 | 3557 | 4157 | 4757 |
| suPAR_DB05_H07 | 1.85E−04 | 5.96E−03 | 558 | 1158 | 1758 | 2358 | 2958 | 3558 | 4158 | 4758 |
| suPAR_DB05_H08 | 2.94E−04 | 3.33E−03 | 559 | 1159 | 1759 | 2359 | 2959 | 3559 | 4159 | 4759 |
| suPAR_DB05_H09 | 3.54E−04 | 4.65E−03 | 560 | 1160 | 1760 | 2360 | 2960 | 3560 | 4160 | 4760 |
| suPAR_DB05_H10 | 1.26E−04 | 3.50E−03 | 561 | 1161 | 1761 | 2361 | 2961 | 3561 | 4161 | 4761 |
| suPAR_DB06_A03 | 5.73E−04 | 6.56E−03 | 562 | 1162 | 1762 | 2362 | 2962 | 3562 | 4162 | 4762 |
| suPAR_DB06_A04 | 6.95E−04 | 7.55E−04 | 563 | 1163 | 1763 | 2363 | 2963 | 3563 | 4163 | 4763 |
| suPAR_DB06_A05 | 2.27E−04 | 3.87E−03 | 564 | 1164 | 1764 | 2364 | 2964 | 3564 | 4164 | 4764 |
| suPAR_DB06_A06 | 1.95E−04 | 4.07E−03 | 565 | 1165 | 1765 | 2365 | 2965 | 3565 | 4165 | 4765 |
| suPAR_DB06_A07 | 3.32E−04 | 4.02E−03 | 566 | 1166 | 1766 | 2366 | 2966 | 3566 | 4166 | 4766 |
| suPAR_DB06_A08 | 9.50E−05 | 2.88E−03 | 567 | 1167 | 1767 | 2367 | 2967 | 3567 | 4167 | 4767 |
| suPAR_DB06_A09 | 4.81E−04 | 6.81E−03 | 568 | 1168 | 1768 | 2368 | 2968 | 3568 | 4168 | 4768 |
| suPAR_DB06_A10 | 4.07E−04 | 2.80E−03 | 569 | 1169 | 1769 | 2369 | 2969 | 3569 | 4169 | 4769 |
| suPAR_DB06_A11 | 5.04E−04 | 5.34E−03 | 570 | 1170 | 1770 | 2370 | 2970 | 3570 | 4170 | 4770 |
| suPAR_DB06_A12 | 4.88E−07 | 4.95E−03 | 571 | 1171 | 1771 | 2371 | 2971 | 3571 | 4171 | 4771 |
| suPAR_DB06_B01 | 4.32E−05 | 9.44E−04 | 572 | 1172 | 1772 | 2372 | 2972 | 3572 | 4172 | 4772 |
| suPAR_DB06_B02 | 4.99E−04 | 3.73E−03 | 573 | 1173 | 1773 | 2373 | 2973 | 3573 | 4173 | 4773 |
| suPAR_DB06_B03 | 2.16E−04 | 4.17E−03 | 574 | 1174 | 1774 | 2374 | 2974 | 3574 | 4174 | 4774 |
| suPAR_DB06_B04 | 4.28E−04 | 3.88E−03 | 575 | 1175 | 1775 | 2375 | 2975 | 3575 | 4175 | 4775 |
| suPAR_DB06_B05 | 3.41E−04 | 4.55E−03 | 576 | 1176 | 1776 | 2376 | 2976 | 3576 | 4176 | 4776 |
| suPAR_DB06_B06 | 2.34E−04 | 4.12E−03 | 577 | 1177 | 1777 | 2377 | 2977 | 3577 | 4177 | 4777 |
| suPAR_DB06_B07 | 1.52E−04 | 5.34E−03 | 578 | 1178 | 1778 | 2378 | 2978 | 3578 | 4178 | 4778 |
| suPAR_DB06_B08 | 1.89E−04 | 3.18E−03 | 579 | 1179 | 1779 | 2379 | 2979 | 3579 | 4179 | 4779 |
| suPAR_DB06_B09 | 6.66E−04 | 6.58E−03 | 580 | 1180 | 1780 | 2380 | 2980 | 3580 | 4180 | 4780 |
| suPAR_DB06_B10 | 5.87E−04 | 1.41E−03 | 581 | 1181 | 1781 | 2381 | 2981 | 3581 | 4181 | 4781 |
| suPAR_DB06_B11 | 1.28E−04 | 4.89E−03 | 582 | 1182 | 1782 | 2382 | 2982 | 3582 | 4182 | 4782 |
| suPAR_DB06_B12 | 2.93E−04 | 2.03E−03 | 583 | 1183 | 1783 | 2383 | 2983 | 3583 | 4183 | 4783 |
| suPAR_DB06_C01 | 1.49E−05 | 2.33E−03 | 584 | 1184 | 1784 | 2384 | 2984 | 3584 | 4184 | 4784 |
| suPAR_DB06_C02 | 1.77E−04 | 2.23E−03 | 585 | 1185 | 1785 | 2385 | 2985 | 3585 | 4185 | 4785 |
| suPAR_DB06_C03 | 3.32E−04 | 3.47E−03 | 586 | 1186 | 1786 | 2386 | 2986 | 3586 | 4186 | 4786 |
| suPAR_DB06_C04 | 1.73E−04 | 3.47E−03 | 587 | 1187 | 1787 | 2387 | 2987 | 3587 | 4187 | 4787 |
| suPAR_DB06_C05 | 6.23E−02 | 3.73E−01 | 588 | 1188 | 1788 | 2388 | 2988 | 3588 | 4188 | 4788 |
| suPAR_DB06_C06 | 2.50E−04 | 3.11E−03 | 589 | 1189 | 1789 | 2389 | 2989 | 3589 | 4189 | 4789 |
| suPAR_DB06_C07 | 3.87E−04 | 4.95E−03 | 590 | 1190 | 1790 | 2390 | 2990 | 3590 | 4190 | 4790 |
| suPAR_DB06_C08 | 8.92E−06 | 3.42E−03 | 591 | 1191 | 1791 | 2391 | 2991 | 3591 | 4191 | 4791 |
| suPAR_DB06_C09 | 5.66E−04 | 3.82E−03 | 592 | 1192 | 1792 | 2392 | 2992 | 3592 | 4192 | 4792 |
| suPAR_DB06_C10 | 4.80E−04 | 5.99E−03 | 593 | 1193 | 1793 | 2393 | 2993 | 3593 | 4193 | 4793 |
| suPAR_DB06_C11 | 6.31E−04 | 2.01E−03 | 594 | 1194 | 1794 | 2394 | 2994 | 3594 | 4194 | 4794 |
| suPAR_DB06_C12 | 7.52E−05 | 4.84E−03 | 595 | 1195 | 1795 | 2395 | 2995 | 3595 | 4195 | 4795 |
| suPAR_DB06_D01 | 1.17E−04 | 3.96E−03 | 596 | 1196 | 1796 | 2396 | 2996 | 3596 | 4196 | 4796 |
| suPAR_DB06_D02 | 2.30E−04 | 4.22E−03 | 597 | 1197 | 1797 | 2397 | 2997 | 3597 | 4197 | 4797 |
| suPAR_DB06_D03 | 8.03E−04 | 3.15E−03 | 598 | 1198 | 1798 | 2398 | 2998 | 3598 | 4198 | 4798 |
| suPAR_DB06_D04 | 7.00E−04 | 2.91E−03 | 599 | 1199 | 1799 | 2399 | 2999 | 3599 | 4199 | 4799 |
| suPAR_DB06_D05 | 2.22E−04 | 5.58E−03 | 600 | 1200 | 1800 | 2400 | 3000 | 3600 | 4200 | 4800 |
| suPAR_DB06_D06 | 2.61E−04 | 4.06E−03 | 601 | 1201 | 1801 | 2401 | 3001 | 3601 | 4201 | 4801 |
| suPAR_DB06_D07 | 5.65E−04 | 2.44E−03 | 602 | 1202 | 1802 | 2402 | 3002 | 3602 | 4202 | 4802 |
| suPAR_DB06_D08 | 7.15E−04 | 1.66E−03 | 603 | 1203 | 1803 | 2403 | 3003 | 3603 | 4203 | 4803 |
| suPAR_DB06_D09 | 3.41E−04 | 1.49E−03 | 604 | 1204 | 1804 | 2404 | 3004 | 3604 | 4204 | 4804 |
| suPAR_DB06_D10 | 3.65E−04 | 4.25E−03 | 605 | 1205 | 1805 | 2405 | 3005 | 3605 | 4205 | 4805 |
| suPAR_DB06_D11 | 4.39E−04 | 2.10E−03 | 606 | 1206 | 1806 | 2406 | 3006 | 3606 | 4206 | 4806 |
| suPAR_DB06_D12 | 1.89E−04 | 5.15E−03 | 607 | 1207 | 1807 | 2407 | 3007 | 3607 | 4207 | 4807 |
| suPAR_DB06_E01 | 1.26E−04 | 4.83E−03 | 608 | 1208 | 1808 | 2408 | 3008 | 3608 | 4208 | 4808 |
| suPAR_DB06_E02 | 5.89E−04 | 1.89E−03 | 609 | 1209 | 1809 | 2409 | 3009 | 3609 | 4209 | 4809 |
| suPAR_DB06_E03 | 3.70E−04 | 2.01E−03 | 610 | 1210 | 1810 | 2410 | 3010 | 3610 | 4210 | 4810 |
| suPAR_DB06_E04 | 3.88E−04 | 5.41E−03 | 611 | 1211 | 1811 | 2411 | 3011 | 3611 | 4211 | 4811 |
| suPAR_DB06_E05 | 9.05E−04 | 3.15E−03 | 612 | 1212 | 1812 | 2412 | 3012 | 3612 | 4212 | 4812 |
| suPAR_DB06_E06 | 3.19E−04 | 1.35E−03 | 613 | 1213 | 1813 | 2413 | 3013 | 3613 | 4213 | 4813 |
| suPAR_DB06_E07 | 3.88E−04 | 4.97E−03 | 614 | 1214 | 1814 | 2414 | 3014 | 3614 | 4214 | 4814 |
| suPAR_DB06_E08 | 2.02E−05 | 3.35E−03 | 615 | 1215 | 1815 | 2415 | 3015 | 3615 | 4215 | 4815 |
| suPAR_DB06_E09 | 3.01E−04 | 3.16E−03 | 616 | 1216 | 1816 | 2416 | 3016 | 3616 | 4216 | 4816 |
| suPAR_DB06_E10 | 2.71E−04 | 5.26E−03 | 617 | 1217 | 1817 | 2417 | 3017 | 3617 | 4217 | 4817 |
| suPAR_DB06_E11 | 7.23E−04 | 1.18E−03 | 618 | 1218 | 1818 | 2418 | 3018 | 3618 | 4218 | 4818 |
| suPAR_DB06_E12 | 1.64E−04 | 3.73E−03 | 619 | 1219 | 1819 | 2419 | 3019 | 3619 | 4219 | 4819 |
| suPAR_DB06_F01 | 1.97E−04 | 3.05E−03 | 620 | 1220 | 1820 | 2420 | 3020 | 3620 | 4220 | 4820 |
| suPAR_DB06_F02 | 2.58E−04 | 1.57E−03 | 621 | 1221 | 1821 | 2421 | 3021 | 3621 | 4221 | 4821 |
| suPAR_DB06_F03 | 5.90E−04 | 4.00E−03 | 622 | 1222 | 1822 | 2422 | 3022 | 3622 | 4222 | 4822 |
| suPAR_DB06_F04 | 3.15E−04 | 5.74E−03 | 623 | 1223 | 1823 | 2423 | 3023 | 3623 | 4223 | 4823 |
| suPAR_DB06_F05 | 3.10E−07 | 5.31E−03 | 624 | 1224 | 1824 | 2424 | 3024 | 3624 | 4224 | 4824 |
| suPAR_DB06_F06 | 5.50E−04 | 3.31E−03 | 625 | 1225 | 1825 | 2425 | 3025 | 3625 | 4225 | 4825 |
| suPAR_DB06_F07 | 4.55E−04 | 4.02E−03 | 626 | 1226 | 1826 | 2426 | 3026 | 3626 | 4226 | 4826 |
| suPAR_DB06_F08 | 5.37E−04 | 1.74E−03 | 627 | 1227 | 1827 | 2427 | 3027 | 3627 | 4227 | 4827 |
| suPAR_DB06_F09 | 3.53E−04 | 3.02E−03 | 628 | 1228 | 1828 | 2428 | 3028 | 3628 | 4228 | 4828 |
| suPAR_DB06_F10 | 7.95E−04 | 2.30E−03 | 629 | 1229 | 1829 | 2429 | 3029 | 3629 | 4229 | 4829 |
| suPAR_DB06_F11 | 5.90E−04 | 9.47E−04 | 630 | 1230 | 1830 | 2430 | 3030 | 3630 | 4230 | 4830 |
| suPAR_DB06_F12 | 1.95E−04 | 4.26E−03 | 631 | 1231 | 1831 | 2431 | 3031 | 3631 | 4231 | 4831 |
| suPAR_DB06_G01 | 1.34E−04 | 1.48E−03 | 632 | 1232 | 1832 | 2432 | 3032 | 3632 | 4232 | 4832 |
| suPAR_DB06_G02 | 3.75E−04 | 3.84E−03 | 633 | 1233 | 1833 | 2433 | 3033 | 3633 | 4233 | 4833 |
| suPAR_DB06_G03 | 2.31E−04 | 3.81E−03 | 634 | 1234 | 1834 | 2434 | 3034 | 3634 | 4234 | 4834 |
| suPAR_DB06_G04 | 2.23E−04 | 5.49E−03 | 635 | 1235 | 1835 | 2435 | 3035 | 3635 | 4235 | 4835 |
| suPAR_DB06_G05 | 4.17E−04 | 3.33E−03 | 636 | 1236 | 1836 | 2436 | 3036 | 3636 | 4236 | 4836 |
| suPAR_DB06_G06 | 5.28E−07 | 4.31E−03 | 637 | 1237 | 1837 | 2437 | 3037 | 3637 | 4237 | 4837 |
| suPAR_DB06_G07 | 1.95E−04 | 3.30E−03 | 638 | 1238 | 1838 | 2438 | 3038 | 3638 | 4238 | 4838 |
| suPAR_DB06_G08 | 6.25E−05 | 1.37E−03 | 639 | 1239 | 1839 | 2439 | 3039 | 3639 | 4239 | 4839 |
| suPAR_DB06_G09 | 4.56E−04 | 2.55E−03 | 640 | 1240 | 1840 | 2440 | 3040 | 3640 | 4240 | 4840 |
| suPAR_DB06_G10 | 3.56E−04 | 6.33E−03 | 641 | 1241 | 1841 | 2441 | 3041 | 3641 | 4241 | 4841 |
| suPAR_DB06_G11 | 3.97E−04 | 2.85E−03 | 642 | 1242 | 1842 | 2442 | 3042 | 3642 | 4242 | 4842 |
| suPAR_DB06_G12 | 2.70E−04 | 1.49E−03 | 643 | 1243 | 1843 | 2443 | 3043 | 3643 | 4243 | 4843 |
| suPAR_DB06_H01 | 6.60E−04 | 9.96E−03 | 644 | 1244 | 1844 | 2444 | 3044 | 3644 | 4244 | 4844 |
| suPAR_DB06_H02 | 4.54E−04 | 4.29E−03 | 645 | 1245 | 1845 | 2445 | 3045 | 3645 | 4245 | 4845 |
| suPAR_DB06_H03 | 4.91E−04 | 2.27E−03 | 646 | 1246 | 1846 | 2446 | 3046 | 3646 | 4246 | 4846 |
| suPAR_DB06_H04 | 7.86E−04 | 5.28E−03 | 647 | 1247 | 1847 | 2447 | 3047 | 3647 | 4247 | 4847 |
| suPAR_DB06_H05 | 5.42E−04 | 2.33E−03 | 648 | 1248 | 1848 | 2448 | 3048 | 3648 | 4248 | 4848 |
| suPAR_DB06_H06 | 1.27E−04 | 6.42E−03 | 649 | 1249 | 1849 | 2449 | 3049 | 3649 | 4249 | 4849 |
| suPAR_DB06_H07 | 1.07E−03 | 2.14E−03 | 650 | 1250 | 1850 | 2450 | 3050 | 3650 | 4250 | 4850 |
| suPAR_DB06_H08 | 2.75E−04 | 5.22E−03 | 651 | 1251 | 1851 | 2451 | 3051 | 3651 | 4251 | 4851 |
| suPAR_DB06_H09 | 1.94E−04 | 5.76E−03 | 652 | 1252 | 1852 | 2452 | 3052 | 3652 | 4252 | 4852 |
| suPAR_DB06_H10 | 2.95E−04 | 4.21E−03 | 653 | 1253 | 1853 | 2453 | 3053 | 3653 | 4253 | 4853 |
| suPAR_DB07_A03 | 1.71E−04 | 4.38E−03 | 654 | 1254 | 1854 | 2454 | 3054 | 3654 | 4254 | 4854 |
| suPAR_DB07_A04 | 1.04E−04 | 2.81E−03 | 655 | 1255 | 1855 | 2455 | 3055 | 3655 | 4255 | 4855 |
| suPAR_DB07_A05 | 7.90E−05 | 1.29E−03 | 656 | 1256 | 1856 | 2456 | 3056 | 3656 | 4256 | 4856 |
| suPAR_DB07_A06 | 1.80E−04 | 4.26E−03 | 657 | 1257 | 1857 | 2457 | 3057 | 3657 | 4257 | 4857 |
| suPAR_DB07_A07 | 7.78E−04 | 2.74E−03 | 658 | 1258 | 1858 | 2458 | 3058 | 3658 | 4258 | 4858 |
| suPAR_DB07_A08 | 4.35E−04 | 8.44E−04 | 659 | 1259 | 1859 | 2459 | 3059 | 3659 | 4259 | 4859 |
| suPAR_DB07_A09 | 5.79E−07 | 3.06E−03 | 660 | 1260 | 1860 | 2460 | 3060 | 3660 | 4260 | 4860 |
| suPAR_DB07_A10 | 1.23E−04 | 4.26E−03 | 661 | 1261 | 1861 | 2461 | 3061 | 3661 | 4261 | 4861 |
| suPAR_DB07_A11 | 5.19E−04 | 4.32E−03 | 662 | 1262 | 1862 | 2462 | 3062 | 3662 | 4262 | 4862 |
| suPAR_DB07_A12 | 3.76E−07 | 4.54E−03 | 663 | 1263 | 1863 | 2463 | 3063 | 3663 | 4263 | 4863 |
| suPAR_DB07_B01 | 2.33E−05 | 3.24E−03 | 664 | 1264 | 1864 | 2464 | 3064 | 3664 | 4264 | 4864 |
| suPAR_DB07_B02 | 5.70E−04 | 4.56E−04 | 665 | 1265 | 1865 | 2465 | 3065 | 3665 | 4265 | 4865 |
| suPAR_DB07_B03 | 2.16E−04 | 1.23E−03 | 666 | 1266 | 1866 | 2466 | 3066 | 3666 | 4266 | 4866 |
| suPAR_DB07_B04 | 5.28E−07 | 2.50E−03 | 667 | 1267 | 1867 | 2467 | 3067 | 3667 | 4267 | 4867 |
| suPAR_DB07_B05 | 1.46E−04 | 4.84E−04 | 668 | 1268 | 1868 | 2468 | 3068 | 3668 | 4268 | 4868 |
| suPAR_DB07_B06 | 1.35E−04 | 2.62E−03 | 669 | 1269 | 1869 | 2469 | 3069 | 3669 | 4269 | 4869 |
| suPAR_DB07_B07 | 5.83E−07 | 7.78E−04 | 670 | 1270 | 1870 | 2470 | 3070 | 3670 | 4270 | 4870 |
| suPAR_DB07_B08 | 5.69E−04 | 1.97E−03 | 671 | 1271 | 1871 | 2471 | 3071 | 3671 | 4271 | 4871 |
| suPAR_DB07_B09 | 3.04E−07 | 3.70E−03 | 672 | 1272 | 1872 | 2472 | 3072 | 3672 | 4272 | 4872 |
| suPAR_DB07_B10 | 4.23E−07 | 3.73E−03 | 673 | 1273 | 1873 | 2473 | 3073 | 3673 | 4273 | 4873 |
| suPAR_DB07_B11 | 1.63E−05 | 7.64E−04 | 674 | 1274 | 1874 | 2474 | 3074 | 3674 | 4274 | 4874 |
| suPAR_DB07_B12 | 1.74E−04 | 1.96E−03 | 675 | 1275 | 1875 | 2475 | 3075 | 3675 | 4275 | 4875 |
| suPAR_DB07_C01 | 1.09E−04 | 3.40E−03 | 676 | 1276 | 1876 | 2476 | 3076 | 3676 | 4276 | 4876 |
| suPAR_DB07_C02 | 1.76E−04 | 1.20E−03 | 677 | 1277 | 1877 | 2477 | 3077 | 3677 | 4277 | 4877 |
| suPAR_DB07_C03 | 2.78E−04 | 4.47E−03 | 678 | 1278 | 1878 | 2478 | 3078 | 3678 | 4278 | 4878 |
| suPAR_DB07_C04 | 6.15E−07 | 1.44E−03 | 679 | 1279 | 1879 | 2479 | 3079 | 3679 | 4279 | 4879 |
| suPAR_DB07_C05 | 6.50E−06 | 2.93E−03 | 680 | 1280 | 1880 | 2480 | 3080 | 3680 | 4280 | 4880 |
| suPAR_DB07_C06 | 5.80E−07 | 3.45E−03 | 681 | 1281 | 1881 | 2481 | 3081 | 3681 | 4281 | 4881 |
| suPAR_DB07_C07 | 4.88E−07 | 3.24E−03 | 682 | 1282 | 1882 | 2482 | 3082 | 3682 | 4282 | 4882 |
| suPAR_DB07_C08 | 4.88E−07 | 2.98E−03 | 683 | 1283 | 1883 | 2483 | 3083 | 3683 | 4283 | 4883 |
| suPAR_DB07_C09 | 4.88E−07 | 3.09E−03 | 684 | 1284 | 1884 | 2484 | 3084 | 3684 | 4284 | 4884 |
| suPAR_DB07_C10 | 1.49E−04 | 3.11E−03 | 685 | 1285 | 1885 | 2485 | 3085 | 3685 | 4285 | 4885 |
| suPAR_DB07_C11 | 1.89E−05 | 4.00E−03 | 686 | 1286 | 1886 | 2486 | 3086 | 3686 | 4286 | 4886 |
| suPAR_DB07_C12 | 6.58E−05 | 4.01E−03 | 687 | 1287 | 1887 | 2487 | 3087 | 3687 | 4287 | 4887 |
| suPAR_DB07_D01 | 3.94E−07 | 4.39E−03 | 688 | 1288 | 1888 | 2488 | 3088 | 3688 | 4288 | 4888 |
| suPAR_DB07_D02 | 3.99E−07 | 2.76E−03 | 689 | 1289 | 1889 | 2489 | 3089 | 3689 | 4289 | 4889 |
| suPAR_DB07_D03 | 3.50E−04 | 2.64E−03 | 690 | 1290 | 1890 | 2490 | 3090 | 3690 | 4290 | 4890 |
| suPAR_DB07_D04 | 5.50E−07 | 5.23E−03 | 691 | 1291 | 1891 | 2491 | 3091 | 3691 | 4291 | 4891 |
| suPAR_DB07_D05 | 2.19E−04 | 3.49E−03 | 692 | 1292 | 1892 | 2492 | 3092 | 3692 | 4292 | 4892 |
| suPAR_DB07_D06 | 3.93E−07 | 1.17E−03 | 693 | 1293 | 1893 | 2493 | 3093 | 3693 | 4293 | 4893 |
| suPAR_DB07_D07 | 8.58E−06 | 4.33E−03 | 694 | 1294 | 1894 | 2494 | 3094 | 3694 | 4294 | 4894 |
| suPAR_DB07_D08 | 4.88E−07 | 2.56E−03 | 695 | 1295 | 1895 | 2495 | 3095 | 3695 | 4295 | 4895 |
| suPAR_DB07_D09 | 5.45E−07 | 3.58E−03 | 696 | 1296 | 1896 | 2496 | 3096 | 3696 | 4296 | 4896 |
| suPAR_DB07_D10 | 7.37E−05 | 4.71E−03 | 697 | 1297 | 1897 | 2497 | 3097 | 3697 | 4297 | 4897 |
| suPAR_DB07_D11 | 5.44E−07 | 4.24E−03 | 698 | 1298 | 1898 | 2498 | 3098 | 3698 | 4298 | 4898 |
| suPAR_DB07_D12 | 2.46E−04 | 3.37E−03 | 699 | 1299 | 1899 | 2499 | 3099 | 3699 | 4299 | 4899 |
| suPAR_DB07_E01 | 2.39E−04 | 3.18E−03 | 700 | 1300 | 1900 | 2500 | 3100 | 3700 | 4300 | 4900 |

In one aspect, disclosed herein are urokinase plasminogen activator receptor (uPAR) binding molecules (such as, for example, soluble urokinase plasminogen activator receptor (suPAR) binding molecules). It is understood and herein contemplated that the suPAR binding molecules can be any binding molecule known in the art including, but not limited to a chimeric antigen receptor (CAR) T cell, CAR NK cell, CAR Macrophage (CARMA), immunotoxin, bispecific antibody, diabody, triabody, Bispecific T cell engager (BiTE), antibody, or antibody fragment.

### 1. Antibodies

### (1) Antibodies Generally

As used herein, the term “antibody” encompasses, but is not limited to, whole immunoglobulin (i.e., an intact antibody) of any class. Native antibodies are usually heterotetrameric glycoproteins, composed of two identical light (L) chains and two identical heavy (H) chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (V(H)) followed by a number of constant domains. Each light chain has a variable domain at one end (V(L)) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy chain variable domains. The light chains of antibodies from any vertebrate species can be assigned to one of two clearly distinct types, called kappa (k) and lambda (1), based on the amino acid sequences of their constant domains. Depending on the amino acid sequence of the constant domain of their heavy chains, immunoglobulins can be assigned to different classes. There are five major classes of human immunoglobulins: IgA, IgD, IgE, IgG and IgM, and several of these may be further divided into subclasses (isotypes), e.g., IgG-1, IgG-2, IgG-3, and IgG-4; IgA-1 and IgA-2. One skilled in the art would recognize the comparable classes for mouse. The heavy chain constant domains that correspond to the different classes of immunoglobulins are called alpha, delta, epsilon, gamma, and mu, respectively.

The term “antibodies” is used herein in a broad sense and includes both polyclonal and monoclonal antibodies. In addition to intact immunoglobulin molecules, also included in the term “antibodies” are fragments or polymers of those immunoglobulin molecules, and human or humanized versions of immunoglobulin molecules or fragments thereof, as long as they are chosen for their ability to interact with a urokinase plasminogen activator receptor (uPAR) and, in particular, soluble urokinase plasminogen activator receptor (suPAR). The antibodies can be tested for their desired activity using the in vitro assays described herein, or by analogous methods, after which their in vivo therapeutic and/or prophylactic activities are tested according to known clinical testing methods.

The term “variable” is used herein to describe certain portions of the variable domains that differ in sequence among antibodies and are used in the binding and specificity of each particular antibody for its particular antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a b-sheet configuration, connected by three CDRs, which form loops connecting, and in some cases forming part of, the b-sheet structure. The CDRs in each chain are held together in close proximity by the FR regions and, with the CDRs from the other chain, contribute to the formation of the antigen binding site of antibodies. The constant domains are not involved directly in binding an antibody to an antigen, but exhibit various effector functions, such as participation of the antibody in antibody-dependent cellular toxicity. The term “monoclonal antibody” as used herein refers to an antibody obtained from a substantially homogeneous population of antibodies, i.e., the individual antibodies within the population are identical except for possible naturally occurring mutations that may be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity.

The disclosed monoclonal antibodies can be made using any procedure which produces monoclonal antibodies. For example, disclosed monoclonal antibodies can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes may be immunized in vitro.

The monoclonal antibodies may also be made by recombinant DNA methods DNA encoding the disclosed monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

As used herein, the term “antibody or fragments thereof” encompasses chimeric antibodies and hybrid antibodies, with dual or multiple antigen or epitope specificities, and fragments, such as F(ab′) 2, Fab′, Fab, Fv, sFv, scFv, and the like, including hybrid fragments. Thus, fragments of the antibodies that retain the ability to bind their specific antigens are provided. For example, fragments of antibodies which maintain uPAR and/or suPAR binding activity are included within the meaning of the term “antibody or fragment thereof.” Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies).

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment may be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

### (2) Human Antibodies

The disclosed human antibodies can be prepared using any technique. The disclosed human antibodies can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993). Specifically, the homozygous deletion of the antibody heavy chain joining region (J (H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

### (3) Humanized Antibodies

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an sFv, Fv, Fab, Fab′, F(ab′) 2, or other antigen-binding portion of an antibody) which contains a portion of an antigen binding site from a non-human (donor) antibody integrated into the framework of a human (recipient) antibody.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies may also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody (Jones et al., Nature, 321:522-525 (1986), Reichmann et al, Nature, 332:323-327 (1988), and Presta, Curr. Opin. Struct. Biol., 2:593-596 (1992)).

Methods for humanizing non-human antibodies are well known in the art. For example, humanized antibodies can be generated according to the methods of Winter and co-workers (Jones et al., Nature, 321:522-525 (1986), Riechmann et al., Nature, 332:323-327 (1988), Verhoeyen et al., Science, 239:1534-1536 (1988)), by substituting rodent CDRs or CDR sequences for the corresponding sequences of a human antibody. Methods that can be used to produce humanized antibodies are also described in U.S. Pat. No. 4,816,567 (Cabilly et al.), U.S. Pat. No. 5,565,332 (Hoogenboom et al.), U.S. Pat. No. 5,721,367 (Kay et al.), U.S. U.S. Pat. No. 5,837,243 (Deo et al.), U.S. Pat. No. 5,939,598 (Kucherlapati et al.), U.S. Pat. No. 6,130,364 (Jakobovits et al.), and U.S. Pat. No. 6,180,377 (Morgan et al.).

### (4) Administration of Antibodies

Administration of the antibodies can be done as disclosed herein. Nucleic acid approaches for antibody delivery also exist. The broadly neutralizing anti suPAR and anti-uPAR antibodies and antibody fragments can also be administered to subjects as a nucleic acid preparation (e.g., DNA or RNA) that encodes the antibody or antibody fragment, such that the subject's own cells take up the nucleic acid and express the encoded antibody or antibody fragment. The delivery of the nucleic acid can be by any means, as disclosed herein.

In one aspect, disclosed herein are soluble urokinase plasminogen activator receptor (suPAR) binding molecules (such as, for example a chimeric antigen receptor (CAR) T cell, CAR NK cell, CAR Macrophage (CARMA), immunotoxin, bispecific antibody, diabody, triabody, Bispecific T cell engager (BiTE), antibody, or antibody fragment) comprising a light chain variable domain, wherein the light chain variable domain comprises 3 complementarity determining regions (CDRs), CDR1, CDR2, and CDR3 as set forth in SEQ ID NOs 71-75 and 1901-2500; SEQ ID NOs: 76, 77, and 2501-3100; and SEQ ID NO: 78 and 3100-3700, respectively or any other CDR as set forth in Tables 3, 4, or 6. In one aspect, the light chain variable domain can comprise a CDR1, CDR2, and CD3, as set forth in SEQ ID Nos 71, 76, and 78, respectively; SEQ ID Nos 72, 76, and 78, respectively; SEQ ID Nos 72, 77, and 78, respectively; SEQ ID Nos 73, 76, and 78, respectively; SEQ ID Nos 74, 76, and 78, respectively; or SEQ ID Nos 75, 76, and 78, respectively. For example, the soluble urokinase plasminogen activator receptor (suPAR) binding molecule can comprise a light chain variable domain (V_{L}) comprising the amino acid sequence as set forth in SEQ ID NOs: 2, 25-44, 4300-4900, and 4904 or as shown in Table 3, 4, or 6.

Also disclosed herein are soluble urokinase plasminogen activator receptor (suPAR) binding molecules, wherein the uPAR binding molecule further comprises a heavy chain variable domain; wherein the heavy chain variable domain comprises 3 complementarity determining regions (CDRs), CDR1, CDR2, and CDR3 as set forth in SEQ ID NOs 45-57 and 101-700; SEQ ID NOs: 58-68 and 701-1300; and SEQ ID NOs: 69, 70, and 1301-1900, respectively or any other CDR as set forth in Tables 3, 4, or 6. In one aspect, the heavy chain variable domain can comprise a CDR1, CDR2, and CD3, as set forth in SEQ ID Nos: 45, 58, and 69, respectively; SEQ ID Nos: 45, 59, and 69, respectively; SEQ ID Nos: 46, 60, and 69, respectively; SEQ ID Nos: 46, 61, and 69, respectively; SEQ ID Nos: 47, 62, and 69, respectively; SEQ ID Nos: 48, 62, and 70, respectively; SEQ ID Nos: 49, 62, and 69, respectively, SEQ ID Nos. 50, 62, and 69, respectively; SEQ ID Nos: 51, 62, and 69, respectively; SEQ ID Nos: 52, 62, and 69, respectively; SEQ ID Nos: 53, 63, and 69, respectively; SEQ ID Nos: 53, 64, and 69, respectively; SEQ ID Nos: 54, 65, and 69, respectively; SEQ ID Nos: 54, 66, and 69, respectively; SEQ ID Nos: 55, 62, and 69, respectively; SEQ ID Nos: 53, 62, and 69, respectively; SEQ ID Nos: 56, 67, and 69, respectively; SEQ ID Nos: 57, 68, and 69, respectively; or SEQ ID Nos: 53, 64, and 69, respectively. For example, the soluble urokinase plasminogen activator receptor (suPAR) binding molecule can comprise a heavy chain variable domain (V_{H}) comprising the amino acid sequence as set forth in SEQ ID NOs: 1, 5-24, 3701-4300 and 4903 or as shown in Table 3, 4, or Thus, in one aspect, the urokinase plasminogen activator receptor (uPAR) binding molecule can comprise a heavy chain CDR1, CDR2 and CDR 3 as set forth in SEQ ID NOs: 48, 62, and 70, respectively; and a light chain CDR1, CDR2, and CDR3 as set forth in SEQ ID Nos 72, 76, and 78, respectively; a heavy chain CDR1, CDR2 and CDR 3 as set forth in SEQ ID NOs: 53, 64, and 69, respectively; and a light chain CDR1, CDR2, and CDR3 as set forth in SEQ ID Nos 71, 76, and 78, respectively. For example, the urokinase plasminogen activator receptor (uPAR) binding molecule can comprise a heavy chain as set forth in SEQ ID NO: 4903 and a light chain as set forth in SEQ ID NO: 4904; a heavy chain as set forth in SEQ ID NO: 1 and a light chain as set forth in SEQ ID NO: 2; a heavy chain as set forth in SEQ ID NO: 10 and a light chain as set forth in SEQ ID NO: 30, a heavy chain as set forth in SEQ ID NO: 12 and a light chain as set forth in SEQ ID NO: 32; a heavy chain as set forth in SEQ ID NO: 24 and a light chain as set forth in SEQ ID NO: 44; a heavy chain as set forth in SEQ ID NO: 5 and a light chain as set forth in SEQ ID NO: 25; a heavy chain as set forth in SEQ ID NO: 6 and a light chain as set forth in SEQ ID NO: 26; a heavy chain as set forth in SEQ ID NO: 7 and a light chain as set forth in SEQ ID NO: 27; a heavy chain as set forth in SEQ ID NO: 8 and a light chain as set forth in SEQ ID NO: 28; a heavy chain as set forth in SEQ ID NO: 9 and a light chain as set forth in SEQ ID NO: 29; a heavy chain as set forth in SEQ ID NO: 11 and a light chain as set forth in SEQ ID NO: 31; a heavy chain as set forth in SEQ ID NO: 13 and a light chain as set forth in SEQ ID NO: 33; a heavy chain as set forth in SEQ ID NO: 14 and a light chain as set forth in SEQ ID NO: 34; a heavy chain as set forth in SEQ ID NO: 15 and a light chain as set forth in SEQ ID NO: 35; a heavy chain as set forth in SEQ ID NO: 16 and a light chain as set forth in SEQ ID NO: 36; a heavy chain as set forth in SEQ ID NO: 17 and a light chain as set forth in SEQ ID NO: 37; a heavy chain as set forth in SEQ ID NO: 18 and a light chain as set forth in SEQ ID NO: 38; a heavy chain as set forth in SEQ ID NO: 19 and a light chain as set forth in SEQ ID NO: 39; a heavy chain as set forth in SEQ ID NO: 20 and a light chain as set forth in SEQ ID NO: 40; a heavy chain as set forth in SEQ ID NO: 21 and a light chain as set forth in SEQ ID NO: 41; a heavy chain as set forth in SEQ ID NO: 22 and a light chain as set forth in SEQ ID NO: 42; a heavy chain as set forth in SEQ ID NO: 23 and a light chain as set forth in SEQ ID NO: 43.

In one aspect, disclosed herein are soluble urokinase plasminogen activator receptor (suPAR) binding molecules (such as, for example a chimeric antigen receptor (CAR) T cell, CAR NK cell, CAR Macrophage (CARMA), immunotoxin, bispecific antibody, diabody, triabody, Bispecific T cell engager (BiTE), antibody, or antibody fragment) comprising a heavy chain variable domain; wherein the heavy chain variable domain comprises 3 complementarity determining regions (CDRs), CDR1, CDR2, and CDR3 as set forth in SEQ ID NOs 45-57 and 101-700; SEQ ID NOs: 58-68 and 701-1300; and SEQ ID NOs: 69, 70, and 1301-1900, respectively or any other CDR as set forth in Tables 3, 4, or 6. In one aspect, the heavy chain variable domain can comprise a CDR1, CDR2, and CD3, as set forth in SEQ ID Nos: 45, 58, and 69, respectively; SEQ ID Nos: 45, 59, and 69, respectively; SEQ ID Nos: 46, 60, and 69, respectively; SEQ ID Nos: 46, 61, and 69, respectively; SEQ ID Nos: 47, 62, and 69, respectively, SEQ ID Nos. 48, 62, and 70, respectively; SEQ ID Nos: 49, 62, and 69, respectively; SEQ ID Nos: 50, 62, and 69, respectively; SEQ ID Nos: 51, 62, and 69, respectively: SEQ ID Nos: 52, 62, and 69, respectively; SEQ ID Nos: 53, 63, and 69, respectively; SEQ ID Nos: 53, 64, and 69, respectively; SEQ ID Nos: 54, 65, and 69, respectively; SEQ ID Nos: 54, 66, and 69, respectively; SEQ ID Nos: 55, 62, and 69, respectively; SEQ ID Nos: 53, 62, and 69, respectively; SEQ ID Nos: 56, 67, and 69, respectively; SEQ ID Nos: 57, 68, and 69, respectively; or SEQ ID Nos: 53, 64, and 69, respectively. For example, the soluble urokinase plasminogen activator receptor (suPAR) binding molecule can comprise a heavy chain variable domain (VA) comprising the amino acid sequence as set forth in SEQ ID NOs: 1, 5-24, 3701-4300 and 4903 or as shown in 3, 4, or 6.

It is understood and herein contemplated that the disclosed binding molecules can comprise constant domains of an antibody including, but not limited to full-length Fc domains. Also disclosed herein are soluble urokinase plasminogen activator receptor (suPAR) binding molecules, wherein the binding molecule further comprises a light chain constant domain as set forth in SEQ ID NO: 4 or SEQ ID NO: 4906. Also disclosed herein are soluble urokinase plasminogen activator receptor (suPAR) binding molecules, wherein the binding molecule further comprises a heavy chain constant domain as set forth in SEQ ID NO: 3 or SEQ ID NO: In some aspects, the heavy or light chain constant region can comprise a mutation. For example, the heavy chain constant domain can comprise a L234A, L235A (LALA mutation), P329A, and/or P329G substitution. In one aspect, the suPAR binding molecule comprises both heavy and light chain constant domains (such as, for example, the heavy chain constant domain as set forth in SEQ ID NO: 3 or SEQ ID NO: 4905 and light chain constant domain as set forth in SEQ ID NO: 4 or SEQ ID NO: 4906.

TABLE 4

| | | SEQ | | SEQ | | SEQ | | SEQ | | SEQ | | SEQ |
|---|---|---|---|---|---|---|---|---|---|---|---|---|
| | | ID | | ID | | ID | | ID | | ID | | ID |
| Wab | CDR-H1 | NO: | CDR-H2 | NO: | CDR-H3 | NO: | CDR-L1 | NO: | CDR-L2 | NO: | CDR-L3 | NO: |
| WAb0001 | YNIKDYYIH | 45 | GWISPDSGVTNYA | 58 | CARGYDVDWFVYW | 69 | SARQSVSYMY | 71 | RTSNLAS | 76 | CQQYHSYPPTF | 78 |
| WAb0002 | YNIKDYYIH | 45 | GWISPSSGVTNYA | 59 | CARGYDVDWFVYW | 69 | SARQSVSYMY | 71 | RTSNLAS | 76 | CQQYHSYPPTF | 78 |
| WAb0003 | FNIKDYYIH | 46 | GWIDPENGSTIYD | 60 | CARGYDIDWFVYW | 69 | SARSSVSYMY | 72 | RTSNLAS | 76 | CQQYHSYPPTF | 78 |
| WAb0004 | FNIKDYYIH | 46 | GWIDPESGSTIYD | 61 | CARGYDIDWFVYW | 69 | SARSSVSYMY | 72 | RTSNLAS | 76 | CQQYHSYPPTF | 78 |
| WAb0005 | YTFIGSYVH | 47 | GWIDHENGNTIYD | 62 | CARGYDVDWFVYW | 69 | SARSSVSYMY | 72 | RTSSLAS | 77 | CQQYHSYPPTF | 78 |
| WAb0006 | YTFTSHYIQ | 48 | GWIDHENGNTIYD | 62 | CARGTDVDWFVYW | 70 | SARSSVSYMY | 72 | RTSNLAS | 76 | CQQYHSYPPTF | 78 |
| WAb0007 | YTFIGSYVH | 47 | GWIDHENGNTIYD | 62 | CARGYDVDWFVYW | 69 | SARSSVSYMY | 72 | RTSSLAS | 77 | CQQYHSYPPTF | 78 |
| WAb0008 | YTFTSHYIQ | 48 | GWIDHENGNTIYD | 62 | CARGTDVDWFVYW | 70 | SARSSVSYMY | 72 | RTSNLAS | 76 | CQQYHSYPPTF | 78 |
| WAb0009 | YTFTGHYVH | 49 | GWIDHENGNTIYD | 62 | CARGYDVDWFVYW | 69 | SARSSVSYMY | 72 | RTSNLAS | 76 | CQQYHSYPPTF | 78 |
| WAb0010 | YTFTGNYVH | 50 | GWIDHENGNTIYD | 62 | CARGYDVDWFVYW | 69 | SARSSVSYMY | 72 | RTSNLAS | 76 | CQQYHSYPPTF | 78 |
| WAb0011 | YTFTGSYVH | 51 | GWIDHENGNTIYD | 62 | CARGYDVDWFVYW | 69 | SARSSVSYMY | 72 | RTSNLAS | 76 | CQQYHSYPPTF | 78 |
| WAb0012 | YTFTGYYMH | 52 | GWIDHENGNTIYD | 62 | CARGYDVDWFVYW | 69 | SARQSISYMY | 73 | RTSNLAS | 76 | CQQYHSYPPTF | 78 |
| WAb0013 | YTFTNYYMH | 53 | GWIDHENGSTIYA | 63 | CARGYDVDWFVYW | 69 | SARQSVSYMY | 71 | RTSNLAS | 76 | CQQYHSYPPTF | 78 |
| WAb0014 | YTFTNYYMH | 53 | GWIDHESGSTIYA | 64 | CARGYDVDWFVYW | 69 | SARQSVSYMY | 71 | RTSNLAS | 76 | CQQYHSYPPTF | 78 |
| WAb0015 | YTFTNYYIH | 54 | GWISPDSGGTKYA | 65 | CARGYDVDWFVYW | 69 | SARSSVSYMY | 72 | RTSNLAS | 76 | CQQYHSYPPTF | 78 |
| WAb0016 | YTFTNYYIH | 54 | GWISPSSGGTKYA | 66 | CARGYDVDWFVYW | 69 | SARSSVSYMY | 72 | RTSNLAS | 76 | CQQYHSYPPTF | 78 |
| WAb0017 | YTFSAYYMH | 55 | GWIDHENGNTIYD | 62 | CARGYDVDWFVYW | 69 | RARQSVSYMY | 74 | RTSNLAS | 76 | CQQYHSYPPTF | 78 |
| WAb0018 | YTFTNYYMH | 53 | GWIDHENGNTIYD | 62 | CARGYDVDWFVYW | 69 | SASSSISYMY | 75 | RTSNLAS | 76 | CQQYHSYPPTF | 78 |
| WAb0019 | YTFSSYYMH | 56 | GWIDHENGNTIYA | 67 | CARGYDVDWFVYW | 69 | SARSSVSYMY | 72 | RTSNLAS | 76 | CQQYHSYPPTF | 78 |
| WAb0020 | YTFTSHYVH | 57 | GWIDHENGNTIYD | 68 | CARGYDVDWFVYW | 69 | SARSSVSYMY | 72 | RTSNLAS | 76 | CQQYHSYPPTF | 78 |
| WAb0022 | YTFTNYYMH | 53 | GWIDHESGSTIYA | 64 | CARGYDVDWFVYW | 69 | SARQSVSYMY | 71 | RTSNLAS | 76 | CQQYHSYPPTF | 78 |

The binding affinities for the antibodies in Table 4 are shown in Table 5.

TABLE 5
binding affinities for antibodies in Table 3

| Wab | human kdis (1/s) |
|---|---|
| WAb0001 | 3.04E−07 |
| WAb0002 | 8.37E−05 |
| WAb0003 | 3.57E−07 |
| WAb0004 | 2.61E−07 |
| WAb0005 | 3.97E−07 |
| WAb0006 | 3.39E−07 |
| WAb0007 | 2.76E−07 |
| WAb0008 | 2.83E−07 |
| WAb0009 | 3.47E−07 |
| WAb0010 | 3.89E−07 |
| WAb0011 | 2.36E−07 |
| WAb0012 | 2.93E−07 |
| WAb0013 | 2.44E−07 |
| WAb0014 | 2.57E−07 |
| WAb0015 | 2.97E−07 |
| WAb0016 | 2.13E−04 |
| WAb0017 | 3.63E−07 |
| WAb0018 | 2.60E−07 |
| WAb0019 | 3.43E−07 |
| WAb0020 | 3.42E−07 |
| WAb0022 | not determined |

### 2. Homology/Identity

It is understood that one way to define any known variants and derivatives or those that might arise, of the disclosed genes and proteins herein is through defining the variants and derivatives in terms of homology to specific known sequences. For example, SEQ ID NOs: 1- and Tables 3 and 4 set forth a particular sequence of suPAR binding molecule (such as, for example an antibody), CDRs of said binding molecules, variable heavy and light chains of said binding molecules, or constant domains of said binding molecules. Specifically disclosed are variants of these and other genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection. The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

### 3. Peptides

### a) Protein Variants

As discussed herein there are numerous variants of the suPAR antibodies that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 1 and 2 and are referred to as conservative substitutions.

TABLE 1
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 2
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 2, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g, lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu: Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains, acetylation of the N-terminal amine and, in some instances, amidation of the C-terminal carboxyl.

It is understood that one way to define the variants and derivatives of the disclosed suPAR binding molecules and uPAR binding molecules herein is through defining the variants and derivatives in terms of homology/identity to specific known sequences. For example, SEQ ID NOs: 1-78 and Tables 3 and 4 set forth a particular sequences of suPAR binding molecule. Specifically disclosed are variants of these and other suPAR binding molecules and uPAR binding molecules herein disclosed which have at least, 70%, 75%, 80%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2:482 (1981), by the homology alignment algorithm of Needleman and Wunsch, J. Mol Biol. 48:443 (1970), by the search for similarity method of Pearson and Lipman, Proc. Natl. Acad. Sci. U.S.A. 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various suPAR binding molecules and uPAR binding molecules and suPAR binding molecules and uPAR binding molecules sequences it is understood that the nucleic acids that can encode those suPAR binding molecules and uPAR binding molecules sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed binding molecule sequence. It is also understood that while no amino acid sequence indicates what particular DNA sequence encodes that suPAR or uPAR binding molecule within an organism, where particular variants of a disclosed suPAR or uPAR binding molecule are disclosed herein, the known nucleic acid sequence that encodes that binding molecule is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1 and Table 2. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO—. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

### 4. Immunoassays and Fluorochromes

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

In general, immunoassays involve contacting a sample suspected of containing a molecule of interest (such as the disclosed biomarkers) with an antibody to the molecule of interest or contacting an antibody to a molecule of interest (such as antibodies to the disclosed biomarkers) with a molecule that can be bound by the antibody, as the case may be, under conditions effective to allow the formation of immunocomplexes. Contacting a sample with the antibody to the molecule of interest or with the molecule that can be bound by an antibody to the molecule of interest under conditions effective and for a period of time sufficient to allow the formation of immune complexes (primary immune complexes) is generally a matter of simply bringing into contact the molecule or antibody and the sample and incubating the mixture for a period of time long enough for the antibodies to form immune complexes with, i.e., to bind to, any molecules (e.g., antigens) present to which the antibodies can bind. In many forms of immunoassay, the sample-antibody composition, such as a tissue section, ELISA plate, dot blot or Western blot, can then be washed to remove any non-specifically bound antibody species, allowing only those antibodies specifically bound within the primary immune complexes to be detected.

Immunoassays can include methods for detecting or quantifying the amount of a molecule of interest (such as the disclosed biomarkers or their antibodies) in a sample, which methods generally involve the detection or quantitation of any immune complexes formed during the binding process. In general, the detection of immunocomplex formation is well known in the art and can be achieved through the application of numerous approaches. These methods are generally based upon the detection of a label or marker, such as any radioactive, fluorescent, biological or enzymatic tags or any other known label.

As used herein, a label can include a fluorescent dye, a member of a binding pair, such as biotin/streptavidin, a metal (e.g., gold), or an epitope tag that can specifically interact with a molecule that can be detected, such as by producing a colored substrate or fluorescence. Substances suitable for detectably labeling proteins include fluorescent dyes (also known herein as fluorochromes and fluorophores) and enzymes that react with colorometric substrates (e.g., horseradish peroxidase). The use of fluorescent dyes is generally preferred in the practice of the invention as they can be detected at very low amounts. Furthermore, in the case where multiple antigens are reacted with a single array, each antigen can be labeled with a distinct fluorescent compound for simultaneous detection. Labeled spots on the array are detected using a fluorimeter, the presence of a signal indicating an antigen bound to a specific antibody.

Fluorophores are compounds or molecules that luminesce. Typically fluorophores absorb electromagnetic energy at one wavelength and emit electromagnetic energy at a second wavelength. Representative fluorophores include, but are not limited to, 1,5 IAEDANS; 1,8-ANS; 4-Methylumbelliferone; 5-carboxy-2,7-dichlorofluorescein; 5-Carboxyfluorescein (5-FAM); 5-Carboxynapthofluorescein; 5-Carboxytetramethylrhodamine (5-TAMRA); 5-Hydroxy Tryptamine (≡-HAT); 5-ROX (carboxy-X-rhodamine); 6-Carboxyrhodamine 6G; 6-CR 6G; 6-JOE; 7-Amino-4-methylcoumarin; 7-Aminoactinomycin D (7-AAD); 7-Hydroxy-4-I methylcoumarin; 9-Amino-6-chloro-2-methoxyacridine (ACMA); ABQ; Acid Fuchsin; Acridine Orange; Acridine Red, Acridine Yellow; Acriflavin; Acriflavin Feulgen SITSA; Aequorin (Photoprotein); AFPs—AutoFluorescent Protein—(Quantum Biotechnologies) see sgGFP, sgBFP; Alexa Fluor 350™; Alexa Fluor 430™; Alexa Fluor 4881M, Alexa Fluor 532™; Alexa Fluor 546™; Alexa Fluor 568™; Alexa Fluor 594™, Alexa Fluor 633™; Alexa Fluor 647™; Alexa Fluor 660™; Alexa Fluor 680™; Alizarin Complexon; Alizarin Red, Allophycocyanin (APC); AMC, AMCA-S; Aminomethylcoumarin (AMCA); AMCA-X; Aminoactinomycin D; Aminocoumarin; Anilin Blue; Anthrocyl stearate; APC-Cy7; APTRA-BTC; APTS; Astrazon Brilliant Red 4G, Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); Blue Fluorescent Protein; BFP/GFP FRET; Bimane; Bisbenzemide; Bisbenzimide (Hoechst); bis-BTC; Blancophor FFG; Blancophor SV; BOBO™-1; BOBO™-3; Bodipy492/515; Bodipy493/503; Bodipy 500/510; Bodipy; 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy F1; Bodipy FL ATP; Bodipy F1-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; BTC; BTC-5N; Calcein; Calcein Blue, Calcium Crimson-; Calcium Green, Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CCF2 (GeneBlazer); CFDA; CFP (Cyan Fluorescent Protein); CFP/YFP FRET; Chlorophyll; Chromomycin A; Chromomycin A; CL-NERF; CMFDA: Coelenterazine; Coelenterazine cp; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM I Methylcoumarin; CTC; CTC Formazan; Cy2™; Cy3.18; Cy3.5™: Cy3™; Cy5.18; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3′DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di 16-ASP); Dichlorodihydrofluorescein Diacetate (DCFH); DiD-Lipophilic Tracer; DiD (DilC18 (5)); DIDS; Dihydorhodamine 123 (DHR); Dil (DilC18 (3)); I Dinitrophenol; DiO (DiOC18 (3)); DIR; DIR (DilC18 (7)); DM-NERF (high pH); DNP; Dopamine; DsRed; DTAF; DY-630-NHS; DY-635-NHS; EBFP; ECFP; EGFP; ELF 97; Eosin; Erythrosin; Erythrosin ITC, Ethidium Bromide, Ethidium homodimer-1 (EthD-1); Euchrysin; EukoLight; Europium (111) chloride; EYFP; Fast Blue; FDA; Feulgen (Pararosaniline); FIF (Formaldehyd Induced Fluorescence); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer; (CCF2); GFP (S65T); GFP red shifted (rsGFP); GFP wild type′ non-UV excitation (wtGFP); GFP wild type, UV excitation (wtGFP); GFPuv; Gloxalic Acid; Granular blue; Haematoporphyrin; Hoechst 33258; Hoechst 33342, Hoechst 34580, HPTS; Hydroxycoumarin; Hydroxystilbamidine (FluoroGold), Hydroxytryptamine; Indo-1, high calcium; Indo-1 low calcium; Indodicarbocyanine (DID); Indotricarbocyanine (DiR); Intrawhite Cf; JC-1; JO JO-1; JO-PRO-1; LaserPro; Laurodan; LDS (DNA); LDS 751 (RNA); Leucophor PAF; Leucophor SF: Leucophor WS; Lissamine Rhodamine; Lissamine Rhodamine B; Calcein/Ethidium homodimer, LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue, I Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxedidole; Noradrenaline; Nuclear Fast Red; i Nuclear Yellow; Nylosan Brilliant lavin E8G; Oregon Green™; Oregon Green™ 488; Oregon Green™ 500; Oregon Green™ 514; Pacific Blue; Pararosaniline (Feulgen); PBFI, PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH₂₆ (Sigma); PKH₆₇; PMIA; Pontochrome Blue Black; POPO-1; POPO-3; PO-PRO-1; PO-I PRO-3; Primuline; Procion Yellow; Propidium lodid (Pl); PyMPO, Pyrene; Pyronine; Pyronine B; Pyrozal Brilliant Flavin 7GF, QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra, Rhodamine BB; Rhodamine BG; Rhodamine Green; Rhodamine Phallicidine; Rhodamine: Phalloidine; Rhodamine Red; Rhodamine WT; Rose Bengal; R-phycocyanine; R-phycoerythrin (PE); rsGFP; S65A; S65C; S65L; S65T; Sapphire GFP; SBFI; Serotonin; Sevron Brilliant Red 2B; Sevron Brilliant Red 4G; Sevron I Brilliant Red B; Sevron Orange; Sevron Yellow L; sgBFP™ (super glow BFP); sgGFP™ (super glow GFP); SITS (Primuline; Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3 sulfopropyl) quinolinium); Stilbene; Sulphorhodamine B and C; Sulphorhodamine Extra; SYTO 11; SYTO 12; SYTO 13; SYTO 14; SYTO 15; SYTO 16; SYTO 17, SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83, SYTO 84; SYTO 85; SYTOX Blue; SYTOX Green; SYTOX Orange; Tetracycline; Tetramethylrhodamine (TRITC); Texas Red™, Texas Red-X™ conjugate; Thiadicarbocyanine (DiSC3); Thiazine Red R; Thiazole Orange; Thioflavin 5; Thioflavin S; Thioflavin TON; Thiolyte; Thiozole Orange; Tinopol CBS (Calcofluor White); TIER; TO-PRO-1; TO-PRO-3; TO-PRO-5; TOTO-1; TOTO-3; TriColor (PE-Cy5); TRITC TetramethylRodaminelsoThioCyanate; True Blue; Tru Red; Ultralite; Uranine B; Uvitex SFC; wt GFP; WW 781; X-Rhodamine; XRITC; Xylene Orange; Y66F; Y66H; Y66W; Yellow GFP; YFP; YO-PRO-1; YO-PRO 3; YOYO-1; YOYO-3; Sybr Green; Thiazole orange (interchelating dyes); semiconductor nanoparticles such as quantum dots; or caged fluorophore (which can be activated with light or other electromagnetic energy source), or a combination thereof.

A modifier unit such as a radionuclide can be incorporated into or attached directly to any of the compounds described herein by halogenation. Examples of radionuclides useful in this embodiment include, but are not limited to, tritium, iodine-125, iodine-131, iodine-123, iodine-124, astatine-210, carbon-11, carbon-14, nitrogen-13, fluorine-18. In another aspect, the radionuclide can be attached to a linking group or bound by a chelating group, which is then attached to the compound directly or by means of a linker. Examples of radionuclides useful in the aspect include, but are not limited to, Tc-99m, Re-186, Ga-68, Re-188, Y-90, Sm-153, Bi-212, Cu-67, Cu-64, and Cu-62. Radiolabeling techniques such as these are routinely used in the radiopharmaceutical industry.

The radiolabeled compounds are useful as imaging agents to diagnose neurological disease (e.g., a neurodegenerative disease) or a mental condition or to follow the progression or treatment of such a disease or condition in a mammal (e.g., a human). The radiolabeled compounds described herein can be conveniently used in conjunction with imaging techniques such as positron emission tomography (PET) or single photon emission computerized tomography (SPECT).

Labeling can be either direct or indirect. In direct labeling, the detecting antibody (the antibody for the molecule of interest) or detecting molecule (the molecule that can be bound by an antibody to the molecule of interest) include a label. Detection of the label indicates the presence of the detecting antibody or detecting molecule, which in turn indicates the presence of the molecule of interest or of an antibody to the molecule of interest, respectively. In indirect labeling, an additional molecule or moiety is brought into contact with, or generated at the site of, the immunocomplex. For example, a signal-generating molecule or moiety such as an enzyme can be attached to or associated with the detecting antibody or detecting molecule. The signal-generating molecule can then generate a detectable signal at the site of the immunocomplex. For example, an enzyme, when supplied with suitable substrate, can produce a visible or detectable product at the site of the immunocomplex ELISAs use this type of indirect labeling.

As another example of indirect labeling, an additional molecule (which can be referred to as a binding agent) that can bind to either the molecule of interest or to the antibody (primary antibody) to the molecule of interest, such as a second antibody to the primary antibody, can be contacted with the immunocomplex. The additional molecule can have a label or signal-generating molecule or moiety. The additional molecule can be an antibody, which can thus be termed a secondary antibody. Binding of a secondary antibody to the primary antibody can form a so-called sandwich with the first (or primary) antibody and the molecule of interest. The immune complexes can be contacted with the labeled, secondary antibody under conditions effective and for a period of time sufficient to allow the formation of secondary immune complexes. The secondary immune complexes can then be generally washed to remove any non-specifically bound labeled secondary antibodies, and the remaining label in the secondary immune complexes can then be detected. The additional molecule can also be or include one of a pair of molecules or moieties that can bind to each other, such as the biotin/avadin pair. In this mode, the detecting antibody or detecting molecule should include the other member of the pair.

Other modes of indirect labeling include the detection of primary immune complexes by a two step approach. For example, a molecule (which can be referred to as a first binding agent), such as an antibody, that has binding affinity for the molecule of interest or corresponding antibody can be used to form secondary immune complexes, as described above. After washing, the secondary immune complexes can be contacted with another molecule (which can be referred to as a second binding agent) that has binding affinity for the first binding agent, again under conditions effective and for a period of time sufficient to allow the formation of immune complexes (thus forming tertiary immune complexes). The second binding agent can be linked to a detectable label or signal-generating molecule or moiety, allowing detection of the tertiary immune complexes thus formed. This system can provide for signal amplification.

Immunoassays that involve the detection of as substance, such as a protein or an antibody to a specific protein, include label-free assays, protein separation methods (i.e., electrophoresis), solid support capture assays, or in vivo detection. Label-free assays are generally diagnostic means of determining the presence or absence of a specific protein, or an antibody to a specific protein, in a sample. Protein separation methods are additionally useful for evaluating physical properties of the protein, such as size or net charge. Capture assays are generally more useful for quantitatively evaluating the concentration of a specific protein, or antibody to a specific protein, in a sample. Finally, in vivo detection is useful for evaluating the spatial expression patterns of the substance, i.e., where the substance can be found in a subject, tissue or cell.

Provided that the concentrations are sufficient, the molecular complexes ([Ab-Ag]) generated by antibody-antigen interaction are visible to the naked eye, but smaller amounts may also be detected and measured due to their ability to scatter a beam of light. The formation of complexes indicates that both reactants are present, and in immunoprecipitation assays a constant concentration of a reagent antibody is used to measure specific antigen ([Ab-Ag]n), and reagent antigens are used to detect specific antibody ([Ab-Ag]n). If the reagent species is previously coated onto cells (as in hemagglutination assay) or very small particles (as in latex agglutination assay), “clumping” of the coated particles is visible at much lower concentrations. A variety of assays based on these elementary principles are in common use, including Ouchterlony immunodiffusion assay, rocket immunoelectrophoresis, and immunoturbidometric and nephelometric assays. The main limitations of such assays are restricted sensitivity (lower detection limits) in comparison to assays employing labels and, in some cases, the fact that very high concentrations of analyte can actually inhibit complex formation, necessitating safeguards that make the procedures more complex. Some of these Group 1 assays date right back to the discovery of antibodies and none of them have an actual “label” (e.g. Ag-enz). Other kinds of immunoassays that are label free depend on immunosensors, and a variety of instruments that can directly detect antibody-antigen interactions are now commercially available. Most depend on generating an evanescent wave on a sensor surface with immobilized ligand, which allows continuous monitoring of binding to the ligand. Immunosensors allow the easy investigation of kinetic interactions and, with the advent of lower-cost specialized instruments, may in the future find wide application in immunoanalysis.

The use of immunoassays to detect a specific protein can involve the separation of the proteins by electophoresis. Electrophoresis is the migration of charged molecules in solution in response to an electric field Their rate of migration depends on the strength of the field; on the net charge, size and shape of the molecules and also on the ionic strength, viscosity and temperature of the medium in which the molecules are moving. As an analytical tool, electrophoresis is simple, rapid and highly sensitive. It is used analytically to study the properties of a single charged species, and as a separation technique.

Generally the sample is run in a support matrix such as paper, cellulose acetate, starch gel, agarose or polyacrylamide gel. The matrix inhibits convective mixing caused by heating and provides a record of the electrophoretic run: at the end of the run, the matrix can be stained and used for scanning, autoradiography or storage. In addition, the most commonly used support matrices-agarose and polyacrylamide-provide a means of separating molecules by size, in that they are porous gels. A porous gel may act as a sieve by retarding, or in some cases completely obstructing, the movement of large macromolecules while allowing smaller molecules to migrate freely. Because dilute agarose gels are generally more rigid and easy to handle than polyacrylamide of the same concentration, agarose is used to separate larger macromolecules such as nucleic acids, large proteins and protein complexes. Polyacrylamide, which is easy to handle and to make at higher concentrations, is used to separate most proteins and small oligonucleotides that require a small gel pore size for retardation.

Proteins are amphoteric compounds; their net charge therefore is determined by the pH of the medium in which they are suspended. In a solution with a pH above its isoelectric point, a protein has a net negative charge and migrates towards the anode in an electrical field. Below its isoelectric point, the protein is positively charged and migrates towards the cathode. The net charge carried by a protein is in addition independent of its size—i.e., the charge carried per unit mass (or length, given proteins and nucleic acids are linear macromolecules) of molecule differs from protein to protein. At a given pH therefore, and under non-denaturing conditions, the electrophoretic separation of proteins is determined by both size and charge of the molecules.

Sodium dodecyl sulphate (SDS) is an anionic detergent which denatures proteins by “wrapping around” the polypeptide backbone- and SDS binds to proteins fairly specifically in a mass ratio of 1.4:1. In so doing, SDS confers a negative charge to the polypeptide in proportion to its length. Further, it is usually necessary to reduce disulphide bridges in proteins (denature) before they adopt the random-coil configuration necessary for separation by size; this is done with 2-mercaptoethanol or dithiothreitol (DTT). In denaturing SDS-PAGE separations therefore, migration is determined not by intrinsic electrical charge of the polypeptide, but by molecular weight.

Determination of molecular weight is done by SDS-PAGE of proteins of known molecular weight along with the protein to be characterized. A linear relationship exists between the logarithm of the molecular weight of an SDS-denatured polypeptide, or native nucleic acid, and its Rf. The Rf is calculated as the ratio of the distance migrated by the molecule to that migrated by a marker dye-front. A simple way of determining relative molecular weight by electrophoresis (Mr) is to plot a standard curve of distance migrated vs. log 10MW for known samples, and read off the logMr of the sample after measuring distance migrated on the same gel.

In two-dimensional electrophoresis, proteins are fractionated first on the basis of one physical property, and, in a second step, on the basis of another. For example, isoelectric focusing can be used for the first dimension, conveniently carried out in a tube gel, and SDS electrophoresis in a slab gel can be used for the second dimension. One example of a procedure is that of O'Farrell, P. H., High Resolution Two-dimensional Electrophoresis of Proteins, J. Biol. Chem. 250:4007-4021 (1975), herein incorporated by reference in its entirety for its teaching regarding two-dimensional electrophoresis methods Other examples include but are not limited to, those found in Anderson, L and Anderson, NG, High resolution two-dimensional electrophoresis of human plasma proteins, Proc. Natl. Acad. Sci. 74:5421-5425 (1977), Ornstein, L., Disc electrophoresis, L. Ann. N.Y. Acad. Sci. 121:321349 (1964), each of which is herein incorporated by reference in its entirety for teachings regarding electrophoresis methods. Laemmli, U. K., Cleavage of structural proteins during the assembly of the head of bacteriophage T4, Nature 227:680 (1970), which is herein incorporated by reference in its entirety for teachings regarding electrophoresis methods, discloses a discontinuous system for resolving proteins denatured with SDS. The leading ion in the Laemmli buffer system is chloride, and the trailing ion is glycine. Accordingly, the resolving gel and the stacking gel are made up in Tris-HCl buffers (of different concentration and pH), while the tank buffer is Tris-glycine. All buffers contain 0.1% SDS.

One example of an immunoassay that uses electrophoresis that is contemplated in the current methods is Western blot analysis. Western blotting or immunoblotting allows the determination of the molecular mass of a protein and the measurement of relative amounts of the protein present in different samples. Detection methods include chemiluminescence and chromagenic detection. Standard methods for Western blot analysis can be found in, for example, D. M. Bollag et al., Protein Methods (2d edition 1996) and E. Harlow & D. Lane, Antibodies, a Laboratory Manual (1988), U.S. Pat. No. 4,452,901, each of which is herein incorporated by reference in their entirety for teachings regarding Western blot methods. Generally, proteins are separated by gel electrophoresis, usually SDS-PAGE The proteins are transferred to a sheet of special blotting paper, e.g., nitrocellulose, though other types of paper, or membranes, can be used. The proteins retain the same pattern of separation they had on the gel. The blot is incubated with a generic protein (such as milk proteins) to bind to any remaining sticky places on the nitrocellulose. An antibody is then added to the solution which is able to bind to its specific protein.

The attachment of specific antibodies to specific immobilized antigens can be readily visualized by indirect enzyme immunoassay techniques, usually using a chromogenic substrate (e.g. alkaline phosphatase or horseradish peroxidase) or chemiluminescent substrates. Other possibilities for probing include the use of fluorescent or radioisotope labels (e.g., fluorescein, ¹²⁵I). Probes for the detection of antibody binding can be conjugated anti-immunoglobulins, conjugated staphylococcal Protein A (binds IgG), or probes to biotinylated primary antibodies (e.g., conjugated avidin/streptavidin).

The power of the technique lies in the simultaneous detection of a specific protein by means of its antigenicity, and its molecular mass. Proteins are first separated by mass in the SDS-PAGE, then specifically detected in the immunoassay step. Thus, protein standards (ladders) can be run simultaneously in order to approximate molecular mass of the protein of interest in a heterogeneous sample.

The gel shift assay or electrophoretic mobility shift assay (EMSA) can be used to detect the interactions between DNA binding proteins and their cognate DNA recognition sequences, in both a qualitative and quantitative manner. Exemplary techniques are described in Ornstein L., Disc electrophoresis—I: Background and theory, Ann. NY Acad. Sci. 121:321-349 (1964), and Matsudiara, PT and DR Burgess, SDS microslab linear gradient polyacrylamide gel electrophoresis, Anal. Biochem. 87:386-396 (1987), each of which is herein incorporated by reference in its entirety for teachings regarding gel-shift assays.

In a general gel-shift assay, purified proteins or crude cell extracts can be incubated with a labeled (e.g., ³²P-radiolabeled) DNA or RNA probe, followed by separation of the complexes from the free probe through a nondenaturing polyacrylamide gel. The complexes migrate more slowly through the gel than unbound probe. Depending on the activity of the binding protein, a labeled probe can be either double-stranded or single-stranded. For the detection of DNA binding proteins such as transcription factors, either purified or partially purified proteins, or nuclear cell extracts can be used. For detection of RNA binding proteins, either purified or partially purified proteins, or nuclear or cytoplasmic cell extracts can be used. The specificity of the DNA or RNA binding protein for the putative binding site is established by competition experiments using DNA or RNA fragments or oligonucleotides containing a binding site for the protein of interest, or other unrelated sequence. The differences in the nature and intensity of the complex formed in the presence of specific and nonspecific competitor allows identification of specific interactions. Refer to Promega, Gel Shift Assay FAQ, available at <http://www.promega.com/faq/gelsbfaq.html> (last visited Mar. 25, 2005), which is herein incorporated by reference in its entirety for teachings regarding gel shift methods.

Gel shift methods can include using, for example, colloidal forms of COOMASSIE (Imperial Chemicals Industries, Ltd) blue stain to detect proteins in gels such as polyacrylamide electrophoresis gels. Such methods are described, for example, in Neuhoff et al., Electrophoresis 6:427-448 (1985), and Neuhoff et al., Electrophoresis 9:255-262 (1988), each of which is herein incorporated by reference in its entirety for teachings regarding gel shift methods. In addition to the conventional protein assay methods referenced above, a combination cleaning and protein staining composition is described in U.S. Pat. No. 5,424,000, herein incorporated by reference in its entirety for its teaching regarding gel shift methods. The solutions can include phosphoric, sulfuric, and nitric acids, and Acid Violet dye.

Radioimmune Precipitation Assay (RIPA) is a sensitive assay using radiolabeled antigens to detect specific antibodies in serum. The antigens are allowed to react with the serum and then precipitated using a special reagent such as, for example, protein A sepharose beads. The bound radiolabeled immunoprecipitate is then commonly analyzed by gel electrophoresis Radioimmunoprecipitation assay (RIPA) is often used as a confirmatory test for diagnosing the presence of HIV antibodies. RIPA is also referred to in the art as Farr Assay, Precipitin Assay, Radioimmune Precipitin Assay; Radioimmunoprecipitation Analysis; Radioimmunoprecipitation Analysis, and Radioimmunoprecipitation Analysis.

While the above immunoassays that utilize electrophoresis to separate and detect the specific proteins of interest allow for evaluation of protein size, they are not very sensitive for evaluating protein concentration. However, also contemplated are immunoassays wherein the protein or antibody specific for the protein is bound to a solid support (e.g., tube, well, bead, or cell) to capture the antibody or protein of interest, respectively, from a sample, combined with a method of detecting the protein or antibody specific for the protein on the support. Examples of such immunoassays include Radioimmunoassay (RIA), Enzyme-Linked Immunosorbent Assay (ELISA), Flow cytometry, protein array, multiplexed bead assay, and magnetic capture.

Radioimmunoassay (RIA) is a classic quantitative assay for detection of antigen-antibody reactions using a radioactively labeled substance (radioligand), either directly or indirectly, to measure the binding of the unlabeled substance to a specific antibody or other receptor system. Radioimmunoassay is used, for example, to test hormone levels in the blood without the need to use a bioassay. Non-immunogenic substances (e.g., haptens) can also be measured if coupled to larger carrier proteins (e.g., bovine gamma-globulin or human serum albumin) capable of inducing antibody formation. RIA involves mixing a radioactive antigen (because of the ease with which iodine atoms can be introduced into tyrosine residues in a protein, the radioactive isotopes ¹²⁵I or ¹³¹I are often used) with antibody to that antigen. The antibody is generally linked to a solid support, such as a tube or beads. Unlabeled or “cold” antigen is then adding in known quantities and measuring the amount of labeled antigen displaced. Initially, the radioactive antigen is bound to the antibodies. When cold antigen is added, the two compete for antibody binding sites—and at higher concentrations of cold antigen, more binds to the antibody, displacing the radioactive variant. The bound antigens are separated from the unbound ones in solution and the radioactivity of each used to plot a binding curve. The technique is both extremely sensitive, and specific.

Enzyme-Linked Immunosorbent Assay (ELISA), or more generically termed EIA (Enzyme ImmunoAssay), is an immunoassay that can detect an antibody specific for a protein. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha.-glycerophosphate dehydrogenase, triose phosphate isomerase, glucose-6-phosphate dehydrogenase, glucoamylase and acetylcholinesterase.

Variations of ELISA techniques are know to those of skill in the art. In one variation, antibodies that can bind to proteins can be immobilized onto a selected surface exhibiting protein affinity, such as a well in a polystyrene microtiter plate. Then, a test composition suspected of containing a marker antigen can be added to the wells. After binding and washing to remove non-specifically bound immunocomplexes, the bound antigen can be detected. Detection can be achieved by the addition of a second antibody specific for the target protein, which is linked to a detectable label. This type of ELISA is a simple “sandwich ELISA.” Detection also can be achieved by the addition of a second antibody, followed by the addition of a third antibody that has binding affinity for the second antibody, with the third antibody being linked to a detectable label.

Another variation is a competition ELISA. In competition ELISA's, test samples compete for binding with known amounts of labeled antigens or antibodies. The amount of reactive species in the sample can be determined by mixing the sample with the known labeled species before or during incubation with coated wells. The presence of reactive species in the sample acts to reduce the amount of labeled species available for binding to the well and thus reduces the ultimate signal.

Regardless of the format employed, ELISAs have certain features in common, such as coating, incubating or binding, washing to remove non-specifically bound species, and detecting the bound immunecomplexes. Antigen or antibodies can be linked to a solid support, such as in the form of plate, beads, dipstick, membrane or column matrix, and the sample to be analyzed applied to the immobilized antigen or antibody. In coating a plate with either antigen or antibody, one will generally incubate the wells of the plate with a solution of the antigen or antibody, either overnight or for a specified period of hours. The wells of the plate can then be washed to remove incompletely adsorbed material. Any remaining available surfaces of the wells can then be “coated” with a nonspecific protein that is antigenically neutral with regard to the test antisera. These include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means rather than a direct procedure can also be used. Thus, after binding of a protein or antibody to the well, coating with a non-reactive material to reduce background, and washing to remove unbound material, the immobilizing surface is contacted with the control clinical or biological sample to be tested under conditions effective to allow immunecomplex (antigen/antibody) formation. Detection of the immunecomplex then requires a labeled secondary binding agent or a secondary binding agent in conjunction with a labeled third binding agent.

Enzyme-Linked Immunospot Assay (ELISPOT) is an immunoassay that can detect an antibody specific for a protein or antigen. In such an assay, a detectable label bound to either an antibody-binding or antigen-binding reagent is an enzyme. When exposed to its substrate, this enzyme reacts in such a manner as to produce a chemical moiety which can be detected, for example, by spectrophotometric, fluorometric or visual means. Enzymes which can be used to detectably label reagents useful for detection include, but are not limited to, horseradish peroxidase, alkaline phosphatase, glucose oxidase, β-galactosidase, ribonuclease, urease, catalase, malate dehydrogenase, staphylococcal nuclease, asparaginase, yeast alcohol dehydrogenase, alpha-glycerophosphate dehydrogenase, triose phosphate isomerase, glucose-6-phosphate dehydrogenase, glucoamylase and acetylcholinesterase. In this assay a nitrocellulose microtiter plate is coated with antigen. The test sample is exposed to the antigen and then reacted similarly to an ELISA assay. Detection differs from a traditional ELISA in that detection is determined by the enumeration of spots on the nitrocellulose plate. The presence of a spot indicates that the sample reacted to the antigen. The spots can be counted and the number of cells in the sample specific for the antigen determined.

“Under conditions effective to allow immunecomplex (antigen/antibody) formation” means that the conditions include diluting the antigens and antibodies with solutions such as BSA, bovine gamma globulin (BGG) and phosphate buffered saline (PBS)/Tween so as to reduce non-specific binding and to promote a reasonable signal to noise ratio.

The suitable conditions also mean that the incubation is at a temperature and for a period of time sufficient to allow effective binding. Incubation steps can typically be from about 1 minute to twelve hours, at temperatures of about 20° to 30° C., or can be incubated overnight at about 0° C. to about 10° C.

Following all incubation steps in an ELISA, the contacted surface can be washed so as to remove non-complexed material. A washing procedure can include washing with a solution such as PBS/Tween or borate buffer. Following the formation of specific immunecomplexes between the test sample and the originally bound material, and subsequent washing, the occurrence of even minute amounts of immunecomplexes can be determined.

To provide a detecting means, the second or third antibody can have an associated label to allow detection, as described above. This can be an enzyme that can generate color development upon incubating with an appropriate chromogenic substrate. Thus, for example, one can contact and incubate the first or second immunecomplex with a labeled antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label can be quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation can then be achieved by measuring the degree of color generation, e.g., using a visible spectra spectrophotometer.

Protein arrays are solid-phase ligand binding assay systems using immobilized proteins on surfaces which include glass, membranes, microtiter wells, mass spectrometer plates, and beads or other particles. The assays are highly parallel (multiplexed) and often miniaturized (microarrays, protein chips). Their advantages include being rapid and automatable, capable of high sensitivity, economical on reagents, and giving an abundance of data for a single experiment Bioinformatics support is important, the data handling demands sophisticated software and data comparison analysis. However, the software can be adapted from that used for DNA arrays, as can much of the hardware and detection systems.

One of the chief formats is the capture array, in which ligand-binding reagents, which are usually antibodies but can also be alternative protein scaffolds, peptides or nucleic acid aptamers, are used to detect target molecules in mixtures such as plasma or tissue extracts. In diagnostics, capture arrays can be used to carry out multiple immunoassays in parallel, both testing for several analytes in individual sera for example and testing many serum samples simultaneously. In proteomics, capture arrays are used to quantitate and compare the levels of proteins in different samples in health and disease, i.e. protein expression profiling. Proteins other than specific ligand binders are used in the array format for in vitro functional interaction screens such as protein-protein, protein-DNA, protein-drug, receptor-ligand, enzyme-substrate, etc. The capture reagents themselves are selected and screened against many proteins, which can also be done in a multiplex array format against multiple protein targets.

For construction of arrays, sources of proteins include cell-based expression systems for recombinant proteins, purification from natural sources, production in vitro by cell-free translation systems, and synthetic methods for peptides. Many of these methods can be automated for high throughput production. For capture arrays and protein function analysis, it is important that proteins should be correctly folded and functional; this is not always the case, e.g. where recombinant proteins are extracted from bacteria under denaturing conditions. Nevertheless, arrays of denatured proteins are useful in screening antibodies for cross-reactivity, identifying autoantibodies and selecting ligand binding proteins.

Protein arrays have been designed as a miniaturization of familiar immunoassay methods such as ELISA and dot blotting, often utilizing fluorescent readout, and facilitated by robotics and high throughput detection systems to enable multiple assays to be carried out in parallel. Commonly used physical supports include glass slides, silicon, microwells, nitrocellulose or PVDF membranes, and magnetic and other microbeads. While microdrops of protein delivered onto planar surfaces are the most familiar format, alternative architectures include CD centrifugation devices based on developments in microfluidics (Gyros, Monmouth Junction, NJ) and specialized chip designs, such as engineered microchannels in a plate (e.g., The Living Chip™, Biotrove, Woburn, MA) and tiny 3D posts on a silicon surface (Zyomyx, Hayward CA). Particles in suspension can also be used as the basis of arrays, providing they are coded for identification; systems include color coding for microbeads (Luminex, Austin, TX; Bio-Rad Laboratories) and semiconductor nanocrystals (e.g., QDots™, Quantum Dot, Hayward, CA), and barcoding for beads (UltraPlex™, SmartBead Technologies Ltd, Babraham, Cambridge, UK) and multimetal microrods (e.g., Nanobarcodes™ particles, Nanoplex Technologies, Mountain View, CA). Beads can also be assembled into planar arrays on semiconductor chips (LEAPS technology, BioArray Solutions, Warren, NJ).

Immobilization of proteins involves both the coupling reagent and the nature of the surface being coupled to. A good protein array support surface is chemically stable before and after the coupling procedures, allows good spot morphology, displays minimal nonspecific binding, does not contribute a background in detection systems, and is compatible with different detection systems. The immobilization method used are reproducible, applicable to proteins of different properties (size, hydrophilic, hydrophobic), amenable to high throughput and automation, and compatible with retention of fully functional protein activity. Orientation of the surface-bound protein is recognized as an important factor in presenting it to ligand or substrate in an active state; for capture arrays the most efficient binding results are obtained with orientated capture reagents, which generally require site-specific labeling of the protein.

Both covalent and noncovalent methods of protein immobilization are used and have various pros and cons. Passive adsorption to surfaces is methodologically simple, but allows little quantitative or orientational control, it may or may not alter the functional properties of the protein, and reproducibility and efficiency are variable. Covalent coupling methods provide a stable linkage, can be applied to a range of proteins and have good reproducibility; however, orientation may be variable, chemical derivatization may alter the function of the protein and requires a stable interactive surface. Biological capture methods utilizing a tag on the protein provide a stable linkage and bind the protein specifically and in reproducible orientation, but the biological reagent must first be immobilized adequately and the array may require special handling and have variable stability.

Several immobilization chemistries and tags have been described for fabrication of protein arrays. Substrates for covalent attachment include glass slides coated with amino- or aldehyde-containing silane reagents. In the Versalinx™ system (Prolinx, Bothell, WA) reversible covalent coupling is achieved by interaction between the protein derivatized with phenyldiboronic acid, and salicylhydroxamic acid immobilized on the support surface. This also has low background binding and low intrinsic fluorescence and allows the immobilized proteins to retain function. Noncovalent binding of unmodified protein occurs within porous structures such as HydroGel™ (PerkinElmer, Wellesley, MA), based on a 3-dimensional polyacrylamide gel; this substrate is reported to give a particularly low background on glass microarrays, with a high capacity and retention of protein function. Widely used biological coupling methods are through biotin/streptavidin or hexahistidine/Ni interactions, having modified the protein appropriately. Biotin may be conjugated to a poly-lysine backbone immobilized on a surface such as titanium dioxide (Zyomyx) or tantalum pentoxide (Zeptosens, Witterswil, Switzerland).

Array fabrication methods include robotic contact printing, ink-jetting, piezoelectric spotting and photolithography. A number of commercial arrayers are available [e.g. Packard Biosciences] as well as manual equipment [V & P Scientific]. Bacterial colonies can be robotically gridded onto PVDF membranes for induction of protein expression in situ.

At the limit of spot size and density are nanoarrays, with spots on the nanometer spatial scale, enabling thousands of reactions to be performed on a single chip less than 1 mm square. BioForce Laboratories have developed nanoarrays with 1521 protein spots in 85 sq microns, equivalent to 25 million spots per sq cm, at the limit for optical detection; their readout methods are fluorescence and atomic force microscopy (AFM).

Fluorescence labeling and detection methods are widely used. The same instrumentation as used for reading DNA microarrays is applicable to protein arrays. For differential display, capture (e.g., antibody) arrays can be probed with fluorescently labeled proteins from two different cell states, in which cell lysates are directly conjugated with different fluorophores (e.g. Cy-3, Cy-5) and mixed, such that the color acts as a readout for changes in target abundance. Fluorescent readout sensitivity can be amplified 10-100 fold by tyramide signal amplification (TSA) (PerkinElmer Lifesciences). Planar waveguide technology (Zeptosens) enables ultrasensitive fluorescence detection, with the additional advantage of no intervening washing procedures. High sensitivity can also be achieved with suspension beads and particles, using phycoerythrin as label (Luminex) or the properties of semiconductor nanocrystals (Quantum Dot). A number of novel alternative readouts have been developed, especially in the commercial biotech arena. These include adaptations of surface plasmon resonance (HTS Biosystems, Intrinsic Bioprobes, Tempe, AZ), rolling circle DNA amplification (Molecular Staging, New Haven CT), mass spectrometry (Intrinsic Bioprobes; Ciphergen, Fremont, CA), resonance light scattering (Genicon Sciences, San Diego, CA) and atomic force microscopy [BioForce Laboratories].

Capture arrays form the basis of diagnostic chips and arrays for expression profiling. They employ high affinity capture reagents, such as conventional antibodies, single domains, engineered scaffolds, peptides or nucleic acid aptamers, to bind and detect specific target ligands in high throughput manner.

Antibody arrays have the required properties of specificity and acceptable background, and some are available commercially (BD Biosciences, San Jose, CA; Clontech, Mountain View, CA; BioRad, Sigma, St. Louis, MO). Antibodies for capture arrays are made either by conventional immunization (polyclonal sera and hybridomas), or as recombinant fragments, usually expressed in E. coli, after selection from phage or ribosome display libraries (Cambridge Antibody Technology, Cambridge, UK; BioInvent, Lund, Sweden; Affitech, Walnut Creek, CA; Biosite, San Diego, CA). In addition to the conventional antibodies, Fab and scFv fragments, single V-domains from camelids or engineered human equivalents (Domantis, Waltham, MA) may also be useful in arrays.

The term “scaffold” refers to ligand-binding domains of proteins, which are engineered into multiple variants capable of binding diverse target molecules with antibody-like properties of specificity and affinity. The variants can be produced in a genetic library format and selected against individual targets by phage, bacterial or ribosome display. Such ligand-binding scaffolds or frameworks include ‘Affibodies’ based on Staph. aureus protein A (Affibody, Bromma, Sweden), ‘Trinectins’ based on fibronectins (Phylos, Lexington, MA) and ‘Anticalins’ based on the lipocalin structure (Pieris Proteolab, Freising-Weihenstephan, Germany). These can be used on capture arrays in a similar fashion to antibodies and may have advantages of robustness and ease of production.

Nonprotein capture molecules, notably the single-stranded nucleic acid aptamers which bind protein ligands with high specificity and affinity, are also used in arrays (SomaLogic, Boulder, CO). Aptamers are selected from libraries of oligonucleotides by the Selex™ procedure and their interaction with protein can be enhanced by covalent attachment, through incorporation of brominated deoxyuridine and UV-activated crosslinking (photoaptamers). Photocrosslinking to ligand reduces the crossreactivity of aptamers due to the specific steric requirements. Aptamers have the advantages of ease of production by automated oligonucleotide synthesis and the stability and robustness of DNA, on photoaptamer arrays, universal fluorescent protein stains can be used to detect binding.

Protein analytes binding to antibody arrays may be detected directly or via a secondary antibody in a sandwich assay. Direct labelling is used for comparison of different samples with different colors. Where pairs of antibodies directed at the same protein ligand are available, sandwich immunoassays provide high specificity and sensitivity and are therefore the method of choice for low abundance proteins such as cytokines; they also give the possibility of detection of protein modifications. Label-free detection methods, including mass spectrometry, surface plasmon resonance and atomic force microscopy, avoid alteration of ligand. What is required from any method is optimal sensitivity and specificity, with low background to give high signal to noise. Since analyte concentrations cover a wide range, sensitivity has to be tailored appropriately; serial dilution of the sample or use of antibodies of different affinities are solutions to this problem. Proteins of interest are frequently those in low concentration in body fluids and extracts, requiring detection in the pg range or lower, such as cytokines or the low expression products in cells.

An alternative to an array of capture molecules is one made through ‘molecular imprinting’ technology, in which peptides (e.g., from the C-terminal regions of proteins) are used as templates to generate structurally complementary, sequence-specific cavities in a polymerizable matrix, the cavities can then specifically capture (denatured) proteins that have the appropriate primary amino acid sequence (ProteinPrint™, Aspira Biosystems, Burlingame, CA) Another methodology which can be used diagnostically and in expression profiling is the ProteinChip® array (Ciphergen, Fremont, CA), in which solid phase chromatographic surfaces bind proteins with similar characteristics of charge or hydrophobicity from mixtures such as plasma or tumour extracts, and SELDI-TOF mass spectrometry is used to detection the retained proteins.

Large-scale functional chips have been constructed by immobilizing large numbers of purified proteins and used to assay a wide range of biochemical functions, such as protein interactions with other proteins, drug-target interactions, enzyme-substrates, etc. Generally they require an expression library, cloned into E. coli, yeast or similar from which the expressed proteins are then purified, e.g. via a His tag, and immobilized. Cell free protein transcription/translation is a viable alternative for synthesis of proteins which do not express well in bacterial or other in vivo systems.

For detecting protein-protein interactions, protein arrays can be in vitro alternatives to the cell-based yeast two-hybrid system and may be useful where the latter is deficient, such as interactions involving secreted proteins or proteins with disulphide bridges. High-throughput analysis of biochemical activities on arrays has been described for yeast protein kinases and for various functions (protein-protein and protein-lipid interactions) of the yeast proteome, where a large proportion of all yeast open-reading frames was expressed and immobilized on a microarray. Large-scale ‘proteome chips’ promise to be very useful in identification of functional interactions, drug screening, etc. (Proteometrix, Branford, CT).

As a two-dimensional display of individual elements, a protein array can be used to screen phage or ribosome display libraries, in order to select specific binding partners, including antibodies, synthetic scaffolds, peptides and aptamers. In this way, ‘library against library’ screening can be carried out. Screening of drug candidates in combinatorial chemical libraries against an array of protein targets identified from genome projects is another application of the approach.

A multiplexed bead assay, such as, for example, the BD™ Cytometric Bead Array, is a series of spectrally discrete particles that can be used to capture and quantitate soluble analytes. The analyte is then measured by detection of a fluorescence-based emission and flow cytometric analysis. Multiplexed bead assay generates data that is comparable to ELISA based assays, but in a “multiplexed” or simultaneous fashion. Concentration of unknowns is calculated for the cytometric bead array as with any sandwich format assay, i.e. through the use of known standards and plotting unknowns against a standard curve. Further, multiplexed bead assay allows quantification of soluble analytes in samples never previously considered due to sample volume limitations. In addition to the quantitative data, powerful visual images can be generated revealing unique profiles or signatures that provide the user with additional information at a glance.

### 5. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue. Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue. In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp 303-357; Smith et al., Antibodies in Human Diagnosis and Therapy, Haber et al., eds., Raven Press, New York (1977) pp. 365-389. A typical dosage of the antibody used alone might range from about 1 μg/kg to up to 100 mg/kg of body weight or more per day, per week, per month, or longer, depending on the factors mentioned above.

### C. Methods of Treating Kidney Disease

It is understood and herein contemplated that the disclosed urokinase plasminogen activator receptor (uPAR) binding molecules (including, but not limited to soluble urokinase plasminogen activator receptor (suPAR) binding molecules) can be used to treat, reduce, decrease, inhibit, ameliorate, and/or prevent a kidney disease or condition. As used herein, “kidney disease” refers to any disease or condition that directly affects the kidneys or their function. “Kidney disease” can also refer to kidney injury that is the result of inflammation from another disease (e.g., multiple myeloma or systemic lupus erythematosus) that effects the kidneys or injury not due to a disease or condition (such as, for example, injury as the result of trauma, contrast agents, infection, surgery, ischemia/reperfusion injury, transplant, or medication). Thus, as used herein, examples of kidney diseases include, but are not limited to proteinuric kidney disease; Focal segmental glomerulosclerosis (FSGS); IgA nephropathy; membranous nephropathy; lupus nephritis; diabetic nephropathy; Autosomal dominant polycystic kidney disease (ADPKD); Alport syndrome, acute kidney injury (AKI) (including, but not limited to COVID-19 AKI); glomerulonephritis; preeclampsia; systemic lupus erythematosus; multiple myeloma; or kidney injury as the result of trauma, contrast agents, infection, surgery, ischemia/reperfusion injury, transplant, or medication.

In one aspect, disclosed herein are methods of treating, decreasing, inhibiting, reducing, ameliorating and/or preventing an inflammatory kidney disease or condition (such as, for example, proteinuric kidney disease; Focal segmental glomerulosclerosis (FSGS); IgA nephropathy; membranous nephropathy; lupus nephritis; diabetic nephropathy; Autosomal dominant polycystic kidney disease (ADPKD); Alport syndrome, acute kidney injury (AKI) (including, but not limited to COVID-19 AKI), glomerulonephritis; preeclampsia; systemic lupus erythematosus; multiple myeloma; or kidney injury as the result of trauma, contrast agents, infection, surgery, ischemia/reperfusion injury, transplant, or medication) in a subject or the symptoms thereof comprising administering to the subject any of the urokinase plasminogen activator receptor (uPAR) binding molecules disclosed herein (such as, for example, any of the soluble urokinase plasminogen activator receptor (suPAR) binding molecules disclosed herein). For example, methods of treating, decreasing, inhibiting, reducing, ameliorating, and/or preventing an inflammatory kidney disease in a subject or the symptoms thereof comprising administering to the subject one or more urokinase plasminogen activator receptor (uPAR) binding molecules, including, but not limited to any of the soluble urokinase plasminogen activator receptor (uPAR) binding molecules disclosed herein (such as, for example a chimeric antigen receptor (CAR) T cell, CAR NK cell, CAR Macrophage (CARMA), immunotoxin, bispecific antibody, diabody, triabody, Bispecific T cell engager (BITE), antibody, or antibody fragment) comprising a light chain variable domain, wherein the light chain variable domain comprises 3 complementarity determining regions (CDRs), CDR1, CDR2, and CDR3 as set forth in SEQ ID NOs 71-75 and 1901-2500; SEQ ID NOs: 76, 77, and 2501-3100, and SEQ ID NO: 78 and 3100-3700, respectively or any other CDR as set forth in Tables 3, 4, or 6. In one aspect, the light chain variable domain can comprise a CDR1, CDR2, and CD3, as set forth in SEQ ID Nos 71, 76, and 78, respectively; SEQ ID Nos 72, 76, and 78, respectively; SEQ ID Nos 72, 77, and 78, respectively; SEQ ID Nos 73, 76, and 78, respectively; SEQ ID Nos 74, 76, and 78, respectively; or SEQ ID Nos 75, 76, and 78, respectively. For example, the urokinase plasminogen activator receptor (uPAR) binding molecule (such as, for example, a soluble urokinase plasminogen activator receptor (uPAR) binding molecule) can comprise a light chain variable domain (V_{L}) comprising the amino acid sequence as set forth in SEQ ID NOs: 2, 25-44, 4300-4900, and 4904 or as shown in Tables 3, 4, or 6. In some aspects, the urokinase plasminogen activator receptor (uPAR) binding molecules (including, but not limited to suPAR binding molecules) used in the disclosed methods of treating, decreasing, inhibiting, reducing, ameliorating, and/or preventing an inflammatory kidney disease or condition or the symptoms thereof can further comprise a heavy chain variable domain; wherein the heavy chain variable domain comprises 3 complementarity determining regions (CDRs), CDR1, CDR2, and CDR3 as set forth in SEQ ID NOs 45-57 and 101-700; SEQ ID NOs: 58-68 and 701-1300; and SEQ ID NOs: 69, 70, and 1301-1900, respectively or any other CDR as set forth in Tables 3 or 4. In one aspect, the heavy chain variable domain can comprise a CDR1, CDR2, and CD3, as set forth in SEQ ID Nos: 45, 58, and 69, respectively; SEQ ID Nos: 45, 59, and 69, respectively; SEQ ID Nos: 46, 60, and 69, respectively; SEQ ID Nos: 46, 61, and 69, respectively; SEQ ID Nos: 47, 62, and 69, respectively; SEQ ID Nos: 48, 62, and 70, respectively; SEQ ID Nos: 49, 62, and 69, respectively; SEQ ID Nos: 50, 62, and 69, respectively; SEQ ID Nos: 51, 62, and 69, respectively; SEQ ID Nos: 52, 62, and 69, respectively; SEQ ID Nos: 53, 63, and 69, respectively; SEQ ID Nos: 53, 64, and 69, respectively; SEQ ID Nos: 54, 65, and 69, respectively; SEQ ID Nos: 54, 66, and 69, respectively; SEQ ID Nos: 55, 62, and 69, respectively; SEQ ID Nos: 53, 62, and 69, respectively, SEQ ID Nos. 56, 67, and 69, respectively; SEQ ID Nos: 57, 68, and 69, respectively; or SEQ ID Nos: 53, 64, and 69, respectively. For example, the urokinase plasminogen activator receptor (uPAR) binding molecule (including, but not limited suPAR binding molecules) can comprise a heavy chain variable domain (Vn) comprising the amino acid sequence as set forth in SEQ ID NOs: 1, 5-24, 3701-4300 and 4903 or as shown in Table 3, 4, or 6. Thus, in one aspect, the urokinase plasminogen activator receptor (uPAR) binding molecule used in the disclosed methods of treating, decreasing, inhibiting, reducing, ameliorating, and/or preventing an inflammatory kidney disease or condition or the symptoms thereof can comprise a heavy chain CDR1, CDR2 and CDR 3 as set forth in SEQ ID NOs: 48, 62, and 70, respectively; and a light chain CDR1, CDR2, and CDR3 as set forth in SEQ ID Nos 72, 76, and 78, respectively; a heavy chain CDR1, CDR2 and CDR 3 as set forth in SEQ ID NOs: 53, 64, and 69, respectively, and a light chain CDR1, CDR2, and CDR3 as set forth in SEQ ID Nos 71, 76, and 78, respectively. For example, the urokinase plasminogen activator receptor (uPAR) binding molecule can comprise a heavy chain as set forth in SEQ ID NO: 4903 and a light chain as set forth in SEQ ID NO: 4904; a heavy chain as set forth in SEQ ID NO: 1 and a light chain as set forth in SEQ ID NO: 2; a heavy chain as set forth in SEQ ID NO: 10 and a light chain as set forth in SEQ ID NO: 30; a heavy chain as set forth in SEQ ID NO: 12 and a light chain as set forth in SEQ ID NO: 32; a heavy chain as set forth in SEQ ID NO: 24 and a light chain as set forth in SEQ ID NO: 44; a heavy chain as set forth in SEQ ID NO: 5 and a light chain as set forth in SEQ ID NO: 25; a heavy chain as set forth in SEQ ID NO: 6 and a light chain as set forth in SEQ ID NO: 26; a heavy chain as set forth in SEQ ID NO: 7 and a light chain as set forth in SEQ ID NO: 27; a heavy chain as set forth in SEQ ID NO: 8 and a light chain as set forth in SEQ ID NO: 28; a heavy chain as set forth in SEQ ID NO: 9 and a light chain as set forth in SEQ ID NO: 29; a heavy chain as set forth in SEQ ID NO: 11 and a light chain as set forth in SEQ ID NO: 31; a heavy chain as set forth in SEQ ID NO: 13 and a light chain as set forth in SEQ ID NO: 33; a heavy chain as set forth in SEQ ID NO: 14 and a light chain as set forth in SEQ ID NO: 34; a heavy chain as set forth in SEQ ID NO: 15 and a light chain as set forth in SEQ ID NO: 35; a heavy chain as set forth in SEQ ID NO: 16 and a light chain as set forth in SEQ ID NO: 36; a heavy chain as set forth in SEQ ID NO: 17 and a light chain as set forth in SEQ ID NO: 37; a heavy chain as set forth in SEQ ID NO: 18 and a light chain as set forth in SEQ ID NO: 38; a heavy chain as set forth in SEQ ID NO: 19 and a light chain as set forth in SEQ ID NO: 39; a heavy chain as set forth in SEQ ID NO: 20 and a light chain as set forth in SEQ ID NO: 40; a heavy chain as set forth in SEQ ID NO: 21 and a light chain as set forth in SEQ ID NO: 41; a heavy chain as set forth in SEQ ID NO: 22 and a light chain as set forth in SEQ ID NO: 42; a heavy chain as set forth in SEQ ID NO: 23 and a light chain as set forth in SEQ ID NO: 43.

In one aspect, disclosed herein are methods of treating, decreasing, inhibiting, reducing, ameliorating, and/or preventing an inflammatory kidney disease or condition (such as, for example, proteinuric kidney disease; Focal segmental glomerulosclerosis (FSGS); IgA nephropathy; membranous nephropathy; lupus nephritis; diabetic nephropathy; Autosomal dominant polycystic kidney disease (ADPKD); Alport syndrome, acute kidney injury (AKI) (including, but not limited to COVID-19 AKI), glomerulonephritis; preeclampsia; systemic lupus erythematosus; multiple myeloma; or kidney injury as the result of trauma, contrast agents, infection, surgery, ischemia/reperfusion injury, transplant, or medication) in a subject or the symptoms thereof comprising administering to the subject one or more urokinase plasminogen activator receptor (uPAR) binding molecules, including, but not limited to suPAR binding molecules (such as, for example a chimeric antigen receptor (CAR) T cell, CAR NK cell, CAR Macrophage (CARMA), immunotoxin, bispecific antibody, diabody, triabody, Bispecific T cell engager (BiTE), antibody, or antibody fragment) comprising a heavy chain variable domain; wherein the heavy chain variable domain comprises 3 complementarity determining regions (CDRs), CDR1, CDR2, and CDR3 as set forth in SEQ ID NOs 45-57 and 101-700; SEQ ID NOs: 58-68 and 701-1300; and SEQ ID NOs: 69, 70, and 1301-1900, respectively or any other CDR as set forth in Tables 3, 4, or 6. In one aspect, the heavy chain variable domain can comprise a CDR1, CDR2, and CD3, as set forth in SEQ ID Nos: 45, 58, and 69, respectively; SEQ ID Nos: 45, 59, and 69, respectively; SEQ ID Nos: 46, 60, and 69, respectively; SEQ ID Nos: 46, 61, and 69, respectively; SEQ ID Nos: 47, 62, and 69, respectively; SEQ ID Nos: 48, 62, and 70, respectively; SEQ ID Nos: 49, 62, and 69, respectively; SEQ ID Nos: 50, 62, and 69, respectively; SEQ ID Nos: 51, 62, and 69, respectively; SEQ ID Nos: 52, 62, and 69, respectively; SEQ ID Nos: 53, 63, and 69, respectively, SEQ ID Nos. 53, 64, and 69, respectively; SEQ ID Nos: 54, 65, and 69, respectively; SEQ ID Nos: 54, 66, and 69, respectively; SEQ ID Nos: 55, 62, and 69, respectively; SEQ ID Nos: 53, 62, and 69, respectively; SEQ ID Nos: 56, 67, and 69, respectively; SEQ ID Nos: 57, 68, and 69, respectively; or SEQ ID Nos: 53, 64, and 69, respectively. For example, the urokinase plasminogen activator receptor (uPAR) binding molecule (such as, for example, the suPAR binding molecule) can comprise a heavy chain variable domain (V_{H}) comprising the amino acid sequence as set forth in SEQ ID NOs: 1, 5-24, 3701-4300 and 4903 or as shown in Table 3, 4, or 6.

In some aspects, the uPAR and/or suPAR binding molecule used in the disclosed methods are antibodies comprising a constant domain for the heavy and light chains. Accordingly, disclosed herein are methods of treating, decreasing, inhibiting, reducing, ameliorating, and/or preventing an inflammatory kidney disease or condition (such as, for example, proteinuric kidney disease; Focal segmental glomerulosclerosis (FSGS); IgA nephropathy; membranous nephropathy; lupus nephritis; diabetic nephropathy; Autosomal dominant polycystic kidney disease (ADPKD); Alport syndrome, acute kidney injury (AKI) (including, but not limited to COVID-19 AKI); glomerulonephritis; preeclampsia; systemic lupus erythematosus, multiple myeloma; or kidney injury as the result of trauma, contrast agents, infection, surgery, ischemia/reperfusion injury, transplant, or medication) in a subject or the symptoms thereof, wherein the binding molecule further comprises a light chain constant domain as set forth in SEQ ID NO: 4 or 4906 and/or wherein the binding molecule further comprises a heavy chain constant domain as set forth in SEQ ID NO: 3 or 4905.

It is understood and herein contemplated that the timing of administration can be important to the successful treatment of a subject. Accordingly, also disclosed herein are methods of treating, decreasing, inhibiting, reducing, ameliorating, and/or preventing an inflammatory kidney disease or condition, wherein the urokinase plasminogen activator receptor (uPAR) binding molecule (such as, for example a suPAR binding molecule) is administered when suPAR levels are elevated relative to a normal control. In some aspects, the uPAR binding molecule and/or suPAR binding molecule is administered prior to the onset of symptoms.

In one aspect, disclosed herein are methods of treating, decreasing, inhibiting, reducing, ameliorating, and/or preventing an inflammatory kidney disease or condition or the symptoms thereof, further comprising obtaining a biological sample (such as, for example, whole blood, plasma, serum, or urine) from the subject and measuring suPAR levels in the sample; wherein 1 ng/ml of suPAR indicates a healthy subject; 2-3 ng/ml of suPAR indicates an acute kidney disease or acute inflammation; 4 ng/ml of suPAR indicates the subject likely has or will develop chronic kidney disease; and 5 ng/ml or greater of suPAR indicates that the subject has chronic kidney disease.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

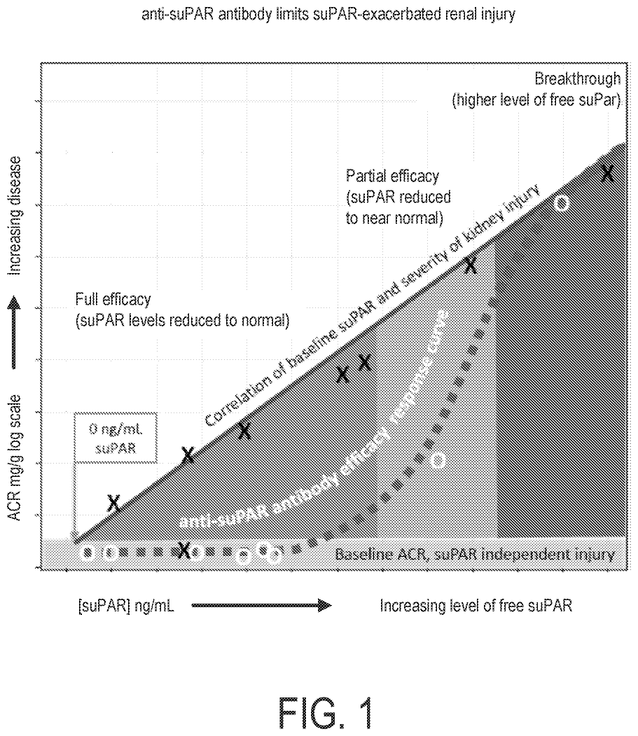

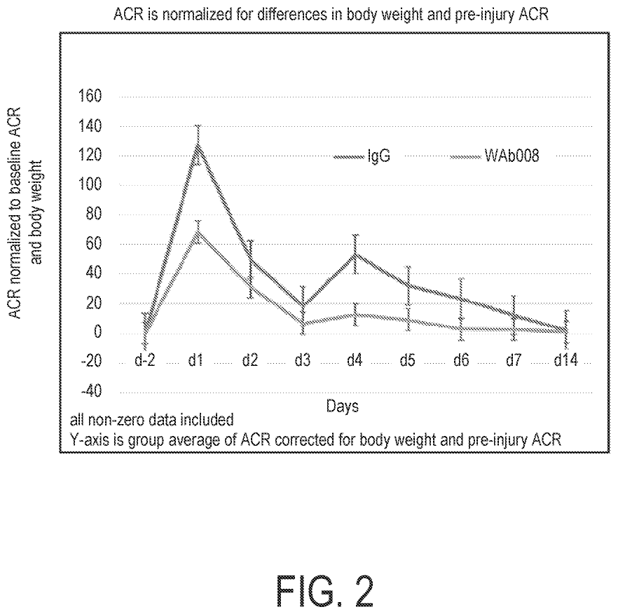

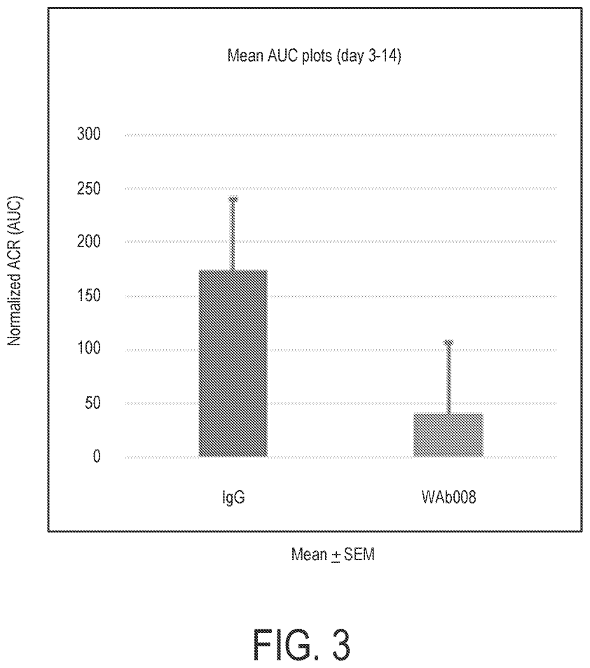

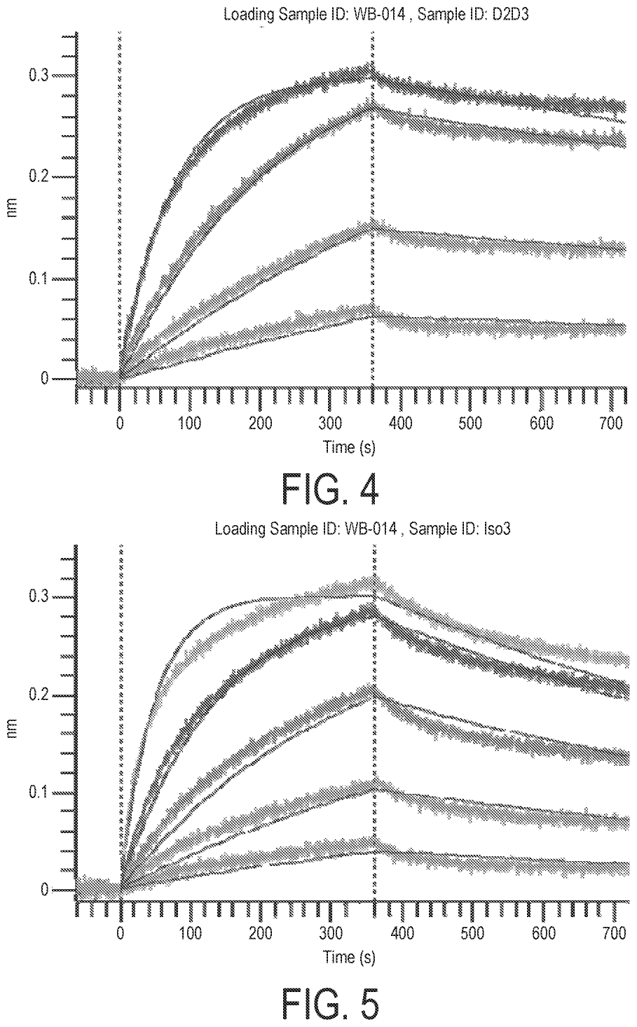

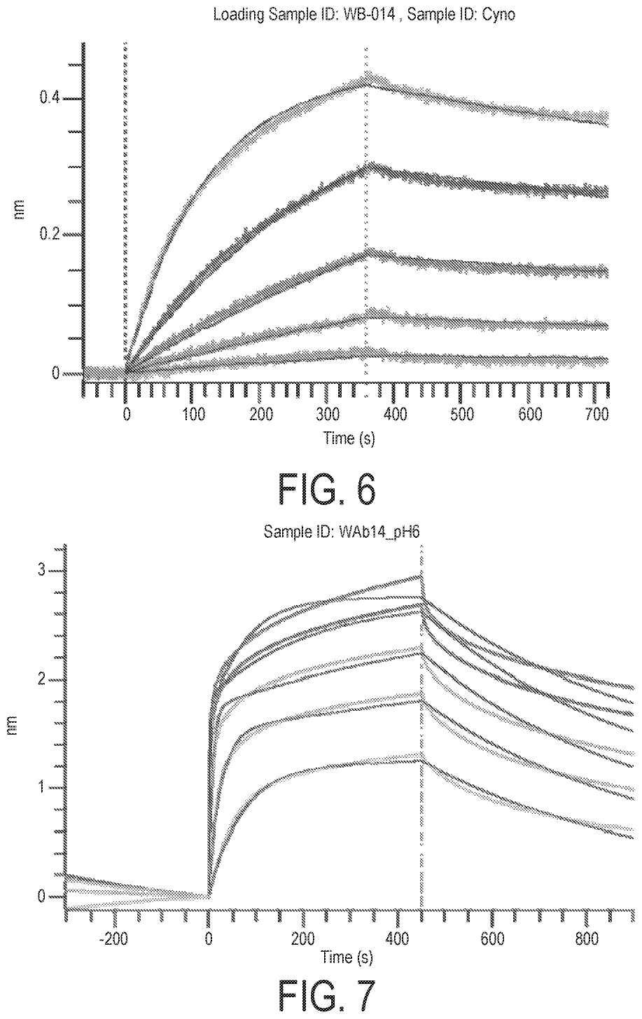

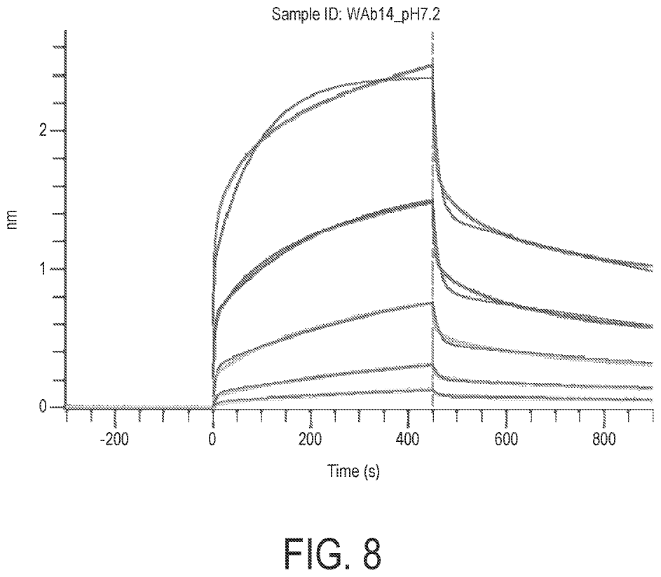

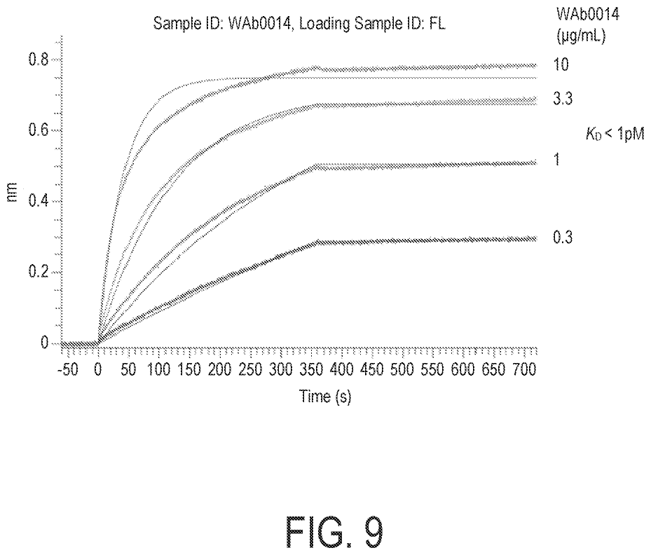

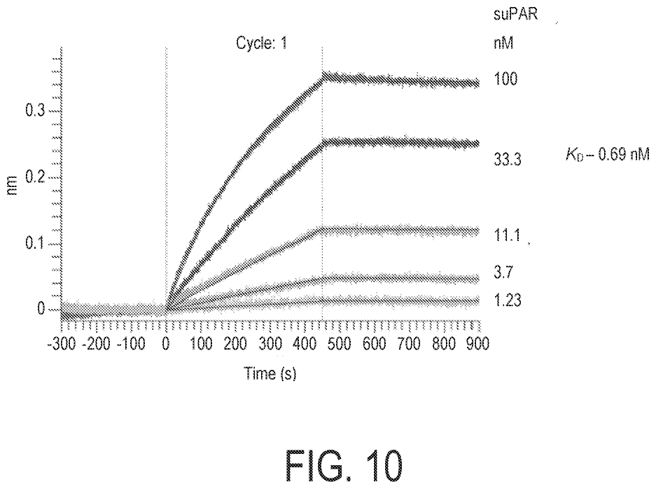

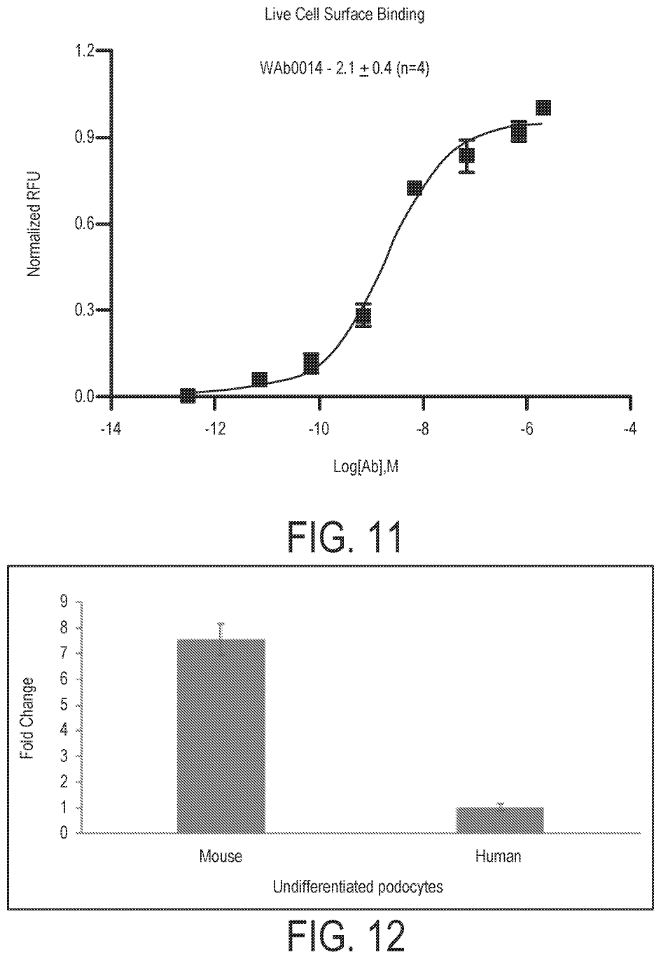

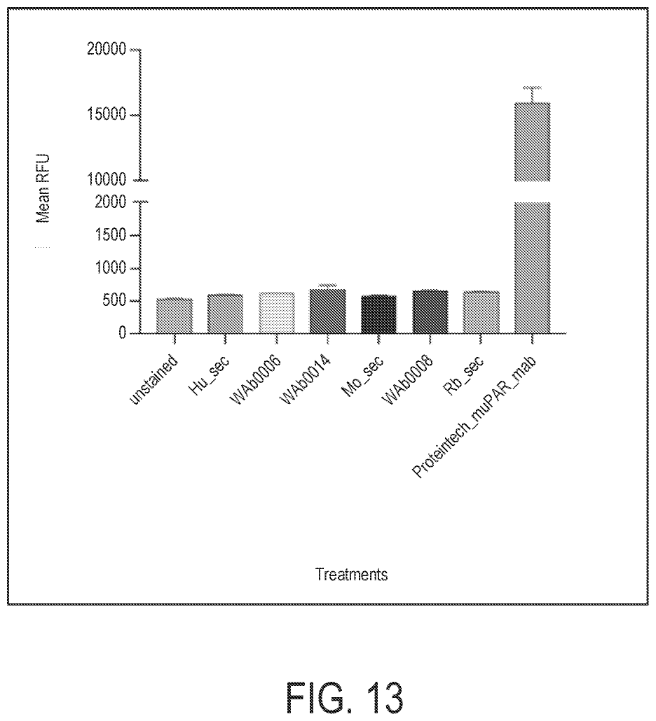

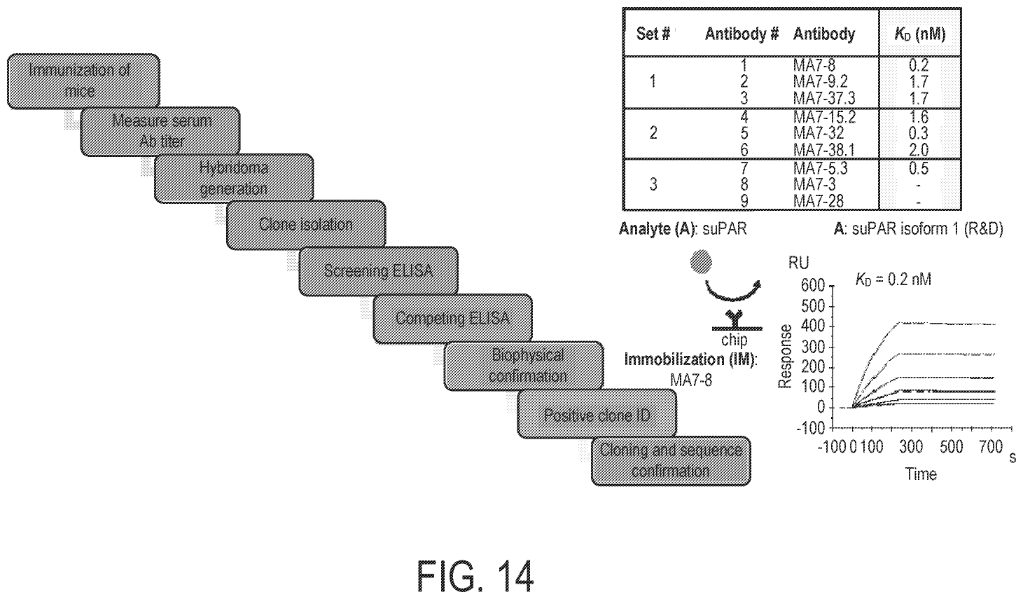

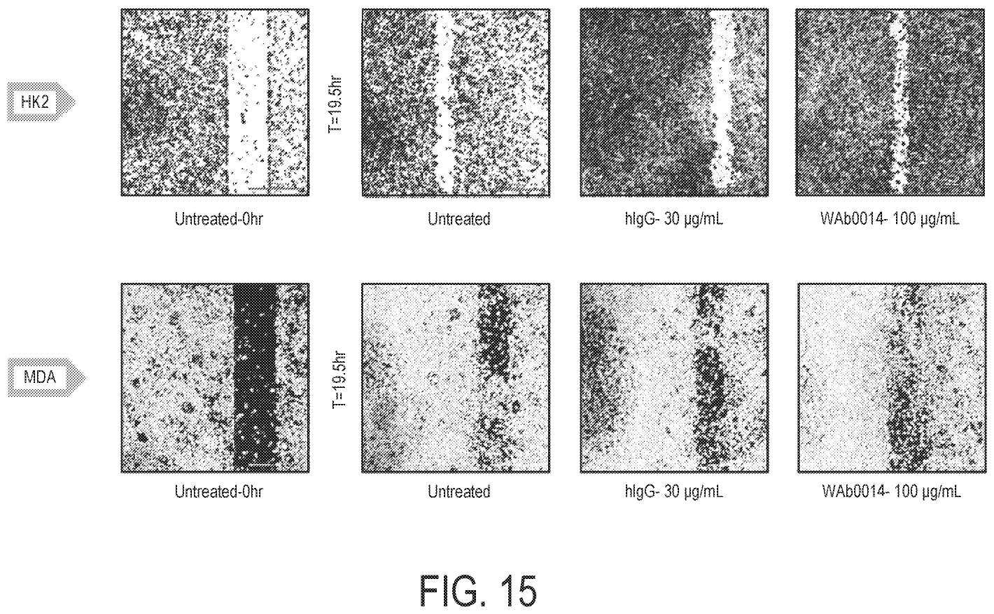

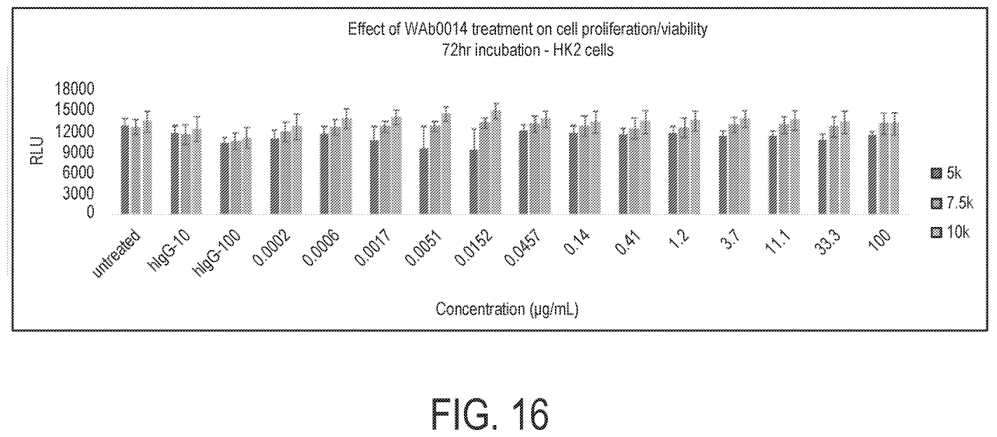

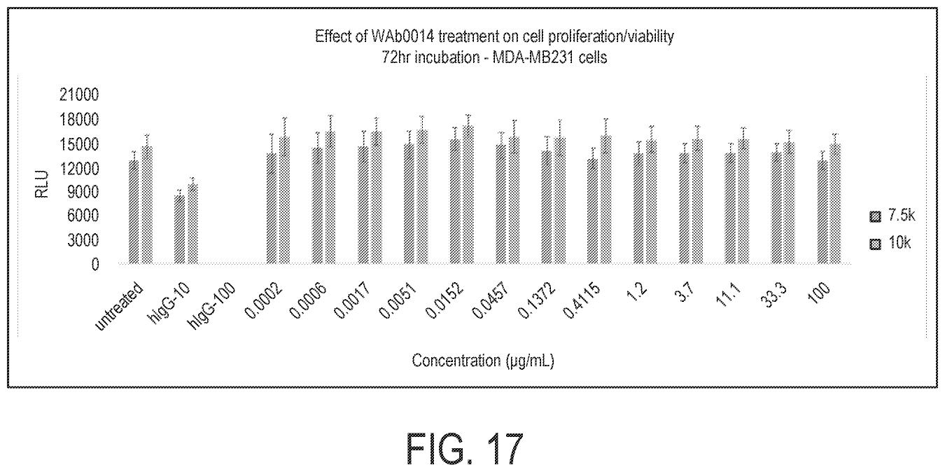

suPAR is well established immunologic risk factor for CKD and AKI in humans. Additionally, breakthrough disease occurs when free suPAR (nonantibody-complexed) exceeds ng/mL in the mouse or 2-3 ng/ml in humans. Using a nephrotoxic model of kidney injury, hu-suPAR transgenic mice demonstrate exacerbated disease scores with a strong correlation (0.87) between baseline suPAR and severity of kidney injury, as measured by ACR (X). Lead anti-suPAR antibody reduces circulating suPAR to normal levels and returns ACR to baseline levels (O) (FIG. 1). Additionally, Anti-suPAR antibody displays beneficial effects in repeat study despite differences in animal baseline characteristics (FIGS. 2 and 3).

### Example 1: Binding Affinity of WAb0014 to Human and Cynomolgus suPAR Isoforms

Multiple isoforms of hsuPAR are suggested to exist in vivo. However, an in-depth pathophysiologic understanding of these isoforms is lacking. Research was undertaken to evaluate the binding specificity profile of WAb0014 to human and cynomolgus suPAR isoforms.

### a) Methods:

Recombinantly expressed proteins for hsuPAR isoform 1-D2D3 fragment and hsuPAR-3 isoforms were custom generated in HEK or CHO cells and purified by Genscript from the known amino-acid sequence of hsuPAR-1 and hsuPAR-3 respectively. Cynomolgus suPAR isoform protein was commercially obtained from Creative Biomart. Binding affinities of WAb0014 to recombinantly expressed suPAR isoform proteins were assessed on an Octet Red 96E BLI platform in the antibody immobilized configuration as detailed above.

### b) Results:

In this study, WAb0014 exhibited a K_{D} (nM) of 2.1, 10.3, 10.5 for hsuPAR-1 D2D3 fragment (Error! Reference source not found.), hsuPAR-3 (Error! Reference source not found.) and cynomolgus suPAR (Error! Reference source not found.) respectively.

### Example 2: Assessment of Binding Affinity of WAb0014 to Neonatal FcRn Receptors

Long circulating half-lives associated with monoclonal antibody therapeutics are suggested to be a result of high affinity to neonatal FcRn receptors in vivo and especially under acidic conditions. To understand whether WAb0014 also exhibits a preferential affinity for human FcRn receptors under acidic conditions, WAb0014-hFcRn interaction on an Octet Red96E BLI biophysical platform was evaluated.

### a) Methods:

Biotinylated hFcRn protein was sourced from Acro Biosystems and immobilized on a SA biosensor. Increasing concentrations of analyte WAb0014 was then applied, and responses measured under pH 6 and pH 7.2 to mimic intracellular environment within recycling endosomes and extracellular environment at FcRn expressing tissues respectively. Binding affinity (K_{D}, nM) was calculated from the response rate constants as described above.

### b) Results:

The results showed that WAb0014 exhibited a very strong, picomolar binding affinity to hFcRn receptors under acidic conditions (K_{D}=25 pM, Error! Reference source not found.) whereas at physiological pH 7.2 it exhibited a comparatively weaker binding affinity of nM (Error! Reference source not found.). These data suggest that WAb0014 is expected to be efficaciously recycled by hFcRn receptors in vivo and as a result exhibit long circulating half-lives characteristic of known monoclonal antibody therapeutics.

### Example 3: Binding Affinity of WAb0014 to Recombinant Human suPAR and Cell Surface Human uPAR

### a) Binding Affinity Assessed by Bilayer Interferometry

A bilayer interferometry (BLI) based biophysical measurement was undertaken to assess the binding affinity of WAb0014 to human suPAR (hsuPAR) protein on an Octet Red96E platform (Sartorius).

### (1) Methods:

hsuPAR protein (R&D catalog UK807) or WAb0014 protein were immobilized to a streptavidin or an AHC biosensor (Sartorius) respectively as the ligand. After a wash step, either WAb0014 (when hsuPAR was the ligand) or hsuPAR (when WAb0014 was the ligand) was added in a dose-dependent manner. Analyte global association (kₐ) and disassociation (kₐ) rates were calculated using in-built functions to determine binding affinity (K_{D})

### (2) Results:

The study result showed that in the hsuPAR immobilized configuration, WAb0014 exhibited a K_{D}=<0.1 nM (Error! Reference source not found.); whereas in the WAb0014 immobilized configuration, we obtained a K_{D}=0.69-2 nM (Error! Reference source not found.).

### b) Binding Affinity Assessed by Flow Cytometry

Apparent binding affinity of WAb0014 to cell surface human uPAR (huPAR) was undertaken using flow cytometry on either differentiated or undifferentiated immortalized human podocytes that are known to endogenously express huPARs.

### (1) Methods:

Human podocytes were incubated with increasing concentrations of WAb0014 at 4° C. for 1 hr. After 2 cycles of wash to remove any unbound antibody, cells were incubated with 10 μg/mL of a goat-anti-human-FITC conjugated secondary antibody for 1 hr at 4° C. After washing any unbound secondary antibody, cell suspensions for each of WAb0014 test concentrations were gated for single cells and population mean fluorescence intensity data was captured and analyzed on a Luminex Flowsight flow cytometer. Apparent K_{D} for interaction of WAb0014 and huPAR was calculated using non-linear regression curve fitting analysis function in GraphPad Prism software by plotting mean fluorescence intensity vs test concentration.

### (2) Results:

The study results demonstrated that WAb0014 exhibited an apparent K_{D} of 1-3 nM for cell surface huPAR (Error! Reference source not found.) which is comparable to WAb0014's binding affinity for recombinant hsuPAR in the antibody immobilized configuration. The data suggests that WAb0014 is likely to bind both hsuPAR and huPAR equivalently.

### 4. Example 4: Binding of WAb0014 to Mouse uPAR Protein

WAb0014 binds to human cell surface as well as cynomolgus suPAR proteins with nanomolar affinity. To further evaluate cross-species specificity of uPAR binding, WAb0006, WAb0008 and WAb0014 were tested for binding to mouse cell surface uPAR (muPAR) using flow cytometry on undifferentiated immortalized mouse podocytes that are known to endogenously express muPARs.

### a) Methods:

Gene expression of mouse uPAR in undifferentiated mouse podocyte cells was undertaken using commercial gene specific qPCR probes for mouse and human uPAR and normalized to 18S housekeeping gene expression using manufacturers RT-qPCR protocol (Thermofisher). Separately, mouse podocytes were incubated with 100 μg/mL concentration of anti-hsuPAR and Proteintech positive control anti-mouse uPAR antibodies at 4° C. for 1 hr. After 2 cycles of wash to remove any unbound antibody, cells were incubated with 10 μg/mL of a goat-anti-human-FITC or goat-anti-mouse-FITC or goat-anti-rabbit-FITC conjugated secondary antibody for 1 hr at 4° C. After washing any unbound secondary antibody, cell suspensions for each sample was gated for single cells and population mean fluorescence intensity was recorded and analyzed on a Luminex Flowsight flow cytometer. Mean fluorescence intensity for test samples was plotted in GraphPad Prism software and compared to positive control sample data.

### b) Results:

The study results demonstrated that undifferentiated mouse podocytes robustly and specifically express mouse uPAR gene (Error! Reference source not found.2). When compared to mean fluorescence intensity associated with binding of unstained or secondary antibody (hu-sec [human], mo-sec [mouse], rb-sec [rabbit]) controls, WAb0006, WAb0008, and WAb0014 binding exhibited comparable mean fluorescence intensity (Error! Reference source not found.) indicating absence of a specific binding signal. In contrast, the Proteintech positive control antibody exhibited a robust binding signal (>25-fold over negative controls) confirming presence of cell surface muPAR on mouse podocyte cell surface. The data suggests that WAb0006, WAb0008, and WAb0014 do not bind muPAR

### Example 5: Generation of an Anti-suPAR Monoclonal Antibody WAb0014

### a) Discovery of Initial Lead Mouse IgG, MA7-8 Antibody

Anti-human suPAR (anti-hsuPAR) antibodies were generated via a hybridoma approach at MedAbome Inc. (Error! Reference source not found.4).

Multiple rounds of immunization of full length recombinant hsuPAR protein into wild type mice were undertaken over a 2-month period. Serum was harvested to confirm high antibody titer followed by harvesting of spleen, isolation of single spleen cells and fusion of antibody producing spleen cells with immortal myeloma cells in 8-azaguanine containing cell culture media in the presence of polyethylene glycol to generate antibody producing hybridoma cells. Fused, viable cells were grown clonally in 96-well plates containing feeder cells from mouse peritoneum. Supernatant from clonal cells were screened using an ELISA for binding to target hsuPAR antigen followed by a confirmation testing against a published anti-hsuPAR antibody in a competitive ELISA. Clones with binding activity in both rounds of testing were rank ordered and top hits screened in a functional surface plasmon resonance (SPR) assay to determine antibody-antigen binding affinity

### (2) Results:

Top 9 antibody hits with highest affinity (<10 nM) were identified, and MA7-8 was selected as the lead antibody candidate (K_{D}=0.2 nM) for affinity maturation and humanization workflow.

### b) Humanization of MA7-8

Complementarity determining regions (CDR) from MA7-8 antibody were grafted with 4 known human VH and VL framework sequences each to generate sixteen unique single chain variable fragment (scFv) constructs and characterized for binding activity to hsuPAR target antigen. The most potent scFv graft construct (MA7-8 CDR, 1-39 VH and 1-46 VL) was identified and used in a tumbler affinity maturation workflow at Distributed Bio Inc. Approximately a billion antibody phage particles displaying unique CDR sequences were generated and progressively panned over 4 rounds of affinity enrichment. Phage particles containing periplasmic extracts were then captured on biosensors and evaluated in a biophysical binding assay using Octet HTX platform to identify 74 tight binding scFv's via measurement of target antigen (hsuPAR and cynomolgus uPAR) off-rates (k_{off}<0.001 s⁻¹) (Table 7). A second round of confirmatory biophysical screening identified 45 tight binding (hsuPAR and cynomolgus uPAR) scFv containing periplasmic extracts with k_{off}<0.001 s⁻¹) with at least 11 scFv clones displaying improved antigen off-rate compared to parental MA7-8 antibody as well as equal or higher % identity to human VH and VK gene sequences. A detailed structural activity relationship analysis of top scFv hits was undertaken to identify tight binding scFv's with favorable amino-acid selection profile in the VH and VL CDRs for reformatting into hIgGI monoclonal antibody. Reformatted antibodies were confirmed for target antigen activity using a Biacore 8K SPR system and material provided to the Sponsor for additional detailed activity characterization.

TABLE 7
Summary of Anti-suPAR scFv Biophysical Screening

| | cynoPLAUR- | Off-rate | cynoPLAUR-His Binders + |
|---|---|---|---|
| | His | Summary | Equivocal Binders |
| Binders | 590 | Tight | 575 |
| Equivocal | 2 | Medium | 13 |
| Binders | | | |
| No Binders | 35 | Weak | 4 |
| Total PPE | 627 | No binders | 35 |
| screened | | | |
| | | Sum | 627 |

| | rhuPAR- | Off-rate | rhuPAR-His Binders + |
|---|---|---|---|
| | His | Summary | Equivocal Binders |
| Binders | 602 | Tight | 45 |
| Equivocal | 4 | Medium | 465 |
| Binders | | | |
| No Binders | 21 | Weak | 95 |
| Total PPE | 627 | No binders | 21 |
| screened | | | |
| | | Sum | 627 |

| | |
|---|---|
| Weak | k_{off} (1/s) > 0.01 |
| Medium | 0.001 < k_{off}(1/s) < 0.01 |
| Tight | k_{off} (1/s) < 0.001 |

### 6. Example 6: Functional Assessment of WAb0014 Treatment on MDA and HK2 Cell Migration/Motility

Role of uPAR in cell migration has been previously described. A study was conducted to evaluate whether WAb0014 treatment could impact time-dependent migration/motility of human proximal tubular (HK2) and human breast cancer (MDA-MB231) in a scratch assay.

### a) Methods:

HK2 and MDA-MB231 cells were plated on clear, sterile 24-well tissue culture treated microplates (Corning) at 100 k and 250 k cells per well and incubated at 37° C. overnight. On the following day, cells were first visually inspected to ensure 100% confluency. Subsequently, a scratch in the center of each well was made using a BioTek Autoscratch instrumentation followed by a gentle wash with cell media to remove dislodged cells that may interfere with the assay. Scratched area was imaged using a Cytation5 (BioTek) plate imager/reader and recorded as T₀ time point. Cells were then treated with WAb0014 and control hIgG antibodies at 3 test concentrations (10/30/100 μg/mL) and plates returned to 37° C. incubator for 18-20 hr. At the end of incubation period, cells were washed once with complete media and imaged again to monitor changes to the scratched area resulting from cell migration/motility and recorded as T₁₈₋₂₀ hr timepoint. Cell migration/motility index was calculated by dividing area of scratch at the end of treatment by initial area of scratch and compared between various treatment conditions.

### b) Results:

In this study, WAb0014 treatment of HK2 and MDA-MB231 (Error! Reference source not found.5) cells for 18-20 hr did not qualitatively impact scratch area closure for either cell lines at any of the tested concentrations compared to untreated and negative control groups. The percent scratch area closure values in the assay were as follows: HK2-60%, 52%, 68%; and MDA-MB231-65%, 61%, and 56%, for untreated control, hIgG control, and WAb0014 experimental conditions, respectively. The data suggest that WAb0014 treatment does not impact HK2 and MDA-MB231 cell migration/motility.

Overall, the results suggest that WAL0921 treatment is not likely to impact uPAR mediated normal homeostatic function of wound healing and immune response that involves cellular migration of leukocytes, macrophages, and keratinocytes.

### Example 7: Functional Assessment of WAb0014 Treatment on MDA and HK2 Cell Proliferation/Viability

Role of uPAR in cell proliferation has been previously described. A study was conducted to evaluate whether anti-hsuPAR antibody WAb0014 treatment could impact proliferation and/or viability of human proximal tubular (HK2) and human breast cancer (MDA-MB231) cell lines that endogenously express cell surface huPAR

### a) Methods:

HK2 and MDA-MB231 cells were plated on black 96-well optically clear bottom microplates at varying cell densities (MDA-7.5/10 k cells per well; HK2-5/7.5/10 k cells per well) and incubated overnight at 37° C. Cells were then treated with increasing concentrations of WAb0014 (0.0002-100 μg/mL) or control (10 and 100 μg/mL) human IgG (hIgG) antibody and incubated at 37° C. for 72 hrs. At the end of treatment period, cell proliferation/viability was assessed on a Cytation5 (BioTek) plate imager/reader platform using Promega's Cell Titer Glo luminescence assay kit, that is based on quantitation of cellular ATP as an indicator of metabolically active/live cells, following manufacturer's protocol recommendation. Relative luminescence unit (RLU) was compared between treatment and control samples and plotted as bar-graph.

### b) Results:

In this study, WAb0014 treatment of HK2 (Error! Reference source not found.6) and MDA-MB231 (Error! Reference source not found.) cells for 72 hr did not impact cellular proliferation/viability of either cell lines at any of the tested concentrations and cell densities when compared to untreated and negative control groups.

### 8. Example 8: Functional Assessment of Effect of Anti-suPAR Antibody, PP13, Treatment on Stimuli-Mediated Regulation of Cell Surface uPAR

Cell surface regulation of uPAR is central to many physiologically relevant homeostatic processes such as cellular adhesion and migration, wound healing, and immune response to infection. A study was conducted to evaluate whether anti-suPAR antibody PP13, predecessor of WAb0014, would modulate pro-mitogenic stimuli (phorbol-13-myristic acid, PMA) induced changes in cell surface uPAR on an immortalized monocytic cell line (U-937) derived from human bone marrow.

### a) Methods:

U-937 cells were sourced from ATCC and cultured as per vendor instructions. On the day of passage, cells were plated in tissue culture 6-well plates and treated with 100 ng/ml PMA or vehicle for four days at 37° C. A set of wells treated with vehicle or PMA were also treated with 10 μg/mL anti-suPAR antibody PP13 and incubated at 37° C. for four days. At the end of the incubation period, cells were harvested, resuspended in flow cytometry assay buffer and maintained at 4° C. Cell suspensions were treated with 10 μg/mL PP13 antibody for 1 hr to stain cell surface uPAR. After 2 cycles of wash to remove any unbound antibody, cells were incubated with 10 μg/mL of a goat-anti-human-FITC conjugated secondary antibody for 1 hr at 4° C. After washing any unbound secondary antibody, cell suspensions for each of treatment conditions were gated for single cells and population mean fluorescence was analyzed on a Luminex Flowsight flow cytometer.

### b) Results:

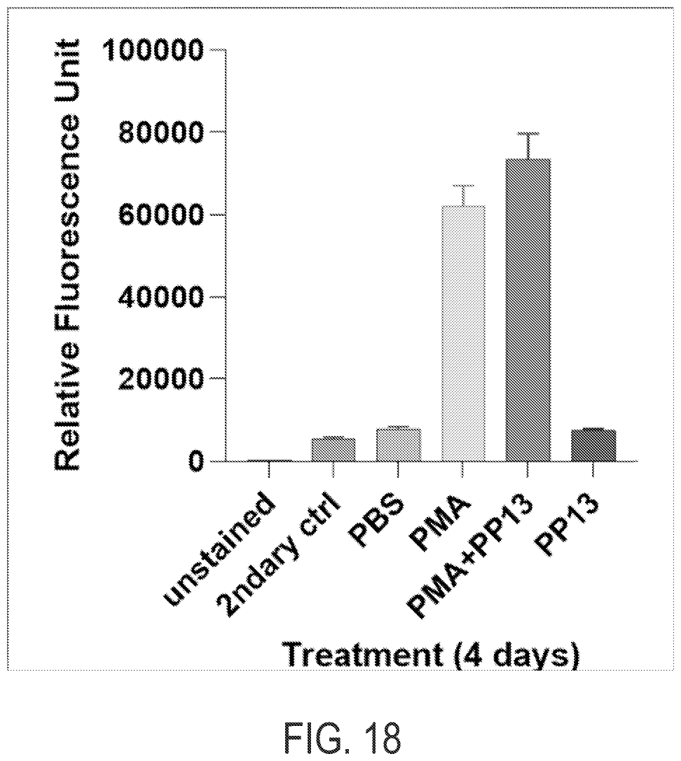

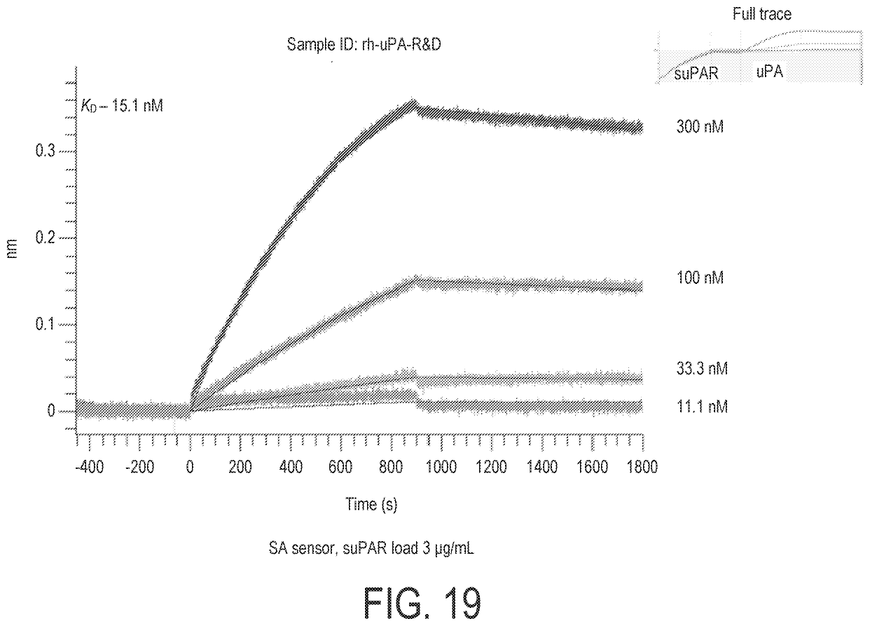

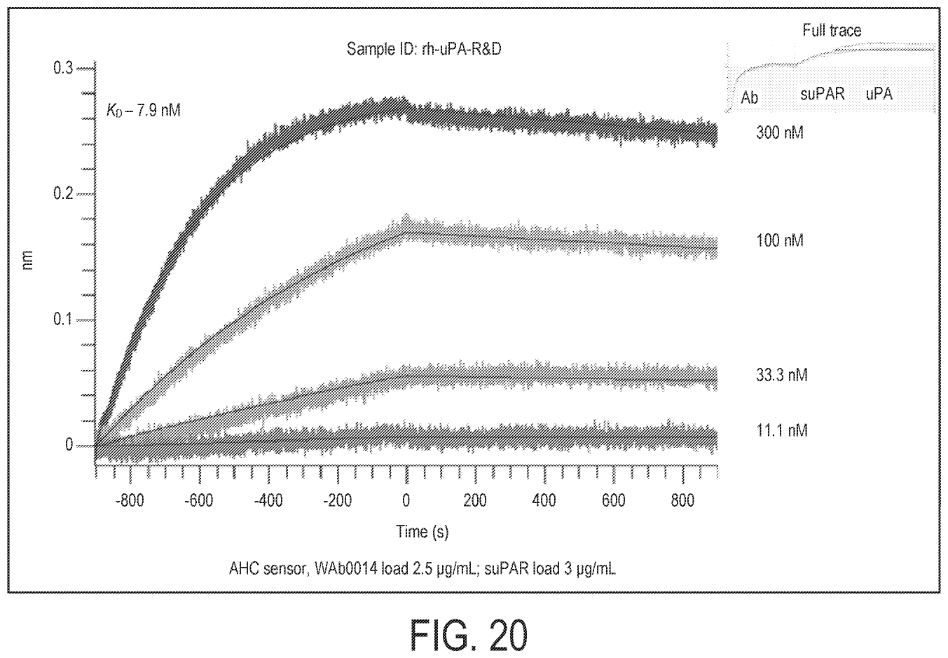

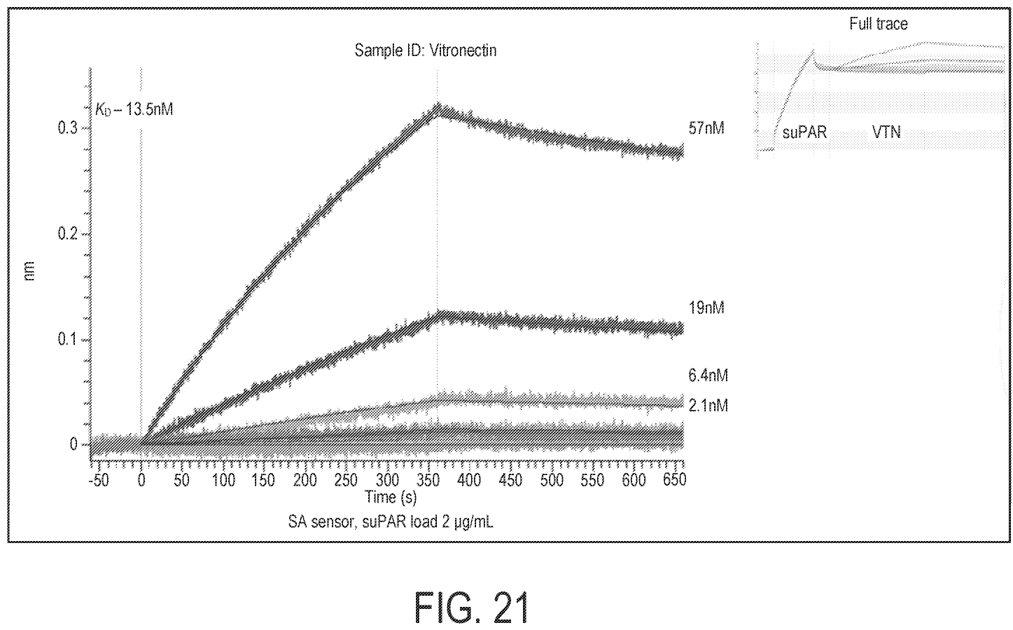

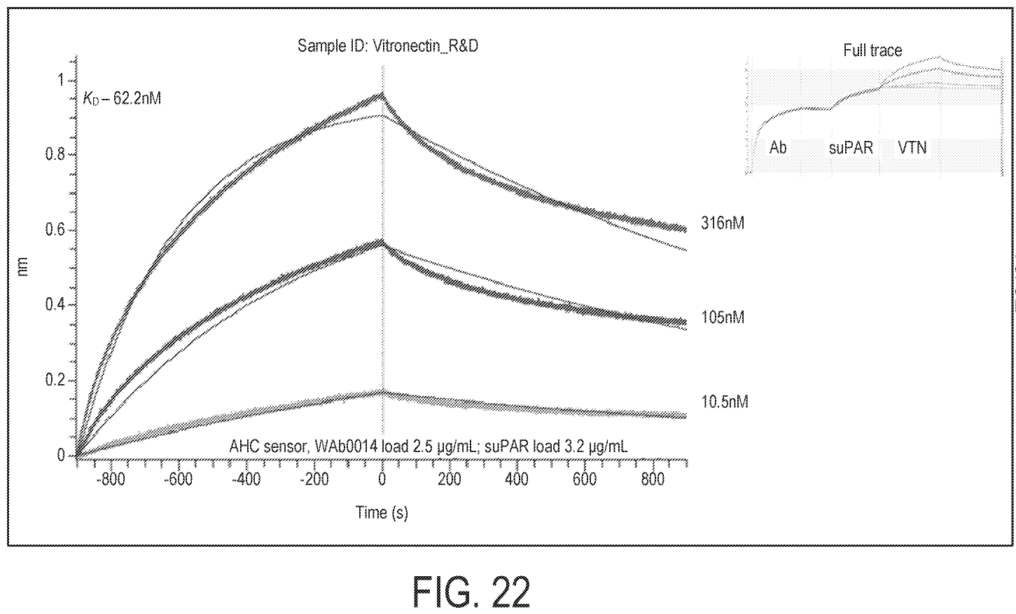

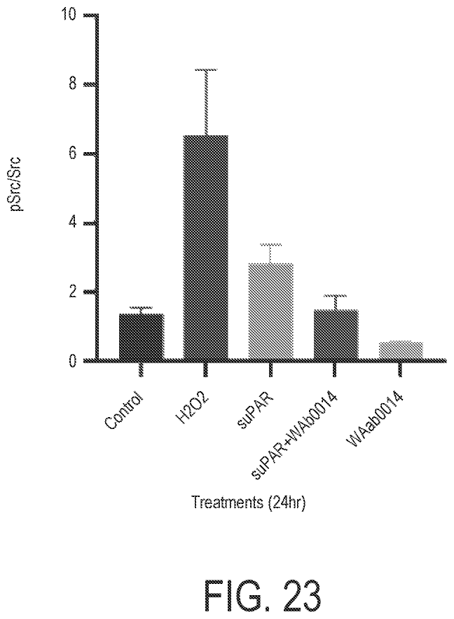

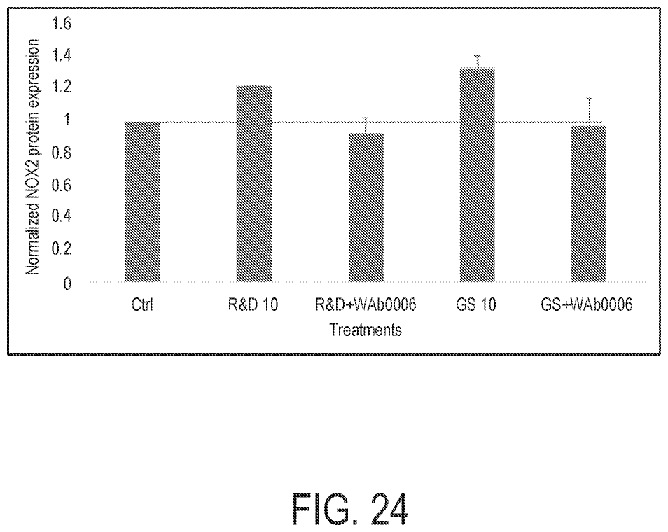

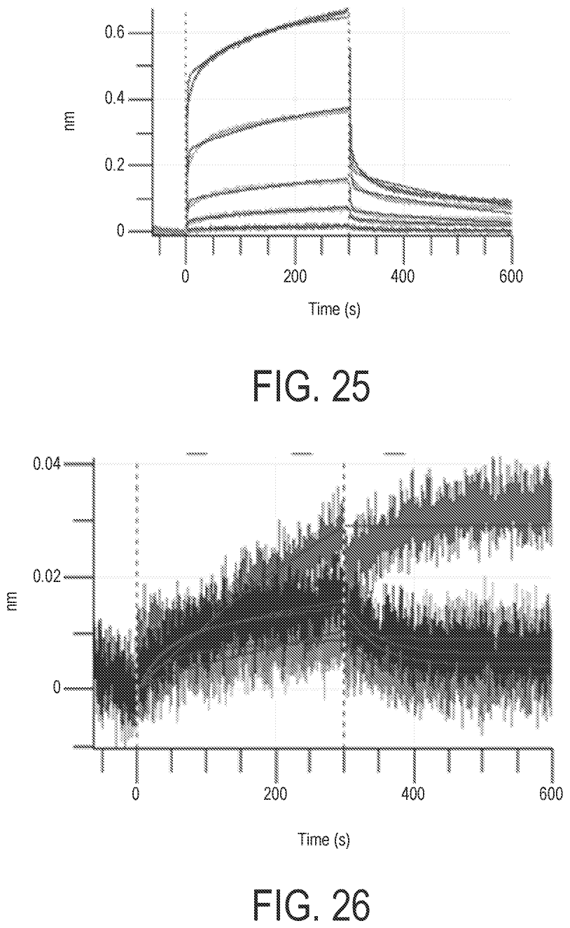

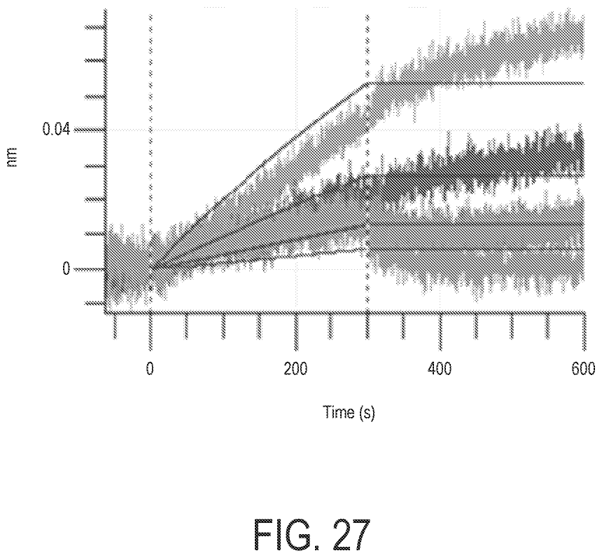

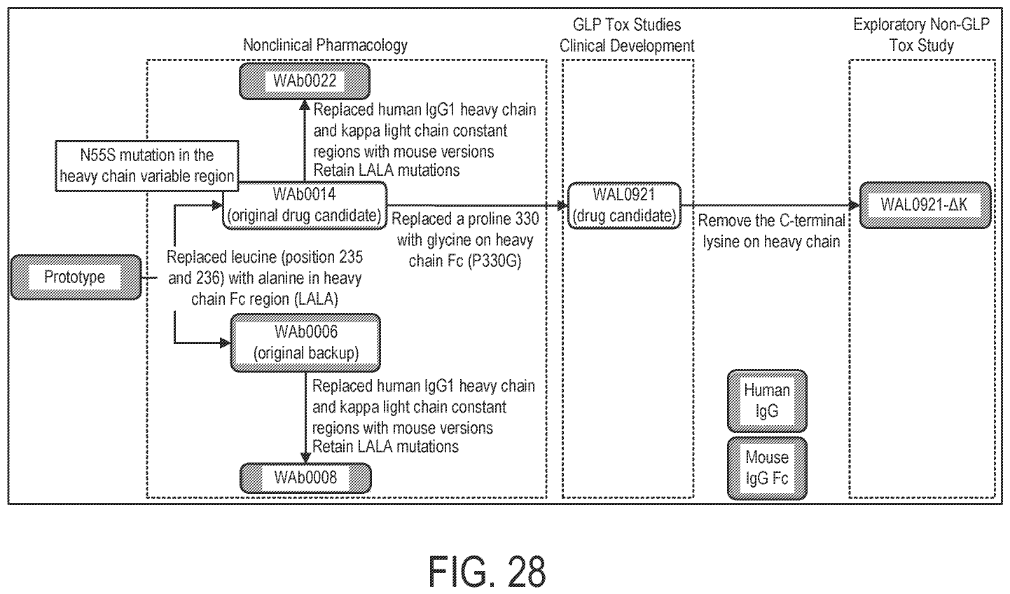

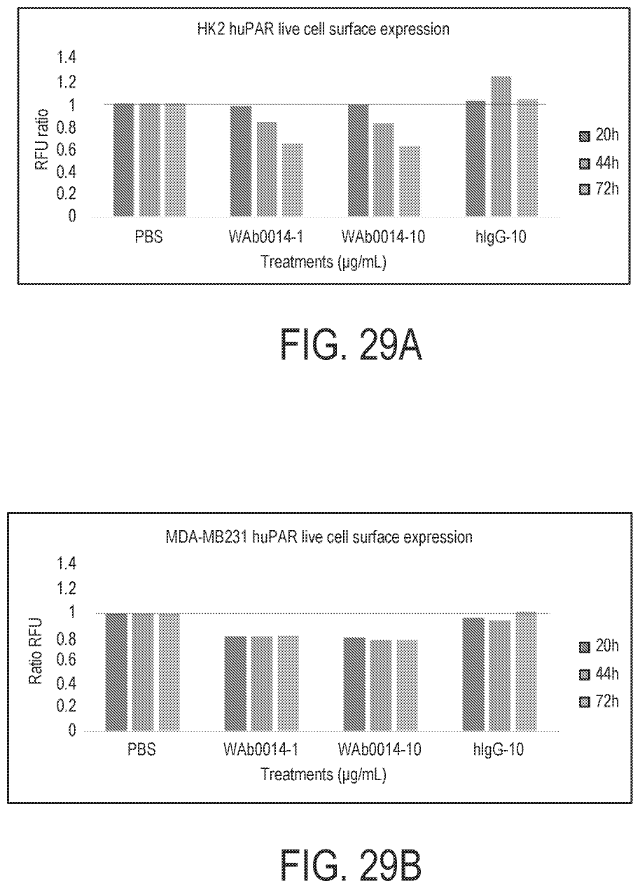

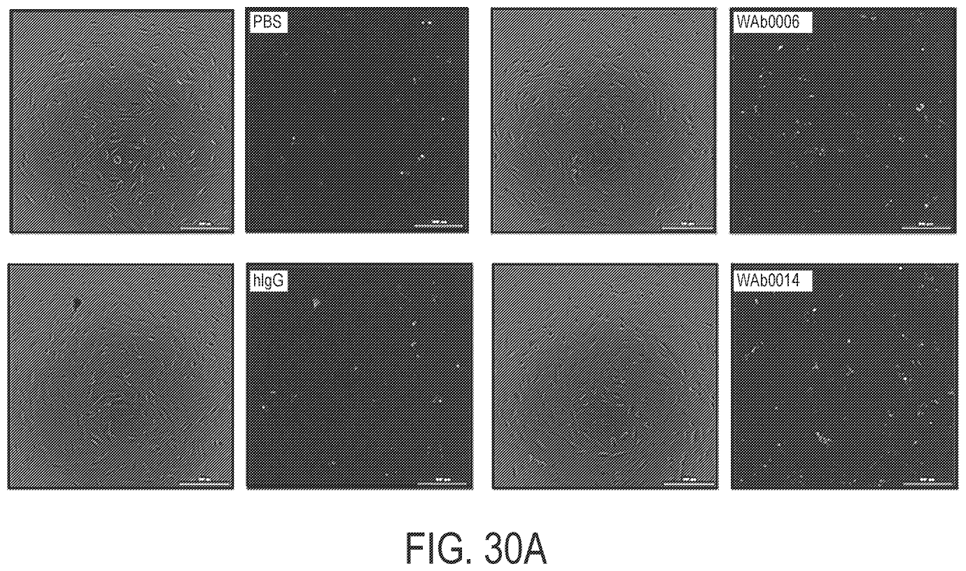

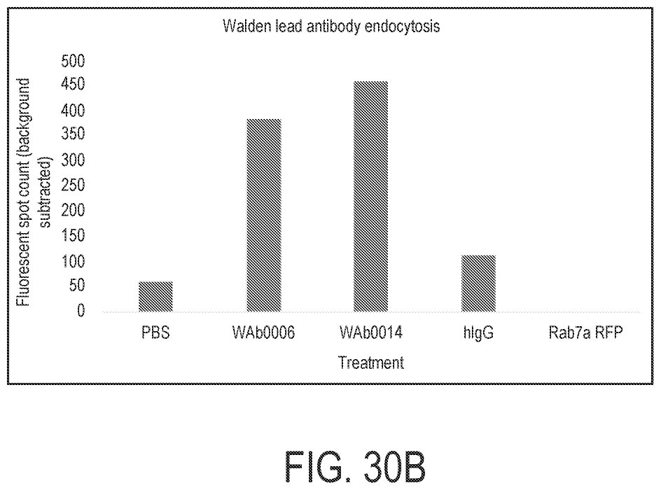

Vehicle only treated U-937 cells exhibit very low levels of cell surface uPAR protein expression compared to secondary antibody only stained controls (5500 vs 7800 mean RFU, respectively, lavender vs pink bars, FIG. 18). Treatment with 10 μg/mL PP13 did not impact basal cell surface uPAR expression in U-937 cells (7483 mean RFU, purple bar, FIG. 18). However, treatment with 100 ng/mL resulted in an 8-fold increase in cell surface uPAR expression (green bar, FIG. 18). Interestingly, co-treatment with PP-13 and PMA resulted in a 1.2-fold increase in cell surface uPAR (62277 vs 73567 mean RFU, respectively, green vs blue bars, FIG. 18).

Overall, the results suggest that treatment with clinical candidate WAL0921 antibody, a derivative of PP13 antibody used in the above experiments, is not likely to impair the immune response through upregulation of cell surface uPAR expression as a physiological response to pathogenic infection or general homeostasis, as described in the literature and is likely to be safe when administered clinically.

### 9. Example 9: Functional Assessment of WAb0014 Treatment on hsuPAR and uPA Binding Interaction

uPA is a serine protease that catalyzes the conversion of plasminogen to plasmin and is the endogenous ligand of uPAR. Plasmin is key to ECM remodeling necessary for cell adhesion, migration and implicated in metastatic cascades. Binding to uPAR focuses proteolysis to the cell surface and serves to inactivate the enzyme when complexed with soluble inhibitors, such as PAI-1. A study was conducted to evaluate whether WAb0014 binding to hsuPAR had an impact on hsuPAR-uPA binding interaction.

### a) Methods:

The interaction between hsuPAR and uPA proteins in the presence or absence of WAb0014 was assessed biophysically on an Octet Red96E platform.

For the hsuPAR-uPA interaction study, hsuPAR (R&D) protein, 3 μg/mL was initially immobilized on a streptavidin biosensor. After a wash step, uPA was added in a dose-dependent manner. Analyte global association (kₐ) and disassociation (kd) rates were calculated using in-built functions to determine binding affinity (K_{D}).

For the evaluation of WAb0014 on hsuPAR-uPA interaction study, WAb0014 protein (2.5 μg/mL) was initially immobilized on an AHC biosensor (Sartorius) followed by a wash step and treatment with 3.0 μg/mL hsuPAR (R&D) protein. After another wash step, uPA was added in a dose-dependent manner. Analyte global association (kₐ) and disassociation (k_{d}) rates were calculated using in-built functions to determine binding affinity (K_{D}).

### b) Results:

The study results show a strong nanomolar binding affinity between hsuPAR and uPA (K_{D}=15.1 nM, Error! Reference source not found.9), confirming the previously described interaction between the 2 proteins. A nanomolar affinity binding interaction between hsuPAR and uPA was also observed in the presence of WAb0014 (K_{D}=7.9 nM, Error! Reference source not found.0). The data indicate that binding of WAb0014 does not prevent endogenous binding interactions between hsuPAR and uPA suggesting non-overlapping binding sites between WAb0014 and uPA on hsuPAR.

Overall, the results suggest that uPA-uPAR mediated remodeling of ECM that is critical for both cell adhesion as well as migration under normal homeostasis is not likely to be impacted by WAL0921 treatment.

### 10. Example 10: Functional Assessment of WAb0014 Treatment on hsuPAR and Vitronectin Binding Interaction

Vitronectin is a well described ECM glycoprotein that is a natural ligand of uPAR. The uPAR-vitronectin interaction is implicated in mediating key cellular signaling pathways associated with cell migration and cell adhesion. A study was conducted to evaluate whether WAb0014 binding to hsuPAR had an impact on hsuPAR-vitronectin binding interaction.

### a) Methods:

The interaction between hsuPAR and vitronectin proteins in the presence or absence of WAb0014 was assessed biophysically on an Octet Red96E platform.

For the hsuPAR-vitronectin interaction study, hsuPAR (R&D) protein, 2 μg/mL was initially immobilized on a strepatvidin biosensor. After a wash step, vitronectin was added in a dose-dependent manner. Analyte global association (kₐ) and disassociation (kd) rates were calculated using in-built functions to determine binding affinity (K_{D}).

For the evaluation of WAb0014 on hsuPAR-vitronectin interaction study, WAb0014 protein (2.5 μg/mL) was initially immobilized on an AHC biosensor (Sartorius) followed by a wash step and treatment with 3.2 μg/mL hsuPAR (R&D) protein. After another wash step, vitronectin was added in a dose-dependent manner. Analyte global association (kₐ) and disassociation (kd) rates were calculated using in-built functions to determine binding affinity (K_{D}).

### b) Results:

The study results show a strong nanomolar binding affinity between hsuPAR and vitronectin (K_{D})=13.5 nM, Error! Reference source not found.) confirming the previously described interaction between the 2 proteins. Interestingly, a nanomolar affinity binding interaction between hsuPAR and vitronectin was also observed in the presence of WAb0014 (K_{D}=62.2 nM, Error! Reference source not found.) though presence of WAb0014 was associated with a small, 5-fold comparative reduction in affinity between hsuPAR and vitronectin proteins. The data indicate that binding of WAb0014 does not prevent endogenous binding interactions between hsuPAR and vitronectin suggesting non-overlapping binding sites between WAb0014 and vitronectin on hsuPAR.

Overall, the results suggest that vitronectin-uPAR mediated remodeling of ECM that is critical for both cell adhesion as well as migration under normal homeostasis is not likely to be impacted by WAL0921 treatment.

### 11. Example 11: Functional Assessment of WAb0014 Treatment on Src Kinase Phosphorylation and Reduction of Nox2 Protein Expression in Podocytes

### a) Src Kinase Phosphorylation Measurement

Src kinase has been previously described to play a role in integrin-focal adhesion physiology in mouse podocytes. Phospho-Src measurement was undertaken via an immunoblot assay described previously by the Dryer lab in differentiated mouse podocytes.

### (1) Methods:

Differentiated mouse podocyte cells between Days 10-14 differentiation and cultured on appropriate culture vessels (tissue culture compatible 6-well plate or 10 cm dishes) were used for the study. Cells were treated with 10 ng/ml hsuPAR (R&D or GenScript) with or without anti-hsuPAR antibody (R&D AF807 or WAb0014) for 24 hr and incubated in a 37° C./5% CO₂ incubator. At the end of the incubation period, cells were removed and collected as per standard procedure in the lab and lysed with a suitable cell lysis buffer supplemented with protease inhibitor. Clarified cell lysate were quantified for total protein levels, denatured further with a reducing SDS based loading dye and loaded on a SDS gel. Separated proteins post electrophoresis were transferred onto a suitable membrane (nitrocellulose or PVDF). Membranes were subsequently blocked with a blocking buffer, probed with primary antibodies for pSrc, tSrc and housekeeping proteins, washed, incubated with horseradish peroxidase conjugated secondary antibodies and visualized using a chemiluminescent substrate as per previously described procedures.

### (2) Results:

The results showed that WAb0014 treatment results in a reduction in hsuPAR induced Src kinase phosphorylation in immortalized mouse differentiated podocytes (Error! Reference source not found.).

### b) NOX2 Protein Expression Analysis

An increase in cytosolic reactive oxygen species has been suggested as an outcome of suPAR modulation via increase in Nox2 protein expression in mouse podocyte cells. A study was conducted to examine whether anti-hsuPAR antibody WAb0006 treatment could functionally prevent NOX2 protein expression in human podocytes.

Western blot based protein quantification analysis following incubation of differentiated human podocyte cells with either 10 ng/mL hsuPAR or 10 ng/mL hsuPAR+10 μg/mL WAb0006 for 24 hr was performed. At the end of treatment, cells were collected, and total protein harvested using standard reagents in presence of protease+phosphatase inhibitors. 20-30 μg of total protein from each of the treatment groups was run in an 8-10% BOLT Bis-Tris gel and transferred onto a polyvinylidene difluoride (PVDF) membrane. After blocking, membranes were incubated overnight in a NOX2+GAPDH housekeeping primary antibody cocktail at 4° C. Subsequently, primary antibody was washed, and membranes incubated in an appropriate secondary antibody cocktail (Licor) following which blots were developed on an Odyssey Licor imager. Effect of each treatment on NOX2 protein signal was quantified by normalization to housekeeping GAPDH protein signal for that group.

### (2) Results:

In this study, WAb0006 treatment resulted in a reduction in hsuPAR induced increase in NOX2 protein expression in immortalized human differentiated podocytes (Error! Reference source not found.4).

### 12. Example 12: Assessment of Immunologic Response Potential of WAL0921-ΔK

Antibody induced immune response is known to be mediated by interaction with Fc-gamma receptors. Immunologic response potential of WAL0921-ΔK was assessed biophysically on an Octet Red96E platform by measuring the binding affinity to Fc-gammaR1, high affinity Fc-gammaR2a and high affinity Fc-gammaR3a receptor recombinant proteins.

### a) Methods:

Biotinylated Fc-gamma proteins were procured from Acro Biosystems and immobilized on a streptavidin biosensor. Increasing concentrations of analyte WAL0921-ΔK were then applied, and responses measured. Binding affinity (K_{D}, nM) was calculated from the response rate constants as described above.

### b) Results:

The results showed that WAL0921-ΔK exhibited a very weak binding profile to Fc-gammaR1 isoform with an affinity of 1.75 μM (Error! Reference source not found.) and did not show a binding response to either of the high affinity Fc-gammaR2a or Fc-gammaR3a proteins (Error! Reference source not found.6 and Error! Reference source not found.).

Overall, the results suggest that WAL0921 is likely to be safe and tolerated in vivo and not likely to exhibit an immuno-modulatory effect and associated safety signal as described for immune-modulating biologics.

### Example 13: WAL0921, Anti-suPAR Antibody Lineage

WAL0921 (also herein referred to as WAb CD0014) is a novel antibody drug candidate comprised of humanized antibody variable domains and a human IgG1 isotype constant region. The development of WAL0921 progressed through a series of optimizations that improved the binding affinity of the monoclonal antibody to antigen (human suPAR). In all cases, each monoclonal antibody in the WAL0921 lineage binds with low nanomolar affinity to human suPAR.

The initial anti-suPAR antibody, WAb0014, was derived from a prototype via phage display technology and contains an N55S mutation in the heavy chain variable region to remove N-linked glycosylation liability as well as 2 mutations in the heavy chain Fc region (L235A and L236A). WAb0014 has been shown to have low nanomolar binding affinity to human suPAR and cell surface human uPAR (huPAR). In order to allow for in vivo efficacy studies in a transgenic mouse model that expresses human suPAR, WAb0014 was modified to contain the mouse Fc region and assigned the laboratory code WAb0022. In parallel, the anti-suPAR antibody, WAb0006, was also developed; it shares an identical Fc region as WAb0014, a highly similar Fab region but with 9 unique amino acid changes and a nanomolar binding affinity to human suPAR. In order to allow for in vivo efficacy studies in a transgenic mouse model that expresses human suPAR, WAb0006 was modified to contain the mouse Fc region and assigned the laboratory code WAb0008. Subsequently, a fourth mutation was introduced in the heavy chain Fc region of WAb0014 replacing proline at position 330 with a glycine (P330G). To enable an exploratory pilot toxicology study in cynomolgus monkeys, antibody WAL0921-ΔK was generated that shares the same target binding properties as WAL0921 except it does not contain the C-terminal lysine on the heavy chain; this deletion, which is commonly removed during antibody production to minimize the heterogeneity associated with endogenous C-terminal lysine clipping observed in host cell lines, does not affect the function of the antibody.

The development of WAL0921 progressed through a series of optimizations that improved upon the binding affinity of the monoclonal antibody to its target. Table 8 provides an overview of the anti-suPAR antibodies used during nonclinical development including the antibody name, description, the studies the antibody was used in/purpose of the study and where the data generated using each anti-suPAR antibody is provided within this meeting package. Preliminary investigations and the exploratory non-GLP toxicology study used WAL0921-ΔK which has the same target and properties of the development molecule, WAL0921 WAL0921 retains the C-terminal lysine on the heavy chain, which is not present in WAL0921-ΔK material. All GLP studies use WAL0921.

TABLE 8
Overview of Anti-suPAR Antibodies Used in Nonclinical Development

| Name | Description | Heavy chain | Light chain | Study Uses | Purpose |
|---|---|---|---|---|---|
| WAL0921 | Drug candidate | Humanized IgG1 with non- | Human kappa | GLP toxicology studies | IND enabling, |
| (WAb | | endogenous mutations - | | (including TK), GMP stability, | development |
| CD0014) | | N55S* L235A*, L236A*, | | bioanalytical method dev, | |
| | | P330G* | | Phase 1 SAD study | |
| WAL0921-ΔK | c-terminal | WAL0921 | Same VL as | Exploratory non-GLP toxicity | Inform GLP toxicity |
| | lysine | Without K448* | WAL0921 | study in cynomolgus monkeys | study doses, assess drug |
| | removed | | | | safety, identify putative |
| | | | | | biomarker signals |
| WAb0014 | Original | WAL0921 without P330G* | Same | Biophysical binding assays, cell | Early pharmacological |
| | candidate | | as WAL0921 | assays, suPAR isoform selectivity | profiling |
| | | | | assay, functional assay, PK in | |
| | | | | Tg32 mice | |
| WAb0006 | Original | WAL0921 without P330G* and | WAL0921 with | Initial lead - biophysical binding | Early pharmacological |
| | backup | with somatic mutations in CDR | Q27S* | assays, cell assay, suPAR isoform | profiling |
| | candidate | regions - N31S*, Y32H*, M34I*, | | selectivity assays | |
| | | H35Q*, S55N*, S57N*, A61D*, | | | |
| | | Y100T* | | | |
| WAb0022 | Mouse IgG2a | WAL0921 VH + mIgG2a Fc with | WAL0921 VL + | PK in C57 mice, NTS study in | In vivo efficacy |
| | version of | L235A* and L236A* | mouse kappa | transgenic mice | |
| | WAb0014 | | constant | | |
| WAb0008 | Mouse IgG2a | WAb0006 VH + mIgG2a Fc with | WAb0006 VL + | PK in C57 mice, NTS study in | In vivo efficacy |
| | version of | L235A* and L236A* | mouse kappa | transgenic mice | |
| | WAb0006 | | constant | | |

CDR = complementarity determining regions;
NA = Not applicable:
*Amino acid numberings

### Example 14: Assessment of Surface uPAR Endocytosis

The effect of WAb0014 incubation on cell surface uPAR protein expression was examined using flow cytometry.

### a) Methods:

Human proximal tubular cells (HK2) and human breast cancer cells (MDA-MB231) with robust cell surface uPAR protein expression were incubated with either 1 and 10 μg/mL WAb0014 or 10 μg/mL hIgG control antibody at 37° C. for 20, 44, and 72 hr, respectively. At the end of treatment period, cells were harvested post trypsinization, resuspended in flow cytometry assay buffer and maintained at 4° C. Cell suspensions were treated with 10 μg/mL WAb0014 for 1 hr to stain cell surface uPAR. After 2 cycles of wash to remove any unbound antibody, cells were incubated with 10 μg/mL of a goat-anti-human-FITC conjugated secondary antibody for 1 hr at 4° C. After washing any unbound secondary antibody, cell suspensions for each of WAb0014 test concentrations were gated for single cells and population mean fluorescence was analyzed on a Luminex Flowsight flow cytometer.

### b) Results:

The data suggest that WAb0014 treatment may result in a modest (≤20%), non-time dependent, decrease in cell surface huPAR protein expression in certain cell types (for ex MDA-MB231) whilst progressively decreasing surface huPAR protein expression (at least 37%) in other (HK2) cell types over a 72 hr time period (Error! Reference source not found.) with no change in surface uPAR expression at 24 hr in both HK2 cells and human podocytes (data not shown).

Overall, the partial (20-37%) cell-type and time-dependent manner reduction of cell surface uPAR in vitro suggest that WAL0921 treatment is not likely to impact key uPAR mediated homeostatic physiological processes of fibrinolysis, cell migration and chemotaxis, wound healing, and immune response.

### 15. Example 15: Assessment of Target Mediated Drug Disposition (TMDD

The Sponsor evaluated whether WAb0014 antibody would exhibit target mediated cellular endocytosis.

### a) Methods:

WAb0006, WAb0014, and hIgG control antibodies were conjugated with a commercial FITC dye conjugation kit (Thermofisher) according to manufacturer instructions. Undifferentiated human podocyte cells expressing cell surface uPAR were subsequently incubated with FITC conjugated antibodies at 10 μg/mL for 1 hr. Cells were washed twice with PBS to remove any unbound antibody and imaged and quantitated for cellular fluorescence in a Cytation5 (BioTek) imaging/plate reader system.

### b) Results:

In this study, WAb0006 and WAb0014 exhibited cellular endocytosis in undifferentiated human podocyte cells. Compared to PBS control and hIgG control wells that exhibit a mean cellular fluorescent signal of 75 and 100 RFU respectively, and WAb0006 and WAb0014 treatment resulted in mean cellular fluorescence signals of 400 and 450 RFU, respectively (Error! Reference source not found.).

### 16. Example 16: Effects of WAb0008 and WAL0921mu on Albuminuria Progression in the Murine NTS Model of Glomerular Nephritis

### A) Results

### (1) WAb008-NTS-001

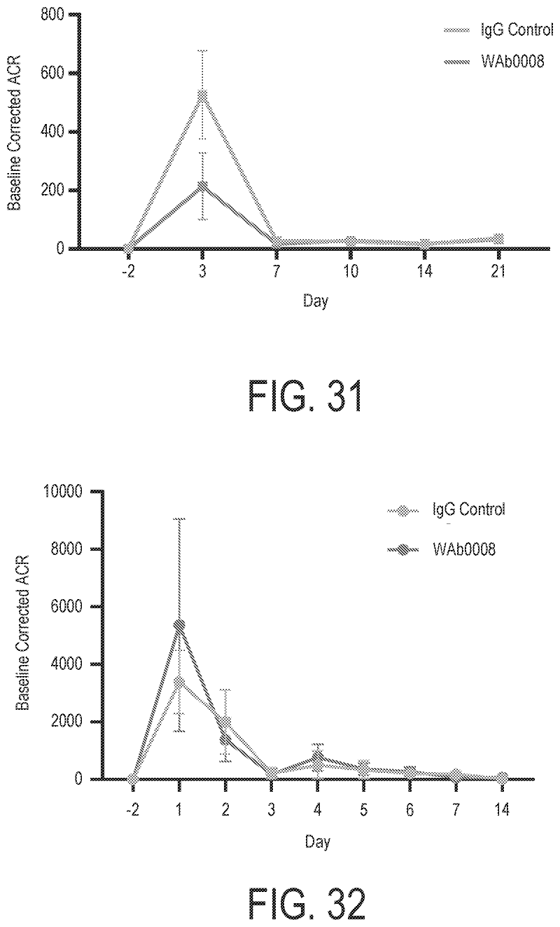

In this study, urine was collected on Day-2, 3, 7, 10 14, and 21. Urinary ACR levels were similar between both the IgG and WAb008 groups at baseline (98.0±48.2 and 60.1±14.4 respectively, Table 11). Peak albuminuria for both IgG and WAb0008 occurred on Day 3 (27679.3±7739.7 mg/g and 14240.6±8100.9 mg/g respectively), resulting in a greater than 500- and 200-fold increase over baseline values (Table 11 and FIG. 31). Though lowering over time, these values remained above baseline for all 21 days of study. On Day 3, IgG control mice had greater average albuminuria and fold change compared to WAb008 treated mice; the values between groups were similar on days 7, 10, 14, and 21. Though numerically lower than the control, Day 3, results were not statically significant by a two-way ANOVA using Graph Pad Prism ver.9. The lack of significance could be due to the heterogenous colony used (varying ages, sex, and diet). In analyzing these variables, diet appeared to be one that caused a large impact on response (data not shown), so for future studies all mice maintained a normal diet. Follow up studies (WAb008-NTS-002 and WAL0921mu-NTS-001) attempted to narrow the range of variables to better understand the potential effect of an anti-suPAR antibody in the NTS model. Also, additional sampling days were added to try and capture the full ACR response profile.

TABLE 11
WAb0008-NTS-001 Body Weight (average +/− SEM)

| WAb008- | | | | | | | | |
|---|---|---|---|---|---|---|---|---|
| NTS-001 | | Day −2 | | | | | | |
| body weight | | (baseline) | Day 0 | Day 2 | Day 3 | Day 7 | Day 10 | Day 14 |
| Control IgG | Weight (g) | 32.7 ± | 32.5 ± | 33.6 ± | 31.5 ± | 31.5 ± | 31.4 ± | 31.3 ± |
| | | 3.1 | 3.0 | 2.9 | 3.0 | 2.6 | 2.6 | 2.5 |
| | % of | 100.0 ± | 99.2 ± | 107.3 ± | 99.6 ± | 97.2 ± | 97.2 ± | 97.0 ± |
| | Baseline | 0.0 | 1.0 | 2.2 | 1.2 | 1.6 | 2.2 | 2.0 |
| WAb0008 | Weight (g) | 30.7 ± | 30.8 ± | 32.1 ± | 30.5 ± | 29.5 ± | 29.7 ± | 29.6 ± |
| | | 2.8 | 2.7 | 2.6 | 2.7 | 2.3 | 2.2 | 2.2 |
| | % of | 100.0 ± | 100.6 ± | 105.5 ± | 100.2 ± | 97.1 ± | 98.0 ± | 97.7 ± |
| | Baseline | 0.0 | 0.7 | 1.7 | 3.2 | 1.4 | 1.9 | 1.9 |

### (2) WAb008-NTS-002

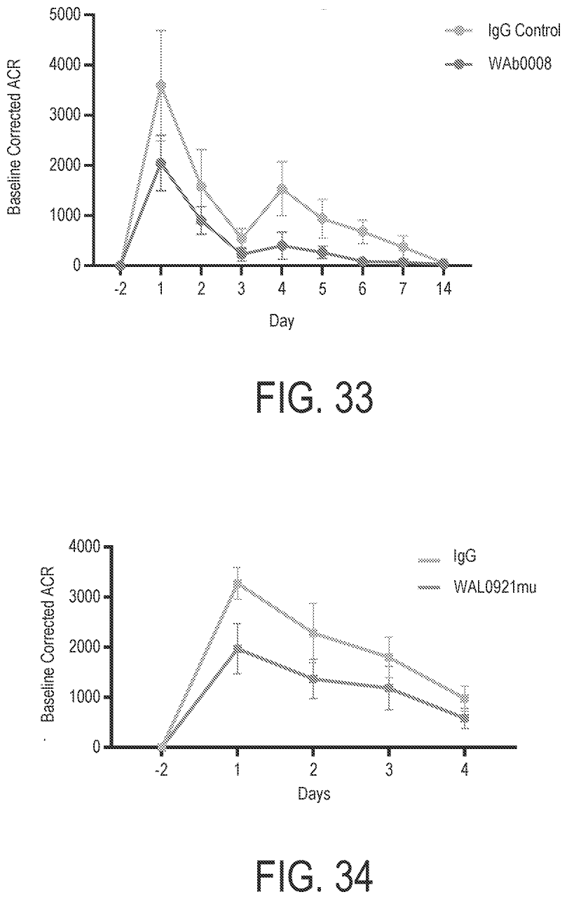

In addition to using Plaur−/− hsuPAR Tg mice, Plaur−/− lacking the hsuPAR TG were studied to understand if there was a difference in response to NTS if no suPAR was in circulation. There was also a single Plaur+/− hsuPAR Tg mouse added to each group as they were available. These mice did not show any difference in response compared to other genotypes but n=1 per group is limited. Urine was collected at baseline and then the first 7 days a and day 14 after NTS. The peak response in this study (and all subsequent studies) was within 24 hours of receiving the NTS challenge. Similar to WAb008-NTS-001, baseline ACR values were similar between the control and WAb008 treated mice, peak albuminuria reached 126662.0±26777.6 mg/g for the IgG and 121045.8±23936.9 mg/g for WAb008 treated mice (Table 12). This ACR increase translated to a significant fold change over baseline for each group as well (greater than and 2800, respectively Table 12, FIG. 33). Starting on day 1 and continuing through day 7, IgG treated mice had greater average ACR and fold over baseline compared to WAb008 treated mice. As in WAb008-NTS001, these results are variable and do not reach statistical significance. When separating out the Plaur−/− and Plaur−/− hsuPAR Tg response, in Plaur−/− mice, there is no difference in average ACR between the two treatment groups at any point in the study (FIG. 32). In Plaur−/− hsuPAR Tg, WAb008 treated mice had lower ACR values throughout study compared to control. Additionally, in the hsuPAR Tg mice there appears to be a second wave of ACR response beginning between days 3-4. This was only observed in the IgG control group, but not present in either the WAb008 group or Plaur−/− groups (FIG. 32, FIG. 33). This response could be suPAR dependent and warrants further evaluation.

TABLE 12
WAb0008-NTS-002 ACR (average +/− SEM)

| | | Day −2 | | | | | | | | |
|---|---|---|---|---|---|---|---|---|---|---|
| | | (baseline) | Day 1 | Day 2 | Day 3 | Day 4 | Day 5 | Day 6 | Day 7 | Day 14 |
| Control | ACR (mg/g) | 54.7 ± | 126662.0 ± | 47293.3 ± | 11291.3 ± | 33236.6 ± | 20608.5 ± | 14412.2 ± | 8188.3 ± | 1206.9 ± |
| IgG | | 14.2 | 26777.6 | 14361.0 | 3968.4 | 10391.5 | 7368.7 | 3585.3 | 3337.6 | 292.1 |
| | Fold Change over | 1.0 ± | 3561.3 ± | 1579.8 ± | 453.9 ± | 1223.1 ± | 790.1 ± | 537.2 ± | 318.4 ± | 46.6 ± |
| | Baseline | 0.0 | 837.9 | 549.2 | 158.5 | 418.1 | 306.7 | 175.0 | 170.8 | 13.4 |
| WAb0008 | ACR (mg/g) | 63.2 ± | 121045.8 ± | 57302.6 ± | 11527.0 ± | 25038.9 ± | 12652.8 ± | 6326.9 ± | 3022.7 ± | 1788.8 ± |
| | | 10.9 | 23936.9 | 13149.7 | 4517.9 | 10515.8 | 4303.3 | 2777.5 | 1417.3 | 754.2 |
| | Fold Change over | 1.0 ± | 2870.6 ± | 1113.7 ± | 211.9 ± | 517.8 ± | 296.1 ± | 139.0 ± | 70.5 ± | 38.4 ± |
| | Baseline | 0.0 | 983.9 | 353.3 | 101.0 | 230.7 | 100.8 | 58.2 | 32.6 | 14.2 |

### (3) WAL0921mu-NTS-001

WAL0921mu was tested in this study to evaluate if the response observed in the WAb008 studies was reproducible with murine construct of the Walden anti-suPAR antibody candidate (WAL0921). The length of this study was more acute compared to the other NTS studies to explore the initial response in ACR and to collect kidneys to understand and morphological changes that are caused by NTS in the first 4 days. To further reduce variability in this model, not only were averages matched between treatment groups as in past studies, but matched pairs were set up, “twin” mice with nearly identically variables (age, weight, sex, genotype, hsuPAR level, and Tg copy number) were established to facilitate direct comparisons between groups. One mouse from each pair was randomly allocated to group A, and the other group B.

Both Plaur+/− and Plaur−/− hsuPAR Tg genotypes were utilized in this study to explore the difference in disease progression when there is endogenous uPAR/suPAR, in addition to human suPAR. In Plaur+/− hsuPAR Tg mice, there was no difference in corrected baseline ACR between the IgG control group and WAL0921mu at any timepoint during the study (Table 13). However, there was also a stark increase in variability compared to the other NTS studies and the peak ACR at 24 hours was only 2,000-fold baseline (Table 13). Further evaluation of that genotype is warranted to understand the response observed in this model and to better understand the relationship when both human and mouse suPAR are present.

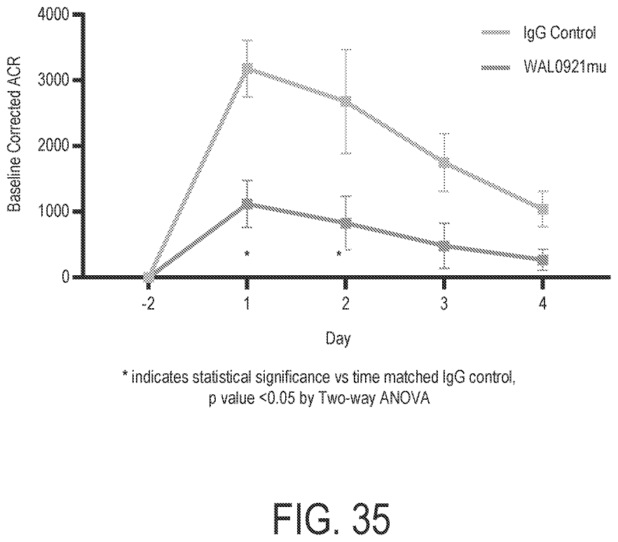

Similar to previous studies, stating on Day 1, WAL0921mu treated mice had lower ACR values (82522 5±6137.9 vs. 68795.9±8270 5 respectively Table 13, FIG. 34) and fold change over baseline study (˜3,000 fold compared to ˜2,000 fold) throughout the study. However, by 2-way ANOVA analysis, the response is not statistically significant when comparing the full data set. However, in the subset analysis of comparing the seven matched pairs with 1 copy of hsuPAR, WAL0921mu treated mice had significantly lower corrected ACR compared to IgG control on days 1 and 2 (FIG. 35).

### 17. Example 17: Nonclinical Study

Antibodies developed by Walden Biosciences were dosed in mice to evaluate their pharmacokinetic properties. The dosing and blood collections for these analyses were conducted at BRI Biopharmaceutical INC. There were 4 arms in the initial study, the analysis in this report only covers TA1-TA3 and excludes TA4. TA4 will not be included in this analysis as it pertains to a different program. WAb0014 was dosed in Tg32 mice while WAb0008 and WAb0022 were dosed in C57BL/6J mice. BRI was blinded as to what each treatment was. Exposure was analyzed using an in-house developed ELISA. Concentrations were interpolated using Excel and pharmacokinetic parameters were calculated using the PKSolver plug-in through Excel.

### a) Experimental Procedure

Antibody concentrations were assessed using an ELISA that was developed in house. Nunc Maxisorp plates were coated with 50 μL of recombinant human uPAR protein that was diluted to 1 μg/mL in PBS. Plates were stored at 4° C. overnight for the coating process. Coated plates were removed from 4C, decanted, and washed with 300 μL of 0.05% TWEEN 20 PBS per well using the Combi Liquid Dispenser from ThermoFisher. The plate was washed 3 times in total. After the final wash, 100 μL of SuperBlock (TBS) Blocking Buffer was added to each well for 1 hr at room temperature. Blocking solution was decanted and any remaining solution was aspirated. Once dry, the plates were covered with film and placed into the 4C until use. On days of sample analysis, serum plates and naïve mouse sera, used for standard curve buffer, were removed from the −80° C. freezer and thawed on ice for at least 30 minutes. The plates were periodically checked for uniform thawing. Coated protein plates were removed from the 4C and allowed to come to room temperature for at least 30 minutes. While the serum plate was thawing standard curves were prepared. Once samples were fully thawed, they were mixed and 4 μL was added to 396 μL of PBS for a 100× dilution in a 1 mL deep-well plate. The 100× plate was mixed and 50 μL of 100× was added and mixed into 700 μL of PBS for a final concentration of 1500×. The standard curve dilution buffer was prepared through diluting naïve mouse sera to 1500× in PBS. 50 μL of diluted sample was dispensed onto the protein coated plate along with the appropriate standard curve. The plate was incubated at room temperature, shielded from light, for at least 1 hour. During the incubation period, the secondary antibody solution was made at a dilution of 1:1000 in SuperBlock™ T20 (TBS) Blocking Buffer. WAb0014 utilized goat anti-human Fc HRP, while WAb0008 and WAb0022 used mouse IgG HRP-conjugated antibody. After the 1-hour incubation, the plate was decanted and washed three times. 50 μL of the secondary antibody solution was added to the plate and incubated at room temperature, shielded from light, for at least 1 hour. After the 1-hour incubation, the plate was decanted and washed three times. 50 μL of TMB was added to the plate and shielded from light 20 minutes for color development. IN HCl was prepared from diluting 5N HCl with deionized water. After the color development period, 50 μL of the IN HCl solution is added to the plate. The absorbance of the plate was read at 450 nM using the Cytation5 instrument from Agilent. Standard curve and concentrations were interpolated using Excel, and figures were made using GraphPad. If sample signals did not fit within the linear portion of the standard curve the experimental procedure was repeated at different dilution factors.

### b) Results

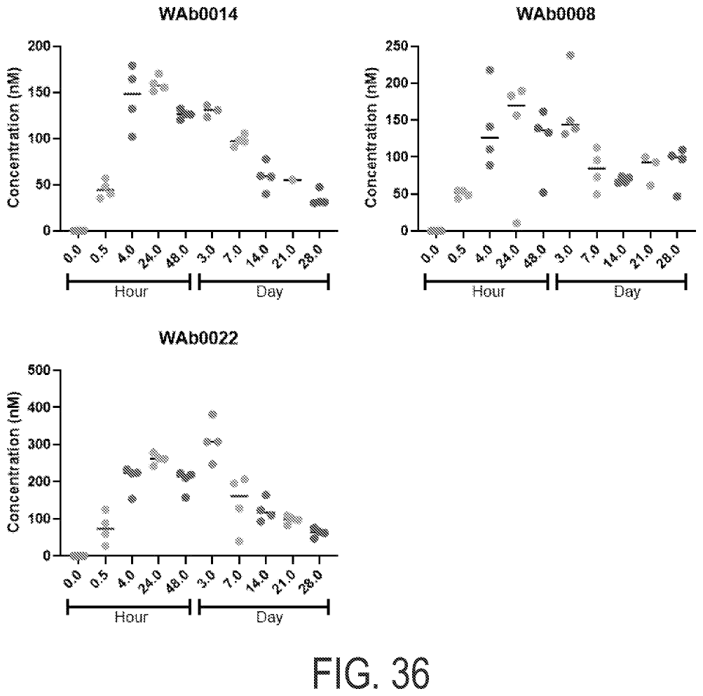

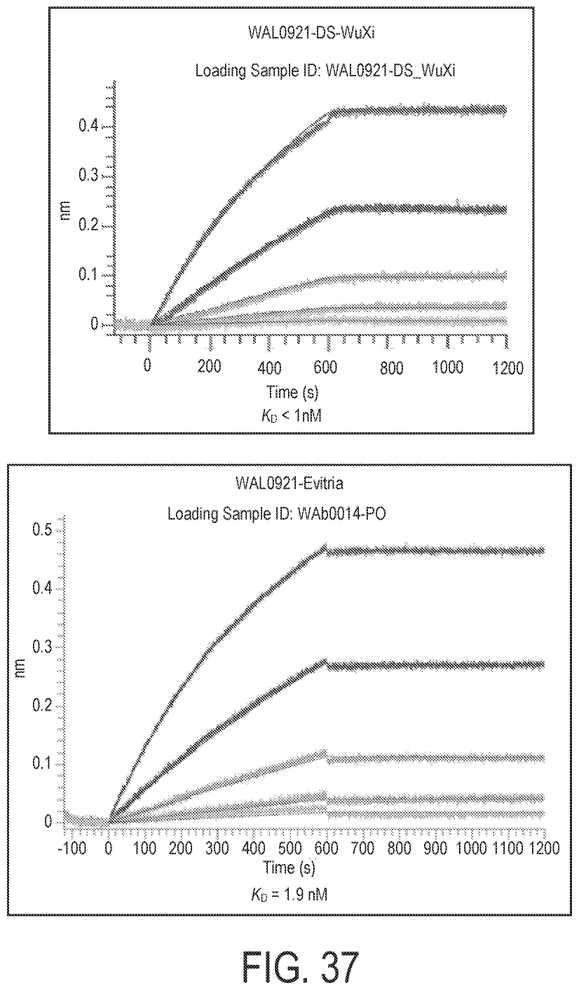

Figures and table of results can be found below. Results were analyzed using the publicly available Excel plug-in PKSolver. WAb0014 displayed a half-life of 346.4 hours and a Cmax of 185.6 nmol/L at 17.3 hours. WAb0014 samples up to and including day 28 were suitable for measurement except for animal 3 at 24 hr timepoint and animals 13, 14, 15 at 21-day timepoint due to signal at background at various dilutions. WAb0008 displayed a half-life of 349.04 hours and a Cmax of 114.54 nmol/L at 72 hours. WAb0008 samples up to and including day 28 were suitable for measurement except for animal 33 at the 24 hr and 21-day timepoint. WAb0022 displayed a half-life of 286.2 hours and a Cmax of 311 nmol/L at 72 hours. WA-b0022 samples up to and including day 28 were suitable for measurement except for animal 47 at 30m timepoint (FIG. 36).

### c) Conclusion

Antibodies developed by Walden Biosciences were dosed in mice to evaluate their pharmacokinetic properties WAb0014 in Tg32 mice, WAb0008 and WAb0022 in CS7BL/6J mice displayed long half-lives that were congruent with antibodies found in literature.

### Example 18

### 18. Example 18: Evaluation of the Hemolytic Potential and Plasma Compatibility Assessment of WAL0921 in Human Whole Blood In Vitro

The objective of this study was to evaluate in vitro whether WAL0921 may induce RBC hemolysis and/or RBC clumping as well as its compatibility with human plasma from human blood.

For assessment of RBC hemolysis, blood samples collected in lithium heparin anti-coagulant tubes from 3 individual human volunteers were incubated with either test (at final concentrations in blood of 750, 75 and 7.5 μg/mL) or control (vehicle control, positive control-saponin, negative control-0.9% saline) solutions. After centrifugation, the amount of hemoglobin in the supernatant was determined spectrophotometrically according to the method described by Cripps (1968). This was expressed as a percentage of the total blood hemoglobin. The test item was graded as non-hemolytic in human blood as defined in ASTM F756-17.

For assessment of RBC clumping, test solutions (at final concentrations in blood of 750, 75 and 7.5 μg/mL), vehicle control and negative control (0.9% saline) were added to whole blood samples from each volunteer to assess red blood cell clumping. No clumping of red blood cells was observed microscopically.

For assessment of compatibility with human plasma, test solutions (at final concentrations in blood of 750, 75 and 7.5 μg/mL), vehicle control, positive control (acetonitrile) and negative control (0.9% saline) were added to plasma samples for each volunteer. No precipitation was observed macroscopically for the test item, vehicle or negative control compared to the positive control.

In conclusion, WAL0921 formulations were found to be compatible with human whole blood and plasma up to the highest tested final assay concentration of 750 μg/mL.

### a) Introduction

The Sponsor has produced WAL0921, a monoclonal antibody for the treatment of proteinuric kidney diseases with elevated suPAR levels. The objective of this study was to evaluate in vitro whether WAL0921 may induce RBC hemolysis and/or RBC clumping as well as its compatibility with human plasma from human blood.

### b) Materials

### (1) Test Item

Identification: WAL0921
Batch (Lot) Number: 20220501
Expiration Date: 20 May 2023
Physical Description: Liquid
Purity: 97.4%
Concentration: 50.3 mg/mL
Storage Conditions: Temperature set to maintain −20° C.
Provided by: Sponsor

### (2) Vehicle Control

Identification: WAL0921 buffer
Ingredients: 20 mM Histidine buffer, 8% (w/v) Sucrose, 0.04% (w/v) PS80, pH 6.0
Storage Conditions: Temperature set to maintain 4° C.
Provided by: Test Facility

### (3) Test Item Characterization

The Sponsor has provided the Test Facility with documentation of the identity, strength, purity, composition and stability of the test item. A Certificate of Analysis has been provided for inclusion in this final report. Preparation of the vehicle control was as described in CRL Study Number 431373.

### (4) Reserve Sample

A reserve sample was not collected and maintained by the Test Facility.

### (5) Test Item Inventory and Disposition

Records of the receipt, distribution and storage of the test item were maintained. All unused WAL0921 and test item vehicle will be discarded prior to finalization of the study report.

### (6) Experimental Procedure

The study followed the procedure described in Charles River Laboratory Standard Operating Procedure SOPLAB717. WAL0921 was supplied as a 50.3 mg/mL solution. The final test item assay concentrations used were 750, 75 and 7.5 μg/mL. For assessment of hemolytic potential, a 20-fold higher concentration of test item was prepared in saline, and for clumping of red blood cells and precipitate formation, a 2-fold higher concentration of test item was prepared in saline.

### (7) Justification of Dose Selection

The highest test concentration of WAL0921 in this in vitro assay (750 μg/mL in the donor blood) was intended to exceed the anticipated maximal clinical blood concentration in the First-in-Human Phase 1 study in healthy subjects.

### (8) Control Matrix

Control blood samples were obtained from 3 healthy human volunteers, who attested to have refrained from any medication for at least 5 days prior to donating blood, according to Charles River Laboratory Standard Operating Procedures and Human Tissue (Scotland) Act 2006. Samples were collected by venipuncture into lithium heparin anticoagulant tubes and used on the day of the assay.

### (9) Other Materials

The following materials were obtained by Charles River. Chemicals were of analytical grade where available (Table 14). All materials were used within the expiry date stated by the manufacturer. Where no expiry date was provided by the supplier, a default expiry of 1 year (or 5 years for Sigma materials) from arrival was assigned to materials upon receipt by the Department of Immunology, Bioanalysis and Biomarkers, and these materials were used within the expiry date assigned to them.

TABLE 14
Materials

| Product Description | Supplier | Catalogue No. |
|---|---|---|
| Lithium Heparin Blood | BD | 367526 |
| Tubes | Vacutainer | |
| Saponin | Sigma | S4521 |
| Saline | Sigma | S8776 |
| Acetonitrile | VWR | 83640.320 |
| Methanol | VWR | 83638.320 |
| Modified Wrights Stain | Siemens | 6689653 |

### (10) Assessment of Hemolytic Potential

### (a) Incubations

For each donor, a triplicate set of labelled tubes was prepared with 50 μL of each test item solution (at final concentration 750, 75 and 7.5 μg/mL), vehicle control, saline (0.9% NaCl) or saponin (80 μg/L). Incubations were initiated by the addition of 950 μL of whole blood to each tube. Samples were mixed by inversion and placed in an incubator set to 37° C. for 1 hour. Blood samples from incubates were centrifuged at 3000×g for 10 minutes and the resultant supernatants were assessed for hemolysis as described below.

### (b) Measurement of Hemolysis

An aliquot of whole blood from each donor was analyzed for total hemoglobin concentration using an Advia 2120i haematology analyser. For each whole blood sample, a lysate was prepared by the addition of one part blood to two parts deionised water. The lysate was centrifuged at 3000×g for 10 minutes and the resultant supernatant from the lysate was analyzed for hemoglobin concentration. The percent hemolysis observed in each donor sample was calculated using the following formula:

$$Hemolysis in Lysate ( \% ) = \frac{Total Haemoglobin Conc . Of Lysate}{Total Haemoglobin Conc . Of Whole Blood} \times Blood / Water Diluting Factor ( 3 ) \times 100$$

Each of the lysate supernatant from the donor samples generated above was further diluted as described in Table 15 to create a mean standard curve from duplicate standard values.

TABLE 15
Lysate Supernatant Standard Curve

| Standard | Lysate Supernatant | d · H₂O | Dilution Factor |
|---|---|---|---|
| S1 | 50 μL | 1.5 mL | 0.032258 |
| S2 | 50 μL | 2.0 mL | 0.024390 |
| S3 | 50 μL | 2.5 mL | 0.019608 |
| S4 | 50 μL | 4.0 mL | 0.012346 |
| S5 | 20 μL | 5.0 mL | 0.003984 |

Dilution Factor = Volume of Lysate Supernatant/Total Volume

A hemolytic index for each of these diluted standards from each donor was calculated by multiplying the appropriate dilution factor in Table 15 with the known percentage of hemolysis for the donor computed above and dividing the obtained results by the initial blood/water diluting factor of 3 as per formula below:

Hemolytic Index=Dilution Factor (Table 15)×Hemolysis in Lysate (%)/3

The absorbance of each lysate supernatant standard (duplicate) generated above and the incubated supernatants (triplicate) from incubations were measured at 560 nm, 576 nm and 592 nm in a 96 well plate with a ABSPlus Spectramax 96 well plate spectrophotometer.

From the absorbance values, an absorbance function (A (f)) was calculated as described in Cripps C. M. (1968), and as shown below:

$$A ( f ) = 2 y - ( x + z )$$

Where: A (f)=absorbance function
x=absorbance at 560 nm
y=absorbance at 576 nm
z=absorbance at 592 nm

A standard curve was produced for each donor by plotting the hemolytic index of the various dilutions against the corresponding average absorbance functions using Microsoft® Office Excel and applying a linear regression curve fit. Hemolytic indices in the incubated supernatant samples were calculated by interpolating the determined absorbance values into the appropriate standard curve.

A hemolytic grade was assigned to the calculated hemolytic indices for the samples according to guidance in ASTM F756-17, and as noted in Table 16 below.

TABLE 16
Assignment of Hemolytic Grade

| Hemolytic Index | Hemolytic Grade |
|---|---|
| 0-2 | Non-hemolytic |
| 2-5 | Slightly Hemolytic |
| >5 | Hemolytic |

### (11) Clumping of Red Blood Cells in Whole Blood

For each donor, 0.3 mL of lithium heparinised whole blood was thoroughly mixed with an equal volume of either test solution (at final concentration 750, 75 and 7.5 μg/mL), vehicle control or control saline. The contents of each tube were spread onto individual slides and left to dry, before being fixed with methanol. Slides were then stained with Modified Wright's Stain using a Hema-Tek 3000 staining machine and examined microscopically.

### (12) Precipitate Formation in Plasma

For each donor, plasma was isolated by centrifuging ca. 5 mL of whole blood at 3000× g for 10 minutes. For each donor, 0.3 mL plasma was mixed thoroughly with an equal volume of either test solution (at final concentration 750, 75 and 7.5 μg/mL), vehicle control, positive control (acetonitrile) or negative control (saline). After 2 minutes the plasma was visually assessed for overt flocculation, precipitation, and coagulation (presence of white cloudiness). After 5 minutes, the tubes were centrifuged at 3000×g for 5 minutes and examined for precipitation.

### (13) Scoring of Red Blood Cells Clumping Reaction and Plasma Precipitation

The following were used to score results: NEG No reaction observed
POS Reaction observed

Saline samples were used as a negative control i.e. no reaction observed and for plasma precipitation, acetonitrile was used as the positive control (in Section 7.6), i.e. reaction observed.

(14) Computerized systems

Critical computerized systems used in the study are listed in Table 17. All computerized systems used in the conduct of this study have been validated for the required use.

TABLE 17
Computerized Systems

| System Name | Description |
|---|---|
| M-Files | Reporting and collection of 21 CFR Part 11 |
| | compliant signatures |
| Docusign ™ | Collection of 21 CFR Part 11 compliant signature |
| Dispense | Inventory Management |
| Advia 2120i | Haematology data acquisition and processing |
| SoftMaxPro GxP | Spectrophotometry 96-well platereader data acquisition |
| 7.1.2 | |

### c) Analysis

Statistical analyses were limited to using Microsoft Excel® and included descriptive statistics such as arithmetic means, standard deviations (SD), coefficient of variance (CV) and percentage difference.

### d) Results

### (1) Assessment of Hemolytic Potential

Absorbance results for lysate supernatant standards and incubated supernatant samples for each donor are shown in Table 18 to Table 20.

TABLE 18
Donor A: ABSPlus Spectramax Plate Reader Absorbance

| | | Absorbance | | | A (f) |
|---|---|---|---|---|---|
| | | 560 nm | 576 nm | 592 nm | 2y − |
| Sample | | (x) | (y) | (z) | (x + z) |
| Standards | S1 | 0.257 | 0.424 | 0.104 | 0.487 |
| | S1 | 0.264 | 0.434 | 0.109 | 0.495 |
| | S2 | 0.211 | 0.338 | 0.091 | 0.374 |
| | S2 | 0.206 | 0.333 | 0.090 | 0.369 |
| | S3 | 0.164 | 0.256 | 0.078 | 0.269 |
| | S3 | 0.169 | 0.265 | 0.078 | 0.284 |
| | S4 | 0.122 | 0.182 | 0.064 | 0.178 |
| | S4 | 0.124 | 0.187 | 0.065 | 0.185 |
| | S5 | 0.062 | 0.080 | 0.046 | 0.052 |
| | S5 | 0.068 | 0.087 | 0.049 | 0.058 |

| Saline | 0.113 | 0.129 | 0.088 | 0.056 |
|---|---|---|---|---|
| Negative Control | 0.112 | 0.125 | 0.087 | 0.052 |
| | 0.115 | 0.126 | 0.088 | 0.048 |
| Saponin^{A} | 0.131 | 0.197 | 0.068 | 0.195 |
| Positive Control | 0.144 | 0.219 | 0.073 | 0.220 |
| | 0.137 | 0.206 | 0.072 | 0.202 |
| WAL0921 | 0.103 | 0.116 | 0.080 | 0.048 |
| 750 μg/mL^{B} | 1.102 | 1.016 | 0.932 | −0.003 |
| | 0.119 | 0.139 | 0.088 | 0.071 |
| WAL0921 | 0.120 | 0.145 | 0.085 | 0.086 |
| 75 μg/mL^{B} | 0.118 | 0.138 | 0.088 | 0.070 |
| | 0.113 | 0.132 | 0.083 | 0.068 |
| WAL0921 | 0.111 | 0.132 | 0.081 | 0.071 |
| 7.5 μg/mL^{B} | 0.096 | 0.112 | 0.073 | 0.054 |
| | 0.107 | 0.127 | 0.079 | 0.069 |
| Vehicle | 0.116 | 0.136 | 0.086 | 0.070 |
| Control | 0.113 | 0.131 | 0.085 | 0.065 |
| | 0.237 | 0.271 | 0.203 | 0.103 |

^{A}= Saponin was analyzed at a 1 in 200 dilution, all other samples were analyzed neat
^{B}= Final assay concentrations

TABLE 19
Donor B: ABSPlus Spectramax Plate Reader Absorbance

| | | Absorbance | | | A (f) |
|---|---|---|---|---|---|
| | | 560 nm | 576 nm | 592 nm | 2y − |
| Sample | | (x) | (y) | (z) | (x + z) |
| Standards | S1 | 0.253 | 0.407 | 0.098 | 0.462 |
| | S1 | 0.259 | 0.416 | 0.100 | 0.473 |
| | S2 | 0.207 | 0.327 | 0.086 | 0.362 |
| | S2 | 0.197 | 0.310 | 0.083 | 0.340 |
| | S3 | 0.161 | 0.249 | 0.074 | 0.262 |
| | S3 | 0.177 | 0.272 | 0.078 | 0.289 |
| | S4 | 0.125 | 0.184 | 0.064 | 0.178 |
| | S4 | 0.126 | 0.185 | 0.065 | 0.178 |
| | S5 | 0.065 | 0.083 | 0.046 | 0.055 |
| | S5 | 0.064 | 0.080 | 0.046 | 0.051 |

| Saline | 0.074 | 0.083 | 0.060 | 0.032 |
|---|---|---|---|---|
| Negative Control | 0.074 | 0.083 | 0.062 | 0.030 |
| | 0.073 | 0.082 | 0.061 | 0.031 |
| Saponin^{A} | 0.142 | 0.211 | 0.071 | 0.210 |
| Positive Control | 0.166 | 0.250 | 0.077 | 0.258 |
| | 0.138 | 0.204 | 0.069 | 0.201 |
| WAL0921 | 0.067 | 0.073 | 0.058 | 0.020 |
| 750 μg/mL^{B} | 0.069 | 0.074 | 0.063 | 0.015 |
| | 0.065 | 0.070 | 0.059 | 0.015 |
| WAL0921 | 0.070 | 0.077 | 0.061 | 0.024 |
| 75 μg/mL^{B} | 0.067 | 0.072 | 0.058 | 0.019 |
| | 0.065 | 0.070 | 0.056 | 0.020 |
| WAL0921 | 0.068 | 0.074 | 0.059 | 0.021 |
| 7.5 μg/mL^{B} | 0.068 | 0.074 | 0.058 | 0.023 |
| | 0.069 | 0.074 | 0.060 | 0.020 |
| Vehicle Control | 0.097 | 0.126 | 0.066 | 0.090 |
| | 0.073 | 0.079 | 0.066 | 0.018 |
| | 0.071 | 0.077 | 0.061 | 0.022 |

^{A}= Saponin was analyzed at a 1 in 200 dilution, all other samples were analyzed neat
^{B}= Final assay concentrations

TABLE 20
Donor C: ABSPlus Spectramax Plate Reader Absorbance

| | | Absorbance | | | A (f) |
|---|---|---|---|---|---|
| | | 560 nm | 576 nm | 592 nm | 2y − |
| Sample | | (x) | (y) | (z) | (x + z) |
| Standards | S1 | 0.223 | 0.363 | 0.090 | 0.413 |
| | S1 | 0.262 | 0.432 | 0.101 | 0.501 |
| | S2 | 0.203 | 0.330 | 0.086 | 0.371 |
| | S2 | 0.199 | 0.322 | 0.085 | 0.361 |
| | S3 | 0.171 | 0.271 | 0.077 | 0.294 |
| | S3 | 0.173 | 0.275 | 0.077 | 0.300 |
| | S4 | 0.126 | 0.189 | 0.065 | 0.187 |
| | S4 | 0.122 | 0.183 | 0.062 | 0.183 |
| | S5 | 0.064 | 0.083 | 0.046 | 0.056 |
| | S5 | 0.064 | 0.083 | 0.046 | 0.055 |

| Saline | 0.234 | 0.353 | 0.111 | 0.362 |
|---|---|---|---|---|
| Negative Control | 0.241 | 0.365 | 0.117 | 0.373 |
| | 0.252 | 0.381 | 0.124 | 0.386 |
| Saponin^{A} | 0.142 | 0.218 | 0.072 | 0.221 |
| Positive Control | 0.145 | 0.223 | 0.071 | 0.229 |
| | 0.149 | 0.230 | 0.072 | 0.239 |
| WAL0921 | 0.229 | 0.347 | 0.116 | 0.348 |
| 750 μg/mL^{B} | 0.247 | 0.372 | 0.124 | 0.374 |
| | 0.191 | 0.278 | 0.105 | 0.260 |
| WAL0921 | 0.199 | 0.290 | 0.108 | 0.273 |
| 75 μg/mL^{B} | 0.201 | 0.291 | 0.112 | 0.268 |
| | 0.172 | 0.249 | 0.093 | 0.233 |
| WAL0921 | 0.203 | 0.301 | 0.105 | 0.294 |
| 7.5 μg/mL^{B} | 0.185 | 0.269 | 0.098 | 0.255 |
| | 0.202 | 0.299 | 0.105 | 0.291 |
| Vehicle Control | 0.200 | 0.289 | 0.108 | 0.270 |
| | 0.189 | 0.274 | 0.102 | 0.257 |
| | 0.133 | 0.174 | 0.086 | 0.129 |

^{A}= Saponin was analyzed at a 1 in 200 dilution, all other samples were analyzed neat
^{B}= Final assay concentrations

The hemoglobin concentrations in whole blood and lysate supernatants are shown in Table 21. These values were used to calculate the percentage of hemolysis in the lysate supernatant standards and are presented together with the absorbance function for each lysate supernatant standard (Table 21).

TABLE 21
Standard Curves for Human Blood Donors

| | | | | | | Mean A |
|---|---|---|---|---|---|---|
| | | Dilution | Hemolytic | A (f) | | (f) |
| Donor | Standard | Factor | Index | Replicate 1 | Replicate 2 | (y) |
| A | S1 | 0.032258 | 1.06 | 0.487 | 0.495 | 0.491 |
| | S2 | 0.024390 | 0.80 | 0.374 | 0.369 | 0.372 |
| | S3 | 0.019608 | 0.64 | 0.269 | 0.284 | 0.277 |
| | S4 | 0.012346 | 0.41 | 0.178 | 0.185 | 0.181 |
| | S5 | 0.003984 | 0.13 | 0.052 | 0.058 | 0.055 |
| | | Hb concentration of Whole Blood = 137 g/L | | | | |
| | | Hb concentration of Lysate Supernatant = 45 g/L | | | | |
| | | Hemolysis in Lysate = 98.5% | | | | |
| B | S1 | 0.032258 | 1.03 | 0.462 | 0.473 | 0.468 |
| | S2 | 0.024390 | 0.78 | 0.362 | 0.340 | 0.351 |
| | S3 | 0.019608 | 0.63 | 0.262 | 0.289 | 0.276 |
| | S4 | 0.012346 | 0.39 | 0.178 | 0.178 | 0.178 |
| | S5 | 0.003984 | 0.13 | 0.055 | 0.051 | 0.053 |
| | | Hb concentration of Whole Blood = 138 g/L | | | | |
| | | Hb concentration of Lysate Supernatant = 44 g/L | | | | |
| | | Hemolysis in Lysate = 95.7% | | | | |
| C | S1 | 0.032258 | 0.94 | 0.413 | 0.501 | 0.457 |
| | S2 | 0.024390 | 0.71 | 0.371 | 0.361 | 0.366 |
| | S3 | 0.019608 | 0.57 | 0.294 | 0.300 | 0.297 |
| | S4 | 0.012346 | 0.36 | 0.187 | 0.183 | 0.185 |
| | S5 | 0.003984 | 0.12 | 0.056 | 0.055 | 0.056 |

Hb concentration of Whole Blood = 151 g/L
Hb concentration of Lysate Supernatant = 44 g/L
Hemolysis in Lysate = 87.4%
Hemolytic Index = (Dilution Factor × Hemolysis in lysate)/3

The percentage of hemolysis and average absorbance function for each of the lysate supernatant standards were used to construct a hemolysis standard curve for each donor. Absorbance values for Donor C were found to be higher than both Donor A and Donor B. As the standards and controls were also relatively high, the calculated hemolytic index value after WAL0921 treatment is not higher than those for the other donors.

The standard curves were subsequently used to calculate the hemolytic index (% hemolysis) of incubated supernatant samples. The mean (n=3) hemolytic index for each human donor is shown in Table 22, together with an overall sample mean of the results from the three donors.

The saponin positive control was graded hemolytic with a mean hemolysis index of 94.2% The test item at final concentrations of 750, 75 and 7.5 μg/mL, the vehicle control and negative control were all found to be non-hemolytic (Table 22).

TABLE 22
Haemolytic Index

| | Hemolytic Index | | | | Hemolytic |
|---|---|---|---|---|---|
| Sample | Donor A | Donor B | Donor C | Mean | Grade |
| Saline | 0.1 | 0.1 | 0.7 | 0.3 | Non- |
| Negative Control | | | | | hemolytic |
| Saponin | 92.2 | 99.7 | 90.7 | 94.2 | Hemolytic |
| Positive Control | | | | | |
| WAL0921 | 0.1 | 0.0 | 0.7 | 0.3 | Non- |
| 750 μg/mL^{A} | | | | | hemolytic |
| WAL0921 | 0.2 | 0.1 | 0.5 | 0.2 | Non- |
| 75 μg/mL^{A} | | | | | hemolytic |
| WAL0921 | 0.2 | 0.1 | 0.6 | 0.3 | Non- |
| 7.5 μg/mL^{A} | | | | | hemolytic |
| Vehicle | 0.2 | 0.1 | 0.4 | 0.2 | Non- |
| | | | | | hemolytic |

^{A}= Final assay concentrations

The data indicated that WAL0921 had a low hemolytic index and therefore classified as non-hemolytic in human blood at final assay concentrations of 750, 75 and 7.5 μg/mL.

### (2) Clumping of Red Blood Cells in Whole Blood

Clumping of red blood cells (RBCs) in whole blood was scored either POS (positive) or NEG (negative). The RBC clumping assessment found the test solutions, vehicle control and negative control did not induce clumping of red blood cells in any of the 3 donor blood samples.

### (3) Precipitate Formation in Plasma

Plasma precipitation was scored either POS or NEG. The plasma compatibility assessment showed precipitation for all 3 donors in the positive control (acetonitrile) only. When compared to the vehicle and negative controls, no visual precipitation was observed in the test solutions prior to, or following, centrifugation in any of the 3 donor blood samples.

### e) Conclusion

WAL0921 at final assay concentrations of 750, 75 and 7.5 μg/mL was graded as non-hemolytic according to the criteria referred to in ASTM-F756-17. Additionally, no red blood cell clumping and/or plasma precipitation was observed in human whole blood at any concentration tested. It can therefore be concluded that WAL0921 is compatible with human whole blood and plasma up to the highest tested assay concentration, 750 μg/mL.

TABLE 13
WAL0921mu-NTS-001 ACR (average +/− SEM)

| | | | Day −2 | Day 1 | Day 2 | Day 3 | Day 4 |
|---|---|---|---|---|---|---|---|
| Plaur−/−; | IgG control | ACR (mg/g) | 25.8 ± | 82522.5 ± | 53971.9 ± | 43597.6 ± | 24179.4 ± |
| hsuPAR | | | 1.2 | 6137.9 | 9844.2 | 8015.2 | 5767.0 |
| TG | | Fold Change | 1.0 ± | 3275.4 ± | 2282.8 ± | 1799.3 ± | 977.0 ± |
| | | over Baseline | 0.0 | 301.3 | 563.1 | 385.9 | 238.0 |
| | WAL0921-mu | ACR (mg/g) | 92.9 ± | 68795.9 ± | 42230.1 ± | 31738.3 ± | 19363.5 ± |
| | | | 44.2 | 8270.5 | 9224.1 | 8762.9 | 5452.7 |
| | | Fold Change | 1.0 ± | 1969.6 ± | 1364.0 ± | 1183.1 ± | 581.4 ± |
| | | over Baseline | 0.0 | 472.0 | 371.7 | 408.3 | 189.0 |
| Plaur−/+; | IgG control | ACR (mg/g) | 31.5 ± | 50491.8 ± | 38144.1 ± | 22574.8 ± | 30506.4 ± |
| hsuPAR | | | 4.5 | 8974.9 | 8247.3 | 6058.1 | 10704.9 |
| TG | | Fold Change | 1.0 ± | 1867.8 ± | 1368.5 ± | 800.6 ± | 1065.4 ± |
| | | over Baseline | 0.0 | 392.6 | 299.5 | 228.1 | 386.9 |
| | WAL0921-mu | ACR (mg/g) | 65.1 ± | 83894.6 ± | 51829.1 ± | 27027.7 ± | 29266.5 ± |
| | | | 12.6 | 11724.1 | 7725.4 | 5693.6 | 9914.2 |
| | | Fold Change | 1.0 ± | 1959.0 ± | 1340.9 ± | 799.1 ± | 773.2 ± |
| | | over Baseline | 0.0 | 424.6 | 334.6 | 270.9 | 321.8 |

### 1) Materials and Methods

### (1) Test and Control Materials

The nephrotoxic sera (NTS) used to induce glomerular nephritis was purchased from Probetex Inc, and the same lot was used for all three studies (catalog #PTX-001S-Ms, lot #530-5T-E). The final concentration of NTS was diluted to 50% sera in filter sterile PBS. This solution was prepared at Walden Biosciences and sent to CRL for dosing purposes. The NTS solution was administered on Day 0 of the experiment, intravenously at a volume of 5 mL/kg based on the individual animal weight on that day.

Both the IgG control (mIgG2a Isotype Control, R&D Systems catalog #MAB0031) and antibody test article were prepared at Walden Biosciences and sent to Charles River Labs (CRL) for dosing. For WAb0008-NTS-001 and WAb0008-NTS-002, WAb0008 was produced by Evitria (catalog #902572.8, batch E16710). WAb0008 and WAL0921-mu have identical antigen-binding domains (Fab) to anti-suPAR antibodies in development (WAb0006 and WAL0921 respectively). However, WAb0008 and WAL0921-mu differ from WAb0006 and WAL0921 in that they both have mouse IgG (Fc) domains which allows dosing in murine species with pharmacokinetic profiles similar to a conventional mouse IgG antibody. Anti-suPAR antibodies were diluted to 0.34 mg/mL in sterile PBS, labeled as either A or B, and sent to CRL blinded for dosing.

### (2) Test Animals

The studies included male and female human suPAR isoform 1 transgenic mice of varying ages, diets, and genetic backgrounds (specific ranges for each study is described in Table 9). Table 10 below defines the different transgenic mice used in the studies. Animals were housed with litter mates at CRL. Animals were given a unique identification number at birth, housed in a 14-10-hour light/dark cycle room, and received ad libitum access to food and water (normal diet: Charles River Rat and Mouse 18% (Auto) 5L79; high fat diet (HFD): Research Diet D12492Rodent Diet With 60 kcal % Fat). Genotype and human suPAR copy number were determined by qPCR by 6 weeks of age. Body weight, urine, and blood were collected monthly to track natural progression of biomarkers as the mice age.

TABLE 9
Transgenic Mouse Definition

| | Mouse urokinase- | | |
|---|---|---|---|
| | type plasminogen | Mouse soluble | Human suPAR |
| | activator receptor | uPAR | Transgenics |
| | (uPAR) | (suPAR) | (hsuPAR Tg)* |
| Plaur^{-/-} | −/− | −/− | −/− |
| Plaur^{+/-} | +/− | +/− | −/− |
| Plaur^{-/-}, hsuPAR^{+/-} | −/− | −/− | +/− |
| Plaur^{+/-}, hsuPAR^{+/-} | +/− | +/− | +/− |

−/− indicates the gene/protein is not present (homozygous, knockout (KO))
+/− indicates the gene/protein is present (heterozygous)
*hsuPAR may be present up to 4 copies

TABLE 10
Group Allocations for NTS Studies

| | | | | | | | | Human |
|---|---|---|---|---|---|---|---|---|
| | | | | Age | | | ACR | suPAR |
| | | | | (Average | | Sex | (Average, | (Average, |
| Study | Group | N | Genotype | days) | Diet | (m/f) | mg/g) | ng/mL) |
| WAb008- | A | 11 | 11 Plaur−/−, | 213 | 6 normal | 8/3 | 77 | 347 |
| NTS-001 | | | hsuPAR+/− | | 5 HFD | | | |
| — | B | 11 | 11 Plaur−/−, | 213 | 6 normal | 8/3 | 81 | 330 |
| | | | hsuPAR+/− | | 5 HFD | | | |
| WAb008- | A | 15 | 9 Plaur−/−, | 217 | normal | 8/7 | 166 | 158 |
| NTS-002 | | | hsuPAR+/− | | | | | |
| | | | 5 Plaur−/− | | | | | |
| | | | 1 Plaur+/−, | | | | | |
| | | | hsuPAR+/− | | | | | |
| — | B | 15 | 9 Plaur−/−, | 197 | normal | 8/7 | 170 | 179 |
| | | | hsuPAR+/− | | | | | |
| | | | 5 Plaur−/− | | | | | |
| | | | 1 Plaur+/−, | | | | | |
| | | | hsuPAR+/− | | | | | |
| WAL0921mu- | A | 10 | 10 Plaur−/−, | 101 | normal | 4/6 | — | 155 |
| NTS-001 | | | hsuPAR+/− | | | | | |
| — | — | 10 | 10 Plaur−/−, | 101 | normal | 4/6 | — | 164 |
| | | | hsuPAR+/− | | | | | |
| — | B | 10 | 10 Plaur+/− , | 100 | normal | 4/6 | — | 45 |
| | | | hsuPAR+/− | | | | | |
| — | — | 10 | 10 Plaur+/−, | 100 | normal | 4/6 | — | 44 |
| | | | hsuPAR+/− | | | | | |

### (3) Group Designation and Treatment

To allocate individuals to groupings for studies we have used a method similar to that described by Grischott (Grischott, BMC Medical Research Methodology (2018) 18:108). This method ensures that imbalance due to bias in the study is minimized. Blood and urine samples were collected approximately 3 weeks prior to study to measure urine ACR and serum suPAR levels.

The variables used to allocate animals for either group A or B were age, weight, sex, ACR, and human suPAR levels. We also tracked transgene copy number and used that to allocate animals in WAL0921mu-NTS-001 Additionally, alternative diet was also assessed in WAb0008-NTS-001. This was to ensure the averages between groups were similar at the start of the study across those variables. The baseline characteristics of each study is depicted in Table Notably, in the WAL0921mu study, 20 matched pairs of animals were established, determined by sex, age, human suPAR copy number, and baseline suPAR. One animal from each pair was randomly assigned to group A and its “twin” to group B to make future direct comparisons between those two matched mice in terms of response (appendices).

### (4) Dosing Regimen

For all studies Walden antibodies or the IgG control were administered on Day-2 (two days prior to NTS challenge) and Day 2 (two days after NTS challenge). The dose of antibody or control in each study was 1.2 mg/kg by intraperitoneal administration at dosing volume of 5 ml/kg per animal.

### (5) Body Weight Measurement

Mice were weighed throughout the study starting on Day-2 and is represented in grams (g). Percent of baseline was calculated by dividing the day X of study weight by the Day-weight and multiplying by 100.

### (6) Urine Collection

For all studies, urine was collected between 6:00 am and 7:00 am by free expression, if mice did not immediately express urine, their abdomens were gently massaged to assist collection. Aliquots were stored at −80 Celsius prior to shipping to Walden. Urine was used to determine the ACR for each sample on each collection day. ELISA methods were applied to measure mouse albumin (Abcam, cat #ab 108792) and creatinine (R&D Systems, KGE005). These values were used to calculate the albumin: creatinine ratio (ACR, mg/g) which was represented as milligrams (mg) of albumin per gram (g) of creatinine

### (7) Blood Collection

Blood was collected by restraining the mouse by the scruff and using the facial vein to collect a maximum of 100 μL of blood into serum separator tubes. These tubes were then left at room temperature for a minimum of 15 minutes, prior to centrifugation. The supernatant was transferred to a 1 mL Eppendorf tube and stored at −80 Celsius until shipment to Walden. Serum was used to determine human suPAR levels using the Virogates suPARnostic (cat #E001) ELISA kit.

### (8) Euthanasia

For WAb008-NTS001 and WAb008-NTS-002 mice were not euthanized at the end of study but returned to the colony once the experiment was completed. In WAL0921mu-NTS-001, mice were euthanized by CO₂ overdose, kidneys were collected and fixed in 10% formalin for future analysis.

### (9) Justification for Selection of Species, Route of Administration and Dosage

Human suPAR transgenic mice were chosen for these experiments as the surrogate antibodies contain a mouse Fc constant region but bind to human suPAR. Route of administration was chosen as intraperitoneal based on antibodies tested in other animal models of kidney disease in the literature. The dose of 1.2 mg/kg was determined by calculating the antibody levels to be at least 10× molar excess in circulation based on the highest serum human suPAR level of 600 ng/mL observed in the test cohorts at baseline. The treatment was administered twice during the experiment to ensure appropriate coverage of target.

### (10) Clinical Observations

Animals were assessed daily for general health and activity throughout the study, there were no clinical observations noted by CRL for any animal during WAb0008-NTS-001, WAb0008-NTS-002, or WAL0921mu-001.

### (11) Body Weights

As described in tables 3-5, body weight was relatively stable in WAb008-NTS001, WAb008-NTS-002 and WAL0921mu-NTS-002. The NTS challenge, IgG control, WAb008 or WAL0921mu treatment did not have a negative impact on body weight in this model (average body weight did not drop below 96% of baseline at any point in the three studies).

### g) Conclusion

In Plaur−/− hsuPAR Tg mice, across the three NTS studies, anti-suPAR antibodies WAb0008 and WAL0921mu treatment trended to lower peak and overall albuminuria compared to an IgG control in the NTS model. This data supports further exploration of anti-suPAR antibodies for the treatment of kidney diseases associated with albuminuria.

### 19. Example 19: Toxicity Study of WAL0921

The objectives of this study in cynomolgus monkeys were to determine the potential toxicity of WAL0921, to evaluate the potential reversibility of any findings, and to determine the toxicokinetic (TK) characteristics of the test item. WAL0921 is a monoclonal antibody for the treatment of inflammation of the kidney and was given via intravenous injection over 30 minutes once weekly for 4 weeks with a total of 5 administrations, that was, dosing on Days 1, 8, 15, 22 and 29.

The study design was as follows:

TABLE 23
Experimental Design

| | | | Dose | Dose | No. of Animals | | | |
|---|---|---|---|---|---|---|---|---|
| Group | Test | Dose Level | Volume^{a} | Concentration | Main Study | | Recovery | |
| No. | Item | (mg/kg/dose) | (mL/kg) | (mg/mL)^{b} | Males | Females | Males | Females |
| 1 | Control | 0 | 5 | 0 | 3 | 3 | 2 | 2 |
| 2 | WAL0921 | 15 | 5 | 3 | 3 | 3 | — | — |
| 3 | WAL0921 | 45 | 5 | 9 | 3 | 3 | 2 | 2 |
| 4 | WAL0921 | 120 | 5 | 24 | 3 | 3 | 2 | 2 |

— = Not applicable.
^{a}Dose was based on the most recent body weight measurement.
Control (and vehicle) = 20 mM Histidine buffer, 8% [w/v] Sucrose, 0.04% [w/v] PS80, pH 6.0
^{b}Analysis indicated that formulations were prepared accurately with regards to concentration and homogeneity.

All animals dosed with WAL0921 showed exposure consistent with intravenous infusion administration. WAL0921 was quantifiable to the last sampling time point of 144.5 hours. Following the final infusion on Day 29, in all recovery animals, quantifiable levels of WAL0921 were observed at 504 hours (the last collected sample on Day 50). The median maximal concentrations (tmax) were achieved 1 hour postdose. Overall, WAL0921 exposure (as measured by Cmax and AUC0-last) was comparable between males and females across the dose range, with any M/F ratio differences between 0.857 to 1.37. Exposure of WAL0921 increased with increasing dose in a manner that was approximately proportional across the dose range. There was evidence of slight accumulation over 29 days of weekly intravenous infusion administration of WAL0921. Male and female combined Cmax and AUC0-last accumulation ranged between 1.29 and 1.48 for Cmax and 1.44 and 1.96 for AUC0-last.

There were no unscheduled deaths and no clinical signs that could be directly attributed to WAL0921. Body weight, body weight gain, the eye, electrocardiology, clinical pathology, urine volume and content, cytokine levels, healing of a lesion and organ weights were unaffected by WAL0921. There were no macro- or microscopic findings related to WAL0921.

In conclusion, administration of up to 120 mg/kg/dose WAL0921 by intravenous infusion (over 30 minutes) was well tolerated in cynomolgus monkeys with no evidence of toxicity. Based on these results, the no-observed-effect level (NOEL) was considered to be 120 mg/kg/dose.

### a) Test Material Identification

TABLE 24
Test Item Identification

| | Test Item | |
|---|---|---|
| Identification: | WAL0921 | |
| Alternate | WB-Ab-01-0014 LALA P329G; | |
| Identification: | WBP2528; | |
| | WAb0014, ENG DP | |
| Batch/Lot No.: | 20220501 | |
| Expiration Date: | 20 May 2023 | |
| Physical | Clear colorless liquid | |
| Description: | | |
| Purity | See record of analysis | |
| Concentration: | 50.3 | mg/mL |
| Density: | 1.04 | g/mL |

| | |
|---|---|
| Storage | In a freezer set to |
| Conditions: | maintain −20° C. |
| Provided by: | Sponsor |

TABLE 25
Vehicle Control Item Identification

| | Vehicle Control Item |
|---|---|
| Identification: | WAL0921 buffer |
| Ingredients | 20 mM Histidine buffer, 8% (w/v) |
| | Sucrose, |
| | 0.04% (w/v) PS80, pH 6.0 |
| Storage | Details retained in the study records |
| Conditions: | |
| Provided by: | Test Facility |

### b) Stability

For the time periods covered by this study, homogeneity, stability and concentration was demonstrated for all samples related to test material formulations, and bioanalytical and biomarker samples were analyzed within their stability periods.

### c) Study Design

TABLE 26
Experimental Design

| | | | Dose | Dose | Animal Numbers | | | |
|---|---|---|---|---|---|---|---|---|
| Group | Test | Dose Level | Volume^{a} | Concentration | Main Study | | Recovery | |
| No. | Item | (mg/kg/dose) | (mL/kg) | (mg/mL) | Males | Females | Males | Females |
| 1 | Control | 0 | 5 | 0 | 1001- | 1501- | 1004, | 1504, |
| | | | | | 1003 | 1503 | 1005 | 1505 |
| 2 | WAL0921 | 15 | 5 | 3 | 2001- | 2501- | — | — |
| | | | | | 2003 | 2503 | | |
| 3 | WAL0921 | 45 | 5 | 9 | 3001- | 3501- | 3004, | 3504, |
| | | | | | 3003 | 3503 | 3005 | 3505 |
| 4 | WAL0921 | 120 | 5 | 24 | 4001- | 4501- | 4004, | 4504, |
| | | | | | 4003 | 4503 | 4005 | 4505 |

— = Not applicable.
^{a}Dose was based on the most recent body weight measurement.
Control (and vehicle) = 20 mM Histidine buffer, 8% [w/v] Sucrose, 0,04% [w/v] PS80, pH 6.0

TABLE 27
Additional Protocol Deliverables

| | |
|---|---|
| Age at Initiation of Dosing | 1 year 10 months-2 years 7 months old |
| Body Weight Range at | 2.7-4.1 kg males (target weight: 1.5-4 kg); |
| Initiation of Dosing | 2.4-3 kg females |
| Number of Acclimation Days | 40 |
| Acclimation to Restraint | All animals underwent conditioning to |
| Procedures | restraint chairs prior to dosing |
| Environmental Conditions | Temperature: 19-24° C. |
| | Humidity: 50-88% (target 40-70%) |
| Animal Identification | Subcutaneously implanted ‘glass-sealed’ |
| | electronic identification chip |

### d) Results

### (1) Dose Formulation Analyses

Formulations were prepared accurately with regards to concentration and homogeneity. All study samples analysed had mean concentrations within or equal to the acceptance criteria of <10% (individual values within or equal to +15%) of their theoretical concentrations, and for homogeneity, the relative standard deviation of concentrations for all samples in each group was within the acceptance criteria of ≤10%. There was no WAL0921 detected in control formulations.

All study samples analysed had mean concentrations within or equal to the acceptance criteria of ±10%. With regards to the individual values, with the exception of 2 samples in Group 3 (24.4%, 34.4%) and one sample in Group 4 (19.6%), all samples had individual values within or equal to ±15% of their theoretical concentrations. As part of the investigation the backup samples were analysed in triplicate, and all individual results were within ±15% of theoretical values and, therefore, were within the acceptance criteria. The investigation demonstrated that Day 1 formulations had been prepared accurately. It was confirmed that Groups 3 and 4 were originally out of specification due to a sample preparation error

For homogeneity, the RSD of concentrations for all samples in each group was within the acceptance criteria of ≤10%, except for Day 1, Group 3 (15.4%); therefore, as part of the investigation, the backup samples were analysed in triplicate, and the result (1.1%) was within the acceptance criteria. The investigation demonstrated that Day 1 formulations had been prepared accurately.

### (2) Mortality

There were no unscheduled deaths.

### (3) Clinical Observations

There were no clinical signs that could be directly attributed to WAL0921.

There was no difference in the healing of the surgical site between controls and animals receiving 120 mg/kg/dose. Each animal had an individual response to healing but generally a scab formed within a few days after the lesion was created on Day 31 and was present until resolution, which was for one animal (4005M) on Day 34 and for the last animal (4004M) on Day 49.

### (4) Body Weights and Body Weight Gains

Body weight and body weight gain were unaffected by WAL0921. There were differences between controls and animals receiving WAL0921, but these were present also pretreatment and the percent differences were maintained through the dosing period. There was no effect on body weight and body weight gain during the recovery period.

### (5) Ophthalmic Examinations

There were no changes in the eye related to WAL0921.

### (6) Electrocardiology

This phase report presents the electrocardiology findings in cynomolgus monkeys assigned to the study with the objective to determine the potential toxicity of WAL0921, a monoclonal antibody for the treatment of inflammation of the kidney. The test item was given at 15, 45 or 120 mg/kg/dose via intravenous (IV) administration over 30 minutes once weekly for 4 weeks with a total of 5 administrations, that was, dosing on Days 1, 8, 15, 22 and 29. For the electrocardiology work detailed in this phase report, the phase start date was 8 Aug. 2022, and the phase completion date was 20 Oct. 2022.

Electrocardiology was performed once during pretreatment and on Day 22. As there were no test item-related electrocardiology findings on Day 22, no measurements were collected during the recovery period. There were no test item-related electrocardiology findings in cynomolgus monkeys on Day 22 (within 30-90 minutes post end of infusion) after the once weekly intravenous administration of 15, 45 or 120 mg/kg/dose of WAL0921.

### (a) ECG Analysis and Heart Rate

Pretreatment values were within those expected for this species and group means were broadly similar between the groups.

There were no test item-related effects in animals receiving 45 or 120 mg/kg/dose WAL0921 with values similar to controls or pretreatment.

### (b) ECG Gross Morphology Analysis

On Day 22, there were no abnormalities or arrhythmias identified that were considered to be associated with administration of 15, 45 or 120 mg/kg/dose WAL0921.

### (c) Conclusion

The once weekly intravenous (over 30 minutes) administration of 15, 45 or 120 mg/kg/dose WAL0921 to cynomolgus monkeys did not result in any test item-related electrocardiology findings at Day 22.

### (7) Haematology

Haematology was unaffected by WAL0921. Any differences noted, including those that achieved statistical significance, were considered to be due to individual changes, to biological variation or lacked true dose relationship, and therefore, were not related to the administration of WAL0921.

### (8) Coagulation

Coagulation was unaffected by WAL0921. D-dimer was higher at the end of the dosing period, compared with pretreatment. This effect was marginal and was also noted in controls. At the end of the recovery period, D-dimer concentrations were similar to pretreatment.

### (9) Clinical Chemistry

Clinical chemistry was unaffected by WAL0921. Any differences noted, including those that achieved statistical significance, were considered to be due to individual changes, to biological variation or lacked true dose relationship, and therefore, were not related to the administration of WAL0921.

### (10) Urinalysis

Urine volume and composition were unaffected by WAL0921.

### (11) Toxicokinetic Evaluations

All animals dosed with WAL0921 showed exposure consistent with intravenous infusion administration WAL0921 was quantifiable to the last sampling time point of 144.5 hours in all dosed animals. Overall, WAL0921 exposure (as measured by Cmax and AUC0-last) was comparable between males and females across the dose range, with any M/F ratio differences between 0.857 to 1.37.

Exposure of WAL0921 increased with increasing dose in a manner that was approximately proportional across the dose range. There was evidence of slight accumulation over 29 days of weekly intravenous infusion administration of WAL0921. Male and female combined Cmax and AUC0-last accumulation ranged between 1.29 and 1.48 for Cmax and 1.44 and 1.96 for AUC0-last.

### (12) Inflammatory Markers: Cytokines

This phase report describes the evaluation of cynomolgus monkey plasma for cytokines IFN-γ, IL-1β, IL-2, IL-4, IL-6, IL-8, MCP-1 and TNFα, the profile being pretreatment (PreT), and 0.5 h, 2 h, 6 h and 24 h after completion of the dose infusion given on Day 1 and Day 29. Cytokines were assayed using a GLP validated bead-based multiplex immunoassay.

The objective of this study phase was to determine the levels of a panel of cytokines (IFN γ, IL-1β, IL-2, IL-4, IL-6, IL-8, MCP-1 and TNFα) on Day 1 and Day 29 after administration of 15, 45 or 120 mg/kg/dose WAL0921, a monoclonal antibody for the treatment of inflammation of the kidney, being given via intravenous injection over 30 minutes once weekly for 4 weeks with a total of 5 administrations, that is, dosing on Days 1, 8, 15, 22 and 29 to cynomolgus monkeys.

In summary, intravenous administration of up to 120 mg/kg/dose WAL0921 once weekly for 4 weeks with a total of 5 administrations had no impact on Day 1 and Day 29 on the levels of any of the measured cytokines.

### (13) Cytokine Analysis

IFN-γ, IL-1β, IL-2, IL-4, IL-6, IL-8, IL-10, MCP-1 and TNFα analysis was completed using the Non-Human Primate Cytokine Kit, Cat. No PRCYTOMAG-40K-09C. Results for IL-10 were not reported due to analytical performance issues. Reagent kits were stored in a refrigerator set to maintain 4° C. when not in use.

The concentration of the upper limit of quantification (ULOQ) and lower limit of quantification (LLOQ) of each standard supplied in the kits The concentration of the upper limit of quantification (ULOQ) and lower limit of quantification (LLOQ) of each standard supplied in the kits is provided in Table 28.

TABLE 28
Calibration Standard Concentration Ranges

| Analyte | LLOQ (pg/mL) | ULOQ (pg/mL) |
|---|---|---|
| IFN-γ | 37.5 | 6000 |
| IL-1β | 37.5 | 6000 |
| IL-2 | 37.5 | 6000 |
| IL-4 | 400 | 12000 |
| IL-6 | 37.3 | 6000 |
| IL-8 | 37.5 | 6000 |
| IL-10 | 188 | 30000 |
| MCP-1 | 37.5 | 2400 |
| TNFα | 37.5 | 6000 |

At the intervals, whole blood samples (approximately 0.5 mL) were collected from the femoral (or other suitable) vein. Samples were collected in K2EDTA tubes and centrifuged. The resultant plasma was separated and transferred to uniquely labelled clear polypropylene tubes and stored in freezer set to maintain −80° C. until analysis.

Plasma cytokine samples were analyzed. The plasma samples were incubated with antibody-coated magnetic beads, after which biotinylated antibody was introduced. The reaction mixture was then incubated with Streptavidin-PE conjugate, which acted as a reporter for detection by a Bio Rad Bio Plex 200 Luminex instrument.

The acceptance criteria of calibration standards, quality control (QC) samples and study samples are provided in Table 29. Calibration standards which did not meet these criteria were removed from the curve. Where possible, study samples were analysed in duplicate and the mean result for each sample is reported. Concentrations below the LLOQ for each cytokine have been reported as such. Study samples that were analyzed in singlicate or did not meet the acceptance criterion have been flagged.

TABLE 29
Acceptance Criteria for IFN-γ, IL-1β, IL-2,
IL-4, IL-6, IL-8, IL-10, MCP-1 and TNFα

| | Relative Error | Coefficient of |
|---|---|---|
| | (%) | Variation (%) |
| LLOQ/ULOQ Standards | ±30.0 | ≤20.0 |
| Non-LLOQ/ULOQ Standards | ±25.0 | ≤20.0 |
| Quality Control Samples | ±25.0 | ≤20.0 |
| Study Samples | N/A | ≤20.0 |

N/A—Not applicable

Levels of IL-1β were below LLOQ at all timepoints across all dose groups. Levels of IFN-γ, IL-2, IL-4, and IL-6 were either below LLOQ or close to LLOQ at all timepoints (pretreatment through 24 h end of infusion) across all dose groups. For IFN-γ, IL-4, and IL-6, the exception to this was seen in a single animal (3504F) in Group 3 (45 mg/kg/dose), which showed consistently high levels across all timepoints, including pretreatment.

Levels of IL-8 and TNFα were mostly within the quantifiable range of the assay, with fluctuations across timepoints and between dose groups, but with no identifiable trend following administration of WAL0921. For TNFα, a single animal (3504F) in Group 3 (45 mg/kg/dose), there were consistently higher levels observed across all timepoints, including pretreatment.

Levels of MCP-1 were all within the quantifiable range of the assay but showed no identifiable trend following administration of WAL0921. MCP-1 levels for Group 2 (15 mg/kg/dose) were broadly similar at each timepoint and fluctuated within a narrow range.

The one exception was animal 2502F, which peaked approximately 10× higher than pretreatment on Day 29, 6 h after the end of infusion.

Results for IL-10 have not been reported due to the analytical performance of the kits resulting in high background readings making results difficult to interpret.

### (14) Macroscopic Pathology

There were no WAL0921-related gross pathology findings. All gross findings observed were of the nature commonly observed in this age of cynomolgus monkey, or occurred at a similar incidence in control and treated animals, and, therefore, were considered not to be test item-related.

### (15) Organ Weights

There were no WAL0921-related organ weight differences.

There were group mean and individual organ weight values that were different from their respective controls. There were, however, no patterns or correlating data (taking into account differences in sexual maturity) to suggest these values were test item-related.

### (16) Microscopic Evaluations

There were no WAL0921-related microscopic findings.

E. Sequences
human suPAR binding molecule WAb CD0014 V_{H} amino acid sequence
SEQ ID NO: 1
QVQLVQSGAEVKKPGASVKVSCKASGYTFTNYYMHWVRQAPGQGLEWMGWIDHESG
STIYAQKFQGRVTMTRDTSTSTVYMELSSLRSEDTAVYYCARGYDVDWFVYWGQGT
LVTVSS
human suPAR binding molecule WAb CD0014 V_{L} amino acid sequence
SEQ ID NO: 2
GSHASDIQMTQSPSSLSASVGDRVTITCSARQSVSYMYWYQQKPGKAPKLLIYRTSNLA
SGVPSRFSGSGSGTDFTLTISSLQPEDFATYYCQQYHSYPPTFGGGTKVEIK
human suPAR binding molecule WAb CD0014 Heavy Chain Constant
Region amino acid sequence
SEQ ID NO: 3
ASTKGPSVFPLAPSSKSTSGGTAALGCLVKDYFPEPVTVSWNSGALTSGVHTFPAVLQS
SGLYSLSSVVTVPSSSLGTQTYICNVNHKPSNTKVDKKVEPKSCDKTHTCPPCPAPEAA
GGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALGAPIEKTISKAKGQPREPQVYTLPP
SRDELTKNQVSLTCLVKGFYPSDIAVEWESNGQPENNYKTTPPVLDSDGSFFLYSKLT
VDKSRWQQGNVFSCSVMHEALHNHYTQKSLSLSPGK
human suPAR binding molecule WAb CD0014 Light Chain Constant
Region amino acid sequence
SEQ ID NO: 4
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC
human suPAR binding molecule WAb CD0001 V_{H} amino acid sequence
SEQ ID NO: 5
QVQLVQSGAEVKKPGASVKVSCKASGYNIKDYYIHWVRQAPGQGLEWMGWISPDSGV
TNYAQKFQGRVTMTRDTSTSTVYMELSSLRSEDTAVYYCARGYDVDWFVYWGQGTL
VTVSS
human suPAR binding molecule WAb CD0002 V_{H} amino acid sequence
SEQ ID NO: 6
QVQLVQSGAEVKKPGASVKVSCKASGYNIKDYYIHWVRQAPGQGLEWMGWISPSSGV
TNYAQKFQGRVTMTRDTSTSTVYMELSSLRSEDTAVYYCARGYDVDWFVYWGQGTL
VTVSS
human suPAR binding molecule WAb CD0003 V_{H} amino acid sequence
SEQ ID NO: 7
QVQLVQSGAEVKKPGASVKVSCKASGFNIKDYYIHWVRQAPGQGLEWMGWIDPENGS
TIYDQKFQGRVTMTRDTSTSTVYMELSSLRSEDTAVYYCARGYDIDWFVYWGQGTLV
TVSS
human suPAR binding molecule WAb CD0004 V_{H} amino acid sequence
SEQ ID NO: 8
QVQLVQSGAEVKKPGASVKVSCKASGFNIKDYYIHWVRQAPGQGLEWMGWIDPESGS
TIYDQKFQGRVTMTRDTSTSTVYMELSSLRSEDTAVYYCARGYDIDWFVYWGQGTLV
TVSS
human suPAR binding molecule WAb CD0005 V_{H} amino acid sequence
SEQ ID NO: 9
QVQLVQSGAEVKKPGASVKVSCKASGYTFIGSYVHWVRQAPGQGLEWMGWIDHENG
NTIYDQKFQGRVTMTRDTSTSTVYMELSSLRSEDTAVYYCARGYDVDWFVYWGQGTL
VTVSS
human suPAR binding molecule WAb CD0006 V_{H} amino acid sequence
SEQ ID NO: 10
QVQLVQSGAEVKKPGASVKVSCKASGYTFTSHYIQWVRQAPGQGLEWMGWIDHENG
NTIYDQKFQGRVTMTRDTSTSTVYMELSSLRSEDTAVYYCARGTDVDWFVYWGQGTL
VTVSS
human suPAR binding molecule WAb CD0007 V_{H} amino acid sequence
SEQ ID NO: 11
QVQLVQSGAEVKKPGASVKVSCKASGYTFIGSYVHWVRQAPGQGLEWMGWIDHENG
NTIYDQKFQGRVTMTRDTSTSTVYMELSSLRSEDTAVYYCARGYDVDWFVYWGQGTL
VTVSS
human suPAR binding molecule WAb CD0008 V_{H} amino acid sequence
SEQ ID NO: 12
QVQLVQSGAEVKKPGASVKVSCKASGYTFTSHYIQWVRQAPGQGLEWMGWIDHENG
NTIYDQKFQGRVTMTRDTSTSTVYMELSSLRSEDTAVYYCARGTDVDWFVYWGQGT
LVTVSS
human suPAR binding molecule WAb CD0009 V_{H} amino acid sequence
SEQ ID NO: 13
QVQLVQSGAEVKKPGASVKVSCKASGYTFTGHYVHWVRQAPGQGLEWMGWIDHENG
NTIYDQKFQGRVTMTRDTSTSTVYMELSSLRSEDTAVYYCARGYDVDWFVYWGQGTL
VTVSS
human suPAR binding molecule WAb CD0010 V_{H} amino acid sequence
SEQ ID NO: 14
QVQLVQSGAEVKKPGASVKVSCKASGYTFTGNYVHWVRQAPGQGLEWMGWIDHENG
NTIYDQKFQGRVTMTRDTSTSTVYMELSSLRSEDTAVYYCARGYDVDWFVYWGQGTL
VTVSS
human suPAR binding molecule WAb CD0011 V_{H} amino acid sequence
SEQ ID NO: 15
QVQLVQSGAEVKKPGASVKVSCKASGYTFTGSYVHWVRQAPGQGLEWMGWIDHENG
NTIYDQKFQGRVTMTRDTSTSTVYMELSSLRSEDTAVYYCARGYDVDWFVYWGQGTL
VTVSS
human suPAR binding molecule WAb CD0012 V_{H} amino acid sequence
SEQ ID NO: 16
QVQLVQSGAEVKKPGASVKVSCKASGYTFTGYYMHWVRQAPGQGLEWMGWIDHEN
GNTIYDQKFQGRVTMTRDTSTSTVYMELSSLRSEDTAVYYCARGYDVDWFVYWGQGT
LVTVSS
human suPAR binding molecule WAb CD0013 V_{H} amino acid sequence
SEQ ID NO: 17
QVQLVQSGAEVKKPGASVKVSCKASGYTFTNYYMHWVRQAPGQGLEWMGWIDHEN
GSTIYAQKFQGRVTMTRDTSTSTVYMELSSLRSEDTAVYYCARGYDVDWFVYWGQGT
LVTVSS
human suPAR binding molecule WAb CD0015 V_{H} amino acid sequence
SEQ ID NO: 18
QVQLVQSGAEVKKPGASVKVSCKASGYTFTNYYIHWVRQAPGQGLEWMGWISPDSGG
TKYAQKFQGRVTMTRDTSTSTVYMELSSLRSEDTAVYYCARGYDVDWFVYWGQGTL
VTVSS
human suPAR binding molecule WAb CD0016 V_{H} amino acid sequence
SEQ ID NO: 19
QVQLVQSGAEVKKPGASVKVSCKASGYTFTNYYIHWVRQAPGQGLEWMGWISPSSGG
TKYAQKFQGRVTMTRDTSTSTVYMELSSLRSEDTAVYYCARGYDVDWFVYWGQGTL
VTVSS
human suPAR binding molecule WAb CD0017 V_{H} amino acid sequence
SEQ ID NO: 20
QVQLVQSGAEVKKPGASVKVSCKASGYTFSAYYMHWVRQAPGQGLEWMGWIDHEN
GNTIYDQKFQGRVTMTRDTSTSTVYMELSSLRSEDTAVYYCARGYDVDWFVYWGQGT
LVTVSS
human suPAR binding molecule WAb CD0018 V_{H} amino acid sequence
SEQ ID NO: 21
QVQLVQSGAEVKKPGASVKVSCKASGYTFTNYYMHWVRQAPGQGLEWMGWIDHEN
GNTIYDQKFQGRVTMTRDTSTSTVYMELSSLRSEDTAVYYCARGYDVDWFVYWGQGT
LVTVSS
human suPAR binding molecule WAb CD0019 V_{H} amino acid sequence
SEQ ID NO: 22
QVQLVQSGAEVKKPGASVKVSCKASGYTFSSYYMHWVRQAPGQGLEWMGWIDHENG
NTIYAQKFQGRVTMTRDTSTSTVYMELSSLRSEDTAVYYCARGYDVDWFVYWGQGTL
VTVSS
human suPAR binding molecule WAb CD0020 V_{H} amino acid sequence
SEQ ID NO: 23
QVQLVQSGAEVKKPGASVKVSCKASGYTFTSHYVHWVRQAPGQGLEWMGWIDHENG
NTIYDQKFQGRVTMTRDTSTSTVYMELSSLRSEDTAVYYCARGYDVDWFVYWGQGTL
VTVSS
human suPAR binding molecule WAb CD0022 V_{H} amino acid sequence
SEQ ID NO: 24
QVQLVQSGAEVKKPGASVKVSCKASGYTFTNYYMHWVRQAPGQGLEWMGWIDHESG
STIYAQKFQGRVTMTRDTSTSTVYMELSSLRSEDTAVYYCARGYDVDWFVYWGQGTL
VTVSS
human suPAR binding molecule WAb CD0001 V_{L} amino acid sequence
SEQ ID NO: 25
GSHASDIQMTQSPSSLSASVGDRVTITCSARQSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0002 V_{L} amino acid sequence
SEQ ID NO: 26
GSHASDIQMTQSPSSLSASVGDRVTITCSARQSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0003 V_{L} amino acid sequence
SEQ ID NO: 27
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0004 V_{L} amino acid sequence
SEQ ID NO: 28
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0005 V_{L} amino acid sequence
SEQ ID NO: 29
GSHASDIQMTQSPSSLSASVGDRVTITCSARSSVSYMYWYQQKPGKAPKLLIYRTSSLAS
GVPSRFSGSGSGTDFTLTISSLQPEDFATYYCQQYHSYPPTFGGGTKVEIKRAAAGS
human suPAR binding molecule WAb CD0006 V_{L} amino acid sequence
SEQ ID NO: 30
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0007 V_{L} amino acid sequence
SEQ ID NO: 31
GSHASDIQMTQSPSSLSASVGDRVTITCSARSSVSYMYWYQQKPGKAPKLLIYRTSSLAS
GVPSRFSGSGSGTDFTLTISSLQPEDFATYYCQQYHSYPPTFGGGTKVEIKRAAAGS
human suPAR binding molecule WAb CD0008 V_{L} amino acid sequence
SEQ ID NO: 32
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0009 V_{L} amino acid sequence
SEQ ID NO: 33
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0010 V_{L} amino acid sequence
SEQ ID NO: 34
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0011 V_{L} amino acid sequence
SEQ ID NO: 35
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0012 V_{L} amino acid sequence
SEQ ID NO: 36
GSHASDIQMTQSPSSLSASVGDRVTITCSARQSISYMYWYQQKPGKAPKLLIYRTSNLAS
GVPSRFSGSGSGTDFTLTISSLQPEDFATYYCQQYHSYPPTFGGGTKVEIKRAAAGS
human suPAR binding molecule WAb CD0013 V_{L} amino acid sequence
SEQ ID NO: 37
GSHASDIQMTQSPSSLSASVGDRVTITCSARQSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0015 V_{L} amino acid sequence
SEQ ID NO: 38
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0016 V_{L} amino acid sequence
SEQ ID NO: 39
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0017 V_{L} amino acid sequence
SEQ ID NO: 40
GSHASDIQMTQSPSSLSASVGDRVTITCRARQSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0018 V_{L} amino acid sequence
SEQ ID NO: 41
GSHASDIQMTQSPSSLSASVGDRVTITCSASSSISYMYWYQQKPGKAPKLLIYRTSNLAS
GVPSRFSGSGSGTDFTLTISSLQPEDFATYYCQQYHSYPPTFGGGTKVEIKRAAAGS
human suPAR binding molecule WAb CD0019 V_{L} amino acid sequence
SEQ ID NO: 42
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0020 V_{L} amino acid sequence
SEQ ID NO: 43
GSHASDIQMTQSPSSLSASVGDRVTITCSARSSVSYMYWYQQKPGKAPKLLIYRTSNLA
SGVPSRFSGSGSGTDFTLTISSLQPEDFATYYCQQYHSYPPTFGGGTKVEIKRAAAGS
human suPAR binding molecule WAb CD0022 V_{L} amino acid sequence
SEQ ID NO: 44
GSHASDIQMTQSPSSLSASVGDRVTITCSARQSVSYMYWYQQKPGKAPKLLIYRTSNLA
SGVPSRFSGSGSGTDFTLTISSLQPEDFATYYCQQYHSYPPTFGGGTKVEIKRAAAGS
Wal0921 without LALPG called WalXXX VH leader sequence
SEQ ID NO: 4901
ATGGAGTTTGGGCTGAGCTGGGTTTTCCTTGTGGCTATTTTAAAAGGTGTCCAGTGT
G
Wal0921 without LALPG called WalXXX VL leader sequence
SEQ ID NO: 4902
ATGGAAGCCCCAGCGCAGCTTCTCTTCCTCCTGCTACTCTGGCTCCCAGATACCACC
GGAG
Wal0921 without LALPG called WalXXX VH sequence
SEQ ID NO: 4903
QVQLVQSGAEVKKPGASVKVSCKASGYTFTNYYMHWVRQAPGQGLEWMGWIDHESG
STIYAQKFQGRVTMTRDTSTSTVYMELSSLRSEDTAVYYCARGYDVDWFVYWGQGTL
VTVSS
Wal0921 without LALPG called WalXXX VL sequence
SEQ ID NO: 4904
DIQMTQSPSSLSASVGDRVTITCSARQSVSYMYWYQQKPGKAPKLLIYRTSNLASGVPS
RFSGSGSGTDFTLTISSLQPEDFATYYCQQYHSYPPTFGGGTKVEIK
Wal0921 without LALPG called WalXXX Heavy Chain Constant
Region
SEQ ID NO: 4905
ASTKGPSVFPLAPSSKSTSGGTAALGCLVKDYFPEPVTVSWNSGALTSGVHTFPAVLQSS
GLYSLSSVVTVPSSSLGTQTYICNVNHKPSNTKVDKKVEPKSCDKTHTCPPCPAPELLGG
PSVFLFPPKPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPREPQVYTLPPS
RDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
Wal0921 without LALPG called WalXXX Light Chain Constant Region
SEQ ID NO: 4905
RTVAAPSVFIFPPSDEQLKSGTASVVCLLNNFYPREAKVQWKVDNALQSGNSQESVTEQ
DSKDSTYSLSSTLTLSKADYEKHKVYACEVTHQGLSSPVTKSFNRGEC

### REFERENCES

Alfano D, Franco P, Stoppelli M P. Modulation of cellular function by the urokinase receptor signalling: A mechanistic view. Front. Cell Dev. Biol. 2022; 10:818616
Eugen-Olsen J, Anderson O, Linneberg A, et al. Circulating soluble urokinase plasminogen activator receptor predicts cancer, cardiovascular disease, diabetes and mortality in the general population. J Intern Med. 2010; 268:296-308
Hayek S S, Sever S, Ko Y A, et al. Soluble Urokinase Receptor and Chronic Kidney Disease. N Engl J Med. 2015; 373 (20): 1916-1925
Hoyer-Hansen G, Rønne E, Solberg H, et al. Urokinase plasminogen activator cleaves its cell surface receptor releasing the ligand-binding domain. J Biol Chem. 1992; 25 (5): 18224-18229
Iverson E, Kallemose T, Hornum M, et al. Soluble urokinase plasminogen activator receptor and decline in kidney function among patients without kidney disease. Clin Kidney J. 2022; 15 (8): 1534-1541
Saleem M. What is the role of soluble urokinase-type plasminogen activator in renal disease. Nephron. 2018; 139 (4): 334-341
Sidenius N, Sier C F M, Blasi F. Shedding and cleavage of the urokinase receptor (uPAR): i dentification and characterization of uPAR fragments in vitro and in vivo. FEBS Letters. 2000; 475 (1): 52-56
Thunø M, Macho B, Eugen-Olsen J. suPAR: the molecular crystal ball. Dis Markers. 2009; 27 (3): 157-172

What is claimed is:

1. A urokinase plasminogen activator receptor (uPAR) binding molecule comprising a light chain variable domain, wherein the light chain variable domain comprises 3 complementarity determining regions (CDRs), CDR1, CDR2, and CDR3 as set forth in SEQ ID NOs 71-75, SEQ ID NOs: 76 and 77, and SEQ ID NO: 78, respectively.

2. The urokinase plasminogen activator receptor (uPAR) binding molecule of claim 1, wherein the light chain variable domain (V_{L}) comprises the amino acid sequence as set forth in SEQ ID NOs: 2, 25-44, or 4904.

3. The urokinase plasminogen activator receptor (uPAR) binding molecule of claim 1, wherein the uPAR binding molecule further comprises a heavy chain variable domain, wherein the heavy chain variable domain comprises 3 complementarity determining regions (CDRs), CDR1, CDR2, and CDR3 as set forth in SEQ ID NOs 45-57, SEQ ID NOs: 58-68, and SEQ ID NOs: 69 and 70, respectively.

4. The urokinase plasminogen activator receptor (uPAR) binding molecule of claim 3, wherein the heavy chain variable domain (V_{H}) comprises the amino acid sequence as set forth in SEQ ID NOs: 1, 5-24, or 4903.

5. The urokinase plasminogen activator receptor (uPAR) binding molecule of claim 3, wherein the light chain variable domain (V_{L}) comprises the amino acid sequence as set forth in SEQ ID NOs: 2, 25-44, or 4904; and wherein the heavy chain variable domain (VA) comprises the amino acid sequence as set forth in SEQ ID NOs: 1, 5-24, or 4903.

6. A urokinase plasminogen activator receptor (uPAR) binding molecule comprising a heavy chain variable domain; wherein the heavy chain variable domain comprises 3 complementarity determining regions (CDRs), CDR1, CDR2, and CDR3 as set forth in SEQ ID NOs 45-57, SEQ ID NOs: 58-68, and SEQ ID NOs: 69 and 70, respectively.

7. The urokinase plasminogen activator receptor (uPAR) binding molecule of claim 6, wherein the heavy chain variable domain (V_{H}) comprises the amino acid sequence as set forth in SEQ ID NOs: 1, 5-24, or 4903.

8. The urokinase plasminogen activator receptor (uPAR) binding molecule of claim 1, wherein the urokinase plasminogen activator receptor (uPAR) binding molecule binds soluble urokinase plasminogen activator receptor (suPAR).

9. The soluble urokinase plasminogen activator receptor (suPAR) binding molecule of claim 1, wherein the binding molecule comprises a chimeric antigen receptor (CAR) T cell, CAR NK cell, CAR Macrophage (CARMA), immunotoxin, bispecific antibody, diabody, triabody, Bispecific T cell engager (BiTE), antibody, or antibody fragment.

10. The soluble urokinase plasminogen activator receptor (suPAR) binding molecule of claim 1, wherein the binding molecule further comprises a light chain constant domain as set forth in SEQ ID NO: 4 or SEQ ID NO: 4906.

11. The urokinase plasminogen activator receptor (uPAR) binding molecule of claim 1, wherein the binding molecule further comprises a heavy chain constant domain as set forth in SEQ ID NO. 3 or SEQ ID NO: 4905.

12. A method of treating an inflammatory kidney disease or condition in a subject or the symptoms thereof comprising administering to the subject the urokinase plasminogen activator receptor (uPAR) binding molecule of claim 1.

13. The method of treating an inflammatory kidney disease or condition or the symptoms thereof of claim 12, wherein the kidney disease or condition comprises proteinuric kidney disease; Focal segmental glomerulosclerosis (FSGS); IgA nephropathy; membranous nephropathy; lupus nephritis; diabetic nephropathy; Autosomal dominant polycystic kidney disease (ADPKD); Alport syndrome, acute kidney injury (AKI) (including, but not limited to COVID-19 AKI); glomerulonephritis; preeclampsia; systemic lupus erythematosus; multiple myeloma; or kidney injury as the result of trauma, contrast agents, infection, surgery, ischemia/reperfusion injury, transplant, or medication.

14. The method of treating an inflammatory kidney disease or condition or the symptoms thereof of claim 12, wherein the urokinase plasminogen activator receptor (uPAR) binding molecule is administered when suPAR levels are elevated relative to a normal control.

15. The method of treating an inflammatory kidney disease or condition or the symptoms thereof of claim 14, wherein the oluble urokinase plasminogen activator receptor (uPAR) binding molecule is administered prior to the onset of symptoms.

16. The method of treating an inflammatory kidney disease or condition or the symptoms thereof of claim 12, further comprising obtaining a biological sample from the subject and measuring suPAR levels in the sample; wherein 1 ng/ml of suPAR indicates a healthy subject; 2-ng/ml of suPAR indicates an acute kidney disease or acute inflammation; 4 ng/ml of suPAR indicates the subject likely has or will develop chronic kidney disease; and 5 ng/ml or greater of suPAR indicates that the subject has chronic kidney disease.

17. The method of claim 16, wherein the biological sample comprises whole blood, plasma, serum, or urine.

18. A method of treating an inflammatory kidney disease or condition in a subject or the symptoms thereof comprising administering to the subject a urokinase plasminogen activator receptor (uPAR) binding molecule comprising a light chain variable domain, wherein the light chain variable domain comprises 3 complementarity determining regions (CDRs), CDR1, CDR2, and CDR3 as set forth in SEQ ID NOs 71-75, SEQ ID NOs: 76 and 77, and SEQ ID NO: 78, respectively.

19. The method of treating an inflammatory kidney disease or condition or the symptoms thereof of claim 18, wherein the light chain variable domain (V_{L}) comprises the amino acid sequence as set forth in SEQ ID NOs: 2, 25-44, or 4904.

20. The method of treating an inflammatory kidney disease or condition or the symptoms thereof of claim 18, wherein the uPAR binding molecule further comprises a heavy chain variable domain; wherein the heavy chain variable domain comprises 3 complementarity determining regions (CDRs), CDR1, CDR2, and CDR3 as set forth in SEQ ID NOs 45-57, SEQ ID NOs: 58-68, and SEQ ID NOs: 69 and 70, respectively.

21. The method of treating an inflammatory kidney disease or condition or the symptoms thereof of claim 20, wherein the heavy chain variable domain (V_{H}) comprises the amino acid sequence as set forth in SEQ ID NOs: 1, 5-24, or 4903.

22. The method of treating an inflammatory kidney disease or condition or the symptoms thereof of claim 20, wherein the light chain variable domain (V_{L}) comprises the amino acid sequence as set forth in SEQ ID NOs: 2, 25-44, or 4904; and wherein the heavy chain variable domain (V_{H}) comprises the amino acid sequence as set forth in SEQ ID NOs: 1, 5-24, or 4903.

23. The method of treating an inflammatory kidney disease or condition or the symptoms thereof of claim 18, wherein the kidney disease or condition comprises proteinuric kidney disease; Focal segmental glomerulosclerosis (FSGS); IgA nephropathy; membranous nephropathy; lupus nephritis; diabetic nephropathy; Autosomal dominant polycystic kidney disease (ADPKD); Alport syndrome, acute kidney injury (AKI) (including, but not limited to COVID-19 AKI); glomerulonephritis; preeclampsia; systemic lupus erythematosus; multiple myeloma; or kidney injury as the result of trauma, contrast agents, infection, surgery, ischemia/reperfusion injury, transplant, or medication.

24. The method of treating an inflammatory kidney disease or condition or the symptoms thereof of claim 18, wherein the urokinase plasminogen activator receptor (uPAR) binding molecule is administered when suPAR levels are elevated relative to a normal control.

25. The method of treating an inflammatory kidney disease or condition or the symptoms thereof of claim 24, wherein the oluble urokinase plasminogen activator receptor (uPAR) binding molecule is administered prior to the onset of symptoms.

26. The method of treating an inflammatory kidney disease or condition or the symptoms thereof of claim 18, further comprising obtaining a biological sample from the subject and measuring suPAR levels in the sample; wherein 1 ng/ml of suPAR indicates a healthy subject; 2-ng/ml of suPAR indicates an acute kidney disease or acute inflammation; 4 ng/ml of suPAR indicates the subject likely has or will develop chronic kidney disease; and 5 ng/ml or greater of suPAR indicates that the subject has chronic kidney disease.

27. The method of claim 26, wherein the biological sample comprises whole blood, plasma, serum, or urine.

28. A method of treating an inflammatory kidney disease or condition in a subject or the symptoms thereof comprising administering to the subject a urokinase plasminogen activator receptor (uPAR) binding molecule comprising a heavy chain variable domain; wherein the heavy chain variable domain comprises 3 complementarity determining regions (CDRs), CDR1, CDR2, and CDR3 as set forth in SEQ ID NOs 45-57, SEQ ID NOs: 58-68, and SEQ ID NOs: 69 and 70, respectively.

29. The method of treating an inflammatory kidney disease or condition or the symptoms thereof of claim 28, wherein the heavy chain variable domain (VI) comprises the amino acid sequence as set forth in SEQ ID NOs: 1, 5-24, or 4903.

30. The method of treating an inflammatory kidney disease or condition or the symptoms thereof of claim 28, wherein the kidney disease or condition comprises proteinuric kidney disease; Focal segmental glomerulosclerosis (FSGS); IgA nephropathy; membranous nephropathy; lupus nephritis; diabetic nephropathy; Autosomal dominant polycystic kidney disease (ADPKD); Alport syndrome, acute kidney injury (AKI) (including, but not limited to COVID-19 AKI); glomerulonephritis; preeclampsia; systemic lupus erythematosus; multiple myeloma; or kidney injury as the result of trauma, contrast agents, infection, surgery, ischemia/reperfusion injury, transplant, or medication.

31. The method of treating an inflammatory kidney disease or condition or the symptoms thereof of claim 28, wherein the urokinase plasminogen activator receptor (uPAR) binding molecule is administered when suPAR levels are elevated relative to a normal control.

32. The method of treating an inflammatory kidney disease or condition or the symptoms thereof of claim 31, wherein the oluble urokinase plasminogen activator receptor (uPAR) binding molecule is administered prior to the onset of symptoms.

33. The method of treating an inflammatory kidney disease or condition or the symptoms thereof of claim 28, further comprising obtaining a biological sample from the subject and measuring suPAR levels in the sample; wherein 1 ng/ml of suPAR indicates a healthy subject; 2-ng/ml of suPAR indicates an acute kidney disease or acute inflammation; 4 ng/ml of suPAR indicates the subject likely has or will develop chronic kidney disease; and 5 ng/ml or greater of suPAR indicates that the subject has chronic kidney disease.

34. The method of claim 33, wherein the biological sample comprises whole blood, plasma, serum, or urine.
